(19) United States
(12) Patent Application Publication
(10) Pub. No.: US 2022/0364114 A1
(43) Pub. Date: Nov. 17, 2022

(54) CONTROLLED EXPRESSION OF VIRAL PROTEINS

(71) Applicant: VOYAGER THERAPEUTICS, INC., Cambridge, MA (US)

(72) Inventors: Jeffrey Morley Slack, Newton, MA (US); Andrade Hendricks, Cambridge, MA (US); Peter Slade, Milton, MA (US); Christopher Nguyen, Cambridge, MA (US)

(73) Assignees: VOYAGER THERAPEUTICS, INC., Cambridge, MA (US); VOYAGER THERAPEUTICS, INC., Cambridge, MA (US)

(21) Appl. No.: 17/638,007

(22) PCT Filed: Aug. 26, 2020
(86) PCT No.: PCT/US2020/047910

§ 371 Date: Feb. 24, 2022

Related U.S. Application Data
(60) Provisional application No. 62/891,621, filed on Aug. 26, 2019.
(60) Provisional application No. 62/981,796, filed on Feb. 26, 2020.
(60) Provisional application No. 63/017,776, filed on Apr. 30, 2020.

(51) Int. Cl. C12N 15/86; C07K 14/195

(52) U.S. Cl. CPC C12N 15/86; C07K 14/195; C12N 2750/14143; C12N 2750/14152; C12N 2840/002

(57) ABSTRACT

The present disclosure describes methods and systems for use in the production of adeno-associated virus (AAV) particles, including recombinant adeno-associated virus (rAAV) particles. In certain embodiments, the production process and system use Spodoptera frugiperda insect cells (such as Sf9 or Sf21) as viral production cells. In certain embodiments, the production process and system use Baculoviral Expression Vectors (BEVs) in the production of AAV particles. In certain embodiments, the production process and system allow for the controlled expression of AAV capsid proteins, such as VP1, VP2 and VP3.

### CROSS REFERENCE TO RELATED APPLICATIONS

The present application claims the benefit of priority to: U.S. Provisional Patent Application No. 62/891,621, filed Aug. 26, 2019; U.S. Provisional Patent Application No. 62/981,796, filed Feb. 26, 2020; and U.S. Provisional Patent Application No. 63/017,776, filed Apr. 30, 2020; the contents of which are each hereby incorporated by reference herein in their entirety.

### REFERENCE TO THE SEQUENCE LISTING

The present application is being filed along with a Sequence Listing in electronic format. The Sequence Listing is provided as a file entitled 20571532PCT_SL.txt, created on Aug. 26, 2020, which is 61,867 bytes in size. The information in the electronic format of the sequence listing is incorporated herein by reference in its entirety.

### FIELD OF THE DISCLOSURE

The present disclosure describes methods and systems for use in the production of adeno-associated virus (AAV) particles, including recombinant adeno-associated virus (rAAV) particles. In certain embodiments, the production process and system use Spodoptera frugiperda insect cells (such as Sf9 or Sf21) as viral production cells. In certain embodiments, the production process and system use Baculoviral Expression Vectors (BEVs) in the production of AAV particles. In certain embodiments, the production process and system allow for the controlled expression of AAV capsid proteins, such as VP1, VP2 and VP3.

### BACKGROUND

AAVs have emerged as one of the most widely studied and utilized viral vectors for gene transfer to mammalian cells. See, e.g., Tratschin et al., Mol. Cell Biol., 5(11):3251-3260 (1985) and Grimm et al., Hum. Gene Ther., 10(15):2445-2450 (1999), the contents of which are herein incorporated by reference in their entirety. Adeno-associated viral (AAV) vectors are promising candidates for therapeutic gene delivery and have proven safe and efficacious in clinical trials. The design and production of improved AAV particles for this purpose is an active field of study.

With the advent of development in the AAV field, there remains a need for improved systems and methods for producing AAV vectors (such as AAV particles) and corresponding gene therapy production materials such as baculovirus infected insect cells (BIICs).

### SUMMARY

The present disclosure presents a transcriptional regulatory system. In certain embodiments, the transcriptional regulatory system comprises one or more regulator elements. In certain embodiments, the transcriptional regulatory system comprises one or more regulator binding sequences. In certain embodiments, the transcriptional regulatory system comprises one or more inducer elements. In certain embodiments, the transcriptional regulatory system comprises one or more regulator elements, one or more regulator binding sequences, and one or more inducer elements

In certain embodiments, the regulator element has a high affinity for binding to the regulator binding sequence. In certain embodiments, the transcriptional regulatory system comprises two regulator binding sequences, and wherein the regulator element has a high affinity for binding to both regulator binding sequences simultaneously. In certain embodiments, the simultaneous binding of the regulator element to the two regulator binding sequences results in the formation of a loop structure in a nucleotide sequence between the two regulator binding sequences.

In certain embodiments, the inducer element can bind to the regulator element, thereby reducing the affinity of the regulator element for binding to the regulator binding sequence. In certain embodiments, the binding of the inducer element to the regulator element causes a conformational change in the regulator element, thereby reducing the affinity of the regulator element for binding to the regulator binding sequence.

In certain embodiments, the transcriptional regulatory system comprises at least one regulator element which is a Lac repressor (LacR) protein selected from a wildtype Lac repressor protein (wLacR) or an engineered Lac repressor protein (eLacr). In certain embodiments, the at least one regulator element is an engineered Lac repressor protein (eLacr). In certain embodiments, the engineered LacR protein is encoded by a nucleotide sequence comprising SEQ ID NO: 2. In certain embodiments, the engineered LacR protein is encoded by a nucleotide sequence which is at least 75%, at least 80%, at least 85%, at least 90%, at least 95%, or at least 99% identical to SEQ ID NO: 2. In certain embodiments, the engineered LacR protein is encoded by a nucleotide sequence selected from SEQ ID NO: 2 or a nucleotide sequence which is at least 75%, at least 80%, at least 85%, at least 90%, at least 95%, or at least 99% identical to SEQ ID NO: 2.

In certain embodiments, the transcriptional regulatory system comprises at least one regulator binding sequence which is a Lac Operator (LacO) nucleotide sequence. In certain embodiments, the at least one regulator binding sequence is a Lac Operator (LacO) nucleotide sequence comprising SEQ ID NO: 4. In certain embodiments, the at least one regulator binding sequence is a Lac Operator (LacO) nucleotide sequence which is at least 75%, at least 80%, at least 85%, at least 90%, at least 95%, or at least 99% identical to SEQ ID NO: 4. In certain embodiments, the at least one regulator binding sequence is a Lac Operator (LacO) nucleotide sequence selected from SEQ ID NO: 4 or a nucleotide sequence which is at least 75%, at least 80%, at least 85%, at least 90%, at least 95%, or at least 99% identical to SEQ ID NO: 4.

In certain embodiments, the at least one inducer element is selected from lactose, allolactose and isopropyl-β-D-thiogalactose (IPTG). In certain embodiments, the at least one inducer element comprises lactose. In certain embodiments, the at least one inducer element comprises allolactose. In certain embodiments, the at least one inducer element comprises isopropyl-β-D-thiogalactose (IPTG).

The present disclosure presents a viral expression construct comprising one or more component of a transcriptional regulatory system of the present disclosure. In certain embodiments, the viral expression construct comprises a first protein-coding region and a first regulator region. In certain embodiments, the first protein-coding region of the viral expression construct comprises a first protein-coding nucleotide sequence and a first expression control sequence operably linked to the first protein-coding nucleotide sequence. In certain embodiments, the first expression control sequence comprises a promoter which regulates the transcription of the first protein-coding nucleotide sequence, and also comprises one or more of the regulator binding sequences of the transcriptional regulatory system. In certain embodiments, the first regulator region of the viral expression construct comprises a first regulator nucleotide sequence which encodes one or more of the regulator elements of the transcriptional regulatory system.

In certain embodiments, the viral expression construct comprises a first protein-coding region and a first regulator region; wherein the first protein-coding region of the viral expression construct comprises a first protein-coding nucleotide sequence and a first expression control sequence operably linked to the first protein-coding nucleotide sequence; wherein the first expression control sequence comprises a promoter which regulates the transcription of the first protein-coding nucleotide sequence, and also comprises one or more of the regulator binding sequences of the transcriptional regulatory system; and wherein the first regulator region of the viral expression construct comprises a first regulator nucleotide sequence which encodes one or more of the regulator elements of the transcriptional regulatory system.

In certain embodiments, the first regulator region comprises a nucleotide sequence which comprises SEQ ID NO: 5. In certain embodiments, the first regulator region comprises a nucleotide sequence which is at least 75%, at least 80%, at least 85%, at least 90%, at least 95%, or at least 99% identical to SEQ ID NO: 5. In certain embodiments, the first regulator region comprises a nucleotide sequence selected from SEQ ID NO: 5 or a nucleotide sequence which is at least 75%, at least 80%, at least 85%, at least 90%, at least 95%, or at least 99% identical to SEQ ID NO: 5.

In certain embodiments, the first regulator region comprises a nucleotide sequence which comprises SEQ ID NO: 10. In certain embodiments, the first regulator region comprises a nucleotide sequence which is at least 75%, at least 80%, at least 85%, at least 90%, at least 95%, or at least 99% identical to SEQ ID NO: 10. In certain embodiments, the first regulator region comprises a nucleotide sequence selected from SEQ ID NO: 10 or a nucleotide sequence which is at least 75%, at least 80%, at least 85%, at least 90%, at least 95%, or at least 99% identical to SEQ ID NO: 10.

In certain embodiments, the first regulator region comprises a nucleotide sequence which comprises SEQ ID NO: 11. In certain embodiments, the first regulator region comprises a nucleotide sequence which is at least 75%, at least 80%, at least 85%, at least 90%, at least 95%, or at least 99% identical to SEQ ID NO: 11. In certain embodiments, the first regulator region comprises a nucleotide sequence selected from SEQ ID NO: 11 or a nucleotide sequence which is at least 75%, at least 80%, at least 85%, at least 90%, at least 95%, or at least 99% identical to SEQ ID NO: 11.

In certain embodiments, the viral expression construct comprises a second regulator region. In certain embodiments, the second regulator region comprises a second regulator nucleotide sequence, wherein the second regulator nucleotide sequence encodes one or more of the regulator elements of the transcriptional regulatory system.

In certain embodiments, the second regulator region comprises a nucleotide sequence which comprises SEQ ID NO: 12. In certain embodiments, the second regulator region comprises a nucleotide sequence which is at least 75%, at least 80%, at least 85%, at least 90%, at least 95%, or at least 99% identical to SEQ ID NO: 12. In certain embodiments, the second regulator region comprises a nucleotide sequence selected from SEQ ID NO: 12 or a nucleotide sequence which is at least 75%, at least 80%, at least 85%, at least 90%, at least 95%, or at least 99% identical to SEQ ID NO: 12.

In certain embodiments, the second regulator region comprises a nucleotide sequence which comprises SEQ ID NO: 22. In certain embodiments, the second regulator region comprises a nucleotide sequence which is at least 75%, at least 80%, at least 85%, at least 90%, at least 95%, or at least 990% identical to SEQ ID NO: 22. In certain embodiments, the second regulator region comprises a nucleotide sequence selected from SEQ ID NO: 22 or a nucleotide sequence which is at least 75%, at least 80%, at least 85%, at least 90%, at least 95%, or at least 99% identical to SEQ ID NO: 22.

In certain embodiments, first protein-coding region comprises a nucleotide sequence which comprises SEQ ID NO: 25. In certain embodiments, the first protein-coding region comprises a nucleotide sequence which is at least 75%, at least 80%, at least 85%, at least 90%, at least 95%, or at least 99% identical to SEQ ID NO: 25. In certain embodiments, first protein-coding region comprises a nucleotide sequence selected from SEQ ID NO: 25 or a nucleotide sequence which is at least 75%, at least 80%, at least 85%, at least 90%, at least 95%, or at least 99% identical to SEQ ID NO: 25.

In certain embodiments, first protein-coding region comprises a nucleotide sequence which comprises SEQ ID NO: 28. In certain embodiments, the first protein-coding region comprises a nucleotide sequence which is at least 75%, at least 80%, at least 85%, at least 90%, at least 95%, or at least 99% identical to SEQ ID NO: 28. In certain embodiments, the first protein-coding region comprises a nucleotide sequence selected from SEQ ID NO: 28 or a nucleotide sequence which is at least 75%, at least 80%, at least 85%, at least 90%, at least 95%, or at least 99% identical to SEQ ID NO: 28.

In certain embodiments, the regulator binding sequence of the first expression control sequence is within 5-100 nucleotides from one end of the promoter of first expression control sequence. In certain embodiments, the first expression control sequence comprises a first regulator binding sequence which is 5-100 nucleotides upstream of the 5′ end of the promoter of the first expression control sequence, and a second regulator binding sequence which is 5-100 nucleotides downstream from the 3′ end of the promoter of first expression control sequence. In certain embodiments, the first regulator binding sequence and the second regulator binding sequence have a space interval of between 150 to 300, between 150 to 250, between 150 to 225, or between 150 to 210 nucleotides between them, as measured from the center nucleotide of each regulator binding sequence.

In certain embodiments, the regulator element encoded by the regulator nucleotide sequence binds to the one or more regulator binding sequences in the first expression control sequence, and inhibits or reduces transcription of the first protein-coding nucleotide sequence from the promoter in the first expression control sequence. In certain embodiments, the regulator element encoded by the regulator nucleotide sequence binds to the first regulator binding sequence and the second regulator binding sequence in the first expression control sequence, resulting in the formation of a loop structure around the promoter in the first expression control sequence, and thereby inhibiting or reducing transcription of the first protein-coding nucleotide sequence from the promoter in the first expression control sequence.

In certain embodiments, the first protein-coding nucleotide sequence encodes a structural AAV capsid protein selected from VP1, VP1 only, VP2 only, VP3 only, or a combination thereof. In certain embodiments, the first protein-coding nucleotide sequence encodes VP1 only.

In certain embodiments, the viral expression construct comprises a second protein-coding region which comprises a second protein-coding nucleotide sequence and a second expression control sequence operably linked to the second protein-coding nucleotide sequence. In certain embodiments, the second expression control sequence comprises a promoter which regulates the transcription of the second protein-coding nucleotide sequence, and also comprises one or more of the regulator binding sequences of the transcriptional regulatory system.

In certain embodiments, the viral expression construct comprises a second protein-coding region which comprises a second protein-coding nucleotide sequence and a second expression control sequence operably linked to the second protein-coding nucleotide sequence; wherein the second expression control sequence comprises a promoter which regulates the transcription of the second protein-coding nucleotide sequence, and also comprises one or more of the regulator binding sequences of the transcriptional regulatory system.

In certain embodiments, second protein-coding region comprises a nucleotide sequence which comprises SEQ ID NO: 41. In certain embodiments, the second protein-coding region comprises a nucleotide sequence which is at least 75%, at least 80%, at least 85%, at least 90%, at least 95%, or at least 99% identical to SEQ ID NO: 41. In certain embodiments, second protein-coding region comprises a nucleotide sequence selected from SEQ ID NO: 41 or a nucleotide sequence which is at least 75%, at least 80%, at least 85%, at least 90%, at least 95%, or at least 99% identical to SEQ ID NO: 41.

In certain embodiments, second protein-coding region comprises a nucleotide sequence which comprises SEQ ID NO: 43. In certain embodiments, the second protein-coding region comprises a nucleotide sequence which is at least 75%, at least 80%, at least 85%, at least 90%, at least 95%, or at least 99% identical to SEQ ID NO: 43. In certain embodiments, the second protein-coding region comprises a nucleotide sequence selected from SEQ ID NO: 43 or a nucleotide sequence which is at least 75%, at least 80%, at least 85%, at least 90%, at least 95%, or at least 99% identical to SEQ ID NO: 43.

In certain embodiments, second protein-coding region comprises a nucleotide sequence which comprises SEQ ID NO: 44. In certain embodiments, the second protein-coding region comprises a nucleotide sequence which is at least 75%, at least 80%, at least 85%, at least 90%, at least 95%, or at least 99% identical to SEQ ID NO: 44. In certain embodiments, second protein-coding region comprises a nucleotide sequence selected from SEQ ID NO: 44 or a nucleotide sequence which is at least 75%, at least 80%, at least 85%, at least 90%, at least 95%, or at least 99% identical to SEQ ID NO: 44.

In certain embodiments, second protein-coding region comprises a nucleotide sequence which comprises SEQ ID NO: 52. In certain embodiments, the second protein-coding region comprises a nucleotide sequence which is at least 75%, at least 80%, at least 85%, at least 90%, at least 95%, or at least 99% identical to SEQ ID NO: 52. In certain embodiments, the second protein-coding region comprises a nucleotide sequence selected from SEQ ID NO: 52 or a nucleotide sequence which is at least 75%, at least 80%, at least 85%, at least 90%, at least 95%, or at least 99% identical to SEQ ID NO: 52.

In certain embodiments, the regulator binding sequence of the second expression control sequence is within 5-100 nucleotides from one end of the promoter of second expression control sequence. In certain embodiments, the second expression control sequence comprises a first regulator binding sequence which is 5-100 nucleotides upstream of the 5′ end of the promoter of the second expression control sequence, and a second regulator binding sequence which is 5-100 nucleotides downstream from the 3′ end of the promoter of second expression control sequence. In certain embodiments, the first regulator binding sequence and the second regulator binding sequence have a space interval of between 150 to 300, between 150 to 250, between 150 to 225, or between 150 to 210 nucleotides between them, as measured from the center nucleotide of each regulator binding sequence.

In certain embodiments, the regulator element encoded by the regulator nucleotide sequence binds to the one or more regulator binding sequences in the second expression control sequence, and inhibits or reduces transcription of the second protein-coding nucleotide sequence from the promoter in the second expression control sequence. In certain embodiments, the regulator element encoded by the regulator nucleotide sequence binds to the second regulator binding sequence and the second regulator binding sequence in the second expression control sequence, resulting in the formation of a loop structure around the promoter in the second expression control sequence, and thereby inhibiting or reducing transcription of the second protein-coding nucleotide sequence from the promoter in the second expression control sequence. In certain embodiments, the second protein-coding nucleotide sequence encodes a structural AAV capsid protein selected from VP1, VP1 only, VP2 only, VP3 only, or a combination thereof. In certain embodiments, the second protein-coding nucleotide sequence encodes VP2 only. In certain embodiments, the second protein-coding nucleotide sequence encodes VP3 only.

In certain embodiments, the viral expression construct comprises a third protein-coding region which comprises a third protein-coding nucleotide sequence and a third expression control sequence operably linked to the third protein-coding nucleotide sequence; wherein the third expression control sequence comprises a promoter which regulates the transcription of the third protein-coding nucleotide sequence.

In certain embodiments, the third protein-coding region comprises a nucleotide sequence which comprises SEQ ID NO: 53. In certain embodiments, the third protein-coding region comprises a nucleotide sequence which is at least 75%, at least 80%, at least 85%, at least 90%, at least 95%, or at least 99% identical to SEQ ID NO: 53. In certain embodiments, the third protein-coding region comprises a nucleotide sequence selected from SEQ ID NO: 53 or a nucleotide sequence which is at least 75%, at least 80%, at least 85%, at least 90%, at least 95%, or at least 99% identical to SEQ ID NO: 53.

In certain embodiments, the third protein-coding nucleotide sequence encodes a structural AAV capsid protein selected from VP1, VP1 only, VP2 only, VP3 only, or a combination thereof. In certain embodiments, the third protein-coding nucleotide sequence encodes VP2 only. In certain embodiments, the third protein-coding nucleotide sequence encodes VP3 only.

In certain embodiments, the viral expression construct comprises a nucleotide sequence which comprises SEQ ID NO: 56. In certain embodiments, the viral expression construct comprises a nucleotide sequence which is at least 75%, at least 80%, at least 85%, at least 90%, at least 95%, or at least 99% identical to SEQ ID NO: 56. In certain embodiments, the viral expression construct comprises a nucleotide sequence selected from SEQ ID NO: 56 or a nucleotide sequence which is at least 75%, at least 80%, at least 85%, at least 90%, at least 95%, or at least 99% identical to SEQ ID NO: 56.

The present disclosure presents a viral production cell which comprises a viral expression construct of the present disclosure.

The present disclosure presents a viral production system which comprises a viral expression construct of the present disclosure. In certain embodiments, the viral production system comprises a viral expression construct of the present disclosure and a transcriptional regulatory system of the present disclosure. In certain embodiments, the viral production system comprises a viral production cell which comprises a viral expression construct of the present disclosure. In certain embodiments, the viral production system comprises a viral production cell which comprises a viral expression construct of the present disclosure, and a transcriptional regulatory system of the present disclosure. In certain embodiments, the one or more regulator binding sequences of the transcriptional regulatory system are comprised in the viral expression construct. In certain embodiments, the one or more regulator elements of the transcriptional regulatory system are encoded by the regulator nucleotide sequences comprised in the viral expression construct.

In certain embodiments, the one or more inducer elements bind to the one or more regulator elements encoded by the regulator nucleotide sequences, and reduce the affinity of the regulator elements for binding to the one or more regulator binding sequences. In certain embodiments, the level of transcription of one or more protein-coding nucleotide sequences is increased or decreased proportionally to the concentration of the inducer elements within the viral production system. In certain embodiments, the one or more inducer elements bind to the one or more regulator elements encoded by the regulator nucleotide sequences, and reduce the affinity of the regulator elements for binding to the one or more regulator binding sequences, such that the level of transcription of one or more protein-coding nucleotide sequences is increased or decreased proportionally to the concentration of the inducer elements within the viral production system. In certain embodiments, the one or more protein-coding nucleotide sequences encode one or more structural AAV capsid proteins.

In certain embodiments, the one or more inducer elements is present in the viral production cell at a target concentration; wherein the target concentration of inducer elements with the viral production cell results in the production of AAV capsids which have a VP1:VP2:VP3 protein ratio of 0.5-2:0.5-2:10. In certain embodiments, the one or more inducer elements is present in the viral production cell at a target concentration; wherein the target concentration of inducer elements with the viral production cell results in the production of AAV capsids which have a VP1:VP2:VP3 protein ratio of 1-2:1-2:10.

In certain embodiments, the one or more protein-coding nucleotide sequences encode one or more structural AAV capsid proteins; wherein the one or more inducer elements is present in the viral production cell at a target concentration; and wherein target concentration of inducer elements with the viral production cell results in the production of AAV capsids which have a VP1:VP2:VP3 protein ratio of 0.5-2:0.5-2:10. In certain embodiments, the one or more protein-coding nucleotide sequences encode one or more structural AAV capsid proteins; wherein the one or more inducer elements is present in the viral production cell at a target concentration; and wherein target concentration of inducer elements with the viral production cell results in the production of AAV capsids which have a VP1:VP2:VP3 protein ratio of 1-2:1-2:10.

In certain embodiments, the inducer element is present at a concentration between about 1.0 μM to about 100 μM; preferably a concentration between about 1.0 μM to about 35 μM.

### BRIEF DESCRIPTION OF THE FIGURES

The foregoing and other objects, features and advantages will be apparent from the following description of particular embodiments of the present disclosure, as illustrated in the accompanying figures. The figures are not necessarily to scale or comprehensive, with emphasis instead being placed upon illustrating the principles of various embodiments of the present disclosure.

FIG. 1 presents a gel column showing separation of a polH-NLS-LacR insert from a polH-NLS-LacR-pUC57 plasmid.

FIG. 2A, FIG. 2B, FIG. 2C, FIG. 2D, FIG. 2E and FIG. 2F present gel columns showing the results of Colony PCR screening for LacR insertion into AvrII-cut bacmids.

FIG. 3A presents a gel column showing REN fragment separation for a forward orientation of LacR insertion into the AvrII-cut bacmids. FIG. 3B presents a gel column showing REN fragment separation for a reverse orientation of LacR insertion into the AvrII-cut bacmids.

FIG. 4A and FIG. 4B present gel columns showing the results of REN digestion analysis for orientation of LacR insertion into AvrII-cut bacmids.

FIG. 5 presents a gel column showing separation of a LacO-p10-LacO-VP1 insert from LacO-p10-LacO-VP1-pUC57 plasmids and pUC57 fragments.

FIG. 6A, FIG. 6B and FIG. 6C present gel columns showing the results of Colony PCR screening for LacO-p10-LacO-VP1 insertion into the I-CeuI-cut 639 Bacmids.

FIG. 7A and FIG. 7B present gel columns showing the results of Western Blot analysis for LacR repression in Colony 1095.

FIG. 8A and FIG. 8B present gel columns showing the results of Western Blot analysis for LacR repression in Colony 1140.

FIG. 9 presents a gel column showing successful production of a LacO-p10-LacO-VP1-FseI insert through PCR amplification from a LacO-p10-LacO-VP1-pUC57 plasmid.

FIG. 10 presents a gel column showing the results of Colony PCR screening for LacO-p10-LacO-VP1 insertion into FseI-cut 601 Bacmids.

FIG. 11 presents a gel column showing the results of Colony PCR screening for LacO-p10-LacO-VP1 insertion into FseI-cut 601 Bacmids.

FIG. 12 presents a gel column showing ligation of an FseI-cut 601 Bacmid and a LacO-p10-LacO-VP2 insert into a combined LacO-p10-LacO-VP2 601 Bacmid.

FIG. 13 presents a gel column showing ligation of an I-CeuI/FseI-cut 601 Bacmid with a LacO-p10-LacO-VP1 insert and a LacO-p10-LacO-VP2 insert into a combined LacO-p10-LacO-VP1/LacO-p10-LacO-VP2 601 Bacmid.

FIG. 14A and FIG. 14B present gel columns showing the results of Colony PCR screening for LacO-p10-LacO-VP2 insertion into FseI-cut 601 Bacmids.

FIG. 15A and FIG. 15B present gel columns showing the results of Colony PCR screening for LacO-p10-LacO-VP2 insertion into FseI-cut 601 Bacmids.

FIG. 16A and FIG. 16B present gel columns showing the results of Colony PCR screening for LacO-p10-LacO-VP2 insertion into FseI-cut 601 Bacmids.

FIG. 17 presents a gel column showing the results of Colony PCR screening for LacO-p10-LacO-VP2 insertion into FseI-cut 601 Bacmids.

FIG. 18 presents a gel column showing successful production of JS142-JS143 fragments (Lane 1) through PCR amplification from a Bacmid 1140, and JS134-JS135 fragments (Lane 2) through PCR amplification from a LacO-p10-LacO-VP2-pUC57 plasmid.

FIG. 19 presents a gel column showing the results of Colony PCR screening for FseI-LacO-p10-LacO-VP2-AClef12-ACgta-FseI insertion into FseI-cut 1140 Bacmids.

FIG. 20A presents a gel column showing the results of Colony PCR screening for FseI-LacO-p10-LacO-VP2-AClef12-ACgta-FseI insertion into FseI-cut 1095 Bacmids. FIG. 20B presents a gel column showing the results of JS61-JS91 (715 bp), JS124-JS92 (1199 bp) and JS124-JS91 (3262 bp) PCR screening of Colonies 1186 and 1191.

FIG. 21 presents a gel column showing the results of Colony PCR screening for opgp64-polH-NLS-LacR insertion into AvrII-cut 1249 Plasmids.

FIG. 22 presents a gel column showing the results of JS95-JS42 (1037 bp), JS124-JS92 (1199 bp) and JS17-JS92 (1092 bp) PCR screening of Colony 1260.

FIG. 23 presents a graphical representation of certain components in Bacmid 1260.

FIG. 24A and FIG. 24B present gel columns showing the results of Western Blot analysis for LacR repression in Colony 1260 at 48 hours (FIG. 24A) and 72 hours (FIG. 24B) post infection.

FIG. 25 presents a line graph showing the VP1:VP3 and VP2:VP3 relative quantification results (Raw Pixel Count Imaging Software, adjusted for local background subtraction) of the Western Blot results shown in FIG. 24B.

FIG. 26 presents gel columns showing the results of Anti-AAV Capsid ECL Western Blot analysis for LacR repression in Colony 1260 using different VPC cell concentrations.

FIG. 27A presents a graphical representation of a transcriptional regulatory system of the present disclosure which includes an inducible regulator element (e.g. homotetrameric LacR protein) bound to a regulator binding region (e.g. LacO nucleotide sequence) on each side of a p10 promoter, thereby constraining the p10 promoter into a transcriptional-repressing loop.

FIG. 27B presents a graphical representation of a transcriptional regulatory system of the present disclosure which includes an inducer element (e.g. IPTG) bound to an inducible regulator element (e.g. homotetrameric LacR protein), thereby preventing the regulator element from binding to the regulator binding regions (e.g. LacO), such that transcription from the p10 protein can proceed.

FIG. 28A and FIG. 28B present gel columns showing the results of Western Blot analysis for Bacmid 1260 co-infection with GFP Bacmid 449 in Co-infection Experiment A (image lanes normalized to VP3). FIG. 28C presents a line graph showing the VP1, VP2 and VP3 quantification results (Raw Pixel Count Imaging Software, adjusted for local background subtraction) of the Western Blot results shown in FIG. 28A.

FIG. 29 presents gel columns showing the results of Western Blot analysis for Bacmid 1260 co-infection with GFP Bacmid 449 in Co-infection Experiment B and varying expressionBIIC-to-payloadBIIC ratios.

FIG. 30A, FIG. 30B and FIG. 30C present gel columns showing the results of Western Blot analysis for Bacmid 1260/Bacmid 420 co-infection with GFP Bacmid 449 in Co-infection Experiment C. FIG. 30A shows anti-Cap Western for AAV capsid proteins; FIG. 30B shows anti-Rep Western for AAV replicase proteins; FIG. 30C shows anti-GP64 Western for BV envelope protein.

FIG. 31 presents gel columns showing the results of Silver Stain/SDS-PAGE analysis of isolated AAV particles from Bacmid 1260 co-infection with GFP Bacmid 449 in Co-infection Experiment C.

FIG. 32A, FIG. 32B, FIG. 32C, and FIG. 32D present images from non-normalized transduction potency analysis in HEK cells of AAV particles produced by Bacmid 1260 co-infection with GFP Bacmid 449 in Co-infection Experiment C.

FIG. 33 presents gel columns showing the results of anti-Cap Western Blot analysis for Bacmid 1260 co-infection with GFP Bacmid 449 in Co-infection Experiment D.

FIG. 34A, FIG. 34B and FIG. 34C present gel columns showing the results of Western Blot analysis for Bacmid 1260/Bacmid 420 co-infection with GFP Bacmid 449 in a Time Course Study of post-infection harvest timing. FIG. 34A shows anti-Cap Western for AAV capsid proteins; FIG. 34B shows anti-Rep Western for AAV replicase proteins; FIG. 34C shows anti-GP64 Western for BV envelope protein.

### DETAILED DESCRIPTION

### I. Adeno-Associated Viruses (AAVs)

### Overview

Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family includes the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in Fields Virology (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

### AAV Viral Genomes

The wild-type AAV viral genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions comprising, but not limited to, acting as an origin for DNA replication by functioning as primers for the endogenous DNA polymerase complex of the host viral replication cell.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the content of which is incorporated herein by reference in its entirety as related to AAV9/hu.14, insofar as it does not conflict with the present disclosure) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

For use as a biological tool, the wild-type AAV viral genome can be modified to replace the rep/cap sequences with a nucleic acid sequence comprising a payload region with at least one ITR region. Typically, in recombinant AAV viral genomes there are two ITR regions. The rep/cap sequences can be provided in trans during production to generate AAV particles.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. See Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entireties as related to AAV variants and equivalents, insofar as they do not conflict with the present disclosure.

In certain embodiments, AAV particles, viral genomes and/or payloads of the present disclosure, and the methods of their use, may be as described in WO2017189963, the content of which is incorporated herein by reference in its entirety as related to AAV particles, viral genomes and/or payloads, insofar as it does not conflict with the present disclosure.

AAV particles of the present disclosure may be formulated in any of the gene therapy formulations of the disclosure comprising any variations of such formulations apparent to those skilled in the art. The reference to “AAV particles”, “AAV particle formulations” and “formulated AAV particles” in the present application refers to the AAV particles which may be formulated and those which are formulated without limiting either.

In certain embodiments, AAV particles of the present disclosure are recombinant AAV (rAAV) viral particles which are replication defective, lacking sequences encoding functional Rep and Cap proteins within their viral genome. These defective AAV particles may lack most or all parental coding sequences and essentially carry only one or two AAV ITR sequences and the nucleic acid of interest (i.e. payload) for delivery to a cell, a tissue, an organ or an organism.

In certain embodiments, the viral genome of the AAV particles of the present disclosure comprises at least one control element which provides for the replication, transcription and translation of a coding sequence encoded therein. Not all of the control elements need always be present as long as the coding sequence is capable of being replicated, transcribed and/or translated in an appropriate host cell. Non-limiting examples of expression control elements comprise sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV particles of the present disclosure may be produced recombinantly and may be based on adeno-associated virus (AAV) parent or reference sequences. As used herein, a “vector” is any molecule or moiety which transports, transduces or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV viral genomes contain DNA strands which anneal together to form double stranded DNA. By skipping second strand synthesis, scAAVs allow for rapid expression in the cell.

In certain embodiments, the AAV viral genome of the present disclosure is a scAAV. In certain embodiments, the AAV viral genome of the present disclosure is a ssAAV.

Methods for producing and/or modifying AAV particles are disclosed in the art, such as pseudotyped AAV particles (PCT Patent Publication Nos. WO200028004; WO200123001; WO2004112727; WO 2005005610 and WO 2005072364, the contents of each of which are incorporated herein by reference in their entireties as related to producing and/or modifying AAV particles, insofar as they do not conflict with the present disclosure).

AAV particles may be modified to enhance the efficiency of delivery. Such modified AAV particles can be packaged efficiently and be used to successfully infect the target cells at high frequency and with minimal toxicity. In certain embodiments the capsids of the AAV particles are engineered according to the methods described in US Publication Number US 20130195801, the content of which is incorporated herein by reference in its entirety as related to modifying AAV particles to enhance the efficiency of delivery, insofar as it does not conflict with the present disclosure.

In certain embodiments, the AAV particles comprise a payload construct and/or region encoding a polypeptide or protein of the present disclosure, and may be introduced into mammalian cells. In certain embodiments, the AAV particles comprise a payload construct and/or region encoding a polypeptide or protein of the present disclosure, and may be introduced into insect cells.

In certain embodiments, the AAV particles of the present disclosure comprise a viral genome with at least one ITR region and a payload region. In certain embodiments, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes of the present disclosure may be comprised of naturally occurring polynucleotide sequences or recombinantly derived polynucleotide sequences.

The ITRs may be derived from the same serotype as the capsid, or a derivative thereof. The ITR may be of a different serotype than the capsid. In certain embodiments, the AAV particle has more than one ITR. In a non-limiting example, the AAV particle has a viral genome comprising two ITRs. In certain embodiments, the ITRs are of the same serotype as one another. In another embodiment, the ITRs are of different serotypes. Non-limiting examples comprise zero, one or both of the ITRs having the same serotype as the capsid. In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 130, 140, 141, 142, 145 nucleotides in length.

In certain embodiments, each ITR may be 141 nucleotides in length. In certain embodiments, each ITR may be 130 nucleotides in length. In certain embodiments, each ITR may be 119 nucleotides in length.

### Viral Genome Size

In certain embodiments, the AAV particle which includes a payload described herein may be single stranded or double stranded viral genome. The size of the viral genome may be small, medium, large or the maximum size. Additionally, the viral genome may include a promoter and a polyA tail.

In certain embodiments, the viral genome which includes a payload described herein may be a small single stranded viral genome. A small single stranded viral genome may be 2.1 to 3.5 kb in size such as about 2.1, 2.2, 2.3, 2.4, 2.5, 2.6, 2.7, 2.8, 2.9, 3.0, 3.1, 3.2, 3.3, 3.4, and 3.5 kb in size. As a non-limiting example, the small single stranded viral genome may be 3.2 kb in size. As another non-limiting example, the small single stranded viral genome may be 2.2 kb in size. Additionally, the viral genome may include a promoter and a polyA tail.

In certain embodiments, the viral genome which includes a payload described herein may be a small double stranded viral genome. A small double stranded viral genome may be 1.3 to 1.7 kb in size such as about 1.3, 1.4, 1.5, 1.6, and 1.7 kb in size. As a non-limiting example, the small double stranded viral genome may be 1.6 kb in size. Additionally, the viral genome may include a promoter and a polyA tail.

In certain embodiments, the viral genome which includes a payload described herein e.g., polynucleotide, siRNA or dsRNA, may be a medium single stranded viral genome. A medium single stranded viral genome may be 3.6 to 4.3 kb in size such as about 3.6, 3.7, 3.8, 3.9, 4.0, 4.1, 4.2 and 4.3 kb in size. As a non-limiting example, the medium single stranded viral genome may be 4.0 kb in size. Additionally, the viral genome may include a promoter and a polyA tail.

In certain embodiments, the viral genome which includes a payload described herein may be a medium double stranded viral genome. A medium double stranded viral genome may be 1.8 to 2.1 kb in size such as about 1.8, 1.9, 2.0, and 2.1 kb in size. As a non-limiting example, the medium double stranded viral genome may be 2.0 kb in size. Additionally, the viral genome may include a promoter and a polyA tail.

In certain embodiments, the viral genome which includes a payload described herein may be a large single stranded viral genome. A large single stranded viral genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded viral genome may be 4.7 kb in size. As another non-limiting example, the large single stranded viral genome may be 4.8 kb in size. As yet another non-limiting example, the large single stranded viral genome may be 6.0 kb in size. Additionally, the viral genome may include a promoter and a polyA tail.

In certain embodiments, the viral genome which includes a payload described herein may be a large double stranded viral genome. A large double stranded viral genome may be 2.2 to 3.0 kb in size such as about 2.2, 2.3, 2.4, 2.5, 2.6, 2.7, 2.8, 2.9 and 3.0 kb in size. As a non-limiting example, the large double stranded viral genome may be 2.4 kb in size. Additionally, the viral genome may include a promoter and a polyA tail.

In certain embodiments, an viral genome of the present disclosure can include at least one filler region. In certain embodiments, an viral genome of the present disclosure can include at least one multiple cloning site (MCS) region. In certain embodiments, an viral genome of the present disclosure can include at least one promoter region. In certain embodiments, an viral genome of the present disclosure can include at least one exon region. In certain embodiments, an viral genome of the present disclosure can include at least one intron region.

### Viral Genome Regions: Inverted Terminal Repeats (ITRs)

The AAV particles of the present disclosure include a viral genome with at least one Inverted Terminal Repeat (ITR) region and a payload region. In certain embodiments, the viral genome has two ITRs. These two ITRs flank the payload region at the 5′ and 3′ ends. The ITRs function as origins of replication including recognition sites for replication. ITRs include sequence regions which can be complementary and symmetrically arranged. ITRs incorporated into viral genomes of the present disclosure may be included of naturally occurring polynucleotide sequences or recombinantly derived polynucleotide sequences.

The ITRs may be derived from the same serotype as the capsid, or a derivative thereof. The ITR may be of a different serotype than the capsid. In certain embodiments, the AAV particle has more than one ITR. In a non-limiting example, the AAV particle has a viral genome including two ITRs. In certain embodiments, the ITRs are of the same serotype as one another. In another embodiment, the ITRs are of different serotypes. Non-limiting examples include zero, one or both of the ITRs having the same serotype as the capsid. In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 130, 140, 141, 142, 145 nucleotides in length, and those having at least 95% identity thereto.

In certain embodiments, each ITR may be 141 nucleotides in length. In certain embodiments, each ITR may be 130 nucleotides in length. In certain embodiments, each ITR may be 119 nucleotides in length.

In certain embodiments, the AAV particles include two ITRs and one ITR is 141 nucleotides in length and the other ITR is 130 nucleotides in length. In certain embodiments, the AAV particles include two ITRs and both ITR are 141 nucleotides in length.

Independently, each ITR may be about 75 to about 175 nucleotides in length. The ITR may, independently, have a length such as, but not limited to, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175 nucleotides. The length of the ITR for the viral genome may be 75-80, 75-85, 75-100, 80-85, 80-90, 80-105, 85-90, 85-95, 85-110, 90-95, 90-100, 90-115, 95-100, 95-105, 95-120, 100-105, 100-110, 100-125, 105-110, 105-115, 105-130, 110-115, 110-120, 110-135, 115-120, 115-125, 115-140,120-125, 120-130, 120-145, 125-130, 125-135, 125-150, 130-135, 130-140, 130-155, 135-140, 135-145, 135-160, 140-145, 140-150,140-165, 145-150, 145-155, 145-170, 150-155, 150-160, 150-175, 155-160, 155-165, 160-165, 160-170, 165-170, 165-175, and 170-175 nucleotides. As a non-limiting example, the viral genome comprises an ITR that is about 105 nucleotides in length. As a non-limiting example, the viral genome comprises an ITR that is about 141 nucleotides in length. As a non-limiting example, the viral genome comprises an ITR that is about 130 nucleotides in length. As a non-limiting example, the viral genome comprises an ITR that is about 105 nucleotides in length and 141 nucleotides in length. As a non-limiting example, the viral genome comprises an ITR that is about 105 nucleotides in length and 130 nucleotides in length. As a non-limiting example, the viral genome comprises an ITR that is about 130 nucleotides in length and 141 nucleotides in length.

### AAV Serotypes

AAV particles of the present disclosure may include or be derived from any natural or recombinant AAV serotype. According to the present disclosure, the AAV particles may utilize or be based on a serotype or include a peptide selected from any of the following: VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV). UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAVrh20, AAVrh32/33, AAVrh39, AAVrh46, AAVrh73, AAVrh74, AAVhu.26, or variants or derivatives thereof.

The AAV-DJ sequence may include two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may include three mutations: (1) K406R where lysine (K; Lys) at amino acid 406 is changed to arginine (R; Arg). (2) R587Q where arginine (R; Arg) at amino acid 587 is changed to glutamine (Q; Gin) and (3) R590T where arginine (R; Arg) at amino acid 590 is changed to threonine (T; Thr).

In certain embodiments, the AAV may be a serotype generated by the AAV9 capsid library with mutations in amino acids 390-627 (VP1 numbering) The serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F4111), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N4577H. T574S), AAV9.14 (T1340A, T1362C, T1560C, G1713A; L447H), AAV9.16 (A1775T; Q592L), AAV9.24 (T1507C. T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V). AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K5281), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrolysine; U (Sec) for Selenocysteine; X (Xaa) for any amino acid; and Z (Glx) for Glutamine or Glutamic acid.

In certain embodiments, the AAV serotype may be, or may include a sequence, insert, modification or mutation as described in Patent Publications WO2015038958, WO2017100671, WO2016134375, WO2017083722, WO2017015102, WO2017058892, WO2017066764, U.S. Pat. Nos. 9,624,274, 9,475,845, US20160369298, US20170145405, the contents of which are herein incorporated by reference in their entirety.

In certain embodiments, the AAV may be a serotype generated by Cre-recombination-based AAV targeted evolution (CREATE) as described by Deverman et al., (Nature Biotechnology 34(2):204-209 (2016)), the contents of which are herein incorporated by reference in their entirety. In certain embodiments, the AAV serotype may be as described in Jackson et al (Frontiers in Molecular Neuroscience 9:154 (2016)), the contents of which are herein incorporated by reference in their entirety.

In certain embodiments, the AAV serotype is selected for use due to its tropism for cells of the central nervous system. In certain embodiments, the cells of the central nervous system are neurons. In another embodiment, the cells of the central nervous system are astrocytes.

In certain embodiments, the AAV serotype is selected for use due to its tropism for cells of the muscle(s).

In certain embodiments, the initiation codon for translation of the AAV VP1 capsid protein may be CTG, TTG, or GTG as described in U.S. Pat. No. 8,163,543, the contents of which are herein incorporated by reference in its entirety.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins including the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in their entirety.

According to the present disclosure, references to capsid proteins is not limited to either clipped (Met−/AA−) or unclipped (Met+/AA+) and may, in context, refer to independent capsid proteins, viral capsids included of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also include VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which includes or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1,” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### Payloads

AAV particles of the present disclosure can comprise, or be produced using, at least one payload construct which comprises at least one payload region. In certain embodiments, the payload region may be located within a viral genome, such as the viral genome of a payload construct. At the 5′ and/or the 3′ end of the payload region there may be at least one inverted terminal repeat (ITR). Within the payload region, there may be a promoter region, an intron region and a coding region.

In certain embodiments, the payload region of the AAV particle comprises one or more nucleic acid sequences encoding one or more payload, such as a payload polypeptide or polynucleotide. In certain embodiments, the payload region of the AAV particle comprises one or more nucleic acid sequences encoding one or more polypeptides or proteins of interest. In certain embodiments, the payload region of the AAV particle comprises one or more nucleic acid sequences encoding one or more modulatory polynucleotides, e.g., RNA or DNA molecules as therapeutic agents. Accordingly, the present disclosure provides viral genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the payload region can be included in a payload construct used for producing AAV particles. In certain embodiments, a payload construct of the present disclosure can be a bacmid, also known as a baculovirus plasmid or recombinant baculovirus genome. In certain embodiments, a payload construct of the present disclosure can be a baculovirus expression vector (BEV). In certain embodiments, a payload construct of the present disclosure can be a BIIC which includes a BEV. As used herein, the term “payloadBac” refers to a bacmid (such as a BEV) comprising a payload construct and/or payload region. Viral production cells (e.g., Sf9 cells) may be transfected with payloadBacs and/or with BIICs comprising payloadBacs.

In certain embodiments, the AAV particles of the present disclosure comprise one or more nucleic acid sequences encoding one or more payload, such as a payload polypeptide or polynucleotide, which are useful in the field of medicine for the treatment, prophylaxis, palliation or amelioration of diseases and/or disorders, including neurological diseases and/or disorders. In certain embodiments, the AAV particles of the present disclosure are useful in the field of medicine for the treatment, prophylaxis, palliation or amelioration of Friedreich's ataxia, or any disease stemming from a loss or partial loss of frataxin protein. In certain embodiments, the AAV particles of the present disclosure are useful in the field of medicine for the treatment, prophylaxis, palliation or amelioration of Parkinson's Disease. In certain embodiments, the AAV particles of the present disclosure are useful in the field of medicine for the treatment, prophylaxis, palliation or amelioration of Amyotrophic lateral sclerosis. In certain embodiments, the AAV particles of the present disclosure are useful in the field of medicine for the treatment, prophylaxis, palliation or amelioration of Huntington's Disease. In certain embodiments, the AAV particles of the present disclosure are useful in the field of medicine for the treatment, prophylaxis, palliation or amelioration of Alzheimer's Disease.

### Payloads: Polypeptides and Variants

In certain embodiments, the payload region of the AAV particle comprises one or more nucleic acid sequences encoding a polypeptide or protein of interest. In certain embodiments, the AAV particle comprises a viral genome with a payload region comprising nucleic acid sequences encoding more than one polypeptide of interest. In certain embodiments, a viral genome encoding one or more polypeptides may be replicated and packaged into a viral particle. A target cell transduced with a viral particle comprising the viral genome may express each of the one or more polypeptides in the single target cell.

Where the AAV particle payload region encodes a polypeptide, the polypeptide may be a peptide, polypeptide or protein. As a non-limiting example, the payload region may encode at least one therapeutic protein of interest. The AAV viral genomes encoding polypeptides described herein may be useful in the fields of human disease, viruses, infections veterinary applications and a variety of in vivo and in vitro settings.

In certain embodiments, administration of the formulated AAV particles (which comprise the viral genome) to a subject will increase the expression of a protein in a subject. In certain embodiments, the increase of the expression of the protein will reduce the effects and/or symptoms of a disease or ailment associated with the polypeptide encoded by the payload.

In certain embodiments, the AAV particle comprises a viral genome with a payload region comprising a nucleic acid sequence encoding a protein of interest (i.e. a payload protein, therapeutic protein).

Amino acid sequences encoded by payload regions of the viral genomes of the disclosure may be translated as a whole polypeptide, a plurality of polypeptides or fragments of polypeptides, which independently may be encoded by one or more nucleic acids, fragments of nucleic acids or variants of any of the aforementioned. As used herein. “polypeptide” refers to a polymer of amino acid residues (natural or unnatural) linked together, most often by peptide bonds. In certain embodiments, polypeptides can include proteins, polypeptides, and peptides of any size, structure, or function. In some instances, the polypeptide encoded is smaller than about 50 amino acids (i.e. peptide). If the polypeptide is a peptide, it will be at least about 2, 3, 4, or at least 5 amino acid residues long. Thus, polypeptides comprise gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

In certain embodiments, the polypeptide can be a polypeptide variant which differs in amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and in certain embodiments, they will be at least about 80%, or at least about 901% identical (homologous) to a native or reference sequence.

In certain embodiments, the payload region of the AAV particle comprises one or more nucleic acid sequences encoding a polypeptide or protein of interest.

In certain embodiments, the AAV particle comprises a viral genome with a payload region comprising nucleic acid sequences encoding more than one polypeptide of interest. In certain embodiments, a viral genome encoding one or more polypeptides may be replicated and packaged into a viral particle. A target cell transduced with a viral particle comprising the viral genome may express each of the one or more polypeptides in the single target cell.

Where the AAV particle payload region encodes a polypeptide, the polypeptide may be a peptide, polypeptide or protein. As a non-limiting example, the payload region may encode at least one therapeutic protein of interest. The AAV viral genomes encoding polypeptides described herein may be useful in the fields of human disease, viruses, infections veterinary applications and a variety of in vivo and in vitro settings.

In certain embodiments, administration of the formulated AAV particles (which comprise the viral genome) to a subject will increase the expression of a protein in a subject. In certain embodiments, the increase of the expression of the protein will reduce the effects and/or symptoms of a disease or ailment associated with the polypeptide encoded by the payload.

In certain embodiments, the formulated AAV particles of the present disclosure may be used to reduce the decline of functional capacity and activities of daily living as measured by a standard evaluation system such as, but not limited to, the total functional capacity (TFC) scale.

In certain embodiments, the AAV particle comprises a viral genome with a payload region comprising a nucleic acid sequence encoding a protein of interest (i.e. a payload protein, therapeutic protein).

In certain embodiments, the payload region comprises a nucleic acid sequence encoding a protein including but not limited to an antibody, Aromatic L-Amino Acid Decarboxylase (AADC), ApoE2. Frataxin, survival motor neuron (SMN) protein, glucocerebrosidase, N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspartoacylase (ASPA), progranulin (GRN). MeCP2, beta-galactosidase (GLB1) and/or gigaxonin (GAN).

In certain embodiments, the AAV particle includes a viral genome with a payload region comprising a nucleic acid sequence encoding AADC or any other payload known in the art for treating Parkinson's disease. As a non-limiting example, the payload may include a sequence such as NM_001082971.1 (GI: 132814447), NM_000790.3 (GI: 132814459), NM_001242886.1 (GI: 338968913), NM_001242887.1 (GI: 338968916), NM_001242888.1 (GI: 338968918), NM_001242889.1 (GI: 338968920), NM_001242890.1 (GI: 338968922) and fragment or variants thereof.

In certain embodiments, the AAV particle comprises a viral genome with a payload region comprising a nucleic acid sequence encoding frataxin or any other payload known in the art for treating Friedreich's Ataxia. As a non-limiting example, the payload may comprise a sequence such as NM_000144.4 (GI: 239787167), NM_181425.2 (GI: 239787185), NM_001161706.1 (GI: 239787197) and fragment or variants thereof.

In certain embodiments, the AAV particle comprises a viral genome with a payload region comprising a nucleic acid sequence encoding SMN or any other payload known in the art for treating spinal muscular atrophy (SMA). As a non-limiting example, the payload may comprise a sequence such as NM_001297715.1 (GI: 663070993), NM_000344.3 (GI: 196115055), NM_022874.2 (GI: 196115040) and fragment or variants thereof.

In certain embodiments, the AAV particle comprises a viral genome with a payload region comprising a nucleic acid sequence encoding any of the disease-associated proteins (and fragment or variants thereof) described in U. S. Patent publication No. 20180258424; the content of which is herein incorporated by reference in its entirety.

In certain embodiments, the AAV particle includes a viral genome with a payload region comprising a nucleic acid sequence encoding any of the disease-associated proteins (and fragment or variants thereof) described in any one of the following International Publications: WO2016073693, WO2017023724, WO2018232055, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety

In certain embodiments, the formulated AAV particles of the present disclosure may be used to improve performance on any assessment used to measure symptoms of a neurodegenerative disorder/disease. Such assessments comprise, but are not limited to ADAS-cog (Alzheimer Disease Assessment Scale—cognitive), MMSE (Mini-Mental State Examination), GDS (Geriatric Depression Scale), FAQ (Functional Activities Questionnaire), ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment). ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B Ther Adv Neurol Disord 5(6):349-358 (2012), the contents of which are herein incorporated by reference in their entirety.

In certain embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

In certain embodiments an “amino acid sequence variant” is provided. The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not be confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least about 70% homology to a native sequence, and in certain embodiments, they will be at least about 80% or at least about 90% homologous to a native sequence. “Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to comprise polypeptide variants which differ by one or more amino acid alterations, e.g., substitutions, additions or deletions of amino acid residues that still maintain the properties of the parent polypeptide.

Sequence tags or amino acids, such as one or more lysines, can be added to the peptide sequences of the disclosure (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support.

In certain embodiments a “substitutional variant” is provided. “Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions comprise the substitution of a non-polar (hydrophobic) residue such as isoleucine, valine and leucine for another non-polar residue. Likewise, examples of conservative substitutions comprise the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions comprise the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In certain embodiments an “insertional variant” is provided. “Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

In certain embodiments a “deletional variant” is provided. “Deletional variants” when referring to proteins, are those with one or more amino acids in the native or starting amino acid sequence removed. Ordinarily, deletional variants will have one or more amino acids deleted in a particular region of the molecule.

As used herein, the term “derivative” is used synonymously with the term “variant” and refers to a molecule that has been modified or changed in any way relative to a reference molecule or starting molecule. In certain embodiments, derivatives comprise native or starting proteins that have been modified with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present disclosure.

Other post-translational modifications comprise hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

“Features” when referring to proteins are defined as distinct amino acid sequence-based components of a molecule. Features of the proteins of the present disclosure comprise surface manifestations, local conformational shape, folds, loops, half-loops, domains, half-domains, sites, termini or any combination thereof.

As used herein when referring to proteins the term “surface manifestation” refers to a polypeptide-based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds comprise beta sheets and alpha helices. Examples of tertiary folds comprise domains and regions formed due to aggregation or separation of energetic forces. Regions formed in this way comprise hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid residues as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions).

As used herein when referring to proteins the term “half-domain” means portion of an identified domain having at least half the number of amino acid residues as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid-based embodiments is used synonymous with “amino acid residue” and “amino acid side chain”. A site represents a position within a peptide or polypeptide that may be modified, manipulated, altered, derivatized or varied within the polypeptide-based molecules of the present disclosure.

As used herein the terms “termini or terminus” when referring to proteins refers to an extremity of a peptide or polypeptide. Such extremity is not limited only to the first or final site of the peptide or polypeptide but may comprise additional amino acids in the terminal regions. The polypeptide-based molecules of the present disclosure may be characterized as having both an N-terminus (terminated by an amino acid with a free amino group (NH2)) and a C-terminus (terminated by an amino acid with a free carboxyl group (COOH)). Proteins of the disclosure are in certain embodiments made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide-based moiety such as an organic conjugate.

Once any of the features have been identified or defined as a component of a molecule of the disclosure, any of several manipulations and/or modifications of these features may be performed by moving, swapping, inverting, deleting, randomizing or duplicating. Furthermore, it is understood that manipulation of features may result in the same outcome as a modification to the molecules of the disclosure. For example, a manipulation which involves deleting a domain would result in the alteration of the length of a molecule just as modification of a nucleic acid to encode less than a full-length molecule would.

Modifications and manipulations can be accomplished by methods known in the art such as site directed mutagenesis. The resulting modified molecules may then be tested for activity using in vitro or in vivo assays such as those described herein, or any other suitable screening assay known in the art.

### Payloads: Modulatory Polynucleotides Targeting a Gene of Interest

The present disclosure comprises the use of formulated AAV particles whose viral genomes encode modulatory polynucleotides, e.g., RNA or DNA molecules as therapeutic agents. Accordingly, the present disclosure provides viral genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the AAV particle comprises a viral genome with a payload region comprising a nucleic acid sequence encoding or comprising one or more modulatory polynucleotides. In certain embodiments, the AAV particle comprises a viral genome with a payload region comprising a nucleic acid sequence encoding a modulatory polynucleotide of interest. In certain embodiments of the present disclosure, modulatory polynucleotides, e.g., RNA or DNA molecules, are presented as therapeutic agents. RNA interference mediated gene silencing can specifically inhibit targeted gene expression.

In certain embodiments, a nucleic acid sequence encoding such siRNA molecules, or a single strand of the siRNA molecules, is inserted into adeno-associated viral vectors and introduced into cells, specifically cells in the central nervous system.

AAV particles have been investigated for siRNA delivery because of several unique features. Non-limiting examples of the features comprise (i) the ability to infect both dividing and non-dividing cells; (ii) a broad host range for infectivity, comprising human cells; (iii) wild-type AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

In certain embodiments, the encoded siRNA duplex of the present disclosure contains an antisense strand and a sense strand hybridized together forming a duplex structure, wherein the antisense strand is complementary to the nucleic acid sequence of the targeted gene of interest, and wherein the sense strand is homologous to the nucleic acid sequence of the targeted gene of interest. In other aspects, there are 0, 1 or 2 nucleotide overhangs at the 3′end of each strand.

The payloads of the formulated AAV particles of the present disclosure may encode one or more agents which are subject to RNA interference (RNAi) induced inhibition of gene expression. Provided herein are encoded siRNA duplexes or encoded dsRNA that target a gene of interest (referred to herein collectively as “siRNA molecules”). Such siRNA molecules, e.g., encoded siRNA duplexes, encoded dsRNA or encoded siRNA or dsRNA precursors can reduce or silence gene expression in cells, for example, astrocytes or microglia, cortical, hippocampal, entorhinal, thalamic, sensory or motor neurons.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2-nucleotide 3′ overhangs and that match the nucleic acid sequence of the target gene. These short RNA species may be naturally produced in vivo by Dicer-mediated cleavage of larger dsRNAs and they are functional in mammalian cells.

Naturally expressed small RNA molecules, known as microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA mediated down regulation of gene expression may be caused by cleavage of the target mRNAs, translational inhibition of the target mRNAs, or mRNA decay. miRNA targeting sequences are usually located in the 3′ UTR of the target mRNAs. A single miRNA may target more than 100 transcripts from various genes, and one mRNA may be targeted by different miRNAs.

siRNA duplexes or dsRNA targeting a specific mRNA may be designed as a payload of an AAV particle and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful tool for genetic analysis in mammalian cells and has the potential to produce novel therapeutics.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases it requires higher concentration of the ss-siRNA to achieve the effective gene silencing potency of the corresponding duplex.

In certain embodiments, the siRNA molecules may be encoded in a modulatory polynucleotide which also comprises a molecular scaffold. As used herein a “molecular scaffold” is a framework or starting molecule that forms the sequence or structural basis against which to design or make a subsequent molecule.

In certain embodiments, the modulatory polynucleotide which comprises the payload (e.g., siRNA, miRNA or other RNAi agent described herein) comprises molecular scaffold which comprises a leading 5′ flanking sequence which may be of any length and may be derived in whole or in part from wild type microRNA sequence or be completely artificial. A 3′ flanking sequence may mirror the 5′ flanking sequence in size and origin. In certain embodiments, one or both of the 5′ and 3′ flanking sequences are absent.

In certain embodiments, the molecular scaffold may comprise one or more linkers known in the art. The linkers may separate regions or one molecular scaffold from another. As a non-limiting example, the molecular scaffold may be polycistronic.

In certain embodiments, the modulatory polynucleotide is designed using at least one of the following properties: loop variant, seed mismatch/bulge/wobble variant, stem mismatch, loop variant and basal stem mismatch variant, seed mismatch and basal stem mismatch variant, stem mismatch and basal stem mismatch variant, seed wobble and basal stem wobble variant, or a stem sequence variant.

In certain embodiments, the present disclosure presents the use of formulated AAV particles whose viral genomes encode modulatory polynucleotides, e.g., RNA or DNA molecules as therapeutic agents. Accordingly, the present disclosure provides viral genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the AAV particle comprises a viral genome with a payload region comprising a nucleic acid sequence encoding or comprising one or more modulatory polynucleotides. In certain embodiments, the AAV particle comprises a viral genome with a payload region comprising a nucleic acid sequence encoding a modulatory polynucleotide of interest. In certain embodiments of the present disclosure, modulatory polynucleotides, e.g., RNA or DNA molecules, are presented as therapeutic agents. RNA interference mediated gene silencing can specifically inhibit targeted gene expression.

In certain embodiments, the payload region comprises a nucleic acid sequence encoding a modulatory polynucleotide which interferes with a target gene expression and/or a target protein production. In certain embodiments, the gene expression or protein production to be inhibited/modified may comprise but are not limited to superoxide dismutase 1 (SOD1), chromosome 9 open reading frame 72 (C90RF72), TAR DNA binding protein (TARDBP), ataxin-3 (ATXN3), huntingtin (HTT), amyloid precursor protein (APP), apolipoprotein E (ApoE), microtubule-associated protein tau (MAPT), alpha-synuclein (SNCA), voltage-gated sodium channel alpha subunit 9 (SCN9A), and/or voltage-gated sodium channel alpha subunit 10 (SCN10A).

The present disclosure provides small interfering RNA (siRNA) duplexes (and modulatory polynucleotides encoding them) that target SOD1 mRNA to interfere with the gene expression and/or protein production of SOD1. The present disclosure also provides methods of their use for inhibiting gene expression and protein production of an allele of SOD1, for treating amyotrophic lateral sclerosis (ALS). In certain embodiments, the siRNA duplexes of the present disclosure may target SOD1 along any segment of the respective nucleotide sequence. In certain embodiments, the siRNA duplexes of the present disclosure may target SOD1 at the location of a SNP or variant within the nucleotide sequence.

The present disclosure provides small interfering RNA (siRNA) duplexes (and modulatory polynucleotides encoding them) that target HTT mRNA to interfere with the gene expression and/or protein production of HTT. The present disclosure also provides methods of their use for inhibiting gene expression and protein production of an allele of HTT, for treating Huntington's disease (HD). In certain embodiments, the siRNA duplexes of the present disclosure may target HTT along any segment of the respective nucleotide sequence. In certain embodiments, the siRNA duplexes of the present disclosure may target HTT at the location of a SNP or variant within the nucleotide sequence.

In certain embodiments, the AAV particle comprises a viral genome with a payload region comprising a nucleic acid sequence encoding any of the modulatory polynucleotides, RNAi molecules, siRNA molecules, dsRNA molecules, and/or RNA duplexes described in any one of the following International Publications: WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety.

In certain embodiments, a nucleic acid sequence encoding such siRNA molecules, or a single strand of the siRNA molecules, is inserted into adeno-associated viral vectors and introduced into cells, specifically cells in the central nervous system.

AAV particles have been investigated for siRNA delivery because of several unique features. Non-limiting examples of the features comprise (i) the ability to infect both dividing and non-dividing cells; (ii) a broad host range for infectivity, comprising human cells; (iii) wild-type AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

In certain embodiments, the encoded siRNA duplex of the present disclosure contains an antisense strand and a sense strand hybridized together forming a duplex structure, wherein the antisense strand is complementary to the nucleic acid sequence of the targeted gene of interest, and wherein the sense strand is homologous to the nucleic acid sequence of the targeted gene of interest. In other aspects, there are 0, 1 or 2 nucleotide overhangs at the 3′end of each strand.

According to the present disclosure, each strand of the siRNA duplex targeting the gene of interest can be about 19 to 25, 19 to 24 or 19 to 21 nucleotides in length, such as about 19 nucleotides, 20 nucleotides, 21 nucleotides, 22 nucleotides, 23 nucleotides, 24 nucleotides, or 25 nucleotides in length.

In certain embodiments, an siRNA or dsRNA comprises at least two sequences that are complementary to each other. The dsRNA comprises a sense strand having a first sequence and an antisense strand having a second sequence. The antisense strand comprises a nucleotide sequence that is substantially complementary to at least part of an mRNA encoding a gene of interest, and the region of complementarity is 30 nucleotides or less, and at least 15 nucleotides in length. Generally, the dsRNA is 19 to 25, 19 to 24 or 19 to 21 nucleotides in length. In certain embodiments, the dsRNA is from about 15 to about 25 nucleotides in length, and in certain embodiments the dsRNA is from about 25 to about 30 nucleotides in length.

The dsRNA encoded in an expression vector upon contacting with a cell expressing protein encoded by the gene of interest, inhibits the expression of protein encoded by the gene of interest by at least 10%, at least 20%, at least 25%, at least 30%, at least 35%, at least 40%, at least 50%, at least 60%, at least 70%, at least 80%, or more, when assayed by methods known in the art or a method as described herein.

According to the present disclosure, the siRNA molecules are designed and tested for their ability in reducing mRNA levels in cultured cells.

In certain embodiments, the siRNA molecules are designed and tested for their ability in reducing levels of the gene of interest in cultured cells.

The present disclosure also provides pharmaceutical compositions comprising at least one siRNA duplex targeting the gene of interest and a pharmaceutically acceptable carrier. In certain embodiments, the siRNA duplex is encoded by a viral genome in an AAV particle.

In certain embodiments, the present disclosure provides methods for inhibiting/silencing gene expression in a cell. In some aspects, the inhibition of gene expression refers to an inhibition by at least about 20%, such as by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 35-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In certain embodiments, the encoded siRNA duplexes may be used to reduce the expression of protein or mRNA encoded by the gene of interest by at least about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95/0, 20-100%, 30-40%, 35-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of protein or mRNA may be reduced 50-90%. As a non-limiting example, the expression of protein or mRNA may be reduced 30-70%. As a non-limiting example, the expression of protein or mRNA may be reduced 40-70%.

In certain embodiments, the encoded siRNA duplexes may be used to reduce the expression of protein encoded by the gene of interest and/or transcribed mRNA in at least one region of the CNS. As a non-limiting example, the region is the neurons (e.g., cortical neurons).

In certain embodiments, the formulated AAV particles comprising such encoded siRNA molecules may be introduced directly into the central nervous system of the subject, for example, by infusion into the putamen.

In certain embodiments, the formulated AAV particles comprising such encoded siRNA molecules may be introduced directly into the central nervous system of the subject, for example, by infusion into the thalamus of a subject.

In certain embodiments, the formulated AAV particles comprising such encoded siRNA molecules may be introduced directly into the central nervous system of the subject, for example, by infusion into the white matter of a subject.

In certain embodiments, the formulated AAV particles comprising such encoded siRNA molecules may be introduced to the central nervous system of the subject, for example, by intravenous administration to a subject.

In certain embodiments, the pharmaceutical composition of the present disclosure is used as a solo therapy. In certain embodiments, the pharmaceutical composition of the present disclosure is used in combination therapy. The combination therapy may be in combination with one or more neuroprotective agents such as small molecule compounds, growth factors and hormones which have been tested for their neuroprotective effect on motor neuron degeneration.

The payloads of the formulated AAV particles of the present disclosure may encode one or more agents which are subject to RNA interference (RNAi) induced inhibition of gene expression. Provided herein are encoded siRNA duplexes or encoded dsRNA that target a gene of interest (referred to herein collectively as “siRNA molecules”). Such siRNA molecules, e.g., encoded siRNA duplexes, encoded dsRNA or encoded siRNA or dsRNA precursors can reduce or silence gene expression in cells, for example, astrocytes or microglia, cortical, hippocampal, entorhinal, thalamic, sensory or motor neurons.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2-nucleotide 3′ overhangs and that match the nucleic acid sequence of the target gene. These short RNA species may be naturally produced in vivo by Dicer-mediated cleavage of larger dsRNAs and they are functional in mammalian cells.

In some embodiments, the modulatory polynucleotides of the viral genome may comprise at least one nucleic acid sequence encoding at least one siRNA molecule. The nucleic acid sequence may, independently if there is more than one, encode 1, 2, 3, 4, 5, 6, 7, 8, 9, or more than 9 siRNA molecules.

Naturally expressed small RNA molecules, known as microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA mediated down regulation of gene expression may be caused by cleavage of the target mRNAs, translational inhibition of the target mRNAs, or mRNA decay. miRNA targeting sequences are usually located in the 3′ UTR of the target mRNAs. A single miRNA may target more than 100 transcripts from various genes, and one mRNA may be targeted by different miRNAs.

siRNA duplexes or dsRNA targeting a specific mRNA may be designed as a payload of an AAV particle and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful tool for genetic analysis in mammalian cells and has the potential to produce novel therapeutics.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases it requires higher concentration of the ss-siRNA to achieve the effective gene silencing potency of the corresponding duplex.

Any of the foregoing molecules may be encoded by an AAV particle or viral genome.

Introduction into Cells

The encoded payload of the present disclosure may be introduced into cells by being encoded by the viral genome of an AAV particle. These AAV particles can be engineered and optimized to facilitate the entry into cells that are not readily amendable to transfection/transduction. Also, some synthetic viral vectors possess an ability to integrate the payload into the cell genome, thereby leading to stable payload expression and long-term therapeutic effect. In this manner, viral vectors are engineered as vehicles for specific delivery while lacking the deleterious replication and/or integration features found in wild-type virus.

In certain embodiments, the encoded payload is introduced into a cell by transfecting, infecting or transducing the cell with an AAV particle comprising nucleic acid sequences capable of producing the payload when processed in the cell. In certain embodiments, the payload is introduced into a cell by injecting into the cell or tissue an AAV particle comprising a nucleic acid sequence capable of producing the payload when processed in the cell.

Other methods for introducing AAV particles comprising the nucleic acid sequence for the payloads described herein may comprise photochemical internalization as described in U. S. Patent publication No. 20120264807, the content of which is incorporated herein by reference in its entirety as related to photochemical internalizations, insofar as it does not conflict with the present disclosure.

In certain embodiments, the formulations described herein may contain at least one AAV particle comprising the nucleic acid sequence encoding the payloads described herein. In certain embodiments, the payloads may target the gene of interest at one target site. In another embodiment, the formulation comprises a plurality of AAV particles, each AAV particle comprising a nucleic acid sequence encoding a payload targeting a gene of interest at a different target site. The gene of interest may be targeted at 2, 3, 4, 5 or more than 5 sites.

In certain embodiments, the AAV particles from any relevant species, such as, but not limited to, human, pig, dog, mouse, rat or monkey may be introduced into cells.

In certain embodiments, the formulated AAV particles may be introduced into cells or tissues which are relevant to the disease to be treated. In certain embodiments, the formulated AAV particles may be introduced into cells which have a high level of endogenous expression of the target gene. In another embodiment, the formulated AAV particles may be introduced into cells which have a low level of endogenous expression of the target gene. In certain embodiments, the cells may be those which have a high efficiency of AAV transduction.

In certain embodiments, formulated AAV particles comprising a nucleic acid sequence encoding a payload of the present disclosure may be used to deliver the payload to the central nervous system (e.g., U.S. Pat. No. 6,180,613; the content of which is incorporated herein by reference in its entirety as related to the delivery and therapeutic use of siRNA molecules and AAV particles, insofar as it does not conflict with the present disclosure).

In certain embodiments, the formulated AAV particles comprising a nucleic acid sequence encoding a payload of the present disclosure may further comprise a modified capsid comprising peptides from non-viral origin. In other aspects, the AAV particle may contain a CNS specific chimeric capsid to facilitate the delivery of encoded siRNA duplexes into the brain and the spinal cord. For example, an alignment of cap nucleotide sequences from AAV variants exhibiting CNS tropism may be constructed to identify variable region (VR) sequence and structure.

In certain embodiments, AAV particle comprising the nucleic acid sequence for the siRNA molecules of the present disclosure may be formulated for CNS delivery. Agents that cross the brain blood barrier may be used. For example, some cell penetrating peptides that can target siRNA molecules to the brain blood barrier endothelium may be used to formulate the siRNA duplexes targeting the gene of interest.

In certain embodiments, the formulated AAV particle comprising a nucleic acid sequence encoding a payload of the present disclosure may be administered directly to the CNS. As a non-limiting example, the vector comprises a nucleic acid sequence encoding an siRNA molecule targeting the gene of interest. As a non-limiting example, the vector comprises a nucleic acid sequence encoding an polypeptide targeting a gene of interest.

In certain embodiments, the formulated AAV particle may be administered to a subject (e.g., to the CNS of a subject) in a therapeutically effective amount.

### II. AAV Production

### General Viral Production Process

Mammalian cells and/or insect cells are often used as viral production cells for the production of rAAV particles. In various embodiments, the methods and systems disclosed herein employ insect cells, e.g., Sf9 cells.

Viral production cells for the production of rAAV particles generally comprise mammalian cell types. However, mammalian cells present several complications to the large-scale production of rAAV particles, comprising general low yield of viral-particles-per-replication-cell as well as high risks for undesirable contamination from other mammalian biomaterials in the viral production cell. As a result, insect cells have become an alternative vehicle for large-scale production of rAAV particles.

AAV production systems using insect cells also present a range of complications. For example, high-yield production of rAAV particles often requires a lower expression of Rep78 compared to Rep52. Controlling the relative expression of Rep78 and Rep52 in insect cells thus requires carefully designed control mechanisms within the Rep operon. These control mechanisms can comprise individually engineered insect cell promoters, such as ΔIEI promoters for Rep78 and PolH promoters for Rep52, or the division of the Rep-encoding nucleotide sequences onto independently engineered sequences or constructs. However, implementation of these control mechanisms often leads to reduced rAAV particle yield or to structurally unstable virions.

In another example, production of rAAV particles requires VP1, VP2 and VP3 proteins which assemble to form the AAV capsid. High-yield production of rAAV particles requires adjusted ratios of VP1, VP2 and VP3, which should generally be around 1:1:10, respectively, but can vary from 1-2 for VP1 and/or 1-2 for VP2, relative to 10 VP3 copies. This ratio is important for the quality of the capsid, as too much VP1 destabilizes the capsid and too little VP1 will decrease the infectivity of the virus.

Wild type AAV use a deficient splicing method to control VP1 expression; a weak start codon (ACG) with special surrounding (“Kozak” sequence) to control VP2; and a standard start codon (ATG) for VP3 expression. However, in some baculovirus systems, the mammalian splicing sequences are not always recognized and unable to properly control the production of VP1, VP2 and VP3. Consequently, neighboring nucleotides and the ACG start sequence from VP2 can be used to drive capsid protein production. Unfortunately, for most of the AAV serotypes, this method creates a capsid with a lower ratio of VP1 compared to VP2 (<1 relative to 10 VP3 copies). To more effectively control the production of VP proteins, non-canonical or start codons have been used, like TTG, GTG or CTG. However, these start codons can be considered suboptimal by those in the art relative to the wild type ATG or ACG start codons (See, WO2007046703 and WO2007148971, the content of which is incorporated herein by reference in its entirety as related to production of AAV capsid proteins, insofar as it does not conflict with the present disclosure).

In another example, production of rAAV particles using a baculovirus/Sf9 system generally requires the widely used bacmid-based Baculovirus Expression Vector System (BEVs), which are not optimized for large-scale AAV production. Aberrant proteolytic degradation of viral proteins in the bacmid-based BEVs is an unexpected issue, precluding the reliable large-scale production of AAV capsid proteins using the baculovirus/Sf9 system.

There is continued need for methods and systems which allow for effective and efficient large scale (commercial) production of rAAV particles in mammalian and insect cells.

The details of one or more embodiments of the present disclosure are set forth in the accompanying description below. Other features, objects, and advantages of the present disclosure will be apparent from the description, drawings, and the claims. In the description, the singular forms also include the plural unless the context clearly dictates otherwise. Unless defined otherwise, all technical and scientific terms used herein have the same meaning as commonly understood by one of ordinary skill in the art to which this present disclosure belongs. In the case of conflict with disclosures incorporated by reference, the present express description will control.

In certain embodiments, the constructs, polynucleotides, polypeptides, vectors, serotypes, capsids formulations, or particles of the present disclosure may be, may comprise, may be modified by, may be used by, may be used for, may be used with, or may be produced with any sequence, element, construct, system, target or process described in one of the following International Publications: WO2016073693, WO2017023724, WO2018232055, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each incorporated herein by reference in their entireties, insofar as they do not conflict with the present disclosure.

AAV production of the present disclosure comprises processes and methods for producing AAV particles and viral vectors which can contact a target cell to deliver a payload, e.g. a recombinant viral construct, which comprises a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

The present disclosure provides methods of producing AAV particles or viral vectors by (a) contacting a viral production cell with one or more viral expression constructs encoding at least one AAV capsid protein and/or at least one AAV replication protein, and one or more payload construct vectors, wherein said payload construct vector comprises a payload construct encoding a payload molecule selected from the group consisting of a transgene, a polynucleotide encoding protein, and a modulatory nucleic acid; (b) culturing said viral production cell under conditions such that at least one AAV particle or viral vector is produced, and (c) isolating said at least one AAV particle or viral vector.

In these methods a viral expression construct may encode at least one structural protein and/or at least one non-structural protein. The structural protein may comprise any of the native or wild type capsid proteins VP1, VP2 and/or VP3 or a chimeric protein. The non-structural protein may comprise any of the native or wild type Rep78, Rep68, Rep52 and/or Rep40 proteins or a chimeric protein.

In certain embodiments, an rAAV production method as disclosed herein comprises transient transfection, viral transduction and/or electroporation.

In certain embodiments, the viral production cell is selected from the group consisting of a mammalian cell and an insect cell. In certain embodiments, the insect cell comprises a Spodoptera frugiperda insect cell. In certain embodiments, the insect cell comprises a Sf9 insect cell. In certain embodiments, the insect cell comprises a Sf21 insect cell.

The payload construct vector of the present disclosure may comprise at least one inverted terminal repeat (ITR) and may comprise mammalian DNA.

Also provided are AAV particles and viral vectors produced according to the methods described herein.

The AAV particles of the present disclosure may be formulated as a pharmaceutical composition with one or more acceptable excipients.

In certain embodiments, an AAV particle or viral vector may be produced by a method described herein.

In certain embodiments, the AAV particles may be produced by contacting a viral production cell (e.g., an insect cell) with at least one viral expression construct encoding at least one capsid protein and at least one AAV replication protein, and at least one payload construct vector. In certain embodiments, separate viral expression constructs encoding the at least one capsid protein and the at least one AAV replication protein may be used. The viral production cell may be contacted by transient transfection, viral transduction and/or electroporation. The payload construct vector may comprise a payload construct encoding a payload molecule such as, but not limited to, a transgene, a polynucleotide encoding protein, and a modulatory nucleic acid. The viral production cell can be cultured under conditions such that at least one AAV particle or viral vector is produced, isolated (e.g., using temperature-induced lysis, mechanical lysis and/or chemical lysis) and/or purified (e.g., using filtration, chromatography and/or immunoaffinity purification). As a non-limiting example, the payload construct vector may comprise mammalian DNA.

In certain embodiments, the AAV particles are produced in an insect cell (e.g., Spodoptera frugiperda (Sf9) cell) using the method described herein. As a non-limiting example, the insect cell is contacted using viral transduction which may comprise baculoviral transduction.

In another embodiment, the AAV particles are produced in a mammalian cell using the method described herein. As a non-limiting example, the mammalian cell is contacted using transient transfection.

In certain embodiments, the viral expression construct may encode at least one structural protein and at least one non-structural protein. As a non-limiting example, the structural protein may comprise VP1, VP2 and/or VP3 capsid proteins. As another non-limiting example, the non-structural protein may comprise Rep78, Rep68, Rep52 and/or Rep40 replication proteins.

In certain embodiments, the AAV particle production method described herein produces greater than 10¹, greater than 10², greater than 10³, greater than 10⁴ or greater than 10⁵ AAV particles in a viral production cell.

In certain embodiments, a process of the present disclosure comprises production of viral particles in a viral production cell using a viral production system which comprises at least one viral expression construct and at least one payload construct. The at least one viral expression construct and at least one payload construct can be co-transfected (e.g. dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, comprising Rep proteins which replicate the payload construct and Cap proteins which assemble to form a capsid that encloses the replicated payload constructs. The resulting AAV particle is extracted from the viral production cells and processed into a pharmaceutical preparation for administration.

In certain embodiments, the AAV particles (after being administered) contact a target cell and enter the cell in an endosome. The AAV particle releases from the endosome and subsequently contacts the nucleus of the target cell to deliver the payload construct. The payload construct, e.g. recombinant viral construct, is delivered to the nucleus of the target cell wherein the payload molecule encoded by the payload construct may be expressed.

In certain embodiments, the process for production of viral particles utilizes seed cultures of viral production cells that comprise one or more baculoviruses (e.g., a Baculoviral Expression Vector (BEV) or baculovirus infected insect cells (BIICs) that have been transfected with a viral expression construct (e.g., comprised in an expressionBac) and a payload construct (e.g., comprised in a payloadBac)). In certain embodiments, the seed cultures are harvested, divided into aliquots and frozen, and may be used at a later time point to initiate an infection of a naïve population of production cells.

Large scale production of AAV particles may utilize a bioreactor. The use of a bioreactor allows for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined time point and AAV particles are purified. In another embodiment, the bioreactor is used for continuous production in which a portion of the culture is harvested at an experimentally determined time point for purification of AAV particles, and the remaining culture in the bioreactor is refreshed with additional growth media components.

In certain embodiments, AAV viral particles can be extracted from viral production cells in a process which comprises cell lysis, clarification, sterilization and purification. Cell lysis comprises any process that disrupts the structure of the viral production cell, thereby releasing AAV particles. In certain embodiments cell lysis may comprise thermal shock, chemical, or mechanical lysis methods. In some embodiments, cell lysis is done chemically. Clarification of the lysed cells can comprise the gross purification of the mixture of lysed cells, media components, and AAV particles. In certain embodiments, clarification comprises centrifugation and/or filtration, comprising but not limited to depth end, tangential flow, and/or hollow fiber filtration.

The end result of viral production is a purified collection of AAV particles which comprise two components: (1) a payload construct (e.g. a recombinant viral genome construct) and (2) a viral capsid.

In certain embodiments, a viral production system or process of the present disclosure comprises steps for producing baculovirus infected insect cells (BIICs) using Viral Production Cells (VPC) and plasmid constructs. Viral Production Cells (VPCs) from a Cell Bank (CB) are thawed and expanded to provide a target working volume and VPC concentration. The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce P1 Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can comprise a single CP Purification step or can comprise multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

In certain embodiments, a viral production system or process of the present disclosure comprises steps for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. This expansion can include one or more small-volume expansion steps up to a working volume of 2000-5000 mL, followed by one or more large-volume expansion steps in large-scale bioreactors (e.g. Wave and/or N−1 bioreactors) up to a working volume of 25-500 L. The working volume of Viral Production Cells is seeded into a Production Bioreactor and can be further expanded to a working volume of 200-2500 L with a target VPC concentration for BIIC infection.

The working volume of VPCs in the Production Bioreactor is then co-infected with Rep/Cap BIICs and Payload BIICs, e.g., with a target VPC:BIIC ratio and a target BIIC:BIIC ratio. VCD infection can also utilize BEVs. The co-infected VPCs are incubated and expanded in the Production Bioreactor to produce a bulk harvest of AAV particles and VPCs.

In certain embodiments, a viral production system or process of the present disclosure comprises steps for producing a Drug Substance by processing, clarifying and purifying a bulk harvest of AAV particles and Viral Production Cells. A bulk harvest of AAV particles and VPCs (within a Production Bioreactor) are processed through cellular disruption and lysis (e.g. chemical lysis and/or mechanical lysis), followed by nuclease treatment of the lysis pool, thereby producing a crude lysate pool. The crude lysate pool is processed through one or more filtration and clarification steps, comprising depth filtration and microfiltration to provide a clarified lysate pool. The clarified lysate pool is processed through one or more chromatography and purification steps, comprising affinity chromatography (AFC) and ion-exchange chromatography (AEX or CEX) to provide a purified product pool. The purified product pool is then optionally processed through nanofiltration, and then through tangential flow filtration (TFF). The TFF process comprises one or more diafiltration (DF) steps and one or more ultrafiltration (UF) steps, either in series or alternating. The product pool is further processed through viral retention filtration (VRF) and another filtration step to provide a drug substance pool. The drug substance pool can be further filtered, then aliquoted into vials for storage and treatment.

### Viral Expression Constructs

The viral production system of the present disclosure comprises one or more viral expression constructs which can be transfected/transduced into a viral production cell (e.g., Sf9). In certain embodiments, a viral expression construct or a payload construct of the present disclosure can be a bacmid, also known as a baculovirus plasmid or recombinant baculovirus genome. In certain embodiments, a viral expression construct of the present disclosure can be a baculovirus expression vector (BEV). In certain embodiments, a viral expression construct of the present disclosure can be a BIIC which includes a BEV. As used herein, the term “expressionBac” or “Rep/Cap Bac” refers to a bacmid (such as a BEV) comprising a viral expression construct and/or viral expression region. Viral production cells (e.g., Sf9 cells) may be transfected with expressionBacs and/or with BIICs comprising expressionBacs.

In certain embodiments, the viral expression region comprises a protein-coding nucleotide sequence and at least one expression control sequence for expression in a viral production cell. In certain embodiments, the viral expression region comprises a protein-coding nucleotide sequence operably linked to least one expression control sequence for expression in a viral production cell. In certain embodiments, the viral expression construct contains parvoviral genes under control of one or more promoters. Parvoviral genes can comprise nucleotide sequences encoding non-structural AAV replication proteins, such as Rep genes which encode Rep52, Rep40, Rep68 or Rep78 proteins, e.g., a combination of Rep78 and Rep52. Parvoviral genes can comprise nucleotide sequences encoding structural AAV proteins, such as Cap genes which encode VP1, VP2 and VP3 proteins.

The viral production system of the present disclosure is not limited by the viral expression vector used to introduce the parvoviral functions into the virus replication cell. The presence of the viral expression construct in the virus replication cell need not be permanent. The viral expression constructs can be introduced by any means known, for example by chemical treatment of the cells, electroporation, or infection.

Viral expression constructs of the present disclosure may comprise any compound or formulation, biological or chemical, which facilitates transformation, transfection, or transduction of a cell with a nucleic acid. Exemplary biological viral expression constructs comprise plasmids, linear nucleic acid molecules, and recombinant viruses comprising baculovirus. Exemplary chemical vectors comprise lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994.); Maniatis et al., eds. Molecular Cloning. CSH Laboratory, NY, N.Y. (1982); and, Philiport and Scluber, eds. Liposomes as tools in Basic Research and Industry. CRC Press, Ann Arbor, Mich. (1995), the contents of which are each incorporated herein by reference in their entireties as related to viral expression constructs and uses thereof, insofar as they do not conflict with the present disclosure.

In certain embodiments, the viral expression construct is an AAV expression construct which comprises one or more nucleotide sequences encoding non-structural AAV replication proteins, structural AAV capsid proteins, or a combination thereof. In certain embodiments, the viral expression region is an AAV expression region of an expression construct which comprises one or more nucleotide sequences encoding non-structural AAV replication proteins, structural AAV capsid proteins, or a combination thereof.

In certain embodiments, the viral expression construct of the present disclosure may be a plasmid vector. In certain embodiments, the viral expression construct of the present disclosure may be a baculoviral construct.

The present disclosure is not limited by the number of viral expression constructs employed to produce AAV particles or viral vectors. In certain embodiments, one, two, three, four, five, six, or more viral expression constructs can be employed to produce AAV particles in viral production cells in accordance with the present disclosure. In one non-limiting example, five expression constructs may individually encode AAV VP1, AAV VP2, AAV VP3, Rep52, Rep78, and with an accompanying payload construct comprising a payload polynucleotide and at least one AAV ITR. In another embodiment, expression constructs may be employed to express, for example, Rep52 and Rep40, or Rep78 and Rep 68. Expression constructs may comprise any combination of VP1, VP2, VP3, Rep52/Rep40, and Rep78/Rep68 coding sequences.

In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, the viral expression construct may encode the components of a Parvoviral capsid with incorporated Gly-Ala repeat region, which may function as an immune invasion sequence, as described in US Patent Application 20110171262, the content of which is incorporated herein by reference in its entirety as related to Parvoviral capsid proteins, insofar as it does not conflict with the present disclosure.

In certain embodiments of the present disclosure, a viral expression construct may be used for the production of AAV particles in insect cells. In certain embodiments, modifications may be made to the wild type AAV sequences of the capsid and/or rep genes, for example to improve attributes of the viral particle, such as increased infectivity or specificity, or to enhance production yields from insect cells.

### VP-Coding Region

In certain embodiments, a viral expression construct can comprise a VP-coding region; a VP-coding region is a nucleotide sequence which comprises a VP nucleotide sequence encoding VP1, VP2, VP3, or a combination thereof. In certain embodiments, a viral expression construct can comprise a VP1-coding region; a VP1-coding region is a nucleotide sequence which comprises a VP1 nucleotide sequence encoding a VP1 protein. In certain embodiments, a viral expression construct can comprise a VP2-coding region; a VP2-coding region is a nucleotide sequence which comprises a VP2 nucleotide sequence encoding a VP2 protein. In certain embodiments, a viral expression construct can comprise a VP3-coding region; a VP3-coding region is a nucleotide sequence which comprises a VP3 nucleotide sequence encoding a VP3 protein.

In certain embodiments, a VP-coding region encodes one or more AAV capsid proteins of a specific AAV serotype. The AAV serotypes for VP-coding regions can be the same or different. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In certain embodiments, the viral expression construct comprises a first VP-coding region which comprises a nucleotide sequence encoding one or more AAV capsid proteins selected from VP1, VP2 and VP3. In certain embodiments, the first VP-coding region comprises a nucleotide sequence encoding one or more AAV capsid proteins selected from VP2 and VP3. In certain embodiments, the first VP-coding region comprises a nucleotide sequence encoding VP1, VP2 and VP3 AAV capsid proteins. In certain embodiments, the first VP-coding region comprises a nucleotide sequence encoding VP2 and VP3 AAV capsid proteins. In certain embodiments, the first VP-coding region comprises a nucleotide sequence encoding only VP2 and VP3 AAV capsid proteins. In certain embodiments, the first VP-coding region comprises a nucleotide sequence encoding VP2 and VP3 AAV capsid proteins, but not VP1.

In certain embodiments, the nucleic acid construct comprises a second VP-coding region which comprises a nucleotide sequence encoding one or more AAV capsid proteins selected from VP1, VP2 and VP3. In certain embodiments, the second VP-coding region comprises a nucleotide sequence encoding VP1 AAV capsid proteins. In certain embodiments, the second VP-coding region comprises a nucleotide sequence encoding only VP1 AAV capsid proteins. In certain embodiments, the second VP-coding region comprises a nucleotide sequence encoding VP1 AAV capsid proteins, but not VP2 or VP3.

In certain embodiments, the viral expression construct is an engineered nucleic acid construct. In certain embodiments, the viral expression construct comprises a first nucleotide sequence which comprises the first VP-coding region and the second VP-coding region. In certain embodiments, the first nucleotide sequence comprises a first open reading frame (ORF) which comprises the first VP-coding region, and a second open reading frame (ORF) which comprises the second VP-coding region.

In certain embodiments, the viral expression construct comprises a first nucleotide sequence which comprises the first VP-coding region and a second nucleotide sequence which comprises the second VP-coding region. In certain embodiments, the first nucleotide sequence comprises a first open reading frame (ORF) which comprises the first VP-coding region, and the second nucleotide sequence comprises a second open reading frame (ORF) which comprises the second VP-coding region. In certain embodiments, the first open reading frame is different from the second open reading frame.

In certain embodiments, the viral expression construct comprises a first VP-coding region which comprises a nucleotide sequence encoding one or more AAV capsid proteins selected from VP1, VP2 and VP3; and a second VP-coding region which comprises a nucleotide sequence encoding one or more AAV capsid proteins selected from VP1, VP2 and VP3. In certain embodiments, the first VP-coding region comprises a nucleotide sequence encoding VP1, VP2 and VP3 AAV capsid proteins; and the second VP-coding region comprises a nucleotide sequence encoding only VP1 AAV capsid proteins. In certain embodiments, the first VP-coding region comprises a nucleotide sequence encoding VP1, VP2 and VP3 AAV capsid proteins; and the second VP-coding region comprises a nucleotide sequence encoding VP1 AAV capsid proteins, but not VP2 or VP3. In certain embodiments, the first VP-coding region comprises a nucleotide sequence encoding only VP2 and VP3 AAV capsid proteins; and the second VP-coding region comprises a nucleotide sequence encoding only VP1 AAV capsid proteins. In certain embodiments, the first VP-coding region comprises a nucleotide sequence encoding VP2 and VP3 AAV capsid proteins, but not VP1; and the second VP-coding region which comprises a nucleotide sequence encoding VP1 AAV capsid proteins, but not VP2 or VP3.

In certain embodiments, the first VP-coding region encodes AAV capsid proteins of an AAV serotype, e.g., AAV2. In certain embodiments, the second VP-coding region encodes AAV capsid proteins of an AAV serotype, e.g., AAV2. In certain embodiments, the AAV serotype of the first VP-coding region is the same as the AAV serotype of the second VP-coding region. In certain embodiments, the AAV serotype of the first VP-coding region is different from the AAV serotype of the second VP-coding region. In certain embodiments, a VP-coding region can be codon optimized for an insect cell. In certain embodiments, a VP-coding region can be codon optimized for a Spodoptera frugiperda cell.

In certain embodiments, the viral expression construct comprises: (i) a first nucleotide sequence which comprises a first expression control region comprising a first promoter sequence, and a first VP-coding region which comprises a nucleotide sequence encoding one or more AAV capsid proteins selected from VP1, VP2 and VP3; and (ii) a second nucleotide sequence which comprises a second expression control region comprising a second promoter sequence, and a second VP-coding region which comprises a nucleotide sequence encoding VP1 AAV capsid proteins, but not VP2 or VP3. In certain embodiments, the viral expression construct comprises: (i) a first nucleotide sequence which comprises a first expression control region comprising a first promoter sequence, and a first VP-coding region which comprises a nucleotide sequence encoding VP2 and VP3 AAV capsid proteins, but not VP1; and (ii) a second nucleotide sequence which comprises a second expression control region comprising a second promoter sequence, and a second VP-coding region which comprises a nucleotide sequence encoding VP1 AAV capsid proteins, but not VP2 or VP3. In certain embodiments, the nucleotide sequence of the second VP-coding region is codon optimized. In certain embodiments, the nucleotide sequence of the second VP-coding region is codon optimized for an insect cell, or more specifically for a Spodoptera frugiperda cell. In certain embodiments, the nucleotide sequence of the second VP-coding region is codon optimized codon optimized to have a nucleotide homology with the reference nucleotide sequence of less than 100%, less than 90%, or less than 80%.

In certain embodiments, the viral expression construct comprises: (i) a first nucleotide sequence which comprises a first expression control region comprising a first promoter sequence, a first start codon region which comprises a first start codon, a first VP-coding region which comprises a nucleotide sequence encoding one or more AAV capsid proteins selected from VP1, VP2 and VP3, and a first stop codon region which comprises a first stop codon; and (ii) a second nucleotide sequence which comprises a second expression control region comprising a second promoter sequence, a second start codon region which comprises a second start codon, a second VP-coding region which comprises a nucleotide sequence encoding VP1 AAV capsid proteins, but not VP2 or VP3, and a second stop codon region which comprises a second stop codon. In certain embodiments, the nucleic acid construct comprises: (i) a first nucleotide sequence which comprises a first expression control region comprising a first promoter sequence, a first start codon region which comprises a first start codon, a first VP-coding region which comprises a nucleotide sequence encoding VP2 and VP3 AAV capsid proteins, but not VP1, and a first stop codon region which comprises a first stop codon; and (ii) a second nucleotide sequence which comprises a second expression control region comprising a second promoter sequence, a second start codon region which comprises a second start codon, a second VP-coding region which comprises a nucleotide sequence encoding VP1 AAV capsid proteins, but not VP2 or VP3, and a second stop codon region which comprises a second stop codon. In certain embodiments, the first start codon is ATG, the second start codon is ATG, or both the first and second start codons are ATG.

In certain embodiments, a nucleotide sequence encoding a VP1 capsid protein can be codon optimized. In certain embodiments, a nucleotide sequence encoding a VP1 capsid protein can be codon optimized for an insect cell. In certain embodiments, a nucleotide sequence encoding a VP2 capsid protein can be codon optimized. In certain embodiments, a nucleotide sequence encoding a VP2 capsid protein can be codon optimized for an insect cell. In certain embodiments, a nucleotide sequence encoding a VP3 capsid protein can be codon optimized. In certain embodiments, a nucleotide sequence encoding a VP3 capsid protein can be codon optimized for an insect cell.

In certain embodiments, a nucleotide sequence encoding a VP1 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 700%, less than 68%, less than 66%, less than 64%, less than 62%, less than 60%, less than 55%, less than 50%, and less than 40%.

In certain embodiments, a nucleotide sequence encoding a VP2 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a nucleotide sequence encoding a VP3 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

Structural VP proteins, VP1, VP2, and VP3 of a viral expression construct can be encoded in a single open reading frame regulated by utilization of both alternative splice acceptor and non-canonical translational initiation codons. VP1, VP2 and VP3 can be transcribed and translated from a single transcript in which both in-frame and/or out-of-frame start codons are engineered to control the VP1:VP2:VP3 ratio produced by the nucleotide transcript. In certain embodiments, VP1 can be produced from a sequence which encodes for VP1 only. As use herein, the terms “only for VP1” or “VP1 only” refers to a nucleotide sequence or transcript which encodes for a VP1 capsid protein and: (i) lacks the necessary start codons within the VP1 sequence (i.e. deleted or mutated) for full transcription or translation of VP2 and VP3 from the same sequence; (ii) comprises additional codons within the VP1 sequence which prevent transcription or translation of VP2 and VP3 from the same sequence; or (iii) comprises a start codon for VP1 (e.g. ATG), such that VP1 is the primary VP protein produced by the nucleotide transcript.

In certain embodiments, VP2 can be produced from a sequence which encodes for VP2 only. As use herein, the terms “only for VP2” or “VP2 only” refers to a nucleotide sequence or transcript which encodes for a VP2 capsid protein and: (i) the nucleotide transcript is a truncated variant of a full VP capsid sequence which encodes only VP2 and VP3 capsid proteins; and (ii) which comprise a start codon for VP2 (e.g. ATG), such that VP2 is the primary VP protein produced by the nucleotide transcript.

In certain embodiments, VP1 and VP2 can be produced from a sequence which encodes for VP1 and VP2 only. As use herein, the terms “only for VP1 and VP2” or “VP1 and VP2 only” refer to a nucleotide sequence or transcript which encodes for VP1 and VP2 capsid proteins and: (i) lacks the necessary start codons within the VP sequence (i.e. deleted or mutated) for full transcription or translation of VP3 from the same sequence; (ii) comprises additional codons within the VP sequence which prevent transcription or translation of VP3 from the same sequence; (iii) comprises a start codon for VP1 (e.g. ATG) and VP2 (e.g. ATG), such that VP1 and VP2 are the primary VP protein produced by the nucleotide transcript; or (iv) comprises VP1-only nucleotide transcript and a VP2-only nucleotide transcript connected by a linker, such as an IRES region.

In certain embodiments, the viral expression construct may contain a nucleotide sequence which comprises a start codon region, such as a sequence encoding AAV capsid proteins which comprise one or more start codon regions. In certain embodiments, the start codon region can be within an expression control sequence. The start codon can be ATG or a non-ATG codon (i.e., a suboptimal start codon where the start codon of the AAV VP1 capsid protein is a non-ATG). In certain embodiments, the viral expression construct used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG. TTG, or GTG, as described in U.S. Pat. No. 8,163,543, the content of which is incorporated herein by reference in its entirety as related to AAV capsid proteins and the production thereof, insofar as it does not conflict with the present disclosure.

### Rep-Coding Region

In certain embodiments, a viral expression construct can comprise a Rep52-coding region. A Rep52-coding region is a nucleotide sequence which comprises a Rep52 nucleotide sequence encoding a Rep52 protein. In certain embodiments, a viral expression construct can comprise a Rep78-coding region. A Rep78-coding region is a nucleotide sequence which comprises a Rep78 nucleotide sequence encoding a Rep78 protein. In certain embodiments, a viral expression construct can comprise a Rep40-coding region. A Rep40-coding region is a nucleotide sequence which comprises a Rep40 nucleotide sequence encoding a Rep40 protein. In certain embodiments, a viral expression construct can comprise a Rep68-coding region. A Rep68-coding region is a nucleotide sequence which comprises a Rep68 nucleotide sequence encoding a Rep68 protein.

In certain embodiments, the viral expression construct comprises a first nucleotide sequence which comprises: a Rep52-coding region which comprises a Rep52 sequence encoding a Rep52 protein, a Rep78-coding region which comprises a Rep78 sequence encoding a Rep78 protein, or a combination thereof. In certain embodiments, the first nucleotide sequence comprises both a Rep52-coding region and a Rep78-coding region. In certain embodiments, the first nucleotide sequence comprises a single open reading frame, consists essentially of a single open reading frame, or consists of a single open reading frame. In certain embodiments, the first nucleotide sequence comprises a first open reading frame which comprises a Rep52-coding region, and a second open reading frame which comprises a Rep78-coding region and which is different from the first open reading frame.

In certain embodiments, non-structural proteins, Rep52 and Rep78, of a viral expression construct can be encoded in a single open reading frame regulated by utilization of both alternative splice acceptor and non-canonical translational initiation codons.

Both Rep78 and Rep52 can be translated from a single transcript: Rep78 translation initiates at a first start codon (AUG or non-AUG) and Rep52 translation initiates from a Rep52 start codon (e.g. AUG) within the Rep78 sequence. Rep78 and Rep52 can also be translated from separate transcripts with independent start codons. The Rep52 initiation codons within the Rep78 sequence can be mutated, modified or removed, such that processing of the modified Rep78 sequence will not produce Rep52 proteins.

In certain embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG and GTG, that effects partial exon skipping upon expression in insect cells, as described in U.S. Pat. No. 8,512,981, the content of which is incorporated herein by reference in its entirety as related to the promotion of less abundant expression of Rep78 as compared to Rep52 to promote high vector yields, insofar as it does not conflict with the present disclosure.

In certain embodiments, the viral expression construct may be a plasmid vector or a baculoviral construct for the expression in insect cells that contains repeating codons with differential codon biases, for example to achieve improved ratios of Rep proteins, e.g. Rep78 and Rep52 thereby improving large scale (commercial) production of viral expression construct and/or payload construct vectors in insect cells, as taught in U.S. Pat. No. 8,697,417, the content of which is incorporated herein by reference in its entirety as related to AAV replication proteins and the production thereof, insofar as it does not conflict with the present disclosure.

In certain embodiment, improved ratios of rep proteins may be achieved using the method and constructs described in U.S. Pat. No. 8,642,314, the content of which is incorporated herein by reference in its entirety as related to AAV replications proteins and the production thereof, insofar as it does not conflict with the present disclosure.

In certain embodiments, the viral expression construct may encode mutant parvoviral Rep polypeptides which have one or more improved properties as compared with their corresponding wild type Rep polypeptide, such as the preparation of higher virus titers for large scale production. Alternatively, they may be able to allow the production of better-quality viral particles or sustain more stable production of virus. In a non-limiting example, the viral expression construct may encode mutant Rep polypeptides with a mutated nuclear localization sequence or zinc finger domain, as described in Patent Application US 20130023034, the content of which is incorporated herein by reference in its entirety as related to AAV replications proteins and the production thereof, insofar as it does not conflict with the present disclosure.

In certain embodiments, the nucleic acid construct comprises a first nucleotide sequence, and a second nucleotide sequence which is separate from the first nucleotide sequence within the nucleic acid construct. In certain embodiments, the nucleic acid construct comprises a first nucleotide sequence which comprises a Rep52-coding region, and a separate second nucleotide sequence which comprises a Rep78-coding region. In certain embodiments, the nucleic acid construct comprises a first nucleotide sequence and a separate second nucleotide sequence; wherein the first nucleotide sequence comprises a Rep52-coding region and a 2A sequence region; and wherein the second nucleotide sequence comprises a Rep78-coding region and a 2A sequence region.

In certain embodiments, a first nucleotide sequence comprises a Rep52-coding region and 2A sequence region. In certain embodiments, a first nucleotide sequence comprises a Rep78-coding region and 2A sequence region. In certain embodiments, a first nucleotide sequence comprises a Rep52-coding region, a Rep78-coding region, and 2A sequence region. In certain embodiments, a first nucleotide sequence comprises a 2A sequence region located between a Rep52-coding region and a Rep78-coding region on the nucleotide sequence. In certain embodiments, a first nucleotide comprises, in order from the 5′-end to the 3′-end, a Rep52-coding region, a 2A sequence region, and a Rep78-coding region. In certain embodiments, a first nucleotide comprises, in order from the 5′-end to the 3′-end, a Rep78-coding region, a 2A sequence region, and a Rep52-coding region.

For example, in certain embodiments, a first nucleotide sequence comprises a start codon region, a Rep52-coding region, 2A sequence region, and a stop codon region. In certain embodiments, a first nucleotide sequence comprises a start codon region, a Rep78-coding region, 2A sequence region, and a stop codon region. In certain embodiments, a first nucleotide sequence comprises a start codon region, a Rep52-coding region, a 2A sequence region, a Rep78-coding region, and a stop codon region. In certain embodiments, a first nucleotide comprises, in order from the 5′-end to the 3′-end, a start codon region, a Rep52-coding region, a 2A sequence region, a Rep78-coding region, and a stop codon region. In certain embodiments, a first nucleotide comprises, in order from the 5′-end to the 3′-end, a start codon region, a Rep78-coding region, a 2A sequence region, a Rep52-coding region, and a stop codon region.

In certain embodiments, the viral expression construct comprises one or more essential-gene regions which comprises an essential-gene nucleotide sequence encoding an essential protein for the nucleic acid construct. In certain embodiments, the essential-gene nucleotide sequence is a baculoviral sequence encoding an essential baculoviral protein. In certain embodiments, the essential baculoviral protein is a baculoviral envelope protein or a baculoviral capsid protein. For example, in certain embodiments, the nucleic acid construct comprises a first nucleotide sequence and a separate second nucleotide sequence; wherein the first nucleotide sequence comprises a Rep52-coding region and a first essential-gene region; and wherein the second nucleotide sequence comprises a Rep78-coding region and a second essential-gene region. In certain embodiments, the nucleic acid construct comprises a first nucleotide sequence and a separate second nucleotide sequence; wherein the first nucleotide sequence comprises a Rep52-coding region, a 2A sequence region, and a first essential-gene region; and wherein the second nucleotide sequence comprises a Rep78-coding region, a 2A sequence region, and a second essential-gene region. In certain embodiments, the nucleic acid construct comprises a first nucleotide sequence and a separate second nucleotide sequence; wherein the first nucleotide sequence comprises, in order, a Rep52-coding region, a 2A sequence region, and a first essential-gene region; and wherein the second nucleotide sequence comprises, in order, a Rep78-coding region, a 2A sequence region, and a second essential-gene region.

In certain embodiments, the essential baculoviral protein is a GP64 baculoviral envelope protein. In certain embodiments, the essential baculoviral protein is a VP39 baculoviral capsid protein.

In certain embodiments, a first nucleotide sequence comprises a Rep52-coding region, a Rep78-coding region, and an IRES sequence region. In certain embodiments, a first nucleotide sequence comprises an IRES sequence region located between a Rep52-coding region and a Rep78-coding region on the nucleotide sequence. In certain embodiments, a first nucleotide comprises, in order from the 5′-end to the 3′-end, a Rep52-coding region, an IRES sequence region, and a Rep78-coding region. In certain embodiments, a first nucleotide comprises, in order from the 5′-end to the 3′-end, a Rep78-coding region, an IRES sequence region, and a Rep52-coding region.

In certain embodiments, the first nucleotide sequence comprises a first open reading frame which comprises a Rep52-coding region, a second open reading frame which comprises a Rep78-coding region, and an IRES sequence region located between the first open reading frame and the second open reading frame. In certain embodiments, a first nucleotide sequence comprises, in order from the 5′-end to the 3′-end, a first open reading frame which comprises a Rep52-coding region, an IRES sequence region, and a second open reading frame which comprises a Rep78-coding region. In certain embodiments, a first nucleotide sequence comprises, in order from the 5′-end to the 3′-end, a first open reading frame which comprises a Rep78-coding region, an IRES sequence region, and a second open reading frame which comprises a Rep52-coding region.

In certain embodiments, a first nucleotide sequence comprises, in order from the 5′-end to the 3′-end: a first open reading frame which comprises a first start codon region, a Rep52-coding region, and a first stop codon region; an IRES sequence region; and a second open reading frame which comprises a second start codon region, a Rep78-coding region, and a second stop codon region. In certain embodiments, a first nucleotide sequence comprises, in order from the 5′-end to the 3-end: a first open reading frame which comprises a first start codon region, a Rep78-coding region, and a first stop codon region; an IRES sequence region; and a second open reading frame which comprises a second start codon region, a Rep52-coding region, and a second stop codon region.

In certain embodiments of the present disclosure, Rep52 or Rep78 is transcribed from the baculoviral derived polyhedron promoter (polh). Rep52 or Rep78 can also be transcribed from a weaker promoter, for example a deletion mutant of the IE-1 promoter, the ΔIE-1 promoter, has about 20% of the transcriptional activity of that IE-1 promoter. A promoter substantially homologous to the ΔIE-1 promoter may be used. In respect to promoters, a homology of at least 50%, 60%, 70%, 80%, 90% or more, is considered to be a substantially homologous promoter.

### Polynucleotide Inserts

In certain embodiments, a viral expression construct or a payload construct of the present disclosure (e.g. bacmid) can include a polynucleotide incorporated by homologous recombination (transposon donor/acceptor system) into the bacmid by standard molecular biology techniques known and performed by a person skilled in the art. In certain embodiments, the polynucleotide incorporated into the bacmid can include an expression control sequence operably linked to a protein-coding nucleotide sequence. In certain embodiments, the polynucleotide incorporated into the bacmid can include an expression control sequence which includes a promoter, such as p10 or polH, and which is operably linked to a nucleotide sequence which encodes a structural AAV capsid protein (e.g. VP1. VP2, VP3 or a combination thereof). In certain embodiments, the polynucleotide incorporated into the bacmid can include an expression control sequence which includes a promoter, such as p10 or polH, and which is operably linked to a nucleotide sequence which encodes a non-structural AAV capsid protein (e.g. Rep78, Rep52, or a combination thereof).

In certain embodiments, the polynucleotide insert can be incorporated into the bacmid at the location of a baculoviral gene. In certain embodiments, the polynucleotide insert can be incorporated into the bacmid at the location of a non-essential baculoviral gene. In certain embodiments, the polynucleotide insert can be incorporated into the bacmid by replacing a baculoviral gene or a portion of the baculoviral gene with the polynucleotide insert. In certain embodiments, the polynucleotide insert can be incorporated into the bacmid by replacing a baculoviral gene or a portion of the baculoviral gene with a fusion-polynucleotide which includes the polynucleotide insert and the baculoviral gene (or portion thereof) being replaced.

In certain embodiments, the polynucleotide can be incorporated into the bacmid at the location of a restriction endonuclease (REN) cleavage site (i.e. REN access point) associated with a baculoviral gene. In certain embodiments, the polynucleotide can be incorporated into the bacmid using one or more endonucleases (e.g. homing endonucleases). See, for example. Lihoradova et al., J Virol Methods, 140(1-2):59-65 (2007), the content of which is incorporated herein by reference in its entirety as related to the direct cloning of foreign DNA into baculovirus genomes, and insofar as it does not conflict with the present disclosure.

In certain embodiments, the REN access point in the bacmid is FseI (corresponding with the global transactivator (gta) baculovirus gene) (ggccggcc). In certain embodiments, the REN access point in the bacmid is SdaI (corresponding with the DNA polymerase baculovirus gene) (cctgcagg). In certain embodiments, the REN access point in the bacmid is MauBI (corresponding with the lef-4 baculovirus gene) (cgcgcgcg). In certain embodiments, the REN access point in the bacmid is SbfI (corresponding with the gp64/gp67 baculovirus gene) (cctgcagg). In certain embodiments, the REN access point in the bacmid is I-CeuI (corresponding with the v-cath baculovirus gene) (SEQ ID NO: 1). In certain embodiments, the REN access point in the bacmid is AvrII (corresponding with the ecdysteroid UDP-glucosyltransferase (egt) baculovirus gene) (cctagg). In certain embodiments, the REN access point in the bacmid is NheI (gctagc). In certain embodiments, the REN access point in the bacmid is SpeI (actagt). In certain embodiments, the REN access point in the bacmid is BstZ17I (gtatac). In certain embodiments, the REN access point in the bacmid is NcoI (ccatgg). In certain embodiments, the REN access point in the bacmid is MluI (acgcgt).

Polynucleotides can be incorporated into these REN access points by: (i) providing a polynucleotide insert which has been engineered to include a target REN cleavage sequence (e.g. a polynucleotide insert engineered to include FseI REN sequences at both ends of the polynucleotide); (ii) proving a bacmid which includes the target REN access point for polynucleotide insertion (e.g. a variant of the AcMNPV bacmid bMON14272 which includes an FseI cleavage site (ii) digesting the REN-engineered polynucleotide with the appropriate REN enzyme (e.g. using FseI enzyme to digesting the REN-engineering polynucleotide which includes the FseI regions at both ends, to produce a polynucleotide-FseI insert); (iii) digesting the bacmid with the same REN enzyme to produce a single-cut bacmid at the REN access point (e.g. using FseI enzyme to produce a single-cut bacmid at the FseI location); and (iv) ligating the polynucleotide insert into the single-cut bacmid using an appropriate ligation enzyme, such as T4 ligase enzyme. The result is engineered bacmid DNA which includes the engineered polynucleotide insert at the target REN access point.

The insertion process can be repeated one or more times to incorporate other engineered polynucleotide inserts into the same bacmid at different REN access points (e.g. insertion of a first engineered polynucleotide insert at the AvrII REN access point in the egt, followed by insertion of a second engineered polynucleotide insert at the I-CeuI REN access point in the cath gene, and followed by insertion of a third engineered polynucleotide insert at the FseI REN access point in the gta gene).

In certain embodiments, the polynucleotide insert can be incorporated into the bacmid by splitting a baculoviral gene with the polynucleotide insert (i.e. the polynucleotide insert is incorporated into the middle of the gene, separating a 5′-portion of the gene from a 3-portion of the bacmid gene). In certain embodiments, the polynucleotide insert can be incorporated into the bacmid by splitting a baculoviral gene with the fusion-polynucleotide which includes the polynucleotide insert and a portion of the baculoviral gene which was split. In certain embodiments, the 3′ end of the fusion-polynucleotide includes the 5′-portion of the gene that was split, such that the 5′-portion of the gene in the fusion-polynucleotide and the 3′-portion of the gene remaining in the bacmid form a full or functional portion of the baculoviral gene. In certain embodiments, the 5′ end of the fusion-polynucleotide includes the 3-portion of the gene that was split, such that the 3-portion of the gene in the fusion-polynucleotide and the 5′-portion of the gene remaining in the bacmid form a full or functional portion of the baculoviral gene. A non-limiting example is presented in Examples 13 and 14, in which fusion-polynucleotides are engineered and produced to include components from the gta gene ORF (full/partial Ac-lef12 promoter, full/partial Ac-gta gene). Non-limiting examples of fusion polynucleotides of the present disclosure include the polynucleotides of SEQ ID NO: 43, 44 and 51.

In certain embodiments, restriction endonuclease (REN) cleavage can be used to remove one or more wild-type genes from a bacmid. In certain embodiments, restriction endonuclease (REN) cleavage can be used to remove one or more engineered polynucleotide insert which has been previously been inserted into the bacmid. In certain embodiments, restriction endonuclease (REN) cleavage can be used to replace one or more engineered polynucleotide inserts with a different engineered polynucleotide insert which includes the same REN cleavage sequences (e.g. an engineered polynucleotide insert at the FseI REN access point can be replaced with a different engineered polynucleotide insert which includes FseI REN cleavage sequences).

### Expression Control

### Expression Control Regions

A viral expression construct (e.g. expressionBac) of the present disclosure can comprise one or more expression control region encoded by expression control sequences. In certain embodiments, the expression control sequences are for expression in a viral production cell, such as an insect cell. In certain embodiments, the expression control sequences are operably linked to a protein-coding nucleotide sequence. In certain embodiments, the expression control sequences are operably linked to a VP coding nucleotide sequence or a Rep coding nucleotide sequence.

Herein, the terms “coding nucleotide sequence”, “protein-encoding gene” or “protein-coding nucleotide sequence” refer to a nucleotide sequence that encodes or is translated into a protein product, such as VP proteins or Rep proteins.

“Expression control sequence” refers to a nucleic acid sequence that regulates the expression of a nucleotide sequence to which it is operably linked. Thus, an expression control sequence can include promoters, enhancers, untranslated regions (UTRs), internal ribosome entry sites (IRES), transcription terminators, a start codon in front of a protein-encoding gene, splicing signal for introns, and stop codons. The term “expression control sequence” is intended to include, at a minimum, a sequence whose presence are designed to influence expression, and can also include additional advantageous components. For example, leader sequences and fusion partner sequences are expression control sequences. The term can also comprise the design of the nucleic acid sequence such that undesirable, potential initiation codons in and out of frame, are removed from the sequence. It can also comprise the design of the nucleic acid sequence such that undesirable potential splice sites are removed. It comprises sequences or polyadenylation sequences (pA) which direct the addition of a polyA tail, i.e., a string of adenine residues at the 3′-end of an mRNA, sequences referred to as polyA sequences. It also can be designed to enhance mRNA stability. Expression control sequences which affect the transcription and translation stability, e.g., promoters, as well as sequences which effect the translation, e.g., Kozak sequences, are known in insect cells. Expression control sequences can be of such nature as to modulate the nucleotide sequence to which it is operably linked such that lower expression levels or higher expression levels are achieved.

In certain embodiments, the expression control sequence can comprise one or more promoters. Promoters can comprise, but are not limited to, baculovirus major late promoters, insect virus promoters, non-insect virus promoters, vertebrate virus promoters, nuclear gene promoters, chimeric promoters from one or more species comprising virus and non-virus elements, and/or synthetic promoters. In certain embodiments, a promoter can be Ctx, Op-EI, EI, ΔEI, EI-1, pH, PIO, polH (polyhedron), ΔpolH, Dmhsp70, Hr1, Hsp70, 4×Hsp27 EcRE+minimal Hsp70, IE, IE-1, ΔIE-1, ΔIE, p10, Δp10 (modified variations or derivatives of p10), p5, p19, p35, p40, p6.9, and variations or derivatives thereof. In certain embodiments, the promoter is a Ctx promoter. In certain embodiments, the promoter is a p10 promoter. In certain embodiments, the promoter is a polH promoter. In certain embodiments, a promoter can be selected from tissue-specific promoters, cell-type-specific promoters, cell-cycle-specific promoters, and variations or derivatives thereof. In certain embodiments, a promoter can be a CMV promoter, an alpha 1-antitrypsin (al-AT) promoter, a thyroid hormone-binding globulin promoter, a thyroxine-binding globulin (LPS) promoter, an HCR-ApoCII hybrid promoter, an HCR-hAAT hybrid promoter, an albumin promoter, an apolipoprotein E promoter, an α1-AT+EaIb promoter, a tumor-selective E2F promoter, a mononuclear blood IL-2 promoter, and variations or derivatives thereof. In certain embodiments, the promoter is a low-expression promoter sequence. In certain embodiments, the promoter is an enhanced-expression promoter sequence. In certain embodiments, the promoter can comprise Rep or Cap promoters as described in US Patent Application 20110136227, the content of which is incorporated herein by reference in its entirety as related to expression promoters, insofar as it does not conflict with the present disclosure.

In certain embodiments, a viral expression construct can comprise the same promoter in all nucleotide sequences. In certain embodiments, a viral expression construct can comprise the same promoter in two or more nucleotide sequences. In certain embodiments, a viral expression construct can comprise a different promoter in two or more nucleotide sequences. In certain embodiments, a viral expression construct can comprise a different promoter in all nucleotide sequences.

In certain embodiments the viral expression construct encodes elements to improve expression in certain cell types. In a further embodiment, the expression construct may comprise polh and/or ΔIE-1 insect transcriptional promoters, CMV mammalian transcriptional promoter, and/or p10 insect specific promoters for expression of a desired gene in a mammalian or insect cell.

More than one expression control sequence can be operably linked to a given nucleotide sequence. For example, a promoter sequence, a translation initiation sequence, and a stop codon can be operably linked to a nucleotide sequence.

In certain embodiments, the viral expression construct can comprise one or more expression control sequence between protein-coding nucleotide sequences. In certain embodiments, an expression control region can comprise an IRES sequence region which comprises an IRES nucleotide sequence encoding an internal ribosome entry sight (IRES). The internal ribosome entry sight (IRES) can be selected from the group consisting or: FMDV-IRES from Foot-and-Mouth-Disease virus, EMCV-IRES from Encephalomyocarditis virus, and combinations thereof.

In certain embodiments, the viral expression construct is as described in PCT/US2019/054600 and/or U.S. Provisional Patent Application No. 62/741,855 the contents of which are each incorporated by reference in their entireties.

In certain embodiments, the viral expression construct may contain a nucleotide sequence which comprises a start codon region, such as a sequence encoding AAV capsid proteins which comprise one or more start codon regions. In certain embodiments, the start codon region can be within an expression control sequence.

In certain embodiments, the translational start site of eukaryotic mRNA can be controlled in part by a nucleotide sequence referred to as a Kozak sequence as described in Kozak, M Cell. 1986 Jan. 31:44(2):283-92 and Kozak, M. J Cell Biol. 1989 February; 108(2):229-41 the contents of each of which are herein incorporated by reference in their entirety as related to Kozak sequences and uses thereof. Both naturally occurring and synthetic translational start sites of the Kozak form can be used in the production of polypeptides by molecular genetic techniques, Kozak, M. Mamm Genome. 1996 August; 7(8):563-74 the contents of which are herein incorporated by reference in their entirety as related to Kozak sequences and uses thereof. Splice sites are sequences on an mRNA which facilitate the removal of parts of the mRNA sequences after the transcription (formation) of the mRNA. Typically, the splicing occurs in the nucleus, prior to mRNA transport into a cell's cytoplasm.

In certain embodiments, the viral expression construct may contain a nucleotide sequence which comprises a stop codon region, such as a sequence encoding AAV capsid proteins which comprise one or more stop codon regions. In certain embodiments, the stop codon region can be within an expression control sequence.

In certain embodiments, the viral expression construct comprises one or more start codon regions which include a start codon. In certain embodiments, the viral expression construct comprises one or more stop codon regions which include a stop codon. In certain embodiments, the viral expression construct comprises one or more start codon regions and one or more stop codon regions. In certain embodiments, the start codon region and/or stop codon region can be within an expression control sequence.

In certain embodiments, the viral expression construct comprises one or more expression control regions which comprise an expression control sequence. In certain embodiments, the expression control region comprises one or more promoter sequences. In certain embodiments, the expression control region comprises one or more promoter sequences selected from the group consisting of: baculovirus major late promoters, insect virus promoters, non-insect virus promoters, vertebrate virus promoters, nuclear gene promoters, chimeric promoters from one or more species including virus and non-virus elements, synthetic promoters, and variations or derivatives thereof. In certain embodiments, the expression control region comprises one or more promoter sequences selected from the group consisting of: Ctx promoter, polh insect transcriptional promoters, ΔIE-1 insect transcriptional promoters, p10 insect specific promoters, Δp10 insect specific promoters (variations or derivatives of p10), CMV mammalian transcriptional promoter, and variations or derivatives thereof. In certain embodiments, the expression control region comprises one or more low-expression promoter sequences. In certain embodiments, the expression control region comprises one or more enhanced-expression promoter sequences.

In certain embodiments, an expression control region can comprise a 2A sequence region which comprises a 2A nucleotide sequence encoding a viral 2A peptide. The sequence allows for co-translation of multiple polypeptides within a single open reading frame (ORF). As the ORF is translated, glycine and proline residues with the 2A sequence prevent the formation of a normal peptide bond, which results in ribosomal “skipping” and “self-cleavage” within the polypeptide chain. The viral 2A peptide can be selected from the group consisting of: F2A from Foot-and-Mouth-Disease virus, T2A from Thosea asigna virus, E2A from Equine rhinitis A virus, P2A from porcine teschovirus-1, BmCPV2A from cytoplasmic polyhedrosis virus, BmIFV 2A from B. mori flacherie virus, and combinations thereof.

In some embodiments, the first and/or second nucleotide sequence comprises a start codon and/or stop codon and/or internal ribosome entry site (IRES). In certain embodiments, the IRES nucleotide sequence encodes an internal ribosome entry site (IRES) selected from the group consisting of: FMDV-IRES from Foot-and-Mouth-Disease virus, EMCV-IRES from Encephalomyocarditis virus, and combinations thereof.

The method of the present disclosure is not limited by the use of specific expression control sequences. However, when a certain stoichiometry of VP products are achieved (close to 1:1:10 for VP1, VP2, and VP3, respectively) and also when the levels of Rep52 or Rep40 (also referred to as the p19 Reps) are significantly higher than Rep78 or Rep68 (also referred to as the p5 Reps), improved yields of AAV in production cells (such as insect cells) may be obtained. In certain embodiments, the p5/p19 ratio is below 0.6 more, below 0.4, or below 0.3, but always at least 0.03. These ratios can be measured at the level of the protein or can be implicated from the relative levels of specific mRNAs.

In certain embodiments, AAV particles are produced in viral production cells (such as mammalian or insect cells) wherein all three VP proteins are expressed at a stoichiometry approaching, about or which is 1:1:10 (VP1:VP2:VP3).

In certain embodiments, AAV particles are produced in viral production cells (such as mammalian or insect cells) wherein all three VP proteins are expressed at a stoichiometry approaching, about or which is 2:2:10 (VP1:VP2:VP3).

In certain embodiments, AAV particles are produced in viral production cells (such as mammalian or insect cells) wherein all three VP proteins are expressed at a stoichiometry approaching, about or which is 2:0:10 (VP1:VP2:VP3).

In certain embodiments, AAV particles are produced in viral production cells (such as mammalian or insect cells) wherein all three VP proteins are expressed at a stoichiometry approaching, about or which is 1-2:0-2:10 (VP1:VP2:VP3).

In certain embodiments, AAV particles are produced in viral production cells (such as mammalian or insect cells) wherein all three VP proteins are expressed at a stoichiometry approaching, about or which is 1-2:1-2:10 (VP1:VP2:VP3).

In certain embodiments, AAV particles are produced in viral production cells (such as mammalian or insect cells) wherein all three VP proteins are expressed at a stoichiometry approaching, about or which is 2-3:0-3:10 (VP1:VP2:VP3).

In certain embodiments, AAV particles are produced in viral production cells (such as mammalian or insect cells) wherein all three VP proteins are expressed at a stoichiometry approaching, about or which is 2-3:2-3:10 (VP1:VP2:VP3).

In certain embodiments, AAV particles are produced in viral production cells (such as mammalian or insect cells) wherein all three VP proteins are expressed at a stoichiometry approaching, about or which is 3:3:10 (VP1:VP2:VP3).

In certain embodiments, AAV particles are produced in viral production cells (such as mammalian or insect cells) wherein all three VP proteins are expressed at a stoichiometry approaching, about or which is 3-5:0-5:10 (VP1:VP2:VP3).

In certain embodiments, AAV particles are produced in viral production cells (such as mammalian or insect cells) wherein all three VP proteins are expressed at a stoichiometry approaching, about or which is 3-5:3-5:10 (VP1:VP2:VP3).

In certain embodiments, the expression control regions are engineered to produce a VP1:VP2:VP3 ratio selected from the group consisting of: about or exactly 1:0:10; about or exactly 1:1:10; about or exactly 2:1:10; about or exactly 2:1:10; about or exactly 2:2:10; about or exactly 3:0:10; about or exactly 3:1:10; about or exactly 3:2:10; about or exactly 3:3:10; about or exactly 4:0:10; about or exactly 4:1:10; about or exactly 4:2:10; about or exactly 4:3:10; about or exactly 4:4:10; about or exactly 5:5:10; about or exactly 1-2:0-2:10; about or exactly 1-2:1-2:10; about or exactly 1-3:0-3:10; about or exactly 1-3:1-3:10; about or exactly 1-4:0-4:10; about or exactly 1-4:1-4:10; about or exactly 1-5:1-5:10; about or exactly 2-3:0-3:10; about or exactly 2-3:2-3:10; about or exactly 2-4:24:10; about or exactly 2-5:2-5:10; about or exactly 3-4:3-4:10; about or exactly 3-5:3-5:10; and about or exactly 4-5:4-5:10.

### Transcriptional Regulatory Systems

The present disclosure presents transcriptional regulatory systems which can be used to regulate the expression of a protein-coding nucleotide sequence. The present disclosure presents viral expression constructs which include a transcriptional regulatory system which can be used to regulate the expression of a protein-coding nucleotide sequence. The present disclosure presents expression control regions which include a transcriptional regulatory system which can be used to regulate the expression of a protein-coding nucleotide sequence (i.e. regulatable expression control region).

In certain embodiments, the transcriptional regulatory system is functional in increasing the expression of a protein-coding nucleotide sequence. In certain embodiments, the transcriptional regulatory system is functional in decreasing or silencing the expression of a protein-coding nucleotide sequence. In certain embodiments, the transcriptional regulatory system is functional in increasing, decreasing or silencing the expression of a nucleotide sequence encoding one or more structural AAV capsid proteins (e.g. VP1, VP2, VP3, or a combination thereof). In certain embodiments, the transcriptional regulatory system is functional in increasing, decreasing or silencing the expression of a nucleotide sequence encoding one or more non-structural AAV replication proteins (e.g. Rep78, Rep52, or a combination thereof). In certain embodiments, the transcriptional regulatory system is functional in increasing, decreasing or silencing the expression of a nucleotide sequence encoding one or more payload polypeptides.

In certain embodiments, the transcriptional regulatory system includes at least one regulator element and at least one regulator binding region. In certain embodiments, the regulator element can bind to the regulator binding region. In certain embodiments, the regulator element has a high affinity for binding to the regulator binding region. In certain embodiments, the regulator element is an inducible regulator element. In certain embodiments, the transcriptional regulatory system includes at least one regulator element, at least one regulator binding region, and at least one inducer element. In certain embodiments, the inducer element can reduce the affinity of the regulator element for binding to the regulator binding region. In certain embodiments, the regulator element has a high affinity for binding to the regulator binding region when the inducer element is not present or present at low concentrations, and a low affinity for binding to the regulator binding region when the inducer element is present or present at high concentrations. In certain embodiments, the inducer element binds to regulator element and causes a conformational change in the regulator element to reduce binding affinity to the regulator binding region.

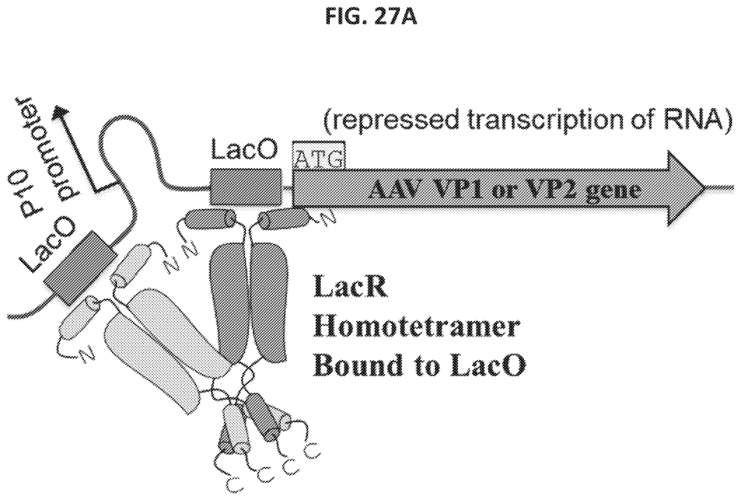

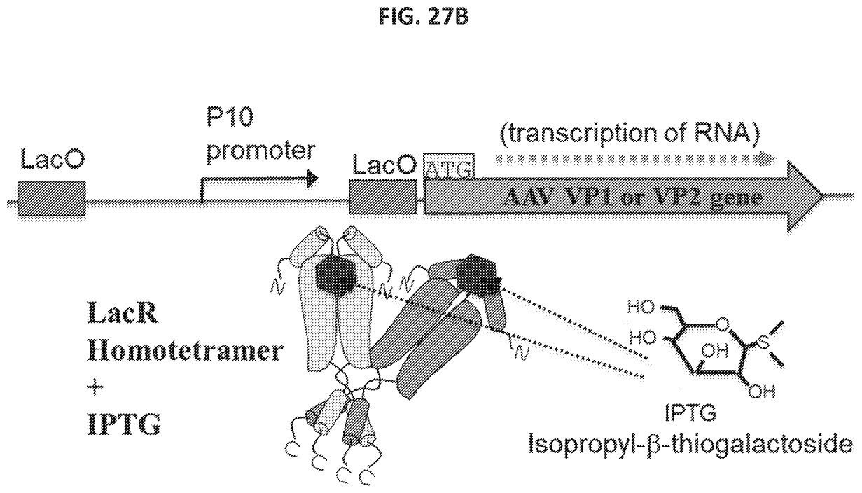

In certain embodiments, the regulator element is a Lac repressor (LacR) protein, the regulator binding region is a Lac Operator (LacO) nucleotide sequence, and the inducer element is a LacR inducer element selected from Lactose, Allolactose and isopropyl-β-D-thiogalactose (IPTG). As shown in FIG. 27A, the LacR protein is a homotetrameric protein which binds to one or more Lac Operator (LacO) nucleotide sequences. The tetrameric LacR protein typically binds to two LacO sequences simultaneously (such as one LacO sequence on each side of a promoter) and constrains the promoter (e.g. p10 promoter) into a loop when acting on the LacO sequences. When this happens, transcription initiation of the promoter is reduced or fully repressed. As shown in FIG. 27B, binding of LacR to LacO can controlled by the presence of an inducer element, such as the sugar allolactose. When allolactose binds to LacR, it causes LacR to conformationally change and to not bind to LacO nucleotide sequences. The synthetic analog of allolactose is isopropyl β-d-1-thiogalactopyranoside (IPTG). In certain embodiments, IPTG is preferred to allolactose because it is not metabolized and thus maintains stable induction of LacR after being added to cell cultures.

In certain embodiments, the regulator element is a Lac repressor (LacR) protein. LacR is typically a 360 amino acid protein with a molecular weight of 38 kDa which is typically encoded by the LacI gene. In certain embodiments, the regulator element is a Lac repressor (LacR) protein encoded by a LacR nucleotide sequence (i.e. LacI gene). In certain embodiments the LacR protein can be wt E. coli LacR from the LacI gene. In certain embodiments the LacR protein is an engineered LacR protein for expression in viral production cells, such as insect cells. Modifications to the LacI gene (and corresponding engineering LacR protein) can include: changing the translation initiation codon to ATG or a Kozak sequence (or modified Kozak sequence) which includes ATG; and the addition of an SV40 nuclear localization signal (NLS) to the N-terminus of LacR. In certain embodiments, the engineered LacR protein is encoded by a sequence which includes an NLS sequence, a linker sequence, and a modified LacI gene which includes a modified Kozak sequence and an ATG start codon. In certain embodiments, the engineered LacR protein is encoded by SEQ ID NO: 2. In certain embodiments, the engineered LacR protein is encoded by nucleotide sequence which has at least 70%, at least 75%, at least 80%, at least 85%, at least 90% or at least 95% identity to SEQ ID NO: 2.

In certain embodiments, the engineered LacR protein is codon optimized. In certain embodiments, the engineered LacR protein is codon optimized for insect cells. In certain embodiments, the engineered LacR protein is codon optimized for Spodoptera frugiperda insect cells. In certain embodiments, the engineered LacR protein is encoded by SEQ ID NO: 3. In certain embodiments, the engineered LacR protein is encoded by nucleotide sequence which has at least 70%, at least 75%, at least 80%, at least 85%, at least 90% or at least 95% identity to SEQ ID NO: 3.

In certain embodiments, the regulator binding region is a Lac Operator (LacO) nucleotide sequence (usually a 35 bp semipalindromic DNA element). In certain embodiments, the inducer element is a LacR inducer element, such as Lactose, Allolactose (intermediate metabolite of lactose), or isopropyl-β-D-thiogalactose (IPTG) (allolactose analogue). In certain embodiments, the LacR inducer element (e.g. IPTG) binds to LacR and causes a conformational change in LacR to reduce binding affinity to LacO.

In certain embodiments, the regulator element is a Tet repressor (TetR) protein or a tetracycline-controlled transactivator protein (tTA) (composed of TetR fused to strong transactivating domain of VP16 from Herpes simplex virus). In certain embodiments, the regulator element is a TetR protein encoded by a TetR nucleotide sequence. In certain embodiments, the regulator element is a tTA fusion protein encoded by a tTA nucleotide sequence. In certain embodiments, the regulator binding region is a Tet Operator (tetO) nucleotide sequence (usually a 19 bp DNA element) or a Tet Response Element (TRE) (which includes a series of two or more (e.g. seven) repeating tetO units). In certain embodiments, the inducer element is a TetR/tTA inducer element, such as tetracycline (Tet) or a tetracycline analog such as doxycycline (Dox). In certain embodiments, the regulator element includes a TetR protein or a tTA fusion protein, the regulator binding region includes at least one tetO nucleotide sequence (such as a TRE region which includes 2-7 repeating tetO units), and the inducer element is a TetR/tTA inducer element selected from tetracycline (Tet) or doxycycline (Dox). In certain embodiments, the TetR/tTA inducer element (e.g. Tet or Dox) binds to the TetR protein or TetR component of the tTA fusion protein, and causes conformational change in the TetR polypeptide to reduce binding affinity to tetO.

In certain embodiments, the transcriptional regulatory system can include one or more components as described in U.S. Pat. No. 6,133,027 (the contents of which are herein incorporated by reference in its entirety as related to transcriptional regulatory systems and components thereof), including specific regulator element, regulator binding regions, and inducer elements.

In certain embodiments, the transcriptional regulatory system includes at least one regulator binding region (i.e. regulator binding sequence) within the expression control region of a viral expression construct. In certain embodiments, the expression control region includes a promoter and at least one regulator binding region. In certain embodiments, the regulator binding region is 5-150 or 5-100 nucleotides from the promoter. In certain embodiments, the regulator binding region is between 5-10, 10-15, 15-20, 20-25, 25-30, 30-35, 35-40, 4045, 45-50, 50-55, 55-60, 60-65, 65-70, 70-80, 80-90, 90-100, 100-110, 110-120, 120-130, 130-140 or 140-150 nucleotides from the promoter. In certain embodiments, the regulator binding region is placed in a region known to be not essential for promoter function. In certain embodiments, the regulator binding region is a Lac Operator (LacO) nucleotide sequence. In certain embodiments, the Lac Operator (LacO) nucleotide sequence is SEQ ID NO: 4. In certain embodiments, the Lac Operator (LacO) nucleotide sequence is a nucleotide sequence which has at least 75%, at least 80%, at least 85%, at least 90% or at least 95% identity to SEQ ID NO: 4. In certain embodiments, the regulator binding region includes at least one tetO nucleotide sequence (such as a TRE region which includes 2-7 repeating tetO units). In certain embodiments, the promoter is a p10 promoter. In certain embodiments, the promoter is a polH promoter. In certain embodiments, the regulator binding region is a Lac Operator (LacO) nucleotide sequence and the promoter is a p10 promoter.

In certain embodiments, the expression control region includes a promoter and 2-7 regulator binding regions. In certain embodiments, the expression control region includes a promoter and two regulator binding regions. In certain embodiments, the expression control region includes a promoter, and upstream regulator binding region which is upstream of the promoter, and a downstream regulator binding region which is downstream from the promoter. In certain embodiments, the two regulator binding regions have a space interval of 100-300 nucleotides between them (measured from the center nucleotide of each regulator binding region). In certain embodiments, the two regulator binding regions have a space interval of 150-300, 150-250, 150-225, or 150-210 nucleotides between them (measured from the center nucleotide of each regulator binding region). In certain embodiments, the two regulator binding regions have a space interval of 100-105, 105-110, 110-115, 115-120, 120-125, 125-130, 130-135, 135-140, 140-145, 145-150, 150-155, 155-160, 160-165, 165-170, 170-175, 175-180, 180-185, 185-190,190-195, 195-200, 200-205, 200-210, 200-215, 205-210, 205-215, 210-215, 215-220, 220-225, 225-230, 230-235, 235-240, 240-245, 245-250, 250-255, 255-260, 260-265, 265-270, 270-275, 275-280, 280-285, 285-290, 290-295 or 295-300 nucleotides from the promoter. In certain embodiments, the two regulator binding regions have a space interval of 112 nucleotides. In certain embodiments, the two regulator binding regions have a space interval of 148 nucleotides. In certain embodiments, the two regulator binding regions have a space interval of 152 nucleotides. In certain embodiments, the two regulator binding regions have a space interval of 200 nucleotides. In certain embodiments, the two regulator binding regions have a space interval of 208 nucleotides.

In certain embodiments, the expression control region includes a promoter and two Lac Operator (LacO) nucleotide sequences. In certain embodiments, the expression control region includes a promoter, an upstream LacO nucleotide sequences which is upstream of the promoter, and a downstream LacO nucleotide sequences which is downstream of the promoter. In certain embodiments, the promoter is a p10 promoter. In certain embodiments, the promoter is a polH promoter. In certain embodiments, the expression control region includes a p10 promoter, an upstream LacO and downstream LacO, wherein the upstream LacO and downstream LacO have a space interval of 200-215 nucleotides (measured from the center nucleotide of each LacO sequence).

In certain embodiments, the transcriptional regulatory system includes at least one regulator element. In certain embodiments, the transcriptional regulatory system includes at least one regulator element. In certain embodiments, the regulator element is a Lac repressor (LacR) protein. In certain embodiments, the regulator element is a Tet repressor (TetR) protein. In certain embodiments, the regulator element is a tetracycline-controlled transactivator protein (tTA) composed of TetR fused to strong transactivating domain of VP16 from Herpes simplex virus.

In certain embodiments, the regulator element is a polypeptide that binds to one or more regulator binding sequences. In certain embodiments, the regulator element is a polypeptide that binds to two regulator binding sequences. In certain embodiments, the regulator element is a polypeptide that binds to 1-7 regulator binding sequences. In certain embodiments, the regulator element is a polypeptide that binds to one or more LacO sequences. In certain embodiments, the regulator element is a polypeptide that binds to two LacO sequences. In certain embodiments, the regulator element is a LacR protein that binds to one or more (e.g. two) LacO sequences. In certain embodiments, the regulator element is a polypeptide that binds to one or more tetO nucleotide sequence (such as a TRE region which includes 2-7 repeating tetO units). In certain embodiments, the regulator element is a TetR protein or tTA fusion protein that binds to one or more tetO nucleotide sequences.

In certain embodiments, the transcriptional regulatory system includes a promoter, at least one regulator binding region within 100 nucleotides from the promoter and at least one regulator element that binds to the regulator binding region. In certain embodiments, the regulator element is functional in decreasing transcription from the promoter when the regulator element is bound to the regulator binding region within 100 nucleotides from the promoter. In certain embodiments, the regulator element is functional in silencing transcription from the promoter when the regulator element is bound to the regulator binding region within 100 nucleotides from the promoter. In certain embodiments, the regulator element is functional in decreasing or silencing the expression of a protein-coding nucleotide sequence from the promoter when the regulator element is bound to the regulator binding region within 100 nucleotides from the promoter. In certain embodiments, the regulator element is functional in decreasing or silencing the expression of a protein-coding nucleotide sequence from the promoter by interfering with RNA polymerase activity at the promoter, thereby inhibiting or reducing transcriptional elongation from the promoter. In certain embodiments, the transcriptional regulatory system includes a p10 promoter, at least one LacO sequence within 100 nucleotides from the p10 promoter, and at least one LacR protein that binds to the LacO sequence. In certain embodiments, the LacR protein is functional in decreasing or silencing the expression of a protein-coding nucleotide sequence from the p10 promoter when the LacR protein is bound to the LacO sequence within 100 nucleotides from the promoter. In certain embodiments, the LacR protein is functional in decreasing or silencing the expression of a protein-coding nucleotide sequence from the p10 promoter when the LacR protein is bound to the LacO sequence by interfering with RNA polymerase activity at the p10 promoter, thereby inhibiting or reducing transcriptional elongation from the p10 promoter.

In certain embodiments, the expression control region includes a promoter, at least two regulator binding regions (i.e. regulator binding sequences) that are within 100 nucleotides from each end of the promoter region and with a space interval of 200-215 nucleotides (measured from the center nucleotide of each regulator binding sequence), and at least one regulator element that binds to the regulator binding region. In certain embodiments, the regulator element is functional in decreasing transcription from the promoter when the regulator element is bound to the regulator binding region within 100 nucleotides from the promoter. In certain embodiments, the regulator element is functional in silencing transcription from the promoter when the regulator element is bound to the regulator binding region within 100 nucleotides from the promoter. In certain embodiments, the regulator element is functional in decreasing or silencing the expression of a protein-coding nucleotide sequence from the promoter when the regulator element is bound to the regulator binding region within 100 nucleotides from the promoter. In certain embodiments, the regulator element is functional in decreasing or silencing the expression of a protein-coding nucleotide sequence from the promoter by interfering with RNA polymerase activity at the promoter, thereby inhibiting or reducing transcriptional elongation from the promoter. In certain embodiments, the transcriptional regulatory system includes a p10 promoter, at least one LacO sequence within 100 nucleotides upstream from the p10 promoter, at least one LacO sequence within 100 nucleotides downstream from the p10 promoter, and at least one LacR protein that simultaneously binds to both the upstream LacO sequence and the downstream LacO sequence. In certain embodiments, the LacR protein is functional in decreasing or silencing the expression of a protein-coding nucleotide sequence from the p10 promoter when the LacR protein is bound to both the upstream LacO sequence and downstream LacO sequence. In certain embodiments, the simultaneous binding of the LacR protein to both the upstream LacO sequence and the downstream LacO sequence results in the formation of a loop structure around the p10 promoter. In certain embodiments, the formation of the loop structure interferes with RNA polymerase activity at the p10 promoter, thereby inhibiting or reducing transcriptional elongation from the p10 promoter.

The present disclosure presents a viral expression construct which includes a nucleotide sequence which encodes a regulator element. In certain embodiments, the viral expression construct includes: (i) a first region or open reading frame (ORF) which includes a protein-coding nucleotide sequence operably linked to an expression control sequence, wherein the expression control sequence includes a promoter and at least one regulator binding region within 100 nucleotides from the promoter; and (ii) a second region or ORF which includes a nucleotide sequence which encodes a regulator element; and wherein the regulator element encoded by the nucleotide sequence in the second region/ORF has a binding affinity for the at least one regulator binding region within the expression control sequence of the first region/ORF. In certain embodiments, the regulator element from the second region/ORF is functional in decreasing or silencing the expression of the protein-coding nucleotide sequence from the promoter in the first region/ORF when the regulator element is bound to the regulator binding region within the expression control sequence of the first region/ORF.

In certain embodiments, the viral expression construct includes a LacI gene (or engineered variation thereof) which encodes a LacR protein (e.g. wt LacR protein or engineered LacR protein). In certain embodiments, the viral expression construct includes: (i) a first region or ORF which includes a protein-coding nucleotide sequence operably linked to an expression control sequence, wherein the expression control sequence includes a p10 promoter and at least one LacO sequence within 100 nucleotides from the promoter; and (ii) a second region or ORF which includes a nucleotide sequence which encodes a LacR protein (e.g. wt LacR protein or engineered LacR protein); and wherein the LacR protein encoded by the nucleotide sequence in the second region/ORF has a binding affinity for the at least one LacO sequence within the expression control sequence of the first region/ORF. In certain embodiments, the LacR protein encoded in the second region/ORF is functional in decreasing or silencing the expression of the protein-coding nucleotide sequence from the p10 promoter in the first region/ORF when the LacR protein is bound to the LacO sequence within the expression control sequence of the first region/ORF. In certain embodiments, the first region/ORF includes at least one LacO sequence within 100 nucleotides upstream from the p10 promoter and at least one LacO sequence within 100 nucleotides downstream from the p10 promoter, wherein the LacR protein can simultaneously bind to both the upstream LacO sequence and the downstream LacO sequence. In certain embodiments, the upstream LacO sequence and downstream LacO sequence have a space interval of 200-215 nucleotides (measured from the center nucleotide of each regulator binding sequence).

In certain embodiments, the viral expression construct includes: (i) a first region or ORF which includes a protein-coding nucleotide sequence operably linked to an expression control sequence, wherein the protein-coding nucleotide sequence includes a nucleotide sequence encoding one or more structural AAV capsid proteins (e.g. VP1, VP2, VP3, or a combination thereof), and wherein the expression control sequence includes a p10 promoter and at least one LacO sequence within 100 nucleotides from the promoter; and (ii) a second region or ORF which includes a nucleotide sequence which encodes a LacR protein (e.g. wt LacR protein or engineered LacR protein); and wherein the LacR protein encoded by the nucleotide sequence in the second region/ORF has a binding affinity for the at least one LacO sequence within the expression control sequence of the first region/ORF. In certain embodiments, the LacR protein encoded in the second region/ORF is functional in decreasing or silencing the expression of the structural AAV capsid proteins from the p10 promoter in the first region/ORF when the LacR protein is bound to the LacO sequence within the expression control sequence of the first ORF. In certain embodiments, the first ORF includes at least one LacO sequence within 100 nucleotides upstream from the p10 promoter and at least one LacO sequence within 100 nucleotides downstream from the p10 promoter, wherein the LacR protein can simultaneously bind to both the upstream LacO sequence and the downstream LacO sequence. In certain embodiments, the upstream LacO sequence and downstream LacO sequence have a space interval of 200-215 nucleotides (measured from the center nucleotide of each regulator binding sequence). In certain embodiments, the protein-coding nucleotide sequence encodes VP1, VP2 and VP3. In certain embodiments, the protein-coding nucleotide sequence encodes VP1 only. In certain embodiments, the protein-coding nucleotide sequence encodes VP2 only. In certain embodiments, the protein-coding nucleotide sequence encodes VP3 only.

In certain embodiments, the viral expression construct includes: (i) a first region or ORF which includes a protein-coding nucleotide sequence operably linked to an expression control sequence, wherein the protein-coding nucleotide sequence includes a nucleotide sequence encoding one or more non-structural AAV replication proteins (e.g. Rep78, Rep52, or a combination thereof), and wherein the expression control sequence includes a p10 promoter and at least one LacO sequence within 100 nucleotides from the promoter; and (ii) a second region or ORF which includes a nucleotide sequence which encodes a LacR protein (e.g. wt LacR protein or engineered LacR protein); and wherein the LacR protein encoded by the nucleotide sequence in the second region/ORF has a binding affinity for the at least one LacO sequence within the expression control sequence of the first region/ORF. In certain embodiments, the LacR protein encoded in the second region/ORF is functional in decreasing or silencing the expression of the non-structural AAV replication proteins from the p10 promoter in the first region/ORF when the LacR protein is bound to the LacO sequence within the expression control sequence of the first region/ORF. In certain embodiments, the first ORF includes at least one LacO sequence within 100 nucleotides upstream from the p10 promoter and at least one LacO sequence within 100 nucleotides downstream from the p10 promoter, wherein the LacR protein can simultaneously bind to both the upstream LacO sequence and the downstream LacO sequence. In certain embodiments, the upstream LacO sequence and downstream LacO sequence have a space interval of 200-215 nucleotides (measured from the center nucleotide of each regulator binding sequence). In certain embodiments, the protein-coding nucleotide sequence encodes Rep78 and Rep52. In certain embodiments, the protein-coding nucleotide sequence encodes Rep78 only. In certain embodiments, the protein-coding nucleotide sequence encodes Rep52 only.

In certain embodiments, the transcriptional regulatory system includes at least one inducer element which reduces the affinity of the regulator element for binding to the regulator binding region. In certain embodiments, the inducer element is a LacR inducer element. In certain embodiments, the LacR inducer element binds to LacR and causes a conformational change in LacR to reduce binding affinity to LacO. In certain embodiments, the LacR inducer element is Lactose. In certain embodiments, the LacR inducer element is Allolactose (intermediate metabolite of lactose). In certain embodiments, the LacR inducer element is isopropyl-β-D-thiogalactose (IPTG) (allolactose analogue). In certain embodiments, the inducer element is a TetR/tTA inducer element. In certain embodiments, the TetR/tTA inducer element binds to TetR (or the TetR component of tTA) and causes a conformational change in TetR to reduce binding affinity to TetO. In certain embodiments, the TetR/tTA inducer element is tetracycline (Tet). In certain embodiments, the TetR/tTA inducer element is a tetracycline analog. In certain embodiments, the TetR/tTA inducer element is doxycycline (Dox).

In certain embodiments, the inducer element is present at a target concentration of the inducer element. In certain embodiments, the inducer element is present at a concentration of about 0.0 μM, about 0.5 μM, about 1.0 μM, about 1.5 μM, about 2.0 μM, about 2.5 μM, about 3.0 μM, about 3.5 μM, about 4.0 μM, about 4.5 μM, about 5.0 μM, about 5.5 μM, about 6.0 μM, about 6.5 μM about 7.0 μM, about 7.5 μM, about 8.0 μM, about 8.5 μM, about 9.0 μM, about 9.5 μM, about 10.0 μM, about 10.5 μM, about 11.0 μM, about 11.5 μM, about 12.0 μM, about 12.5 μM, about 13.0 μM, about 13.5 μM, about 14.0 μM, about 14.5 μM, about 15.0 μM, about 15.5 μM, about 16.0 μM, about 16.5 μM, about 17.0 μM, about 17.5 μM, about 18.0 μM, about 18.5 μM, about 19.0 μM, about 19.5 μM, about 20.0 μM, about 20.5 μM, about 21.0 μM, about 21.5 μM, about 22.0 μM, about 22.5 μM, about 23.0 μM, about 23.5 μM, about 24.0 μM, about 24.5 μM, about 25.0 μM, about 25.5 μM, about 26.0 μM, about 26.5 μM, about 27.0 μM, about 27.5 μM, about 28.0 μM, about 28.5 μM, about 29.0 μM, about 29.5 μM, or about 30 μM.

In certain embodiments, the inducer element is present at a concentration of about 0.0 μM, about 5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, about 100 μM, about 105 μM, about 110 μM, about 115 μM, about 120 μM, about 125 μM, about 130 μM, about 135 μM, about 140 μM, about 145 μM, about 150 μM, about 155 μM, about 160 μM, about 165 μM, about 170 μM, about 175 μM, about 180 μM, about 185 μM, about 190 μM, about 195 μM, about 200 μM, about 205 μM, about 210 μM, about 215 μM, about 220 μM, about 225 μM, about 230 μM, about 235 μM, about 240 μM, about 245 μM, about 250 μM, about 255 μM, about 260 μM, about 265 μM, about 270 μM, about 275 μM, about 280 μM, about 285 μM, about 290 μM, about 295 μM, or about 300 μM.

In certain embodiments, the inducer element is present at a concentration between about 1.0 μM to about 200 μM. In certain embodiments, the inducer element is present at a concentration between about 1.0 μM to about 100 μM. In certain embodiments, the inducer element is present at a concentration between about 1.0 μM to about 50 μM. In certain embodiments, the inducer element is present at a concentration between about 1.0 μM to about 40 μM. In certain embodiments, the inducer element is present at a concentration between about 1.0 μM to about 35 μM. In certain embodiments, the inducer element is present at a concentration between about 10 μM to about 35 μM. In certain embodiments, the inducer element is present at a concentration about 10 μM to about 25 μM. In certain embodiments, the inducer element is present at a concentration between about 12.5 μM to about 22.5 μM. In certain embodiments, the inducer element is present at a concentration between about 13 μM to about 17 μM. In certain embodiments, the inducer element is present at a concentration between about 5 μM to about 15 μM. In certain embodiments, the inducer element is present at a concentration between about 8 μM to about 12 μM.

In certain embodiments, the transcriptional regulatory system includes a controlled amount or concentration of the inducer element. In certain embodiments, the amount of the inducer element included within the transcriptional regulatory system is proportional to the effect the inducer element has on the binding affinity between the regulator element and the regulator binding sequence. In certain embodiments, controlling the concentration of the inducer element within the transcriptional regulatory system allows for corresponding control of the expression of a protein-coding nucleotide sequence from the promoter.

In certain embodiments, the inducer element is not present or present at low concentrations. As a result, the regulator element has a high affinity for binding to the regulator binding region and expression of a protein-coding nucleotide sequence from the promoter is decreased or silenced. In certain embodiments, the inducer element is present or present at high concentrations. As a result, the regulator element has a low affinity for binding to the regulator binding region and expression of a protein-coding nucleotide sequence from the promoter is not decreased or minimally decreased. In certain embodiments, the concentration of the regulator element present in the transcriptional regulatory system is proportional to the affinity of the regulator element for binding to the regulator binding region. In certain embodiments, the concentration of the regulator element present in the transcriptional regulatory system is proportional to the level of decreased expression of a protein-coding nucleotide sequence resulting from the binding of regulator elements to regulator binding regions. In certain embodiments, the concentration of the regulator element present in the transcriptional regulatory system is proportional to amount of protein material produce by the expression of the protein-coding nucleotide sequence from a promoter.

In certain embodiments, the transcriptional regulatory system is operable with a nucleotide sequence encoding one or more structural AAV capsid proteins (e.g. VP1, VP2, VP3, or a combination thereof), such that the concentration of the regulator element present in the transcriptional regulatory system is proportional to amount of the AAV capsid protein material produce by the expression of the protein-coding nucleotide sequence from a promoter. In certain embodiments, the transcriptional regulatory system is operable with a nucleotide sequence encoding VP1 only. In certain embodiments, the transcriptional regulatory system is operable with a nucleotide sequence encoding VP2 only. In certain embodiments, the transcriptional regulatory system is operable with a nucleotide sequence encoding VP3 only. In certain embodiments, a transcriptional regulatory system is engineered to provide a VP protein ratio (VP1:VP2:VP3) of about 1-2:1-2:10 when the viral expression construct is processed by a viral production cell. In certain embodiments, the transcriptional regulatory system is engineered to include a concentration of a regulator element which results in a VP protein ratio (VP1:VP2:VP3) of about 1-2:1-2:10 when the viral expression construct is processed by a viral production cell.

In certain embodiments, the transcriptional regulatory system is operable with a nucleotide sequence encoding one or more non-structural AAV replication proteins (e.g. Rep78, Rep52, or a combination thereof), such that the concentration of the regulator element present in the transcriptional regulatory system is proportional to amount of the AAV replication protein material produce by the expression of the protein-coding nucleotide sequence from a promoter. In certain embodiments, the transcriptional regulatory system is operable with a nucleotide sequence encoding Rep78 only. In certain embodiments, the transcriptional regulatory system is operable with a nucleotide sequence encoding Rep52 only. In certain embodiments, a transcriptional regulatory system is engineered to provide a ratio of p5 Rep proteins (Rep78 and Rep68) to p19 Rep proteins (Rep52 and Rep40) of about 1:1-10 when the viral expression construct is processed by a viral production cell. In certain embodiments, the transcriptional regulatory system is engineered to include a concentration of a regulator element which results in a ratio of p5 Rep proteins (Rep78 and Rep68) to p19 Rep proteins (Rep52 and Rep40) of about 1:1-10 when the viral expression construct is processed by a viral production cell.

In certain embodiments, the transcriptional regulatory system can include one or more regulatable elements presented in WO2016137949 or WO2017075335, the contents of each of which are herein incorporated by reference in their entireties insofar as they do not conflict with the present disclosure.

### Viral Production Cells and Vectors

### Mammalian Cells

Viral production of the present disclosure disclosed herein describes processes and methods for producing AAV particles or viral vector that contacts a target cell to deliver a payload construct, e.g. a recombinant AAV particle or viral construct, which comprises a nucleotide encoding a payload molecule. The viral production cell may be selected from any biological organism, comprising prokaryotic (e.g., bacterial) cells, and eukaryotic cells, comprising, insect cells, yeast cells and mammalian cells.

In certain embodiments, the AAV particles of the present disclosure may be produced in a viral production cell that comprises a mammalian cell. Viral production cells may comprise mammalian cells such as A549, WEH1, 3T3, 10T1/2, BHK, MDCK, COS 1, COS 7, BSC 1, BSC 40, BMT 10, VERO. W138, HeLa, HEK293, HEK293T (293T), Saos, C2C12, L cells, HT1080, HepG2 and primary fibroblast, hepatocyte and myoblast cells derived from mammals. Viral production cells can comprise cells derived from mammalian species comprising, but not limited to, human, monkey, mouse, rat, rabbit, and hamster or cell type, comprising but not limited to fibroblast, hepatocyte, tumor cell, cell line transformed cell, etc.

AAV viral production cells commonly used for production of recombinant AAV particles comprise, but is not limited to HEK293 cells, COS cells, C127, 3T3, CHO, HeLa cells, KB cells, BHK, and other mammalian cell lines as described in U.S. Pat. Nos. 6,156,303, 5,387,484, 5,741,683, 5,691,176, 6,428,988 and 5,688,676; U.S. patent application 2002/0081721, and International Patent Publication Nos. WO 00/47757, WO 00/24916, and WO %/17947, the contents of which are each incorporated herein by reference in their entireties, insofar as they do not conflict with the present disclosure. In certain embodiments, the AAV viral production cells are trans-complementing packaging cell lines that provide functions deleted from a replication-defective helper virus, e.g., HEK293 cells or other Ea trans-complementing cells.

In certain embodiments, the packaging cell line 293-10-3 (ATCC Accession No. PTA-2361) may be used to produce the AAV particles, as described in U.S. Pat. No. 6,281,010, the content of which is incorporated herein by reference in its entirety as related to the 293-10-3 packaging cell line and uses thereof, insofar as it does not conflict with the present disclosure.

In certain embodiments, of the present disclosure a cell line, such as a HeLA cell line, for trans-complementing E1 deleted adenoviral vectors, which encoding adenovirus E1a and adenovirus E1b under the control of a phosphoglycerate kinase (PGK) promoter can be used for AAV particle production as described in U.S. Pat. No. 6,365,394, the content of which is incorporated herein by reference in its entirety as related to the HeLA cell line and uses thereof, insofar as it does not conflict with the present disclosure.

In certain embodiments, AAV particles are produced in mammalian cells using a triple transfection method wherein a payload construct, parvoviral Rep and parvoviral Cap and a viral expression construct are comprised within three different constructs. The triple transfection method of the three components of AAV particle production may be utilized to produce small lots of virus for assays comprising transduction efficiency, target tissue (tropism) evaluation, and stability.

AAV particles to be formulated may be produced by triple transfection or baculovirus mediated virus production, or any other method known in the art. Any suitable permissive or packaging cell known in the art may be employed to produce the vectors. In certain embodiments, trans-complementing packaging cell lines are used that provide functions deleted from a replication-defective helper virus, e.g., 293 cells or other E1a trans-complementing cells.

The gene cassette may contain some or all of the parvovirus (e.g., AAV) cap and rep genes. In certain embodiments, some or all of the cap and rep functions are provided in trans by introducing a packaging vector(s) encoding the capsid and/or Rep proteins into the cell. In certain embodiments, the gene cassette does not encode the capsid or Rep proteins. Alternatively, a packaging cell line is used that is stably transformed to express the cap and/or rep genes.

Recombinant AAV virus particles are, in certain embodiments, produced and purified from culture supernatants according to the procedure as described in US2016/0032254, the content of which is incorporated herein by reference in its entirety as related to the production and processing of recombinant AAV virus particles, insofar as it does not conflict with the present disclosure. Production may also involve methods known in the art comprising those using 293T cells, triple transfection or any suitable production method.

In certain embodiments, mammalian viral production cells (e.g. 293T cells) can be in an adhesion/adherent state (e.g. with calcium phosphate) or a suspension state (e.g. with polyethyleneimine (PEI)). The mammalian viral production cell is transfected with plasmids required for production of AAV, (i.e., AAV rep/cap construct, an adenoviral viral expression construct, and/or ITR flanked payload construct). In certain embodiments, the transfection process can comprise optional medium changes (e.g. medium changes for cells in adhesion form, no medium changes for cells in suspension form, medium changes for cells in suspension form if desired). In certain embodiments, the transfection process can comprise transfection mediums such as DMEM or F17. In certain embodiments, the transfection medium can comprise serum or can be serum-free (e.g. cells in adhesion state with calcium phosphate and with serum, cells in suspension state with PEI and without serum).

Cells can subsequently be collected by scraping (adherent form) and/or pelleting (suspension form and scraped adherent form) and transferred into a receptacle. Collection steps can be repeated as necessary for full collection of produced cells. Next, cell lysis can be achieved by consecutive freeze-thaw cycles (−80 C to 37 C), chemical lysis (such as adding detergent triton), mechanical lysis, or by allowing the cell culture to degrade after reaching ˜0% viability. Cellular debris is removed by centrifugation and/or depth filtration. The samples are quantified for AAV particles by DNase resistant genome titration by DNA qPCR.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039; Veldwijk et al. (2002) Mol. Ther., 6:272-278, the contents of which are each incorporated herein by reference in their entireties as related to the measurement of particle concentrations, insofar as they do not conflict with the present disclosure).

### Insect Cells

Viral production of the present disclosure comprises processes and methods for producing AAV particles or viral vectors that contact a target cell to deliver a payload construct, e.g. a recombinant viral construct, which comprises a nucleotide encoding a payload molecule. In certain embodiments, the AAV particles or viral vectors of the present disclosure may be produced in a viral production cell that comprises an insect cell.

Growing conditions for insect cells in culture, and production of heterologous products in insect cells in culture are well-known in the art, see U.S. Pat. No. 6,204,059, the content of which is incorporated herein by reference in its entirety as related to the growth and use of insect cells in viral production, insofar as it does not conflict with the present disclosure.

Any insect cell which allows for replication of parvovirus and which can be maintained in culture can be used in accordance with the present disclosure. AAV viral production cells commonly used for production of recombinant AAV particles comprise, but is not limited to, Spodoptera frugiperda, comprising, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, N J (1995); O'Reilly et al., Baculovirus Expression Vectors, A Laboratory Manual, Oxford Univ. Press (1994); Samulski et al., J. Vir.63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir.219:37-44 (1996); Zhao et al., Vir.272:382-93 (2000); and Samulski et al., U.S. Pat. No. 6,204,059, the contents of which are each incorporated herein by reference in their entireties as related to the use of insect cells in viral production, insofar as they do not conflict with the present disclosure.

In one embodiment, the AAV particles are made using the methods described in WO2015/191508, the content of which is incorporated herein by reference in its entirety, insofar as it does not conflict with the present disclosure.

In certain embodiments, insect host cell systems, in combination with baculoviral systems (e.g., as described by Luckow et al., Bio/Technology 6: 47 (1988)) may be used. In certain embodiments, an expression system for preparing chimeric peptide is Trichoplusia ni, Tn 5B1-4 insect cells/baculoviral system, which can be used for high levels of proteins, as described in U.S. Pat. No. 6,660,521, the content of which is incorporated herein by reference in its entirety, insofar as it does not conflict with the present disclosure.

Expansion, culturing, transfection, infection and storage of insect cells can be carried out in any cell culture media, cell transfection media or storage media known in the art, including Hyclone SFX Insect Cell Culture Media, Expression System ESF AF Insect Cell Culture Medium, ThermoFisher Sf900II media, ThermoFisher Sf900III media, or ThermoFisher Grace's Insect Media. Insect cell mixtures of the present disclosure can also include any of the formulation additives or elements described in the present disclosure, including (but not limited to) salts, acids, bases, buffers, surfactants (such as Poloxamer 188/Pluronic F-68), and other known culture media elements. Formulation additives can be incorporated gradually or as “spikes” (incorporation of large volumes in a short time).

### Baculovirus Production Systems

In certain embodiments, processes of the present disclosure can comprise production of AAV particles or viral vectors in a baculoviral system using a viral expression construct and a payload construct vector. In certain embodiments, the baculoviral system comprises Baculovirus expression vectors (BEVs) and/or baculovirus infected insect cells (BIICs). In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure can be polynucleotide incorporated by homologous recombination (transposon donor/acceptor system) into a bacmid by standard molecular biology techniques known and performed by a person skilled in the art. Transfection of separate viral replication cell populations produces two or more groups (e.g. two, three) of baculoviruses (BEVs), one or more group which can comprise the viral expression construct (e.g., the baculovirus is an “Expression BEV” or “expressionBac”), and one or more group which can comprise the payload construct (e.g., the baculovirus is a “Payload BEV” or “payloadBac”). The baculoviruses may be used to infect a viral production cell for production of AAV particles or viral vector.

In certain embodiments, the process comprises transfection of a single viral replication cell population to produce a single baculovirus (BEV) group which comprises both the viral expression construct and the payload construct. These baculoviruses may be used to infect a viral production cell for production of AAV particles or viral vector.

In certain embodiments, BEVs are produced using a Bacmid Transfection agent, such as Promega FuGENE HD, WFI water, or ThermoFisher Cellfectin II Reagent. In certain embodiments, BEVs are produced and expanded in viral production cells, such as an insect cell.

In certain embodiments, the method utilizes seed cultures of viral production cells that comprise one or more BEVs, comprising baculovirus infected insect cells (BIICs). The seed BIICs have been transfected/transduced/infected with an Expression BEV which comprises a viral expression construct, and also a Payload BEV which comprises a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN₂ vapor.

Baculoviruses are made of several essential proteins which are essential for the function and replication of the Baculovirus, such as replication proteins, envelope proteins and capsid proteins. The Baculovirus genome thus comprises several essential-gene nucleotide sequences encoding the essential proteins. As a non-limiting example, the genome can comprise an essential-gene region which comprises an essential-gene nucleotide sequence encoding an essential protein for the Baculovirus construct. The essential protein can comprise: GP64 baculovirus envelope protein, VP39 baculovirus capsid protein, or other similar essential proteins for the Baculovirus construct.

Baculovirus expression vectors (BEV) for producing AAV particles in insect cells, comprising but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the content of which is incorporated herein by reference in its entirety as related to the production and use of BEVs and viral particles, insofar as it does not conflict with the present disclosure.

In certain embodiments, the production system of the present disclosure addresses baculovirus instability over multiple passages by utilizing a titerless infected-cells preservation and scale-up system. Small scale seed cultures of viral producing cells are transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture Wasilko D J et al. Protein Expr Purif. 2009 June; 65(2):122-32, the content of which is incorporated herein by reference in its entirety as related to the production and use of BEVs and viral particles, insofar as it does not conflict with the present disclosure.

A genetically stable baculovirus may be used to produce a source of the one or more of the components for producing AAV particles in invertebrate cells. In certain embodiments, defective baculovirus expression vectors may be maintained episomally in insect cells. In such an embodiment the corresponding bacmid vector is engineered with replication control elements, comprising but not limited to promoters, enhancers, and/or cell-cycle regulated replication elements.

In certain embodiments, baculoviruses may be engineered with a marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In certain embodiments, stable viral producing cells permissive for baculovirus infection are engineered with at least one stable integrated copy of any of the elements necessary for AAV replication and vector production comprising, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In certain embodiments, the Baculovirus expression vectors (BEV) are based on the Autographa californica multicapsid nucleopolyhedrosis virus (AcMNPV baculovirus) or BmNPV baculovirus. In certain embodiments, a bacmid of the present disclosure is based on (i.e. engineered variant of) an AcMNPV bacmid such as bmon14272, vAce25ko or vAclef11KO.

In certain embodiments, the Baculovirus expression vectors (BEV) is a BEV in which the baculoviral v-cath gene has been partially or fully deleted (“v-cath deleted BEV”) or mutated. In certain embodiments, the BEVs lack the v-cath gene or comprise a mutationally inactivated version of the v-cath gene. In certain embodiments, the BEVs lack the v-cath gene. In certain embodiments, the BEVs comprise a mutationally inactivated version of the v-cath gene.

Viral production bacmids of the present disclosure can comprise deletion of certain baculoviral genes or loci.

The present disclosure presents methods for producing a baculovirus infected insect cell (BIIC), e.g., expression BIICs and/or payload BIICs. In certain embodiments, the present disclosure presents methods for producing a baculovirus infected insect cell (BIIC) which comprises the following steps: (a) introducing a volume of cell culture medium into a bioreactor; (b) introducing at least one viral production cell (VPC) into the bioreactor and expanding the number of VPCs in the bioreactor to a target VPC cell density; (c) introduction at least one Baculoviral Expression Vector (BEV) into the bioreactor, wherein the BEV comprises an AAV viral expression construct or an AAV payload construct; (d) incubating the mixture of VPCs and BEVs in the bioreactor under conditions which allow at least one BEV to infect at least one VPC to produce a baculovirus infected insect cell (BIIC); (e) incubating the bioreactor under conditions which allow the number of BIICs in the bioreactor to reach a target BIIC cell density; and (f) harvesting the BIICs from the bioreactor. In certain embodiments, the bioreactor has a volume of at least 5 L, 10 L, 20 L, 50 L, 100 L. or 200 L. In certain embodiments, the volume of cell culture medium (i.e. working volume) in the bioreactor is at least 5 L, 10 L, 20 L, 50 L, 100 L, or 200 L.

In certain embodiments, the VPC density at BEV introduction is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at BEV introduction is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, the target VPC cell density at BEV introduction is 1.5-4.0×10⁶ cells/mL. In certain embodiments, the target VPC cell density at BEV introduction is 2.0-3.5×10⁶ cells/mL.

In certain embodiments, the BEVs are introduced into the bioreactor at a target Multiplicity of Infection (MOI) of BEVs to VPCs. In certain embodiments, the BEV MOI is 0.0005-0.003, or more specifically 0.001-0.002.

In certain embodiments, the BIICs are harvested from the bioreactor at a specific BIIC cell density. In certain embodiments, the BIICs harvested from the bioreactor have a specific BIIC cell density. In certain embodiments, the BIIC cell density at harvesting is 6.0-18.0×10⁶ cells/mL, 8.0-16.5×10⁶ cells/mL, 10.0-16.5×10⁶ cells/mL.

In certain embodiments, BIICs (expression BIICs, payload BIICs) are used to transfect viral production cells, e.g., Sf9 cells. In some embodiments, baculoviruses comprising bacmids such as BEVs (expressionBacs, payloadBacs) are used to transfect viral production cells, e.g., Sf9 cells.

### Other

In certain embodiments expression hosts comprise, but are not limited to, bacterial species within the genera Escherichia, Bacillus, Pseudomonas, or Salmonella.

In certain embodiments, a host cell which comprises AAV rep and cap genes stably integrated within the cell's chromosomes, may be used for AAV particle production. In a non-limiting example, a host cell which has stably integrated in its chromosome at least two copies of an AAV rep gene and AAV cap gene may be used to produce the AAV particle according to the methods and constructs described in U.S. Pat. No. 7,238,526, the content of which is incorporated herein by reference in its entirety as related to the production of viral particles, insofar as it does not conflict with the present disclosure.

In certain embodiments, the AAV particle can be produced in a host cell stably transformed with a molecule comprising the nucleic acid sequences which permit the regulated expression of a rare restriction enzyme in the host cell, as described in US20030092161 and EP1183380, the contents of which are each incorporated herein by reference in their entireties as related to the production of viral particles, insofar as they do not conflict with the present disclosure.

In certain embodiments, production methods and cell lines to produce the AAV particle may comprise, but are not limited to those taught in PCT/US1996/010245, PCT/US1997/015716, PCT/US1997/015691, PCT/US1998/019479, PCT/US1998/019463, PCT/US2000/000415, PCT/US2000/040872, PCT/US2004/016614, PCT/US2007/010055, PCT/US1999/005870, PCT/US2000/004755, U.S. Ser. No. 08/549,489, U.S. Ser. No. 08/462,014, U.S. Ser. No. 09/659,203, U.S. Ser. No. 10/246,447, U.S. Ser. No. 10/465,302, U.S. Pat. Nos. 6,281,010, 6,270,996, 6,261,551, 5,756,283, 6,428,988, 6,274,354, 6,943,019, 6,482,634, (Assigned to NIH: U.S. Pat. Nos. 7,238,526, 6,475,769), U.S. Pat. No. 6,365,394 (Assigned to NIH), U.S. Pat. Nos. 7,491,508, 7,291,498, 7,022,519, 6,485,966, 6,953,690, 6,258,595, EP2018421, EP1064393, EP1163354, EP835321, EP931158, EP950111, EP1015619, EP1183380, EP2018421, EP1226264, EP1636370, EP1163354, EP1064393, US20030032613, US20020102714, US20030073232, US20030040101 (Assigned to NIH), US20060003451, US20020090717, US20030092161, US20070231303, US20060211115, US20090275107, US2007004042, US20030119191, US20020019050, the contents of which are each incorporated herein by reference in their entireties, insofar as they do not conflict with the present disclosure.

### Viral Production Systems

### Large-Scale Production

In certain embodiments, AAV particle production may be modified to increase the scale of production. Large scale viral production methods according to the present disclosure may comprise any of the processes or processing steps taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference by reference in their entirety.

Methods of increasing AAV particle production scale typically comprise increasing the number of viral production cells. In certain embodiments, viral production cells comprise adherent cells. To increase the scale of AAV particle production by adherent viral production cells, larger cell culture surfaces are required. In certain embodiments, large-scale production methods comprise the use of roller bottles to increase cell culture surfaces. Other cell culture substrates with increased surface areas are known in the art. Examples of additional adherent cell culture products with increased surface areas comprise, but are not limited to iCELLis (Pall Corp, Port Washington, N.Y.), CELLSTACK®, CELLCUBE® (Corning Corp., Corning, N.Y.) and NUNC™ CELL FACTORY™ (Thermo Scientific, Waltham, Mass.) In certain embodiments, large-scale adherent cell surfaces may comprise from about 1,000 cm² to about 100,000 cm².

In certain embodiments, large-scale viral production methods of the present disclosure may comprise the use of suspension cell cultures. Suspension cell culture can allow for significantly increased numbers of cells. Typically, the number of adherent cells that can be grown on about 10-50 cm² of surface area can be grown in about 1 cm³ volume in suspension.

In certain embodiments, large-scale cell cultures may comprise from about 10⁷ to about 10⁹ cells, from about 10⁸ to about 10¹⁰ cells, from about 10⁹ to about 10¹² cells or at least 10¹² cells. In certain embodiments, large-scale cultures may produce from about 10⁹ to about 10¹², from about 10¹⁰ to about 10¹³, from about 10¹¹ to about 10¹⁴, from about 10¹² to about 10¹⁵ or at least 10¹⁵ AAV particles.

Transfection of replication cells in large-scale culture formats may be carried out according to any methods known in the art. For large-scale adherent cell cultures, transfection methods may comprise, but are not limited to the use of inorganic compounds (e.g. calcium phosphate,) organic compounds (e.g. polyethyleneimine (PEI)) or the use of non-chemical methods (e.g. electroporation). With cells grown in suspension, transfection methods may comprise, but are not limited to the use of inorganic compounds (e.g. calcium phosphate,) organic compounds (e.g. polyethyleneimine (PEI)) or the use of non-chemical methods (e.g. electroporation). In certain embodiments, transfection of large-scale suspension cultures may be carried out according to the section entitled “Transfection Procedure” described in Feng, L. et al., 2008. Biotechnol Appl Biochem. 50:121-32, the contents of which are herein incorporated by reference in their entirety. According to such embodiments, PEI-DNA complexes may be formed for introduction of plasmids to be transfected. In certain embodiments, cells being transfected with PEI-DNA complexes may be ‘shocked’ prior to transfection. This comprises lowering cell culture temperatures to 4° C. for a period of about 1 hour. In certain embodiments, cell cultures may be shocked for a period of from about 10 minutes to about 5 hours. In certain embodiments, cell cultures may be shocked at a temperature of from about 0° C. to about 20° C.

In certain embodiments, transfections may comprise one or more vectors for expression of an RNA effector molecule to reduce expression of nucleic acids from one or more payload construct. Such methods may enhance the production of AAV particles by reducing cellular resources wasted on expressing payload constructs. In certain embodiments, such methods may be carried according to those taught in US Publication No. US2014/0099666, the contents of which are herein incorporated by reference in their entirety.

### Bioreactors

In certain embodiments, cell culture bioreactors may be used for large scale production of AAV particles. In certain embodiments, bioreactors comprise stirred tank reactors. Such reactors generally comprise a vessel, typically cylindrical in shape, with a stirrer (e.g. impeller.) In certain embodiments, such bioreactor vessels may be placed within a water jacket to control vessel temperature and/or to minimize effects from ambient temperature changes.

Bioreactor vessel volume may range in size from about 500 ml to about 2 L, from about 1 L to about 5 L, from about 2.5 L to about 20 L, from about 10 L to about 50 L, from about 25 L to about 100 L, from about 75 L to about 500 L, from about 250 L to about 2,000 L, from about 1,000 L to about 10,000 L, from about 5,000 L to about 50,000 L or at least 50,000 L. Vessel bottoms may be rounded or flat. In certain embodiments, animal cell cultures may be maintained in bioreactors with rounded vessel bottoms.

In certain embodiments, bioreactor vessels may be warmed through the use of a thermocirculator. Thermocirculators pump heated water around water jackets. In certain embodiments, heated water may be pumped through pipes (e.g. coiled pipes) that are present within bioreactor vessels. In certain embodiments, warm air may be circulated around bioreactors, comprising, but not limited to air space directly above culture medium. Additionally, pH and CO₂ levels may be maintained to optimize cell viability.

In certain embodiments, bioreactors may comprise hollow-fiber reactors. Hollow-fiber bioreactors may support the culture of both anchorage dependent and anchorage independent cells. Further bioreactors may comprise, but are not limited to, packed-bed or fixed-bed bioreactors. Such bioreactors may comprise vessels with glass beads for adherent cell attachment. Further packed-bed reactors may comprise ceramic beads.

In certain embodiments, viral particles are produced through the use of a disposable bioreactor. In certain embodiments, bioreactors may comprise GE WAVE bioreactor, a GE Xcellerex Bioreactor, a Sartorius Biostat Bioreactor, a ThermoFisher Hyclone Bioreactor, or a Pall Allegro Bioreactor.

In certain embodiments, AAV particle production in cell bioreactor cultures may be carried out according to the methods or systems taught in U.S. Pat. Nos. 5,064,764, 6,194,191, 6,566,118, 8,137,948 or US Patent Application No. US2011/0229971, the contents of each of which are herein incorporated by reference in their entirety.

### Expansion of Viral Production Cell (VPC) Mixtures

In certain embodiments, an AAV particle or viral vector of the present disclosure may be produced in a viral production cell (VPC), such as an insect cell. Production cells can be sourced from a Cell Bank (CB) and are often stored in frozen cell banks.

In certain embodiments, a viral production cell from a Cell Bank is provided in frozen form. The vial of frozen cells is thawed, typically until ice crystal dissipate. In certain embodiments, the frozen cells are thawed at a temperature between 10-50° C., 15-40° C., 20-30° C., 25-50° C., 30-45° C., 35-40° C., or 37-39° C. In certain embodiments, the frozen viral production cells are thawed using a heated water bath.

In certain embodiments, a thawed CB cell mixture will have a cell density of 1.0×10⁴-1.0×10⁹ cells/mL. In certain embodiments, the thawed CB cell mixture has a cell density of 1.0×10⁴-2.5×10⁴ cells/mL, 2.5×10⁴-5.0×10⁴ cells/mL, 5.0×10⁴-7.5×10⁴ cells/mL, 7.5×10⁴-1.0×10⁵ cells/mL, 1.0×10⁵-2.5×10⁵ cells/mL, 2.5×10⁵-5.0×10⁵ cells/mL, 5.0×10⁵-7.5×10⁵ cells/mL, 7.5×10⁵-1.0×10⁶ cells/mL, 1.0×10⁶-2.5×10⁶ cells/mL, 2.5×10⁶-5.0×10⁶ cells/mL, 5.0×10⁶-7.5×10⁶ cells/mL, 7.5×10⁶-1.0×10⁷ cells/mL, 10×10⁷-2.5×10⁷ cells/mL, 2.5×10⁷-5.0×10⁷ cells/mL, 5.0×10⁷-7.5×10⁷ cells/mL, 7.5×10⁷-1.0×10⁸ cells/mL, 1.0×10⁸-2.5×10⁸ cells/mL, 2.5×10⁸-5.0×10⁸ cells/mL, 5.0×10⁸-7.5×10⁸ cells/mL, or 7.5×10⁸-1.0×10⁹ cells/mL.

In certain embodiments, the volume of the CB cell mixture is expanded. This process is commonly referred to as a Seed Train, Seed Expansion, or CB Cellular Expansion. Cellular/Seed expansion can comprise successive steps of seeding and expanding a cell mixture through multiple expansion steps using successively larger working volumes. In certain embodiments, cellular expansion can comprise one, two, three, four, five, six, seven, or more than seven expansion steps. In certain embodiments, the working volume in the cellular expansion can comprise one or more of the following working volumes or working volume ranges: 5 mL, 10 mL, 20 mL, 5-20 mL, 25 mL, 30 mL, 40 mL, 50 mL, 20-50 mL, 75 mL, 100 mL, 125 mL, 150 mL, 175 mL, 200 mL, 50-200 mL, 250 mL, 300 mL, 400 mL, 500 mL, 750 mL, 1000 mL, 250-1000 mL, 1250 mL, 1500 mL, 1750 mL, 2000 mL, 1000-2000 mL, 2250 mL, 2500 mL, 2750 mL, 3000 mL, 2000-3000 mL, 3500 mL, 4000 mL, 4500 mL, 5000 mL, 3000-5000 mL, 5.5 L, 6.0 L, 7.0 L, 8.0 L, 9.0 L, 10.0 L, and 5.0-10.0 L.

In certain embodiments, a volume of cells from a first expanded cell mixture can be used to seed a second, separate Seed Train/Seed Expansion (instead of using thawed CB cell mixture). This process is commonly referred to as rolling inoculum. In certain embodiments, rolling inoculum is used in a series of two or more (e.g. two, three, four or five) separate Seed Trains/Seed Expansions.

In certain embodiments, large-volume cellular expansion can comprise the use of a bioreactor, such as a GE WAVE bioreactor, a GE Xcellerex Bioreactor, a Sartorius Biostat Bioreactor, a ThermoFisher Hyclone Bioreactor, or a Pall Allegro Bioreactor.

In certain embodiments, the cell density within a working volume is expanded to a target output cell density. In certain embodiments, the output cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10′, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, the output cell density of a working volume provides a seeding cell density for a larger, successive working volume. In certain embodiments, the seeding cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, cellular expansion can last for 1-50 days. Each cellular expansion step or the total cellular expansion can last for 1-10 days, 1-5 days, 1-3 days, 2-3 days, 2-4 days, 2-5 days, 2-6 days, 3-4 days, 3-5 days, 3-6 days, 3-8 days, 4-5 days, 4-6 days, 4-8 days, 5-6 days, or 5-8 days. In certain embodiments, each cellular expansion step or the total cellular expansion can last for 1-100 generations, 1-1000 generations, 100-1000 generation, 100 generations or more, or 1000 generation or more.

In certain embodiments, infected or transfected production cells can be expanded in the same manner as CB cell mixtures, as set forth in the present disclosure.

### Infection of Viral Production Cells

In certain embodiments, AAV particles of the present disclosure are produced in a viral production cell (VPC), such as an insect cell, by infecting the VPC with a viral vector which comprises an AAV expression construct and/or a viral vector which comprises an AAV payload construct. In certain embodiments, the VPC is infected with an Expression BEV, which comprises an AAV expression construct and a Payload BEV which comprises an AAV payload construct.

In certain embodiments, AAV particles are produced by infecting a VPC with a viral vector which comprises both an AAV expression construct and an AAV payload construct. In certain embodiments, the VPC is infected with a single BEV which comprises both an AAV expression construct and an AAV payload construct.

In certain embodiments, VPCs (such as insect cells) are infected using Infection BIICs in an infection process which comprises the following steps: (i) A collection of VPCs are seeded into a Production Bioreactor; (ii) The seeded VPCs can optionally be expanded to a target working volume and cell density; (iii) Infection BIICs which comprise Expression BEVs and Infection BIICs which comprise Payload BEVs are injected into the Production Bioreactor, resulting in infected viral production cells; and (iv) incubation of the infected viral production cells to produce AAV particles within the viral production cells.

In certain embodiments, the VPC density at infection is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-3.4×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at infection is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.1×10⁶, 3.2×10⁶, 3.3×10⁶, 3.4×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL. In certain embodiments, the VPC density at infection is 2.0-3.5×10⁶ cells/mL. In certain embodiments, the VPC density at infection is 3.5-5.0×10⁶ cells/mL. In certain embodiments, the VPC density at infection is 5.0-7.5×10⁶ cells/mL. In certain embodiments, the VPC density at infection is 5.0-10.0×10⁶ cells/mL.

In certain embodiments, the VPC density at infection is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-3.4×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at infection is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.1×10⁶, 3.2×10⁶, 3.3×10⁶, 3.4×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL. In certain embodiments, the VPC density at infection is 2.0-3.5×10⁶ cells/mL. In certain embodiments, the VPC density at infection is 3.5-5.0×10⁶ cells/mL. In certain embodiments, the VPC density at infection is 5.0-7.5×10⁶ cells/mL. In certain embodiments, the VPC density at infection is 5.0-10.0×10⁶ cells/mL.

In certain embodiments, Infection BIICs are combined with the VPCs in target ratios of VPC-to-BIIC. In certain embodiments, the VPC-to-BIIC infection ratio (volume to volume) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.×10⁷ (VPC volume to BIIC volume). In certain embodiments, the VPC-to-BIIC infection ratio (volume to volume) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC volume to BIIC volume). In certain embodiments, the VPC-to-BIIC infection ratio (cell to cell) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC cells to BIIC cells). In certain embodiments, the VPC-to-BIIC infection ratio (cell to cell) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC cells to BIIC cells).

In certain embodiments, Infection BIICs which comprise Expression BEVs are combined with the VPCs in target ratios of VPC-to-expressionBIIC. In certain embodiments, the VPC-to-expressionBIIC infection ratio (volume to volume) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC volume to expressionBIIC volume). In certain embodiments, the VPC-to-expressionBIIC infection ratio (volume to volume) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC volume to expressionBIIC volume). In certain embodiments, the VPC-to-expressionBIIC infection ratio (cell to cell) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC cells to expressionBIIC cells). In certain embodiments, the VPC-to-expressionBIIC infection ratio (cell to cell) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁴, about 1.5×10⁵, about 2.0×10⁵, about 2.5×10, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC cells to expressionBIIC cells).

In certain embodiments, Infection BIICs which comprise Payload BEVs are combined with the VPCs in target ratios of VPC-to-payloadBIIC. In certain embodiments, the VPC-to-payloadBIIC infection ratio (volume to volume) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10³-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC volume to payloadBIIC volume). In certain embodiments, the VPC-to-payloadBIIC infection ratio (volume to volume) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC volume to payloadBIIC volume). In certain embodiments, the VPC-to-payloadBIIC infection ratio (cell to cell) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³, 1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 10.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC cells to payloadBIIC cells). In certain embodiments, the VPC-to-payloadBIIC infection ratio (cell to cell) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁴, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC cells to payloadBIIC cells).

In certain embodiments, Infection BIICs which comprise Expression BEVs and Infection BIICs which comprise Payload BEVs are combined with the VPCs in target expressionBIIC-to-payloadBIIC ratios. In certain embodiments, the ratio of expressionBIICs to payloadBIICs is 10:1, 9:1, 8:1, 7:1, 6:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:9, or 1:10. In certain embodiments, the ratio of expressionBIICs to payloadBIICs is between 6.5-7.5:1, 6-7:1, 5.5-6.5:1, 5-6:1, 4.5-5.5:1, 4-5:1, 3.5-4.5:1, 3-4:1, 2.5-3.5:1, 2-3:1, 1.5-2.5:1, 1-2:1, 1-1.5:1, 1:1-1.5, 1:1-2, 1:1.5-2.5, 1:2-3, 1:2.5-3.5, 1:3-4, 1:3.54.5, 1:4-5, 1:4.5-5.5, 1:5-6, 1:5.5-6.5, 1:6-7, or 1:6.5-7.5.

In certain embodiments, infected Viral Production Cells are incubated under a certain Dissolved Oxygen (DO) Content (DO %). In certain embodiments, infected Viral Production Cells are incubated under a DO % between 10%-50%, 20%-40%, 10%-20%, 15%-25%, 20%-30%, 25%-35%, 30%-40%, 35%-45%, 40%-50%, 10%-15%, 15%-20%, 20%-25%, 25%-30%, 30%-35%, 35%-40%, 40%-45%, or 45%-50%. In certain embodiments, infected Viral Production Cells are incubated under a DO % of about 10%, about 15%, about 20%, about 25%, about 30%, about 35%, about 40%, about 45%, about 50%. In certain embodiments, infected Viral Production Cells are incubated under a DO % between 20%-30% or about 25%. In certain embodiments, infected Viral Production Cells are incubated under a DO % between 25%-35% or about 30%. In certain embodiments, infected Viral Production Cells are incubated under a DO % between 30%-40% or about 35%. In certain embodiments, infected Viral Production Cells are incubated under a DO % between 35%-45% or about 40%.

### Cell Lysis

Cells of the present disclosure, comprising, but not limited to viral production cells, may be subjected to cell lysis according to any methods known in the art. Cell lysis may be carried out to obtain one or more agents (e.g. viral particles) present within any cells of the disclosure. In certain embodiments, a bulk harvest of AAV particles and viral production cells is subjected to cell lysis according to the present disclosure.

In certain embodiments, cell lysis may be carried out according to any of the methods or systems presented in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Cell lysis methods and systems may be chemical or mechanical. Chemical cell lysis typically comprises contacting one or more cells with one or more chemical lysis agent under chemical lysis conditions. Mechanical lysis typically comprises subjecting one or more cells to cell lysis carried out by mechanical force. Lysis can also be completed by allowing the cells to degrade after reaching ˜0% viability.

In certain embodiments, chemical lysis may be used to lyse cells. As used herein, the term “chemical lysis agent” refers to any agent that may aid in the disruption of a cell. In certain embodiments, lysis agents are introduced in solutions, termed lysis solutions or lysis buffers. As used herein, the term “chemical lysis solution” refers to a solution (typically aqueous) comprising one or more lysis agent. In addition to lysis agents, lysis solutions may comprise one or more buffering agents, solubilizing agents, surfactants, preservatives, cryoprotectants, enzymes, enzyme inhibitors and/or chelators. Lysis buffers are lysis solutions comprising one or more buffering agent. Additional components of lysis solutions may comprise one or more solubilizing agent. As used herein, the term “solubilizing agent” refers to a compound that enhances the solubility of one or more components of a solution and/or the solubility of one or more entities to which solutions are applied. In certain embodiments, solubilizing agents enhance protein solubility. In certain embodiments, solubilizing agents are selected based on their ability to enhance protein solubility while maintaining protein conformation and/or activity.

Exemplary lysis agents may comprise any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In certain embodiments, lysis agents may be selected from lysis salts, amphoteric agents, cationic agents, ionic detergents and non-ionic detergents. Lysis salts may comprise, but are not limited to, sodium chloride (NaCl) and potassium chloride (KCl.) Further lysis salts may comprise any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety.

In certain embodiments, cell lysates agents include amino acids such as arginine, or acidified amino acid mixtures such as arginine HCl.

In certain embodiments, the cell lysate solution comprises a stabilizing additive. In certain embodiments, the stabilizing additive can comprise trehalose, glycine betaine, mannitol, potassium citrate, CuCl₂, proline, xylitol, NDSB 201, CTAB and K₂PO₄. In certain embodiments, the stabilizing additive can comprise amino acids such as arginine, or acidified amino acid mixtures such as arginine HCl. In certain embodiments, the stabilizing additive can comprise 0.1 M arginine or arginine HCl. In certain embodiments, the stabilizing additive can comprise 0.2 M arginine or arginine HCl. In certain embodiments, the stabilizing additive can comprise 0.25 M arginine or arginine HCl. In certain embodiments, the stabilizing additive can comprise 0.3 M arginine or arginine HCl. In certain embodiments, the stabilizing additive can comprise 0.4 M arginine or arginine HCl. In certain embodiments, the stabilizing additive can comprise 0.5 M arginine or arginine HCl. In certain embodiments, the stabilizing additive can comprise 0.6 M arginine or arginine HCl. In certain embodiments, the stabilizing additive can comprise 0.7 M arginine or arginine HCl. In certain embodiments, the stabilizing additive can comprise 0.8 M arginine or arginine HCl. In certain embodiments, the stabilizing additive can comprise 0.9 M arginine or arginine HCl. In certain embodiments, the stabilizing additive can comprise 1.0M arginine or arginine HCl.

Concentrations of salts may be increased or decreased to obtain an effective concentration for the rupture of cell membranes. Amphoteric agents, as referred to herein, are compounds capable of reacting as an acid or a base. Amphoteric agents may comprise, but are not limited to lysophosphatidylcholine, 3-((3-Cholamidopropyl) dimethylammonium)-1-propanesulfonate (CHAPS), ZWITTERGENT® and the like. Cationic agents may comprise, but are not limited to, cetyltrimethylammonium bromide (C (16) TAB) and Benzalkonium chloride. Lysis agents comprising detergents may comprise ionic detergents or non-ionic detergents.

Detergents may function to break apart or dissolve cell structures comprising, but not limited to cell membranes, cell walls, lipids, carbohydrates, lipoproteins and glycoproteins. Exemplary ionic detergents comprise any of those taught in U.S. Pat. Nos. 7,625,570 and 6,593,123 or US Publication No. US2014/0087361, the contents of each of which are herein incorporated by reference in their entirety. In certain embodiments, the lysis solution comprises one or more ionic detergents. Example of ionic detergents for use in a lysis solution comprise, but are not limited to, sodium dodecyl sulfate (SDS), cholate and deoxycholate. In certain embodiments, ionic detergents may be comprised in lysis solutions as a solubilizing agent. In certain embodiments, the lysis solution comprises one or more nonionic detergents. Non-ionic detergents for use in a lysis solution may comprise, but are not limited to, octylglucoside, digitonin, lubrol, C12E8, TWEEN®-20, TWEEN®-80, Triton X-100, Triton X-114, Brij-35, Brij-58, and Noniodet P-40. Non-ionic detergents are typically weaker lysis agents but may be comprised as solubilizing agents for solubilizing cellular and/or viral proteins. In certain embodiments, the lysis solution comprises one or more zwitterionic detergents. Zwitterionic detergents for use in a lysis solution may comprise, but are not limited to: Lauryl dimethylamine N-oxide (LDAO); N,N-Dimethyl-N-dodecylglycine betaine (Empigen® BB); 3-(N,N-Dimethylmyristylammonio) propanesulfonate (Zwittergent® 3-10); n-Dodecyl-N,N-dimethyl-3-ammonio-1-propanesulfonate (Zwittergent® 3-12); n-Tetradecyl-N,N-dimethyl-3-ammonio-1-propanesulfonate (Zwittergent® 3-14); 3-(N,N-Dimethyl palmitylammonio) propanesulfonate (Zwittergent® 3-16); 3-((3-cholamidopropyl) dimethylammonio)-1-propanesulfonate (CHAPS); and 3-([3-Cholamidopropyl] dimethylammonio)-2-hydroxy-1-propanesulfonate (CHAPSO).

In certain embodiments, the lysis solution comprises Triton X-100 (octyl phenol ethoxylate), such as 0.5% w/v of Triton X-100. In certain embodiments, the lysis solution comprises Lauryldimethylamine N-oxide (LDAO), such as 0.184% w/v (4×CMC) of LDAO. In certain embodiments, the lysis solution comprises a seed oil surfactant such as Ecosurf™ SA-9. In certain embodiments, the lysis solution comprises N,N-Dimethyl-N-dodecylglycine betaine (Empigen® BB). In certain embodiments, the lysis solution comprises a Zwittergent® detergent, such as Zwittergent® 3-12 (n-Dodecyl-N,N-dimethyl-3-ammonio-1-propanesulfonate), Zwittergent® 3-14 (n-Tetradecyl-N,N-dimethyl-3-ammonio-1-propanesulfonate), or Zwittergent® 3-16 (3-(N,N-Dimethyl palmitylammonio)propanesulfonate).

Further lysis agents may comprise enzymes and urea. In certain embodiments, one or more lysis agents may be combined in a lysis solution in order to enhance one or more of cell lysis and protein solubility. In certain embodiments, enzyme inhibitors may be comprised in lysis solutions in order to prevent proteolysis that may be triggered by cell membrane disruption.

In certain embodiments, the lysis solution comprises between 0.1-1.0% w/v, between 0.2-0.8% w/v, between 0.3-0.7% w/v, between 0.4-0.6% w/v, or about 0.5% w/v of a cell lysis agent (e.g. detergent). In certain embodiments, the lysis solution comprises between 0.3-0.35% w/v, between 0.35-0.4% w/v, between 0.4-0.45% w/v, between 0.45-0.5% w/v, between 0.5-0.55% w/v, between 0.55-0.6% w/v, between 0.6-0.65% w/v, or between 0.65-0.7% w/v of a cell lysis agent (e.g. detergent).

In certain embodiments, cell lysates generated from adherent cell cultures may be treated with one more nuclease, such as Benzonase nuclease (Grade I, 99% pure) or c-LEcta Denarase nuclease (formerly Sartorius Denarase). In certain embodiments, nuclease is added to lower the viscosity of the lysates caused by liberated DNA.

In certain embodiments, chemical lysis uses a single chemical lysis mixture. In certain embodiments, chemical lysis uses several lysis agents added in series to provide a final chemical lysis mixture.

In certain embodiments, a chemical lysis mixture comprises an acidified amino acid mixture (such as arginine HCl), a non-ionic detergent (such as Triton X-100), and a nuclease (such as Benzonase nuclease). In certain embodiments, the chemical lysis mixture can comprise an acid or base to provide a target lysis pH.

In certain embodiments, the lysis solution comprises 0.5% w/v Triton X-100 (octyl phenol ethoxylate) and 200 mM arginine hydrochloride. In certain embodiments, the lysis solution comprises 0.5% w/v Triton X-100 (octyl phenol ethoxylate) and 200 mM arginine hydrochloride, and lacks detectable nuclease. In certain embodiments, the lysis solution consists of 0.5% w/v Triton X-100 (octyl phenol ethoxylate) and 200 mM arginine hydrochloride.

In certain embodiments, chemical lysis is conducted under chemical lysis conditions. As used herein, the term “chemical lysis conditions” refers to any combination of environmental conditions (e.g., temperature. pressure, pH, etc.) in which targets cells can be lysed by a chemical lysis agent.

In certain embodiments, the lysis pH is between 3.0-3.5, 3.5-4.0, 4.0-4.5, 4.5-5.0, 5.0-5.5, 5.5-6.0, 6.0-6.5, 6.5-7.0, 7.0-7.5, or 7.5-8.0. In certain embodiments, the lysis pH is between 6.0-7.0, 6.5-7.0, 6.5-7.5, or 7.0-7.5.

In certain embodiments, the lysis temperature is between 15-35° C., between 20-30° C., between 25-39° C., between 20-21° C., between 20-22° C., between 21-22° C., between 21-23° C., between 22-23° C., between 22-24° C., between 23-24° C., between 23-25° C. between 24-25° C., between 24-26° C., between 25-26° C., between 25-27° C., between 26-27° C., between 26-28° C., between 27-28° C., between 27-29° C., between 28-29° C., between 28-30° C., between 29-30° C., between 29-31° C., between 30-31° C., between 30-32° C., between 31-32° C., or between 31-33° C.

In certain embodiments, the lysis solution comprises 0.5% w/v Triton X-100 (octyl phenol ethoxylate) and 200 mM arginine hydrochloride, and lysis conditions comprise a duration of at least 4 hours (e.g., 4-6 hours, e.g., 4 hours) at 26° C.-28° C. (e.g., 27° C.). In certain embodiments, the lysis solution comprises 0.5% w/v Triton X-100 (octyl phenol ethoxylate) and 200 mM arginine hydrochloride, and lacks detectable nuclease, and lysis conditions comprise a duration of at least 4 hours (e.g., 4-6 hours, e.g., 4 hours) at 26° C.-28° C. (e.g., 27° C.). In certain embodiments, the lysis solution consists of 0.5% w/v Triton X-100 (octyl phenol ethoxylate) and 200 mM arginine hydrochloride, and lysis conditions comprise a duration of at least 4 hours (e.g., 4-6 hours, e.g., 4 hours) at 26° C.-28° C. (e.g., 27° C.).

In certain embodiments, mechanical cell lysis is carried out. Mechanical cell lysis methods may comprise the use of one or more lysis condition and/or one or more lysis force. As used herein, the term “lysis condition” refers to a state or circumstance that promotes cellular disruption. Lysis conditions may comprise certain temperatures, pressures, osmotic purity, salinity and the like. In certain embodiments, lysis conditions comprise increased or decreased temperatures. According to certain embodiments, lysis conditions comprise changes in temperature to promote cellular disruption. Cell lysis carried out according to such embodiments may comprise freeze-thaw lysis. As used herein, the term “freeze-thaw lysis” refers to cellular lysis in which a cell solution is subjected to one or more freeze-thaw cycle. According to freeze-thaw lysis methods, cells in solution are frozen to induce a mechanical disruption of cellular membranes caused by the formation and expansion of ice crystals. Cell solutions used according freeze-thaw lysis methods, may further comprise one or more lysis agents, solubilizing agents, buffering agents, cryoprotectants, surfactants, preservatives, enzymes, enzyme inhibitors and/or chelators. Once cell solutions subjected to freezing are thawed, such components may enhance the recovery of desired cellular products. In certain embodiments, one or more cryoprotectants are comprised in cell solutions undergoing freeze-thaw lysis. As used herein, the term “cryoprotectant” refers to an agent used to protect one or more substance from damage due to freezing. Cryoprotectants may comprise any of those taught in US Publication No. US2013/0323302 or U.S. Pat. Nos. 6,503,888, 6,180,613, 7,888,096, 7,091,030, the contents of each of which are herein incorporated by reference in their entirety. In certain embodiments, cryoprotectants may comprise, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose and urea. In certain embodiments, freeze-thaw lysis may be carried out according to any of the methods described in U.S. Pat. No. 7,704,721, the contents of which are herein incorporated by reference in their entirety.

As used herein, the term “lysis force” refers to a physical activity used to disrupt a cell. Lysis forces may comprise, but are not limited to mechanical forces, sonic forces, gravitational forces, optical forces, electrical forces and the like. Cell lysis carried out by mechanical force is referred to herein as “mechanical lysis.” Mechanical forces that may be used according to mechanical lysis may comprise high shear fluid forces. According to such methods of mechanical lysis, a microfluidizer may be used. Microfluidizers typically comprise an inlet reservoir where cell solutions may be applied. Cell solutions may then be pumped into an interaction chamber via a pump (e.g. high-pressure pump) at high speed and/or pressure to produce shear fluid forces. Resulting lysates may then be collected in one or more output reservoir. Pump speed and/or pressure may be adjusted to modulate cell lysis and enhance recovery of products (e.g. viral particles.) Other mechanical lysis methods may comprise physical disruption of cells by scraping.

Cell lysis methods may be selected based on the cell culture format of cells to be lysed. For example, with adherent cell cultures, some chemical and mechanical lysis methods may be used. Such mechanical lysis methods may comprise freeze-thaw lysis or scraping. In another example, chemical lysis of adherent cell cultures may be carried out through incubation with lysis solutions comprising surfactant, such as Triton-X-100.

In certain embodiments, a method for harvesting AAV particles without lysis may be used for efficient and scalable AAV particle production. In a non-limiting example, AAV particles may be produced by culturing an AAV particle lacking a heparin binding site, thereby allowing the AAV particle to pass into the supernatant, in a cell culture, collecting supernatant from the culture; and isolating the AAV particle from the supernatant, as described in US Patent Application 20090275107, the contents of which are incorporated herein by reference in their entirety.

### Clarification and Purification: General

Cell lysates comprising viral particles may be subjected to clarification and purification. Clarification generally refers to the initial steps taken in the purification of viral particles from cell lysates and serves to prepare lysates for further purification by removing larger, insoluble debris from a bulk lysis harvest. Viral production can comprise clarification steps at any point in the viral production process. Clarification steps may comprise, but are not limited to, centrifugation and filtration. During clarification. centrifugation may be carried out at low speeds to remove larger debris only. Similarly, filtration may be carried out using filters with larger pore sizes so that only larger debris is removed.

Purification generally refers to the final steps taken in the purification and concentration of viral particles from cell lysates by removing smaller debris from a clarified lysis harvest in preparing a final Pooled Drug Substance. Viral production can comprise purification steps at any point in the viral production process. Purification steps may comprise, but are not limited to, filtration and chromatography. Filtration may be carried out using filters with smaller pore sizes to remove smaller debris from the product or with larger pore sizes to retain larger debris from the product. Filtration may be used to alter the concentration and/or contents of a viral production pool or stream. Chromatography may be carried out to selectively separate target particles from a pool of impurities.

Large-scale production of high-concentration AAV formulations is complicated by the tendency for high concentrations of AAV particles to aggregate or agglomerate. Small scale clarification and concentration systems, such as dialysis cassettes or spin centrifugation, are generally not sufficiently scalable for large-scale production. The present disclosure provides embodiments of a clarification, purification and concentration system for processing large volumes of high-concentration AAV production formulations. In certain embodiments, the large-volume clarification system comprises one or more of the following processing steps: Depth Filtration, Microfiltration (e.g. 0.2 μm Filtration), Affinity Chromatography, Ion Exchange Chromatography such as anion exchange chromatography (AEX) or cation exchange chromatography (CEX), a tangential flow filtration system (TFF), Nanofiltration (e.g. Virus Retentive Filtration (VRF)), Final Filtration (FF), and Fill Filtration.

Objectives of viral clarification and purification comprise high throughput processing of cell lysates and to optimize ultimate viral recovery. Advantages of comprising clarification and purification steps of the present disclosure comprise scalability for processing of larger volumes of lysate. In certain embodiments, clarification and purification may be carried out according to any of the methods or systems presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

In certain embodiments, the compositions comprising at least one AAV particle may be isolated or purified using the methods or systems described in U.S. Pat. Nos. 6,146,874, 6,660,514, 8,283,151 or U.S. Pat. No. 8,524,446, the contents of which are herein incorporated by reference in their entirety.

### Clarification and Purification: Centrifugation

According to certain embodiments, cell lysates may be clarified by one or more centrifugation steps. Centrifugation may be used to pellet insoluble particles in the lysate. During clarification, centrifugation strength (which can be expressed in terms of gravitational units (g), which represents multiples of standard gravitational force) may be lower than in subsequent purification steps. In certain embodiments, centrifugation may be carried out on cell lysates at a gravitation force from about 200 g to about 800 g, from about 500 g to about 1500 g, from about 1000 g to about 5000 g, from about 1200 g to about 10000 g or from about 8000 g to about 15000 g. In certain embodiments, cell lysate centrifugation is carried out at 8000 g for 15 minutes. In certain embodiments, density gradient centrifugation may be carried out in order to partition particulates in the cell lysate by sedimentation rate. Gradients used according to methods or systems of the present disclosure may comprise, but are not limited to, cesium chloride gradients and iodixanol step gradients. In certain embodiments, centrifugation uses a decanter centrifuge system. In certain embodiments, centrifugation uses a disc-stack centrifuge system. In certain embodiments, centrifugation comprises ultracentrifugation, such two-cycle CsCl gradient ultracentrifugation or iodixanol discontinuous density gradient ultracentrifugation.

### Clarification and Purification: Filtration

In certain embodiments, one or more microfiltration, nanofiltration and/or ultrafiltration steps may be used during clarification, purification and/or sterilization. The one or more microfiltration, nanofiltration or ultrafiltration steps can comprise the use of a filtration system such as EMD Millipore Express SHC XL 10 0.5/0.2 μm filter, EMD Millipore Express SHCXL6000 0.5/0.2 μm filter, EMD Millipore Express SHCXL150 filter, EMD Millipore Millipak Gamma Gold 0.22 μm filter (dual-in-line sterilizing grade filters), a Pall Supor EKV, 0.2 μm sterilizing-grade filter, Asahi Planova 35N, Asahi Planova 20N, Asahi Planova 75N, Asahi Planova BioEx, Millipore Viresolve NFR or a Sartorius Sartopore 2XLG, 0.8/0.2 μm.

In certain embodiments, one or more microfiltration steps may be used during clarification, purification and/or sterilization. Microfiltration utilizes microfiltration membranes with pore sizes typically between 0.1 μm and 10 μm. Microfiltration is generally used for general clarification, sterilization, and removal of microparticulates. In certain embodiments, microfiltration is used to remove aggregated clumps of viral particles. In certain embodiments, a production process or system of the present disclosure comprises at least one microfiltration step. The one or more microfiltration steps can comprise a Depth Filtration step with a Depth Filtration system, such as EMD Millipore Millistak⁺ POD filter (DOHC media series), Millipore MCOSP23CL3 filter (C0SP media series), or Sartorius Sartopore filter series. Microfiltration systems of the present disclosure can be pre-rinsed, packed, equilibrated, flushed, processed. eluted, washed or cleaned with formulations known to those in the art, comprising AAV pharmaceutical, processing and storage formulations of the present disclosure. In certain embodiments, clarification comprises use of a C0SP media series filter. In some embodiments, the C0SP media series filter is effective to reduce or prevent 0.2-micron filter clogging.

In certain embodiments, one or more ultrafiltration steps may be used during clarification and purification. The ultrafiltration steps can be used for concentrating, formulating, desalting or dehydrating either processing and/or formulation solutions of the present disclosure. Ultrafiltration utilizes ultrafiltration membranes, with pore sizes typically between 0.001 and 0.1 μm. Ultrafiltration membranes can also be defined by their molecular weight cutoff (MWCO) and can have a range from 1 kD to 500 kD. Ultrafiltration is generally used for concentrating and formulating dissolved biomolecules such as proteins, peptides, plasmids, viral particles, nucleic acids, and carbohydrates. Ultrafiltration systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, one or more nanofiltration steps may be used during clarification and purification. Nanofiltration utilizes nanofiltration membranes, with pore sizes typically less than 100 nm. Nanofiltration is generally used for removal of unwanted endogenous viral impurities (e.g. baculovirus). In certain embodiments, nanofiltration can comprise viral removal filtration (VRF). VRF filters can have a filtration size typically between 15 nm and 100 nm. Examples of VRF filters comprise (but are not limited to): Planova 15N, Planova 20N, and Planova 35N (Asahi-Kasei Corp, Tokyo, Japan); and Viresolve NFP and Viresolve NFR (Millipore Corp, Billerica, Mass., USA). Nanofiltration systems of the present disclosure can be pre-rinsed, packed, equilibrated, flushed, processed, eluted, washed or cleaned with formulations known to those in the art, comprising AAV pharmaceutical, processing and storage formulations of the present disclosure. In certain embodiments, nanofiltration is used to remove aggregated clumps of viral particles.

In certain embodiments, one or more tangential flow filtration (TFF) (also known as cross-flow filtration) steps may be used during clarification and purification. Tangential flow filtration is a form of membrane filtration in which a feed stream (which comprises the target agent/particle to be clarified and concentrated) flows from a feed tank into a filtration module or cartridge. Within the TFF filtration module, the feed stream passes parallel to a membrane surface, such that one portion of the stream passes through the membrane (permeate/filtrate) while the remainder of the stream (retentate) is recirculated back through the filtration system and into the feed tank.

In certain embodiments, the TFF filtration module can be a flat plate module (stacked planar cassette), a spiral wound module (spiral-wound membrane layers), or a hollow fiber module (bundle of membrane tubes). Examples of TFF systems for use in the present disclosure comprise, but are not limited to: Spectrum mPES Hollow Fiber TFF system (0.5 mm fiber ID, 100 kDA MWCO) or Millipore Ultracel PLCTK system with Pellicon-3 cassette (0.57 m², 30 kDA MWCO).

New buffer materials can be added to the TFF feed tank as the feed stream is circulated through the TFF filtration system. In certain embodiments, buffer materials can be fully replenished as the flow stream circulates through the TFF filtration system. In this embodiment, buffer material is added to the stream in equal amounts to the buffer material lost in the permeate, resulting in a constant concentration. In certain embodiments, buffer materials can be reduced as the flow stream circulates through the filtration system. In this embodiment, a reduced amount of buffer material is added to the stream relative to the buffer material lost in the permeate, resulting in an increased concentration. In certain embodiments, buffer materials can be replaced as the flow stream circulates through the filtration system. In this embodiment, the buffer added to stream is different from buffer materials lost in the permeate, resulting in an eventual replacement of buffer material in the stream. TFF systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, a TFF load pool can be spiked with an excipient or diluent prior to filtration. In certain embodiments, a TFF load pool is spiked with a high-salt mixture (such as sodium chloride or potassium chloride) prior to filtration. In certain embodiments, a TFF load pool is spiked with a high-sugar mixture (such as 50% w/v sucrose) prior to filtration.

The effectiveness of TFF processing can depend on several factors, comprising (but not limited to): shear stress from flow design, cross-flow rate, filtrate flow control. transmembrane pressure (TMP), membrane conditioning, membrane composition (e.g. hollow fiber construction) and design (e.g. surface area), system flow design, reservoir design, and mixing strategy. In certain embodiment, the filtration membrane can be exposed to pre-TFF membrane conditioning.

In certain embodiments, TFF processing can comprise one or more microfiltration stages. In certain embodiments, TFF processing can comprise one or more ultrafiltration stages. In certain embodiments, TFF processing can comprise one or more nanofiltration stages.

In certain embodiments, TFF processing can comprise one or more concentration stages, such as an ultrafiltration (UF) or microfiltration (MF) concentration stage. In the concentration stage, a reduced amount of buffer material is replaced as the stream circulates through the filtration system (relative to the amount of buffer material lost as permeate). The failure to completely replace all of the buffer material lost in the permeate results in an increased concentration of viral particles within the filtration stream. In certain embodiments, an increased amount of buffer material is replaced as the stream circulates through the filtration system. The incorporation of excess buffer material relative to the amount of buffer material lost in the permeate results in a decreased concentration of viral particles within the filtration stream.

In certain embodiments, TFF processing can comprise one or more diafiltration (DF) stages. The diafiltration stage comprises replacement of a first buffer material (such as a high salt material) within a second buffer material (such a low-salt or zero-salt material). In this embodiment, a second buffer is added to flow stream which is different from a first buffer material lost in the permeate, resulting in an eventual replacement of buffer material in the stream.

In certain embodiments, TFF processing can comprise multiple stages in series. In certain embodiments, a TFF processing process can comprise an ultrafiltration (UF) concentration stage followed by a diafiltration stage (DF). In certain embodiments, TFF comprising UF followed by DF results in increased rAAV recovery relative to TFF comprising DF followed by UF.

In certain embodiments, a TFF processing can comprise a diafiltration stage followed by an ultrafiltration concentration stage. In certain embodiments, a TFF processing can comprise a first diafiltration stage, followed by an ultrafiltration concentration stage, followed by a second diafiltration stage. In certain embodiments, a TFF processing can comprise a first diafiltration stage which incorporates a high-salt-low-sugar buffer material into the flow stream, followed by an ultrafiltration/concentration stage which results in a high concentration of the viral material in the flow stream, followed by a second diafiltration stage which incorporates a low-salt-high-sugar or zero-salt-high-sugar buffer material into the flow stream. In certain embodiments, the salt can be sodium chloride, sodium phosphate, potassium chloride, potassium phosphate, or a combination thereof. In certain embodiments, the sugar can be sucrose, such as a 5% w/v sucrose mixture or a 7% w/v sucrose mixture.

In certain embodiments, the one or more TFF steps can comprise a formulation diafiltration step in which at least a portion of the liquid media of the viral production pool is replaced with a high-sucrose formulation buffer. In certain embodiments, the high-sucrose formulation buffer comprises between 6-8% w/v of a sugar or sugar substitute and between 90-100 mM of an alkali chloride salt. In certain embodiments, the high-sucrose formulation buffer comprises 7% w/v of sucrose and between 90-100 mM sodium chloride. In certain embodiments, the high-sucrose formulation buffer comprises 7% w/v of sucrose, 10 mM Sodium Phosphate, between 95-100 mM sodium chloride, and 0.001% (w/v) Poloxamer 188. In certain embodiments, the formulation diafiltration step is the final diafiltration step in the one or more TFF steps. In certain embodiments, the formulation diafiltration step is the only diafiltration step in the one or more TFF steps.

In certain embodiments, TFF processing can comprise multiple stages which occur contemporaneously. As a non-limiting example, a TFF clarification process can comprise an ultrafiltration stage which occurs contemporaneously with a concentration stage.

Methods of cell lysate clarification and purification by filtration are well understood in the art and may be carried out according to a variety of available methods comprising, but not limited to passive filtration and flow filtration. Filters used may comprise a variety of materials and pore sizes. For example, cell lysate filters may comprise pore sizes of from about 1 μM to about 5 μM, from about 0.5 μM to about 2 μM, from about 0.1 μM to about 1 μM, from about 0.05 μM to about 0.05 μM and from about 0.001 μM to about 0.1 μM. Exemplary pore sizes for cell lysate filters may comprise, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In certain embodiments, clarification may comprise filtration through a filter with 2.0 μM pore size to remove large debris, followed by passage through a filter with 0.45 μM pore size to remove intact cells.

Filter materials may be composed of a variety of materials. Such materials may comprise, but are not limited to, polymeric materials and metal materials (e.g. sintered metal and pored aluminum.) Exemplary materials may comprise, but are not limited to nylon, cellulose materials (e.g. cellulose acetate), polyvinylidene fluoride (PVDF), polyethersulfone, polyamide, polysulfone, polypropylene, and polyethylene terephthalate. In certain embodiments, filters useful for clarification of cell lysates may comprise, but are not limited to ULTIPLEAT PROFILE™ filters (Pall Corporation, Port Washington, N.Y.), SUPOR™ membrane filters (Pall Corporation, Port Washington. N.Y.).

In certain embodiments, flow filtration may be carried out to increase filtration speed and/or effectiveness. In certain embodiments, flow filtration may comprise vacuum filtration. According to such methods, a vacuum is created on the side of the filter opposite that of cell lysate to be filtered. In certain embodiments, cell lysates may be passed through filters by centrifugal forces. In certain embodiments, a pump is used to force cell lysate through clarification filters. Flow rate of cell lysate through one or more filters may be modulated by adjusting one of channel size and/or fluid pressure.

### Clarification and Purification: Chromatography

In certain embodiments, AAV particles in a formulation may be clarified and purified from cell lysates through one or more chromatography steps using one or more different methods of chromatography. Chromatography refers to any number of methods known in the art for selectively separating out one or more elements from a mixture. Such methods may comprise, but are not limited to, ion exchange chromatography (e.g. cation exchange chromatography and anion exchange chromatography), affinity chromatography (e.g. immunoaffinity chromatography. metal affinity chromatography, pseudo affinity chromatography such as Blue Sepharose resins), hydrophobic interaction chromatography (HIC), size-exclusion chromatography, and multimodal chromatography (MMC) (chromatographic methods that utilize more than one form of interaction between the stationary phase and analytes). In certain embodiments, methods or systems of viral chromatography may comprise any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691. WO2000055342, WO2000075353 and WO2001023597, the contents of each of which are herein incorporated by reference in their entirety.

Chromatography systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, one or more ion exchange (IEX) chromatography steps may be used to isolate viral particles. The ion exchange step can comprise anion exchange (AEX) chromatography. cation exchange (CEX) chromatography, or a combination thereof. In certain embodiments, ion exchange chromatography is used in a bind/elute mode. Bind/elute IEX can be used by binding viral particles to a stationary phase based on charge-charge interactions between capsid proteins (or other charged components) of the viral particles and charged sites present on the stationary phase. This process can comprise the use of a column through which viral preparations (e.g. clarified lysates) are passed. After application of viral preparations to the charged stationary phase (e.g. column), bound viral particles may then be eluted from the stationary phase by applying an elution solution to disrupt the charge-charge interactions. Elution solutions may be optimized by adjusting salt concentration and/or pH to enhance recovery of bound viral particles. In certain embodiments, the elution solution can comprise a nuclease such as Benzonase nuclease. Depending on the charge of viral capsids being isolated, cation or anion exchange chromatography methods may be selected. In certain embodiments, ion exchange chromatography is used in a flow-through mode. Flow-through IEX can be used by binding non-viral impurities or unwanted viral particles to a stationary phase (based on charge-charge interactions) and allowing the target viral particles in the viral preparation to “flow through” the IEX system into a collection pool.

Methods or systems of ion exchange chromatography may comprise, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety.

In certain embodiments, the IEX process uses an AEX chromatography system such as a Sartorius Sartobind Q membrane, a Sartorius Sartobind STIC membrane, a Millipore Fractogel TMAE HiCap(m) Flow-Through membrane, a GE Q Sepharose HP membrane, Poros XQ or Poros HQ. In certain embodiments, the IEX process uses a CEX system such as a Poros XS membrane. In certain embodiments, the AEX system comprises a stationary phase which comprises a trimethylammoniumethyl (TMAE) functional group. In certain embodiments, the IEX process uses a Multimodal Chromatography (MMC) system such as a Nuvia Prime 4A membrane.

In certain embodiments, one or more affinity chromatography steps, such as immunoaffinity chromatography, may be used to isolate viral particles. Immunoaffinity chromatography is a form of chromatography that utilizes one or more immune compounds (e.g. antibodies or antibody-related structures) to retain viral particles. Immune compounds may bind specifically to one or more structures on viral particle surfaces, comprising, but not limited to one or more viral coat protein. In certain embodiments, immune compounds may be specific for a particular viral variant. In certain embodiments, immune compounds may bind to multiple viral variants. In certain embodiments, immune compounds may comprise recombinant single-chain antibodies. Such recombinant single chain antibodies may comprise those described in Smith, R. H. et al., 2009. Mol. Ther. 17(11):1888-96, the contents of which are herein incorporated by reference in their entirety. Such immune compounds (e.g., recombinant protein ligands) are capable of binding to several AAV capsid variants, comprising, but not limited to AAV1, AAV2, AAV3, AAV5, AAV6 and/or AAV8 or any of those taught herein. In some embodiments, such immune compounds (e.g., recombinant protein ligands) are capable of binding to at least AAV2. In certain embodiments, the AFC process uses a GE AVB Sepharose HP column resin, Poros CaptureSelect AAV8 resins (ThermoFisher), Poros CaptureSelect AAV9 resins (ThermoFisher) and Poros CaptureSelect AAVX resins (ThermoFisher).

In certain embodiments, one or more affinity chromatography steps precedes one or more anion exchange chromatography steps. In certain embodiments, one or more anion exchange chromatography steps precedes one or more affinity chromatography steps.

In certain embodiments, one or more size-exclusion chromatography (SEC) steps may be used to isolate viral particles. SEC may comprise the use of a gel to separate particles according to size. In viral particle purification, SEC filtration is sometimes referred to as “polishing.” In certain embodiments, SEC may be carried out to generate a final product that is near-homogenous. Such final products may in certain embodiments be used in pre-clinical studies and/or clinical studies (Kotin, R. M. 2011. Human Molecular Genetics. 20(1):R2-R6, the contents of which are herein incorporated by reference in their entirety.) In certain embodiments, SEC may be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

### III. Definitions

At various places in the present disclosure, substituents or properties of compounds of the present disclosure are disclosed in groups or in ranges. It is specifically intended that the present disclosure comprise each and every individual or sub-combination of the members of such groups and ranges.

Unless stated otherwise, the following terms and phrases have the meanings described below. The definitions are not meant to be limiting in nature and serve to provide a clearer understanding of certain aspects of the present disclosure.

Adeno-associated virus: The term “adeno-associated virus” or “AAV” as used herein refers to members of the dependovirus genus comprising any particle, sequence, gene, protein, or component derived therefrom.

AAV Particle: As used herein, an “AAV particle” is a virus which comprises a capsid and a viral genome with at least one payload region and at least one ITR region. AAV particle may be derived from any serotype, described herein or known in the art, comprising combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions of the present disclosure may have activity and this activity may involve one or more biological events.

Administering: As used herein, the term “administering” refers to providing a pharmaceutical agent or composition to a subject.

Amelioration: As used herein, the term “amelioration” or “ameliorating” refers to a lessening of severity of at least one indicator of a condition or disease. For example, in the context of neurodegeneration disorder, amelioration comprises the reduction of neuron loss.

Animal: As used herein, the term “animal” refers to any member of the animal kingdom. In certain embodiments, “animal” refers to humans at any stage of development. In certain embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In certain embodiments, animals comprise, but are not limited to, mammals, birds, reptiles, amphibians, fish, and worms. In certain embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. The term may refer to +/−10% of the recited value. In certain embodiments, the term refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked.” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Baculoviral expression vector (BEV): As used herein a BEV is a baculoviral expression vector, i.e., a polynucleotide vector of baculoviral origin. A baculovirus expression vector (BEV) is a recombinant baculovirus that has been genetically modified to lead the expression of a foreign gene. Systems using BEVs are known as baculoviral expression vector systems (BEVSs).

mBEV or modified BEV: As used herein, a modified BEV is an expression vector of baculoviral origin which has been altered from a starting BEV (whether wild type or artificial) by the addition and/or deletion and/or duplication and/or inversion of one or more: genes; gene fragments; cleavage sites; restriction sites; sequence regions; sequence(s) encoding a payload or gene of interest; or combinations of the foregoing.

BIIC: As used herein a BIIC is a baculoviral infected insect cell.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, an AAV particle of the present disclosure may be considered biologically active if even a portion of the encoded payload is biologically active or mimics an activity considered biologically relevant.

Capsid: As used herein, the term “capsid” refers to the protein shell of a virus particle.

Codon optimized: As used herein, the terms “codon optimized” or “codon optimization” refers to a modified nucleic acid sequence which encodes the same amino acid sequence as a parent/reference sequence, but which has been altered such that the codons of the modified nucleic acid sequence are optimized or improved for expression in a particular system (such as a particular species or group of species). As a non-limiting example, a nucleic acid sequence which comprises an AAV capsid protein can be codon optimized for expression in insect cells or in a particular insect cell such Spodoptera frugiperda cells. Codon optimization can be completed using methods and databases known to those in the art.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity.

Compound: Compounds of the present disclosure comprise all of the isotopes of the atoms occurring in the intermediate or final compounds. “Isotopes” refers to atoms having the same atomic number but different mass numbers resulting from a different number of neutrons in the nuclei. For example, isotopes of hydrogen comprise tritium and deuterium.

The compounds and salts of the present disclosure can be prepared in combination with solvent or water molecules to form solvates and hydrates by routine methods.

Conserved: As used herein, the term “conserved” refers to nucleotides or amino acid residues of a polynucleotide sequence or polypeptide sequence. respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In certain embodiments, two or more sequences are said to be “completely conserved” if they are 100% identical to one another. In certain embodiments, two or more sequences are said to be “highly conserved” if they are at least 70% identical, at least 80% identical, at least 90% identical, or at least 95% identical to one another. In certain embodiments, two or more sequences are said to be “highly conserved” if they are about 70% identical, about 80% identical, about 90% identical, about 95%, about 98%, or about 99% identical to one another. In certain embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In certain embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Delivery: As used herein, “delivery” refers to the act or manner of delivering an AAV particle, a compound, substance, entity, moiety, cargo or payload.

Delivery Agent: As used herein, “delivery agent” refers to any substance which facilitates, at least in part, the in vivo delivery of an AAV particle to targeted cells.

Destabilized: As used herein, the term “destabilize,” or “destabilizing region” means a region or molecule that is less stable than a starting, wild-type or native form of the same region or molecule.

Digest: As used herein, the term “digest” means to break apart into smaller pieces or components. When referring to polypeptides or proteins, digestion results in the production of peptides.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments of the present disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

ExpressionBac: As used herein, “expressionBac” or “rep/cap bac” refers to a baculovirus comprising an adeno-associated virus (AAV) viral expression construct and/or region. In some embodiments, the viral expression construct of the expressionBac comprises one or more polynucleotides encoding capsid and/or replication genes for an AAV, such as but not limited to AAV2. For example, the one or more polynucleotides encoding capsid and/or replication genes for an AAV may encode VP1, VP2, VP3, Rep52, and/or Rep78, and these polynucleotides may be present in the construct in one or more open reading frames, e.g., in two open reading frames.

Expression BIIC: As used herein, “expression BIIC” or “rep/cap BIIC” refers to an insect cell comprising one or more baculoviruses (e.g. expressionBac) which comprise bacmids comprising a viral expression construct. In some embodiments, the expression construct comprises one or more polynucleotides encoding capsid and/or replication genes for an AAV, such as but not limited to AAV2. For example, the one or more polynucleotides encoding capsid and/or replication genes for an AAV may encode VP1, VP2, VP3, Rep52, and/or Rep78, and these polynucleotides may be present in the construct in one or more open reading frames, e.g., in two open reading frames. In some embodiments, the insect cell is an Sf9 cell.

Feature: As used herein, a “feature” refers to a characteristic, a property, or a distinctive element.

Formulation: As used herein, a “formulation” comprises at least one AAV particle and a delivery agent or excipient.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In certain embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% for at least one stretch of at least about 20 amino acids. In certain embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology. Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of which are each incorporated herein by reference in their entireties, insofar as they do not conflict with the present disclosure. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences comprise, but are not limited to those disclosed in Carillo, H., and Lipman, D., SIAM J Applied Math., 48:1073 (1988); the contents of which are each incorporated herein by reference in their entireties, insofar as they does not conflict with the present disclosure. Techniques for determining identity are codified in publicly available computer programs. Exemplary computer software to determine homology between two sequences comprise, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In certain embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, the term “substantially isolated” is meant that a substance is substantially separated from the environment in which it was formed or detected. Partial separation can comprise, for example, a composition enriched in the substance or AAV particles of the present disclosure. Substantial separation can comprise compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein “linker” refers to a molecule or group of molecules which connects two molecules. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule of the present disclosure. Molecules may be modified in many ways comprising chemically, structurally, and functionally. As used herein, embodiments of the disclosure are “modified” when they have or possess a feature or property, whether structural or chemical, that varies from a starting point, wild type or native molecule.

Mutation: As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes.

Naturally Occurring: As used herein, “naturally occurring” or “wild-type” means existing in nature without artificial aid, or involvement of the hand of man.

Non-human animal: As used herein, a “non-human animal” comprises all animals except Homo sapiens (e.g. non-human vertebrates), comprising wild and domesticated species. Examples of non-human vertebrates comprise, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak. Non-human animals include non-human primates.

Nucleic Acid: As used herein, the term “nucleic acid”, “polynucleotide” and ‘oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms comprise double- and single-stranded DNA, as well as double- and single stranded RNA.

Open reading frame: As used herein, “open reading frame” or “ORF” refers to a sequence which does not contain a stop codon within the given reading frame, other than at the end of the reading frame.

Operably linked: As used herein, the phrase “operably linked” refers to a functional connection between two or more molecules, constructs, transcripts, entities, moieties or the like. As a non-limiting example, a promoter is “operably linked” to a nucleotide sequence when the promoter sequence controls and/or regulates the transcription of the nucleotide sequence.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, or a modulatory nucleic acid or regulatory nucleic acid.

PayloadBac: As used herein, “payloadBac” refers to a baculovirus comprising a payload construct and/or region. In some embodiments, the payload construct and/or region of the payloadBac comprises a polynucleotide encoding the payload.

Payload BIC: As used herein, “payloadBIIC” refers to an insect cell comprising one or more baculovirus (e.g. payloadBac) comprising a payload construct and/or region. In some embodiments, the payload construct and/or region comprises a polynucleotide encoding the payload. In some embodiments, the insect cell is an Sf9 cell.

Payload construct: As used herein, “payload construct” is one or more vector construct which comprises a polynucleotide region encoding or comprising a payload that is flanked on one or both sides by an inverted terminal repeat (ITR) sequence. The payload construct may present a template that is replicated in a viral production cell to produce a therapeutic viral genome.

Payload construct vector: As used herein, “payload construct vector” is a vector encoding or comprising a payload construct, and regulatory regions for replication and expression of the payload construct in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may comprise, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients comprise, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also comprises pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts comprise, but are not limited to, mineral or organic acid salts of basic residues such as amines; alkali or organic salts of acidic residues such as carboxylic acids; and the like. Representative acid addition salts comprise acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate. laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts comprise sodium, lithium, potassium, calcium, magnesium, and the like, as well as nontoxic ammonium, quaternary ammonium, and amine cations, comprising, but not limited to ammonium, tetramethylammonium, tetraethylammonium, methylamine, dimethylamine, trimethylamine, triethylamine, ethylamine, and the like. The pharmaceutically acceptable salts of the present disclosure comprise the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile can be used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), the contents of which are each incorporated herein by reference in their entireties, insofar as they do not conflict with the present disclosure.

Pharmaceutically acceptable solvate: The term “pharmaceutically acceptable solvate,” as used herein, means a compound of the present disclosure wherein molecules of a suitable solvent are incorporated in the crystal lattice. A suitable solvent is physiologically tolerable at the dosage administered. For example, solvates may be prepared by crystallization, recrystallization, or precipitation from a solution that comprises organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Polypeptide: As used herein, “polypeptide” refers to a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Protein of interest: As used herein, the terms “proteins of interest” or “desired proteins” comprise those provided herein and fragments, mutants, variants, and alterations thereof.

Purified: As used herein, “purify,” “purified.” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In certain embodiments, when referring to a protein or protein module, a region may comprise a linear sequence of amino acids along the protein or protein module or may comprise a three-dimensional area, an epitope and/or a cluster of epitopes. In certain embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In certain embodiments. N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In certain embodiments, N-terminal regions may comprise any length of amino acids that comprises the N-terminus but does not comprise the C-terminus. In certain embodiments, C-terminal regions may comprise any length of amino acids, which comprise the C-terminus, but do not comprise the N-terminus.

In certain embodiments, when referring to a polynucleotide, a region may comprise a linear sequence of nucleic acids along the polynucleotide or may comprise a three-dimensional area, secondary structure, or tertiary structure. In certain embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In certain embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In certain embodiments, 5′ regions may comprise any length of nucleic acids that comprises the 5′ terminus but does not comprise the 3′ terminus. In certain embodiments, 3′ regions may comprise any length of nucleic acids, which comprise the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, comprising plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

Sample: As used herein, the term “sample” or “biological sample” refers to a subset of its tissues, cells or component parts (e.g. body fluids, comprising but not limited to blood, mucus, lymphatic fluid, synovial fluid, cerebrospinal fluid, saliva, amniotic fluid, amniotic cord blood, urine, vaginal fluid and semen). A sample further may comprise a homogenate, lysate or extract prepared from a whole organism or a subset of its tissues, cells or component parts, or a fraction or portion thereof, comprising but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle comprised of at least two components, a protein capsid and a polynucleotide sequence encoding a self-complementary genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” comprises a siRNA strand having sufficient complementarity to a section of about 10-50 nucleotides of the mRNA of the gene targeted for silencing and a siRNA strand having sufficient complementarity to form a duplex with the other siRNA strand.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. In certain embodiments, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, such as 21 nucleotides (or nucleotide analogs), for example, 19, 20, 21 or 22 nucleotides. The term “long” siRNA refers to a siRNA comprising 24-60 nucleotides, such as about 24-25 nucleotides, for example, 23, 24, 25 or 26 nucleotides. Short siRNAs may, in some instances. comprise fewer than 19 nucleotides, e.g., 16, 17 or 18 nucleotides, or as few as 5 nucleotides, provided that the shorter siRNA retains the ability to mediate RNAi. Likewise, long siRNAs may, in some instances, comprise more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Signal Sequences: As used herein, the phrase “signal sequences” refers to a sequence which can direct the transport or localization of a protein.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Stable: As used herein “stable” refers to a compound that is sufficiently robust to survive isolation to a useful degree of purity from a reaction mixture, and in certain embodiments, capable of formulation into an efficacious therapeutic agent.

Stabilized. As used herein, the term “stabilize”, “stabilized.” “stabilized region” means to make or become stable.

Subject: As used herein, the term “subject” or “patient” refers to any organism to which a composition in accordance with the present disclosure may be administered, e.g., for experimental, diagnostic, prophylactic, and/or therapeutic purposes. Typical subjects comprise animals (e.g., mammals such as mice, rats, rabbits, non-human primates, and humans) and/or plants. The subject may seek or need treatment, require treatment, is receiving treatment, will receive treatment, or is under care by a trained professional for a particular disease or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In certain embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease. disorder, and/or condition; and (6) exposure to and/or infection with a microbe associated with development of the disease, disorder, and/or condition. In certain embodiments, an individual who is susceptible to a disease, disorder, and/or condition will develop the disease, disorder, and/or condition. In certain embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, such as a mammal, a human, or a human patient.

Terminal region: As used herein, the term “terminal region” refers to a region on the 5′ or 3′ end of a region of linked nucleosides or amino acids (polynucleotide or polypeptide, respectively).

Terminally optimized: The term “terminally optimized” when referring to nucleic acids means the terminal regions of the nucleic acid are improved in some way, e.g., codon optimized, over the native or wild type terminal regions.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In certain embodiments, a therapeutically effective amount is provided in a single dose. In certain embodiments, a therapeutically effective amount is administered in a dosage regimen comprising a plurality of doses. Those skilled in the art will appreciate that in certain embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Transfection: As used herein, the term “transfection” refers to methods to introduce exogenous nucleic acids into a cell. Methods of transfection comprise, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may comprise adeno-associated virus (AAV) parent or reference sequence. Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral genome: As used herein, a “viral genome” or “vector genome” refers to the nucleic acid sequence(s) encapsulated in an AAV particle.

Those skilled in the art will recognize or be able to ascertain using no more than routine experimentation, many equivalents to the specific embodiments in accordance with the present disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any antibiotic, therapeutic or active ingredient: any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

### EXAMPLES

### Example 1. polH-NLS-LacR Polynucleotide

Polynucleotides were engineered to include a polH promoter (very late), an SV40 nuclear localization signal (NLS), and a Lac Repressor (LacR) sequence. The polh promoter was included to drive the expression of the Lac Repressor sequence. The SV40 NLS was included to improve localization of the expressed Lac Repressor proteins into the nucleus of a cell.

The start and stop positions of various component regions of the polH-NLS-LacR polynucleotide are given in Table 1.

TABLE 1
Component regions of polH-NLS-LacR Region

| | polH-NLS-LacR Region (SEQ ID NO: 5) | | | |
|---|---|---|---|---|
| | | | Length of | SEQ ID NO |
| Region | Start | Stop | Region | of region |
| polH promoter | 1 | 92 | 92 | SEQ ID NO: 6 |
| SV40 NLS region | 93 | 122 | 30 | SEQ ID NO: 7 |
| Linker | 123 | 137 | 15 | SEQ ID NO: 8 |
| LacR (Lac Repressor) | 138 | 1220 | 1083 | SEQ ID NO: 9 |
| NLS-LacR Region | 93 | 1220 | 1128 | SEQ ID NO: 2 |

The polynucleotide was engineered to be inserted/cloned into suitable baculovirus plasmids or vectors. AvrII cleavage sequences (cctagg) were included at both ends of the polynucleotide to enable cloning of the polynucleotide into the AvrII site in the non-essential egt gene ORF of a donor baculovirus plasmid (such as a variant of the AcMNPV bacmid bMON14272). Cleavage sequences for NheI (gctagc), SpeI (actagt) and BstZ17I (gtatac) were also included to enable cloning into other sites of the donor baculovirus plasmid, such as the Tn7 locus.

A polH-NLS-LacR polynucleotide (with cleavage sequences) is presented in SEQ ID NO: 10. A polH-NLS-LacR polynucleotide with only AvrII, NheI and SpeI cleavage sequences was also engineered, and is presented in SEQ ID NO: 11.

### Example 2. Opgp64-polH-NLS-LacR Polynucleotide

Polynucleotides were engineered to include both an opgp64 promoter (earl/late) and polH promoter (very late), an SV40 nuclear localization signal (NLS), and a Lac Repressor (LacR) sequence. The opgp64 and polh promoters were included to drive the expression of the Lac Repressor sequence. The SV40 NLS was included to improve localization of the expressed Lac Repressor proteins into the nucleus of a cell.

The start and stop positions of various component regions of the opgp64-polH-NLS-LacR polynucleotide are given in Table 2.

TABLE 2
Component regions of opgp64-polH-NLS-LacR Region

| | opgp64-polH-NLS-LacR Region (SEQ ID NO: 12) | | | |
|---|---|---|---|---|
| | | | Length of | SEQ ID NO |
| Region | Start | Stop | Region | of region |
| Opgp64 promoter | 1 | 166 | 166 | SEQ ID NO: 13 |
| polH promoter | 178 | 270 | 92 | SEQ ID NO: 6 |
| SV40 NLS region | 271 | 300 | 30 | SEQ ID NO: 7 |
| Linker | 301 | 315 | 15 | SEQ ID NO: 8 |
| LacI (Lac | 316 | 1398 | 1083 | SEQ ID NO: 9 |
| Repressor) | | | | |

The opgp64-polH-NLS-LacR polynucleotide was produced by ligating an opgp64 sequence onto the 5′ end of the polH-NLS-LacR polynucleotide (with cleavage sequences) from Example 1 (SEQ ID NO: 10).

The opgp64 sequence was produced by PCR amplification from a larger plasmid comprising the OpMNPV gp64 promoter. The PCR amplification used a combination of Primer JS146-AvrIIOpgp64promLP (SEQ ID NO: 14) and Primer JS147-NheIOpgp64promRP (SEQ ID NO: 15) to provide a JS146-JS147 opgp64 sequence which included AvrII (5′) and NheI (3′) cleavage sequences (SEQ ID NO: 16). Alternatively, Primer JS148-NheIOpgp64promLP (SEQ ID NO: 17) and Primer JS147 were used to provide a JS148-JS147 opgp64 sequence which included NheI cleavage sequences at both ends (SEQ ID NO: 18).

A plasmid which included the polH-NLS-LacR polynucleotide from Example 1 (SEQ ID NO: 10) was provided. The plasmid was digested using Nhei enzyme in water, and then purified. The Nhei-cut polH-NLS-LacR plasmid was then combined with the JS148-JS147 opgp64 sequence (SEQ ID NO: 18) in a mixture which included ligase buffer and T4 ligase enzyme, and then incubating at 37° C. The resulting plasmid was purified to provide a plasmid which included the opgp64-polH-NLS-LacR region (SEQ ID NO: 12).

In one alternative, a polH-NLS-LacR sequence was produced by PCR amplification from the polH-NLS-LacR polynucleotide from Example 1 (SEQ ID NO: 10). The PCR amplification used a combination of Primer JS55-polh-Mut1-NheI (SEQ ID NO: 19) and Primer JS156-NLSLacR-RP (SEQ ID NO: 20) to provide a JS155-JS156 polH-NLS-LacR sequence (1459 bp) which included NheI (5′) and AvrII (3′) cleavage sequences (SEQ ID NO: 21). The JS155-JS156 polH-NLS-LacR sequence was then combined with the JS146-JS147 opgp64 sequence (SEQ ID NO: 16) in a mixture which included ligase buffer and T4 ligase enzyme, and then incubating at 37° C. The resulting polynucleotides were purified to provide an opgp64-polH-NLS-LacR polynucleotide insert with AvrII cleavage sequences (SEQ ID NO: 22) and which included the opgp64-polH-NLS-LacR region (SEQ ID NO: 12).

The AvrII-opgp64-polH-NLS-LacR-AvrII polynucleotide insert (SEQ ID NO: 22) can be inserted into an AvrII REN access point within a target plasmid or bacmid.

### Example 3. Cloning polH-NLS-LacR Polynucleotide into AvrII Egt Locus of Bacmid

### Digestion

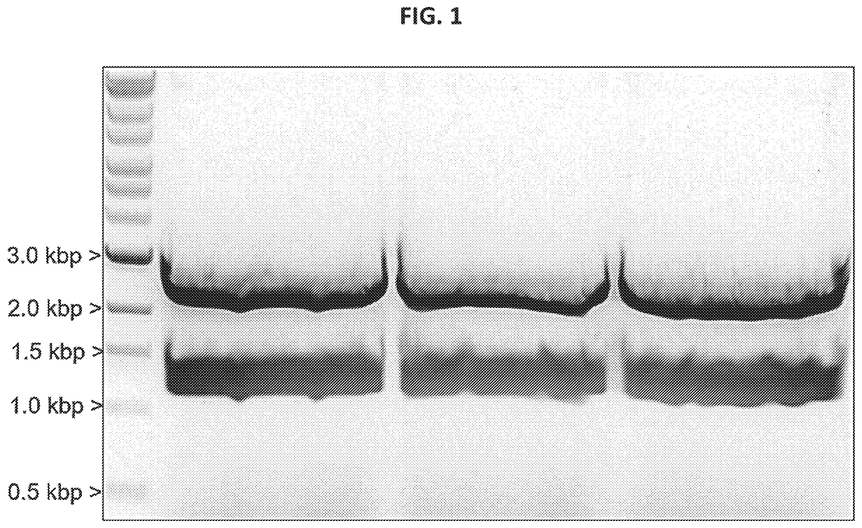

A plasmid containing the AvrII-polH-NLS-LacR-AvrII polynucleotide of Example 1 (SEQ ID NO: 11) was produced using pUC57 production plasmid vector (GenScript Biotech Corp). The polH-NLS-LacR-pUC57 (2,716 bp) was then digested overnight using 10× Cut Smart Buffer (New England Biolabs, Inc.) and AvrII enzyme (5U/μL) in water (20 ng/μL final concentration). The resulting polH-NLS-LacR insert (1,379 bp) was purified using gel purification. The process was repeated to collect additional polH-NLS-LacR insert as needed. FIG. 1 shows clear separation of the polH-NLS-LacR insert (1,379 bp) from the polH-NLS-LacR-pUC57 plasmid (2,716 bp).

A donor baculovirus plasmid (i.e. bacmid) was provided which included an AvrII egt region, such as the AcMNPV bacmid bMON14272 (Invitrogen Life Technologies) or a variant thereof. The bacmid was then digested using 10× Cut Smart Buffer (New England Biolabs, Inc.) and AvrII enzyme (5U/μL) in water (50 ng/μL final concentration) at 37° C. for 2 hours, resulting in a single-cut bacmid at the AvrII egt locus. The process was repeated to collect additional AvrII-cut bacmid as needed.

### Ligation

The AvrII-cut bacmid was ligated with the polH-NLS-LacR insert by combining 10 μL AvrII-cut bacmid (500 ng), 30 μL polH-NLS-LacR insert (600 ng), 5 μL 10×T4 ligase buffer and 2 μL T4 ligase enzyme (400U/μL), and then incubating at 37° C. for 4 hours. In one alternative, the AvrII-cut bacmid was ligated with the polH-NLS-LacR insert by combining 10 μL AvrII-cut bacmid (500 ng), 10 μL polH-NLS-LacR insert (200 ng), 25 μL H₂O, 5 μL 10×T4 ligase buffer and 2 μL T4 ligase enzyme (400U/μL), and then incubating at 37° C. for 4 hours. In one alternative, the AvrII-cut bacmid was ligated with the polH-NLS-LacR insert by combining 20 μL AvrII-cut bacmid (1000 ng), 10 μL polH-NLS-LacR insert (200 ng), 20 μL H₂O, 5.5 μL 10×T4 ligase buffer, 2.5 μL T4 ligase enzyme (400U/μL) and 2.0 μL of 10 mM ATP, and then incubating at 37° C. for 4 hours.

The resulting aqueous phase was then combined with 2 μL sodium acetate and 100-120 μL of ice-cold ethanol. Precipitated DNA pellets were collected and resuspended in 40 μL Tris-EDTA buffer. The resulting ligated plasmid DNA was then transformed into electroporated NEB 10-Beta E. coli (New England Biolabs, Inc.).

Bacterial colonies were grown and screened by colony-pick PCR to test for LacR insertion into the AvrII-cut bacmid.

### PCR Screening and Analysis

Colony PCR screening was completed using a combination of two primers: Primer 101-JS101-LP-EGT upstream (SEQ ID NO: 23) and Primer 102-JS102-RP-EGT downstream (SEQ ID NO: 24). Positive PCR results had a target of about 3111 bp based on the primers used. It was noted that the use of primer 102 resulted in an artifact amplicon fragment of about ˜2 kb which appeared in several of the PCR screening columns.

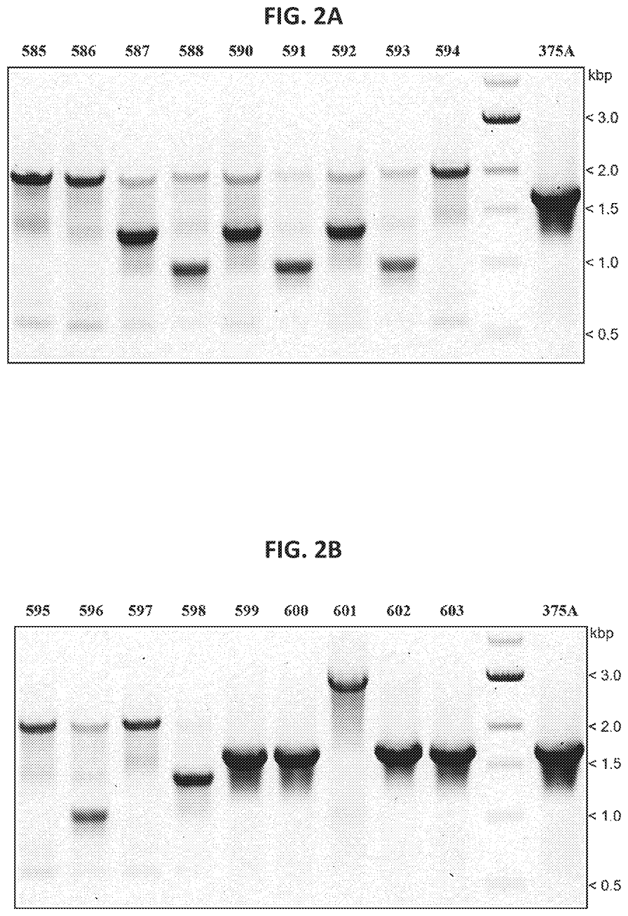

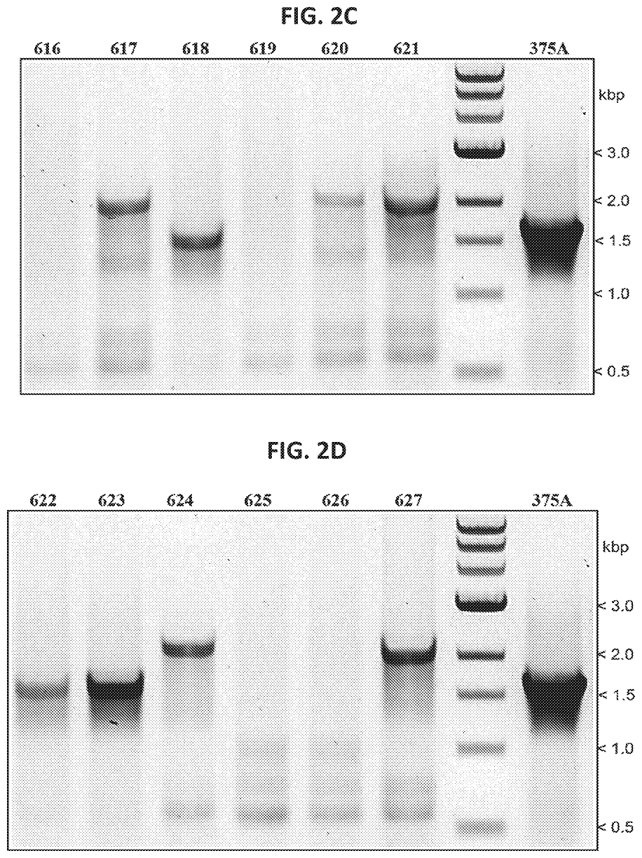

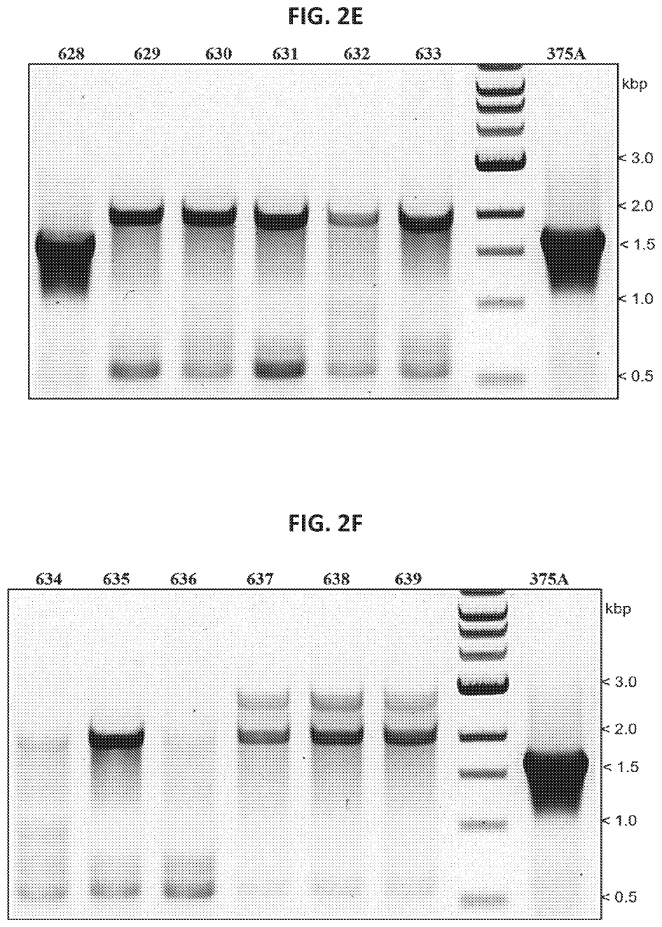

Results of PCR screening of the bacterial colonies for LacR insertion into the AvrII-cut bacmid are shown in FIGS. 2A-2F. PCR screening showed that Colony 601 (FIG. 2B) and Colonies 637-639 (FIG. 2F) had strong bands around 3111 bp. indicating possible LacR insertion into the AvrII-cut bacmids of those bacterial colonies.

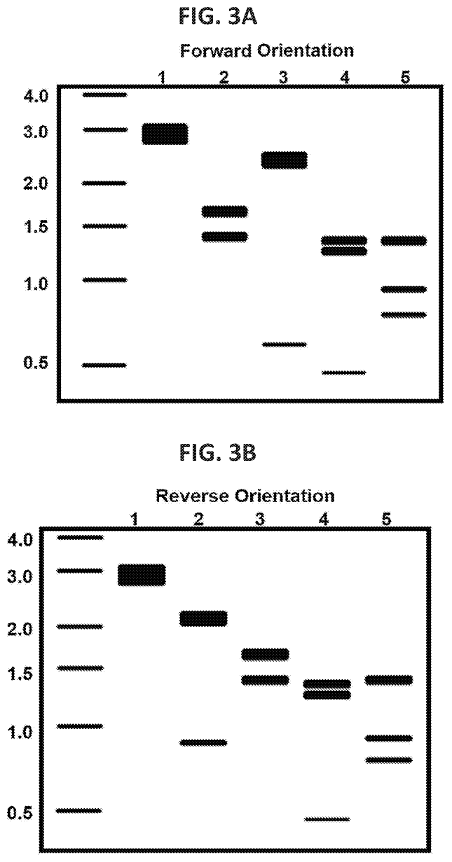

The 3111 bp JS101-JS102 PCR products for Colony 601 and Colony 639 were then gel purified and screened for LacR cassette orientation by REN digestion analysis. Forward orientation was designated as the same orientation as the Ac-egt ORF; Reverse orientation was designated as the opposite orientation as the Ac-egt ORF. REN fragments for the forward orientation of LacR insertion are shown in Table 3 and FIG. 3A; REN fragments for the reverse orientation of LacR insertion are shown in Table 3 and FIG. 3B. A noticeable difference was present between the EcoRV digest fragments (lane 2) and SmaI digest fragments (lane 3) for the two orientations.

TABLE 3
Component regions of LacO-p10-LacO Expression Control Region

| | |
|---|---|
| Forward Orientation | Reverse Orientation |

| | | | | |
|---|---|---|---|---|
| Uncut (1) | 3111 | bp | 3111 | bp |
| EcoRV digest (2) | 1413/1698 | bp | 886/2225 | bp |
| SmaI digest (3) | 600/2511 | bp | 1699/1412 | bp |
| AvrII digest (4) | 458/1379/1274 | bp | 458/1379/1274 | bp |
| BssHII digest (5) | 1374/951/786 | bp | 921/1404/786 | bp |

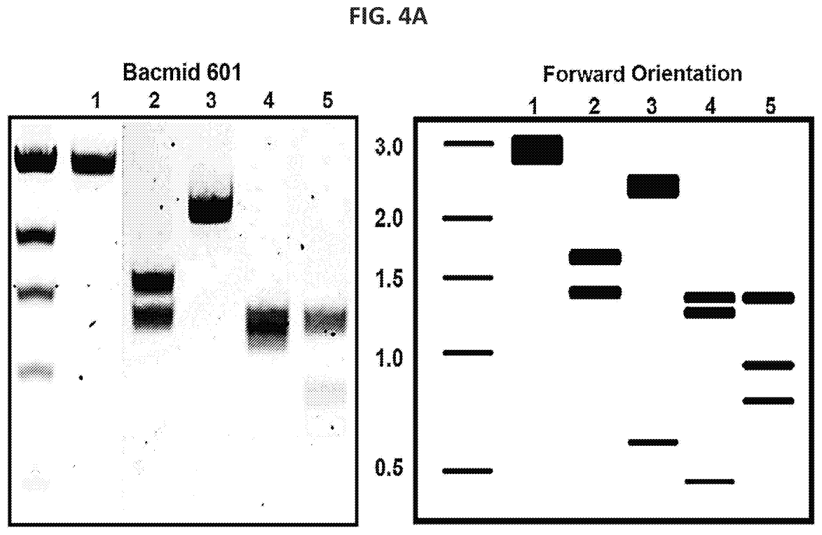

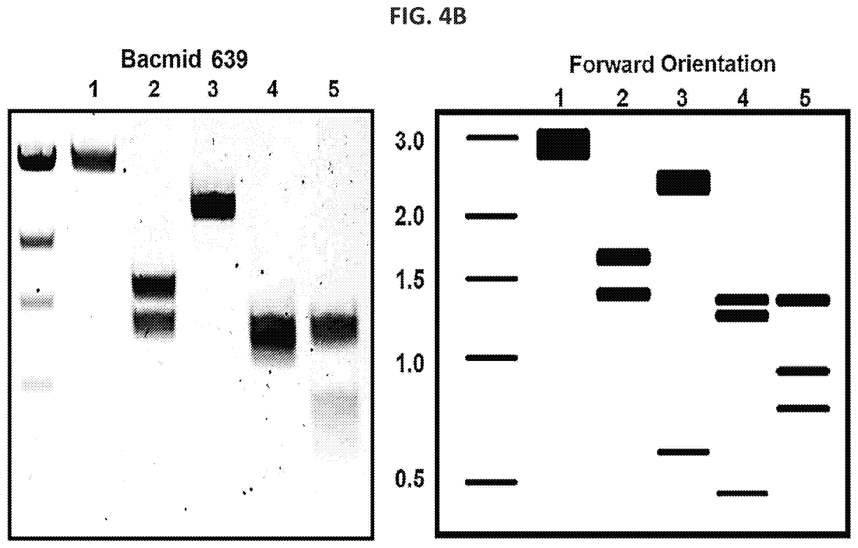

REN digestion analysis of both Colony 601 (FIG. 4A) and Colony 639 (FIG. 4B) showed that both Colony 601 and Colony 639 corresponded with the forward orientation of LacR insertion and the same orientation as the Ac-egt ORF.

### Example 4. LacO-p10-LacO-VP1-PHPN Polynucleotide

Polynucleotides were engineered to include a regulatable expression control region and a protein-coding nucleotide sequence such as a VP1 sequence. The expression control region was engineered to include a p10 promoter flanked by a Lac Operator (LacO) region on each side. A p10 promoter was included to drive the expression of a corresponding protein-coding nucleotide sequence (such as a PHPN VP1 sequence with a normal ATG start codon). Flanking LacO regions were included to allow for regulatable expression from the p10 promoter. A first bridge sequence (Ac-p26 bridge sequence) was included between the 5′ LacO region and the p10 promoter. A second bridge sequence (ctg nucleotides) was included between the p10 promoter and the 3′ LacO region. The two bridge sequences were included to provide a separation between the 5′ LacO region and the 3′ LacO region of 208 bp (measured from the center of each LacO region).

The start and stop positions of various component regions of the LacO-p10-LacO expression control region are given in Table 4.

TABLE 4
Component regions of LacO-p10-LacO Expression Control Region

| | LacO-p10-LacO Expression Control Region | | | |
|---|---|---|---|---|
| | (SEQ ID NO: 25) | | | |
| | | | Length of | SEQ ID NO |
| Region | Start | Stop | Region | of region |
| LacO 5′ | 1 | 20 | 20 | SEQ ID NO: 4 |
| Ac-p26 bridge | 21 | 95 | 75 | SEQ ID NO: 26 |
| p10 promoter | 96 | 205 | 110 | SEQ ID NO: 27 |
| LacO 3′ | 209 | 228 | 20 | SEQ ID NO: 4 |

The expression control region was then operably linked to a VP1-coding nucleotide sequence (VP1 only). The VP1 sequence for AAV.PHPN capsids was used as an exemplary construct. A small bridge sequence (ccc) was included between the expression control region and the AAV.PHPN VP1 sequence. The polynucleotide also included an HSV TK poly(A) signal.

The polynucleotide was engineered to be inserted/cloned into suitable baculovirus plasmids or vectors. I-CeuI cleavage sequences (SEQ ID NO: 1) were included to enable cloning of the polynucleotide into the I-CeuI site of a donor baculovirus plasmid. Cleavage sequences for NheI (getage), SpeI (actagt) and BstZ17I (gtatac) were also included to enable cloning into other sites of a donor baculovirus plasmid, such as the Tn7 locus. SpeI (actagt) and NcoI (ccatgg) cleavage sequences were positioned to allow for swapping-out of LacO expression control regions. NcoI (ccatgg) and MluI (acgcgt) cleavage sequences were positioned to allow for swapping-out of VP constructs (e.g. for replacing the AAV.PHPN VP1 sequence with another AAV serotype, such as AAV.PHPB, AAVrh10, AAV9 or AAV2).

A LacO-p10-LacO-VP1-PHPN polynucleotide (with VP1 only, poly(A) and cleavage sequences) is presented in SEQ ID NO: 28.

### Example 5. Cloning LacO-p10-LacO-VP1-PHPN Polynucleotide into I-CeuI Locus of Bacmid

### Digestion

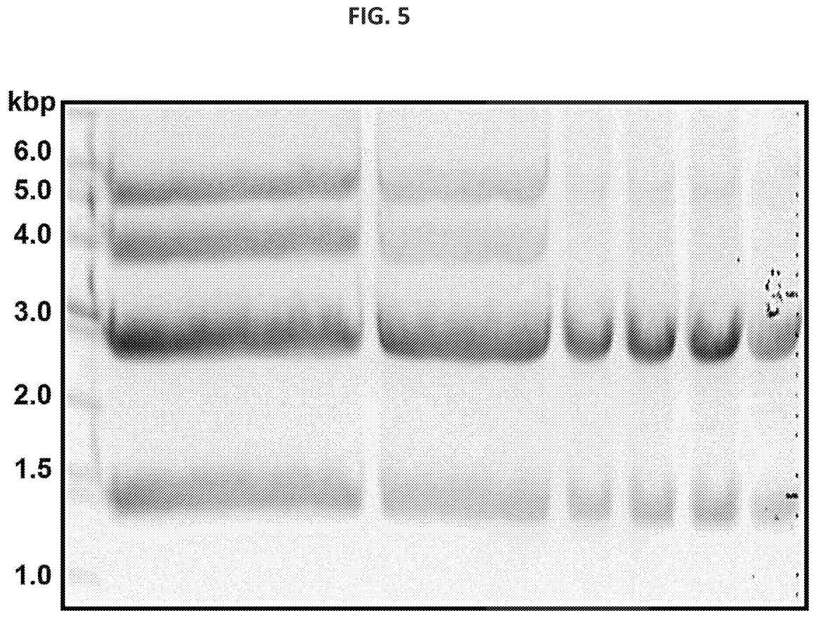

A plasmid containing the LacO-p10-LacO-VP1 polynucleotide from Example 4 (SEQ ID NO: 28) was produced using pUC57 production plasmid vector (Thermo Fisher Scientific Inc). 50 μg of LacO-p10-LacO-VP1-pUC57 was then digested using 10× Cut Smart Buffer (New England Biolabs, Inc.), I-CeuI enzyme (5 U/μL) and BsaI enzyme (20 U/μL) in water (167 ng/L final concentration) at 37° C. for 2 hours, followed by exposure to 75° C. for 10 minutes to inactivate the enzymes. The resulting LacO-p10-LacO-VP1 insert (2,679 bp) was purified using gel purification (electrophoresis in a 0.8% w/v agarose, 1× TAE gel, 80 min, 120V), with 7800 ng of recovered product. The process was repeated to collect additional LacO-p10-LacO-VP1 insert as needed. The gel presented in FIG. 5 shows clear separation of the LacO-p10-LacO-VP1 insert (2,679 bp) from remaining LacO-p10-LacO-VP1-pUC57 plasmids (5,365 bp) and pUC57 fragments (˜1300 bp after being cut in half by BsaI enzyme).

Bacmids from Colony 639 in Example 3 were provided, with each having an I-CeuI region. 6 sg of the 639 Bacmid was digested using 10× Cut Smart Buffer (New England Biolabs, Inc.) and I-CeuI enzyme (5 U/μL) in water (76 ng/μL final concentration) at 37° C. for 2 hours, followed by exposure to 75° C. for 10 minutes to inactivate the enzymes, resulting in a 639 Bacmid single-cut at the I-CeuI locus. The process was repeated to collect additional I-CeuI-cut 639 Bacmid as needed.

### Ligation

The I-CeuI-cut 639 Bacmid was ligated with the LacO-p10-LacO-VP1 insert by combining 25 μL I-CeuI-cut 639 Bacmid (600 ng), 25 μL LacO-p10-LacO-VP1 insert (1275 ng), 1 μL 100 mM ATP, 0 μL 1× Cut Smart buffer and 3 μL T4 ligase enzyme (400U/μL), and then incubating at 37° C. In one alternative, the 1-CeuI-cut 639 Bacmid was ligated with the LacO-p10-LacO-VP1 insert by combining 25 μL I-CeuI-cut 639 Bacmid (600 ng), 10 μL LacO-p10-LacO-VP1 insert (500 ng), 1 μL 100 mM ATP, 15 μL 1× Cut Smart buffer and 3 μL T4 ligase enzyme (400U/μL), and then incubating at 37° C. In one alternative, the I-CeuI-cut 639 Bacmid was ligated with the LacO-p10-LacO-VP1 insert by combining 25 μL I-CeuI-cut 639 Bacmid (600 ng), 5 μL LacO-p10-LacO-VP1 insert (250 ng), 1 μL 100 mM ATP, 20 μL 1× Cut Smart buffer and 3 μL T4 ligase enzyme (400U/μL), and then incubating at 37° C. In one alternative, the I-CeuI-cut 601 Bacmid was ligated with the LacO-p10-LacO-VP1 insert by combining 25 μL I-CeuI-cut 639 Bacmid (600 ng), 2 μL LacO-p10-LacO-VP1 insert (100 ng), 1 μL 100 mM ATP, 20 μL 1× Cut Smart buffer and 3 μL T4 ligase enzyme (400U/μL), and then incubating at 37° C.

The resulting aqueous phases were then mixed with 2 μL 3M sodium acetate and 2 volumes of ice-cold ethanol, then chilled at −20° C. for 20 minutes. Precipitated DNA pellets were collected by centrifuge and resuspended in 80 μL Tris-EDTA buffer. The resulting ligated plasmid DNA was then transformed into electroporated NEB 10-Beta E. coli (New England Biolabs, Inc.).

Bacterial colonies were grown and screened by colony-pick PCR to test for LacO-p10-LacO-VP1 insertion into the I-CeuI-cut 601 Bacmid.

### PCR Screening and Analysis

Colony PCR screening was completed using a combination of four primers: Primer JS16-Lef7-LP1 (SEQ ID NO: 29), Primer JS17-gp64UTR-RP (SEQ ID NO: 30), Primer JS61-VP3-primer2 (SEQ ID NO: 31) and Primer JS92-AAP-RP1 (SEQ ID NO: 32). Positive PCR results had a target of about 3838 bp (JS16-JS17), 1398 bp (JS16-JS61) or 1092 bp (JS92-JS17) based on the primers used.

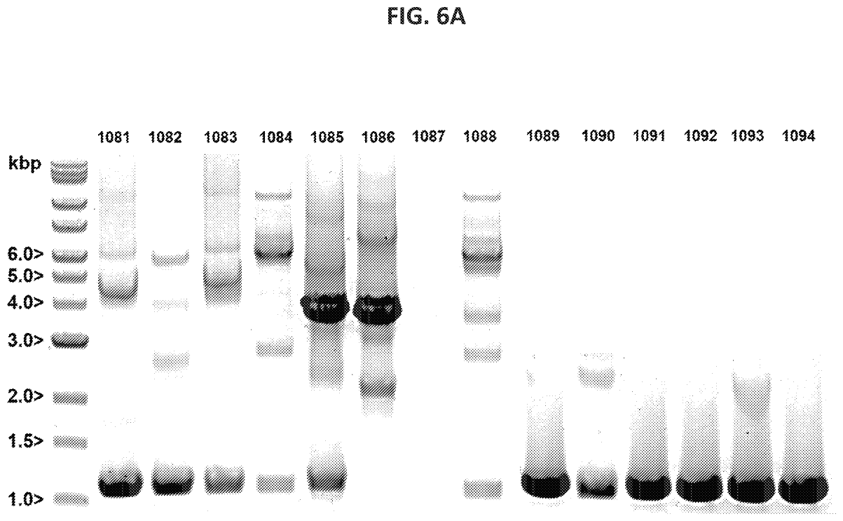

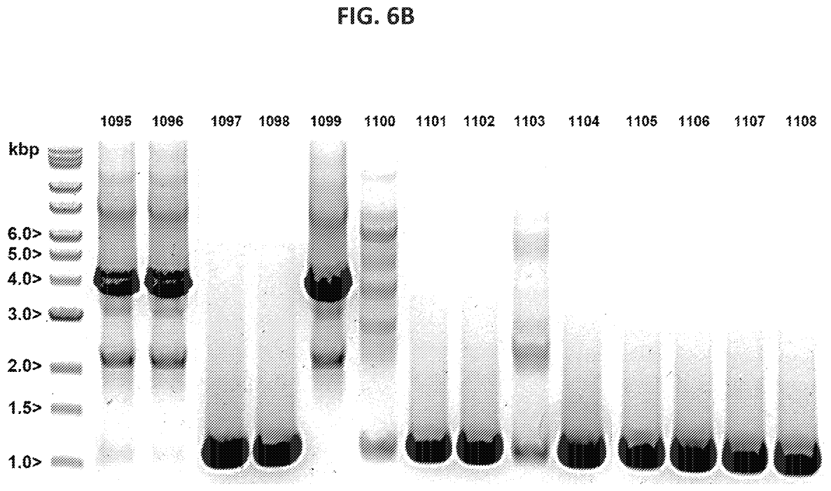

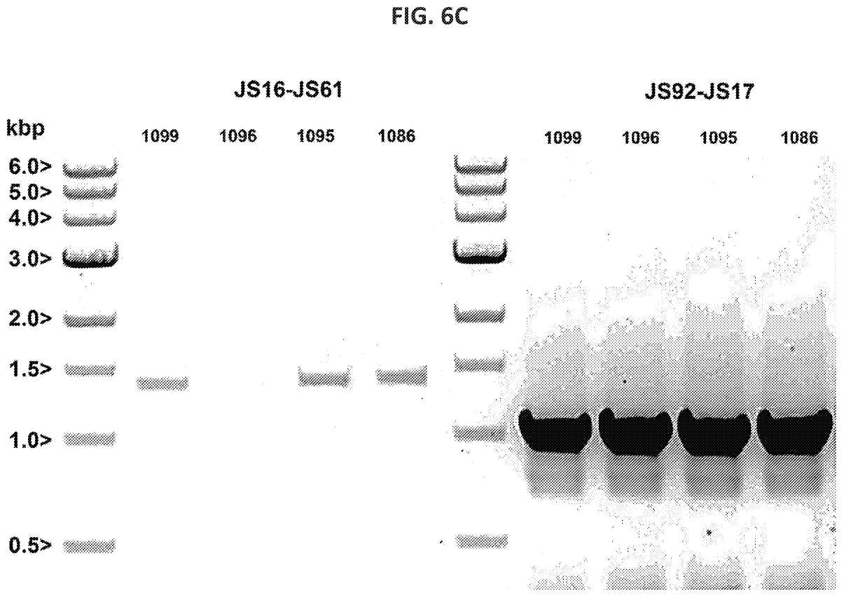

Results of JS16-JS17 PCR screening of the bacterial colonies for LacO-p10-LacO-VP1 insertion into the I-CeuI-cut 639 Bacmid are shown in FIGS. 6A-6C. PCR screening showed that Colonies 1085-1086 (FIG. 6A), Colonies 1095-1096 (FIG. 6B) and Colony 1099 (FIG. 6B) had strong bands around 3838 bp, indicating likely insertion of LacO-p10-LacO-VP1 insertion into the I-CeuI-cut 639 Bacmid of those bacterial colonies. Strong bands for the remaining colonies around 1159 bp correlated with empty I-CeuI sites in the 1-CeuI-cut 639 Bacmid.

Results of JS16-JS61 and JS92-JS17 PCR screening of Colonies 1086, 1095, 1096 and 1099 for LacO-p10-LacO-VP1 insertion into the I-CeuI-cut 639 Bacmid are shown in FIG. 6C. PCR screening showed that Colonies 1086, 1095 and 1099 had strong bands around 1398 bp and 1092 bp, indicating likely insertion of LacO-p10-LacO-VP1 insertion into the I-CeuI-cut 639 Bacmids. Colony 1096 had a strong band around 1398 bp but no discernable band around 1092 bp.

### Western Blot Testing

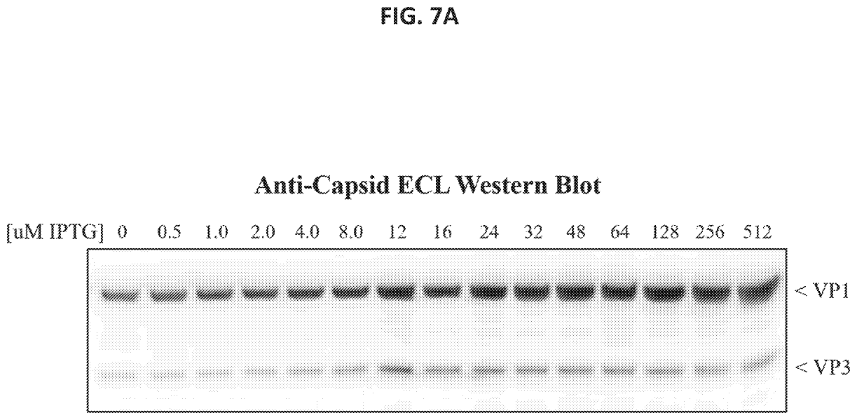

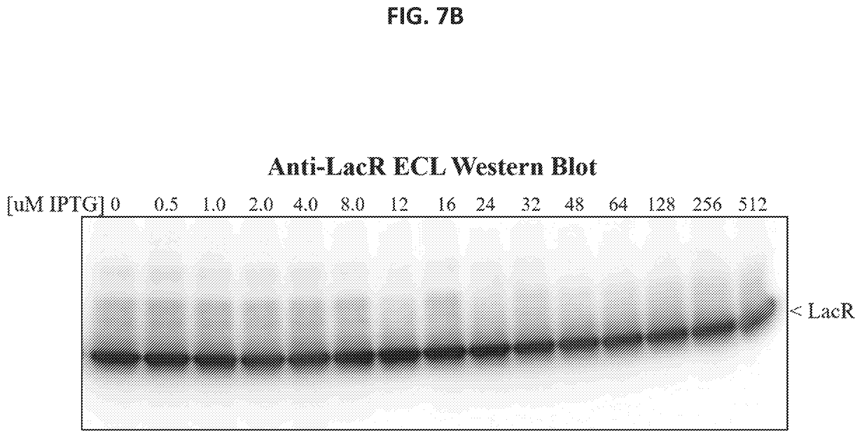

Colony 1095 was tested using Anti-AAV Capsid ECL Western Blot and Anti-LacR ECL Western Blot, with isopropyl-β-D-thiogalactose (IPTG) being used as the inducer element. Bacmids from Colony 1095 were infected into Sf9 cells under different IPTG concentrations, and the total cell lysates at 3 days post infection were analyzed using Western Blot. Results are shown in FIG. 7A and FIG. 7B.

Results in FIG. 7A show that VP1 production was being regulated by the LacR, with a lower concentration of IPTG resulting in lower VP1 production and a higher concentration of IPTG resulting in higher VP1 production.

### Example 6. Producing PHPN-LacOVP1^{ICeu}-LacR^{AvrII} Bacmid

A donor plasmid containing an AAV viral expression construct was provided. The viral expression construct included an AAV Rep sequence (encoding Rep78 and Rep52 proteins) under a polH promoter, and an AAVPHPN Cap sequence (encoding VP1, VP2 and VP3) under a p10 promoter.

Bacmid 1095 from Example 5 was provided. The AAV viral expression construct from the donor plasmid was inserted into Bacmid 1095 by Tn7 transposition, using methods and helper plasmids known to those in the art. Bacterial colonies (including Colony 1140) were grown and screened by colony-pick PCR to test for insertion of the AAV viral expression construct into the Bacmid 1095 to produce PHPN-LacOVP1^{ICeu}-LacR^{AvrII} bacmids.

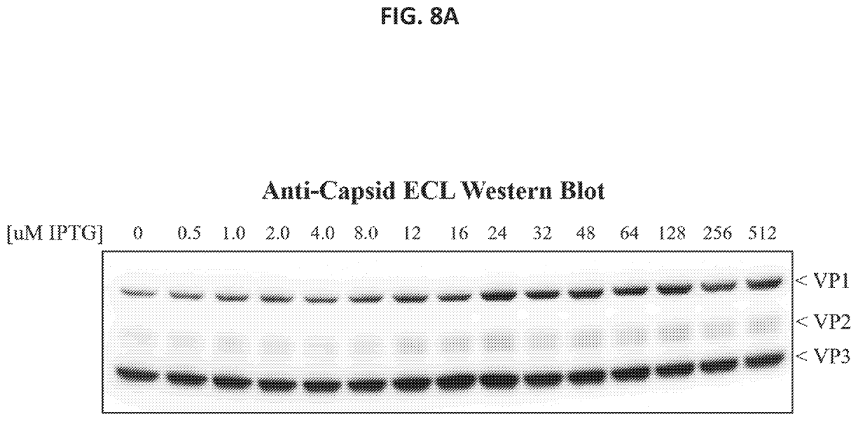

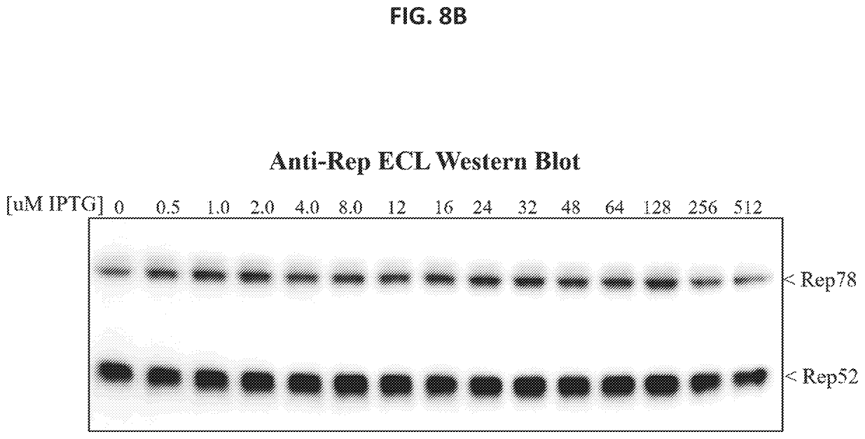

Colony 1140 was tested using Anti-AAV Capsid ECL Western Blot and Anti-LacR ECL Western Blot, with isopropyl-β-D-thiogalactose (IPTG) being used as the inducer element. Bacmids from Colony 1140 were infected into Sf9 cells under different IPTG concentrations, and the total cell lysates at 3 days post infection were analyzed using Western Blot. Results are shown in FIG. 8A and FIG. 8B.

Results in FIG. 8A show that VP1 production was being regulated by the LacR, with a lower concentration of IPTG resulting in lower VP1 production and a higher concentration of IPTG resulting in higher VP1 production. Concentrations of VP2 and VP3 were consistent (FIG. 8A). Concentrations of Rep78 and Rep52 were also consistent (FIG. 8B).

### Example 7. Cloning LacO-p10-LacO-VP1-PHPN Polynucleotide into FseI Locus of Bacmid

### PCR Amplification and Digestion

A plasmid containing the full LacO-p10-LacO-VP1 polynucleotide from Example 4 (SEQ ID NO: 28) was produced using pUC57 production plasmid vector (Thermo Fisher Scientific Inc). The resulting LacO-p10-LacO-VP1-pUC57 plasmid included both an 1-CeuI locus (from the LacO-p10-LacO-VP1 polynucleotide) and a FseI locus (from the pUC57 plasmid).

PCR amplification from the LacO-p10-LacO-VP1-pUC57 plasmid was used to produce LacO-p10-LacO-VP1-FseI inserts, using a combination of two primers: Primer JS134-FseI (SEQ ID NO: 33) and Primer JS135-FseI (SEQ ID NO: 34). The process yielded approximately 32 μg of LacO-p10-LacO-VP1-FseI insert material (target of 3,541 bp).

The PCR product was purified using QiaQuick PCR purification kit with four 180 μL volumes each including 8 μg of LacO-p10-LacO-VP1-FseI insert. 900 μL PB buffer was added to each volume, followed by 20 μL 3M NaAcetate pH 5.5. Product was then centrifuged and processed through four QiaQuick columns, washed with 750 μL of PB buffer, and then eluted with 60 μL TE buffer (1st elution) and 10 μL water (2nd elution). Final volume pool was 270 μl with a 32 μg yield of LacO-p10-LacO-VP1-FseI insert.

The 270 μl product pool was then digested using 30 μL 10× Cut Smart buffer and 10 μL FseI enzyme (2U/μL) at 37° C. for 3 hours. Purification using the QiaQuick PCR purification kit was repeated for the digestion product using 75 μL FseI REN digest, 375 μL PB buffer and 20 μL 3M NaAcetate. Product was centrifuged and processed through four QiaQuick columns, washed with 750 μL of PB buffer, and then eluted with 20 μL TE buffer (1st elution) and 20 μL water (2nd elution). Final volume pool was 160 μl with a 29.3 μg yield of LacO-p10-LacO-VP1-FseI insert.

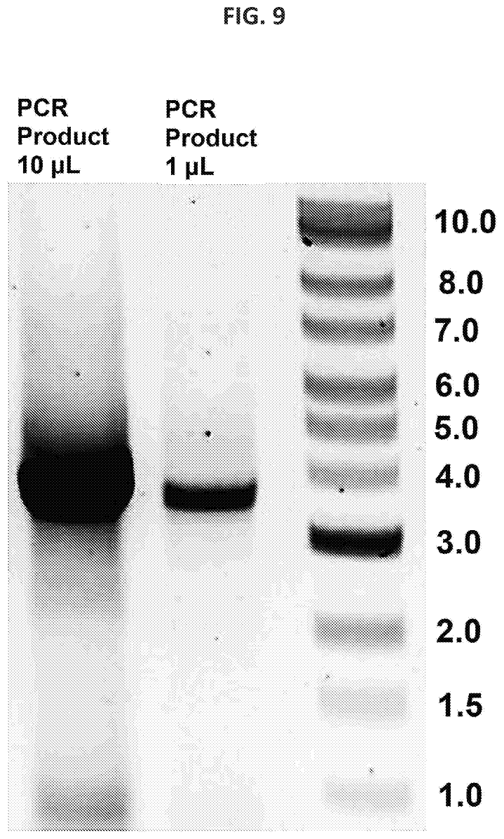

Gel analysis of the LacO-p10-LacO-VP1-FseI insert production pool (FIG. 9) shows strong bands around 3,541 bp, indicating the successful production of LacO-p10-LacO-VP1-FseI insert through PCR amplification from the LacO-p10-LacO-VP1-pUC57 plasmid.

Bacmids from Colony 639 in Example 3 were provided, with each having an FseI region. The 639 Bacmid was digested using 10× Cut Smart Buffer (New England Biolabs, Inc.) and FseI enzyme (5 U/μL) in water (50 ng/μL final concentration) at 37° C. for 2 hours, followed by exposure to 75° C. for 10 minutes to inactivate the enzymes, resulting in a 639 Bacmid single-cut at the FseI locus. The process was repeated to collect additional FseI-cut 639 Bacmid as needed.

### Ligation

The FseI-cut 639 Bacmid was ligated with the LacO-p10-LacO-VP1-FseI insert by combining 25 μL FseI-cut 639 Bacmid (1000 ng), 10 μL LacO-p10-LacO-VP1-FseI insert (1830 ng), 10 μL H₂O, 5 μL 10×T4 ligase buffer and 2 μL T4 ligase enzyme (400U/μL), and then incubating at 37° C. for 4 hours. In one alternative, the FseI-cut 639 Bacmid was ligated with the LacO-p10-LacO-VP1-FseI insert by combining 25 μL FseI-cut 639 Bacmid (1000 ng), 20 μL LacO-p10-LacO-VP1-FseI insert (3660 ng), 5 μL 10×T4 ligase buffer and 2 μL T4 ligase enzyme (400U/μL), and then incubating at 37° C. for 4 hours. In one alternative, the FseI-cut 639 Bacmid was ligated with the LacO-p10-LacO-VP1-FseI insert by combining 25 μL FseI-cut 639 Bacmid (1000 ng), 5 μL LacO-p10-LacO-VP1-FseI insert (915 ng), 15 μL H₂O, 5 μL 10×T4 ligase buffer and 2 μL T4 ligase enzyme (400U/μL), and then incubating at 37° C. for 4 hours. In one alternative, the FseI-cut 639 Bacmid was ligated with the LacO-p10-LacO-VP1-FseI insert by combining 25 μL FseI-cut 639 Bacmid (1000 ng), 2 μL LacO-p10-LacO-VP1-FseI insert (366 ng), 18 μL H₂O, 5 μL 10×T4 ligase buffer and 2 μL T4 ligase enzyme (400U/μL), and then incubating at 37° C. for 4 hours. In one alternative, the FseI-cut 639 Bacmid was ligated with the LacO-p10-LacO-VP1-FseI insert by combining 25 μL FseI-cut 639 Bacmid (1000 ng), 1 μL LacO-p10-LacO-VP1-FseI insert (183 ng), 19 μL H₂O, 5 μL 10×T4 ligase buffer and 2 μL T4 ligase enzyme (400U/IL), and then incubating at 37° C. for 4 hours.

The resulting aqueous phase was combined with 2 μL 3M sodium acetate and 100 μL of ethanol. Precipitated DNA pellets were collected by centrifuge and resuspended in 60 L Tris-EDTA buffer. The resulting ligated plasmid DNA was then transformed into electroporated NEB 10-Beta E. coli (New England Biolabs, Inc.).

Bacterial colonies were grown and screened by colony-pick PCR to test for LacO-p10-LacO-VP1-FseI insertion into the FseI-cut 639 Bacmid.

### PCR Screening and Analysis

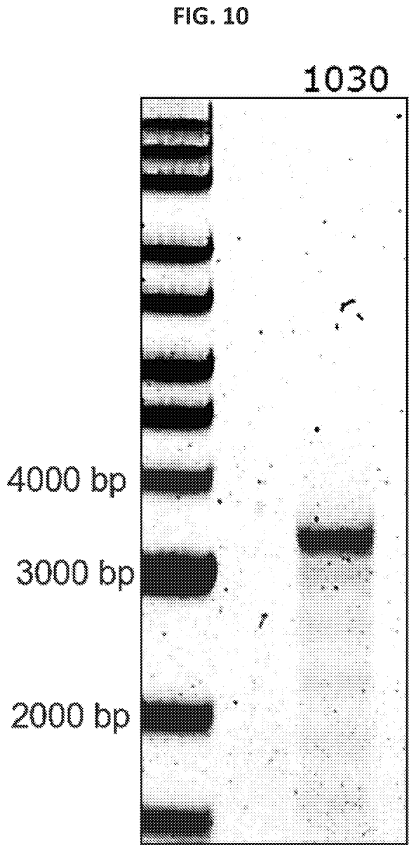

Colony PCR screening was completed using a combination of two primers: Primer JS91-gta-RP1 (SEQ ID NO: 35) and Primer JS122-gta-LP9 (SEQ ID NO: 36). Positive PCR results had a target of about 3,729 bp based on the primers used. Positive results of PCR screening of the bacterial colonies for LacO-p10-LacO-VP1-FseI insertion into the FseI-cut 639 Bacmid are shown in FIG. 10. PCR screening showed that Colony 1030 (FIG. 10) had strong band around 3,729 bp, indicating LacO-p10-LacO-VP1-FseI insertion into the FseI-cut 639 Bacmid of those bacterial colonies.

Further PCR screening was completed for Colony 1030 using a combination of two primers: Primer JS138 (SEQ ID NO: 37) and Primer JS139 (SEQ ID NO: 38). Positive PCR results had a target of about 1070 bp based on the primers used. Further PCR screening was also completed for Colony 1030 using a combination of two primers: Primer JS140 (SEQ ID NO: 39) and Primer JS141 (SEQ ID NO: 40). Positive PCR results had a target of about 621 bp based on the primers used.

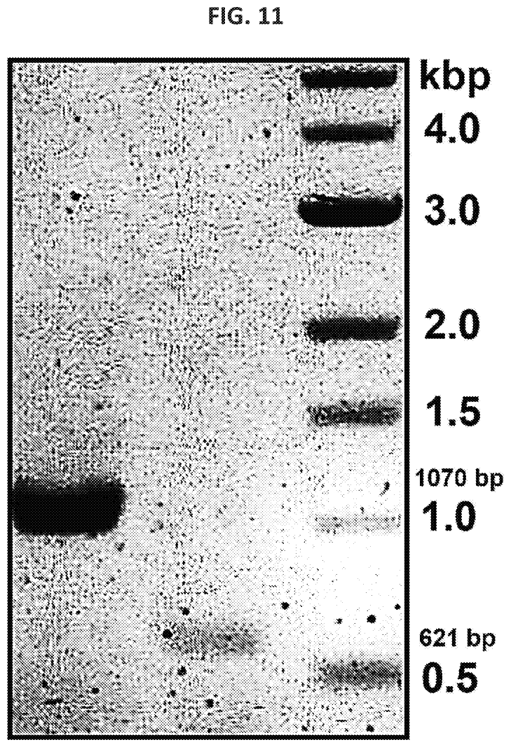

Results of both PCR screens of Colony 1030 for LacO-p10-LacO-VP1-FseI insertion into the FseI-cut 639 Bacmid are shown in FIG. 11. PCR screening showed that Colony 1030 had strong band around 1070 bp (FIG. 11), indicating LacO-p10-LacO-VP1-FseI insertion into the FseI-cut 639 Bacmid of those bacterial colonies. PCR screening also showed that Colony 1030 had weak band around 621 bp (FIG. 11), indicating likely LacO-p10-LacO-VP1-FseI insertion into the FseI-cut 639 Bacmid of those bacterial colonies.

### Example 8. LacO-p10-LacO-VP2-PHPN Polynucleotide

Polynucleotides were engineered to include a regulatable expression control region and a VP2-only coding sequence. The expression control region from Example 4 (SEQ ID NO: 25) was operably linked to a VP2-coding nucleotide sequence (VP2 only). The VP2 sequence for AAV.PHPN capsids was used as an exemplary construct. A small bridge sequence (ccc) was included between the expression control region and the AAV.PHPN VP2 sequence. The polynucleotide also included an HSV TK poly(A) signal.

The polynucleotide was engineered to be inserted/cloned into suitable baculovirus plasmids or vectors. FseI cleavage sequences (ggccggcc) were included to enable cloning of the polynucleotide into the FseI site in the non-essential gta gene ORF of a donor baculovirus plasmid. Cleavage sequences for NheI (gctagc), SpeI (actagt) and BstZ17I (gtatac) were also included to enable cloning into other sites of a donor baculovirus plasmid, such as the Tn7 locus. SpeI (actagt) and NcoI (ccatgg) cleavage sequences were positioned to allow for swapping-out of LacO expression control regions. NcoI (ccatgg) and MluI (acgcgt) cleavage sequences were positioned to allow for swapping-out of VP constructs (e.g. for replacing the AAV.PHPN VP2 sequence with another AAV serotype, such as AAV.PHPB, AAVrh10, AAV9 or AAV2).

A LacO-p10-LacO-VP2-PHPN polynucleotide (with VP2 only, poly(A) and cleavage sequences) is presented in SEQ ID NO: 41.

### Example 9. Cloning LacO-p10-LacO-VP2-PHPN Polynucleotide into FseI Locus of Bacmid

### Digestion

A plasmid containing the full LacO-p10-LacO-VP2 polynucleotide from Example 8 (SEQ ID NO: 41) was produced using pUC57 production plasmid vector (Thermo Fisher Scientific Inc). LacO-p10-LacO-VP2-pUC57 was then digested using 10× Cut Smart Buffer (New England Biolabs, Inc.) and FseI enzyme in water at 37° C. The resulting LacO-p10-LacO-VP2 insert was purified using gel purification. The process was repeated to collect additional LacO-p10-LacO-VP2 insert as needed.

Bacmids from Colony 601 in Example 3 were provided, with each having an FseI region. 6 μg of the 601 Bacmid was digested using 10× Cut Smart Buffer (New England Biolabs, Inc.) and FseI enzyme (5 U/μL) in water (50 ng/μL final concentration) at 37° C. for 2 hours, followed by exposure to 75° C. for 10 minutes to inactivate the enzymes, resulting in a 601 Bacmid single-cut at the FseI locus. The process was repeated to collect additional FseI-cut 601 Bacmid as needed.

### Ligation

The FseI-cut 601 Bacmid was ligated with the LacO-p10-LacO-VP2 insert by combining 40 μL FseI-cut 601 Bacmid (3,040 ng), 20 μL LacO-p10-LacO-VP2 insert (800 ng), 30 μL H₂O, 4 μL 10 mM ATP, 10 μL 10×T4 ligase buffer and 2.5 μL T4 ligase enzyme (400U/μL), and then incubating at 37° C. for 3 hours. The resulting aqueous phase was then combined with 2 μL 3M sodium acetate and 2 volumes of ice-cold ethanol, then chilled at −20° C. for 20 minutes. Precipitated DNA pellets were collected by centrifuge and resuspended in 80 μL Tris-EDTA buffer. The resulting ligated plasmid DNA was then transformed into electroporated NEB 10-Beta E. coli (New England Biolabs, Inc.).

The gel presented in FIG. 12 shows clear ligation of the FseI-cut 601 Bacmid and the LacO-p10-LacO-VP2 (Lane 1 of FIG. 12) into a combined LacO-p10-LacO-VP2 Bacmid (Lane 2 of FIG. 12).

Bacterial colonies were grown and screened by colony-pick PCR to test for LacO-p10-LacO-VP2 insertion into the FseI-cut 601 Bacmid.

### Example 10. Cloning LacO-p10-LacO-VP1 and LacO-p10-LacO-VP2 Polynucleotides into Bacmid

### Digestion

LacO-p10-LacO-VP1 inserts were produced according to Example 5. LacO-p10-LacO-VP2 inserts were produced according to Example 9.

Bacmids from Colony 601 in Example 3 were provided, with each having an FseI region and an I-CeuI region. The 601 Bacmid was digested using 10× Cut Smart Buffer (New England Biolabs, Inc.), I-CeuI enzyme (5 U/μL) and FseI enzyme (5 U/μL) in water at 37° C. for 2 hours, followed by exposure to 75° C. for 10 minutes to inactivate the enzymes, resulting in a 601 Bacmid cut at both the I-CeuI locus and the FseI locus. The process was repeated to collect additional I-CeuI/FseI-cut 601 Bacmid as needed.

### Ligation

The I-CeuI/FseI-cut 601 Bacmid was ligated with the LacO-p10-LacO-VP1-I-CeuI insert and LacO-p10-LacO-VP2-FseI insert by combining 70 μL I-CeuI/FseI-cut 601 Bacmid, 20 μL LacO-p10-LacO-VP1 insert, 20 μL LacO-p10-LacO-VP2 insert, 7 μL 10 mM ATP, 12 μL 10×T4 ligase buffer and 2.5 μL T4 ligase enzyme (400U/μL), and then incubating at 37° C. for 3 hours. The resulting aqueous phase was then combined with 2 μL 3M sodium acetate and 2 volumes of ice-cold ethanol, then chilled at −20° C. for 20 minutes. Precipitated DNA pellets were collected by centrifuge and resuspended in 80 μL Tris-EDTA buffer. The resulting ligated plasmid DNA was then transformed into electroporated NEB 10-Beta E. coli (New England Biolabs, Inc.).

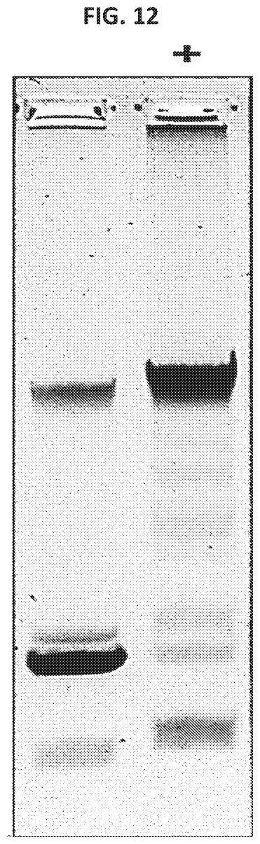

The gel presented in FIG. 13 shows clear ligation of the I-CeuI/FseI-cut 601 Bacmid, the LacO-p10-LacO-VP1 and the LacO-p10-LacO-VP2 (Lane 1 of FIG. 13) into a combined LacO-p10-LacO-VP1/LacO-p10-LacO-VP2 Bacmid (Lane 2 of FIG. 13).

Bacterial colonies were grown and screened by colony-pick PCR to test for LacO-p10-LacO-VP1-I-CeuI and LacO-p10-LacO-VP2-FseI insertion into the I-CeuI/FseI-cut 601 Bacmid.

### Example 11. PCR Screening and Analysis of Bacmids

### JS61JS91 PCR for LacO-p10-LacO-VP2

Colonies 750-758 from Example 9 and Colonies 759-767 from Example 10 were subjected to Colony PCR screening for LacO-p10-LacO-VP2-FseI insertion using a combination of two primers: Primer JS61-VP3-primer2 (SEQ ID NO: 31) and Primer JS91-gta-RP1 (SEQ ID NO: 35). Positive PCR results had a target of about 388 bp based on the primers used.

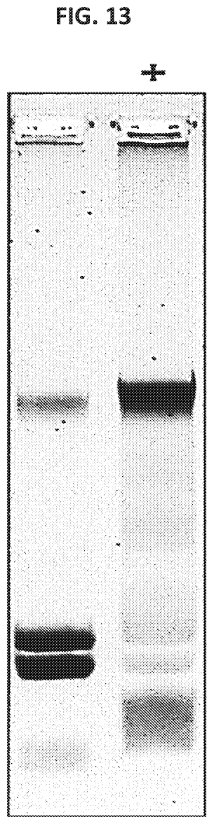

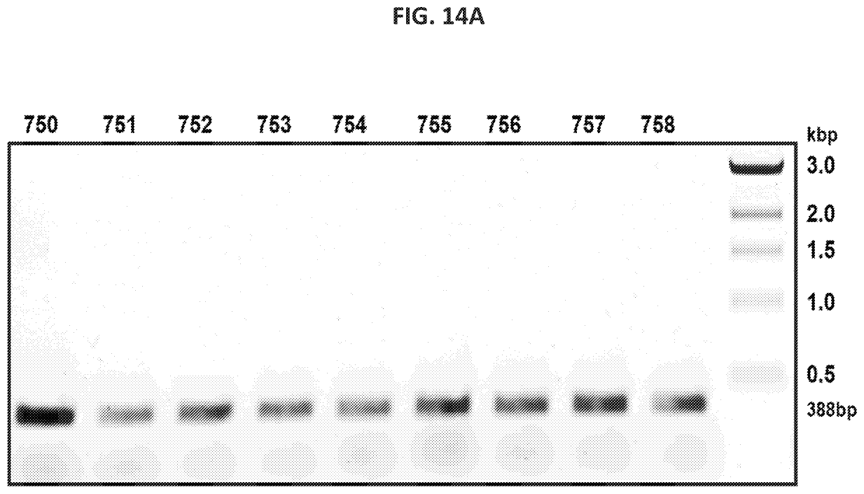

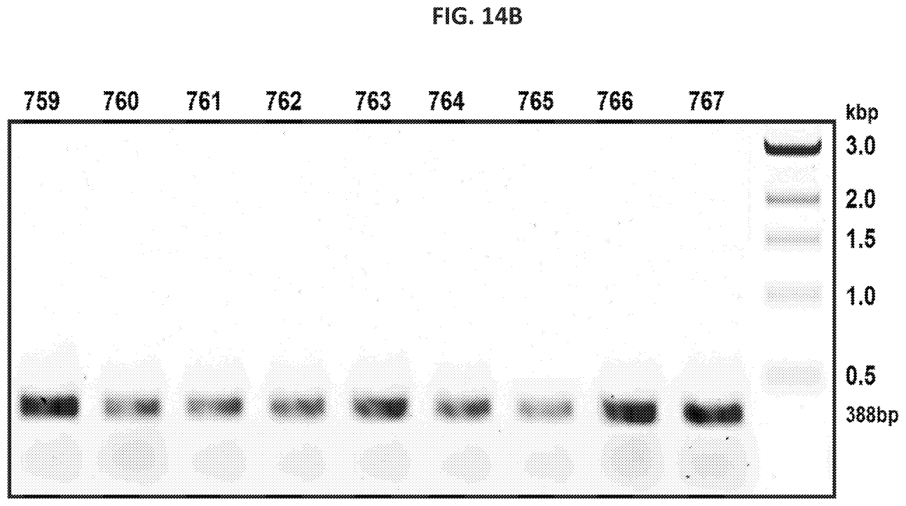

Results of PCR screening of the bacterial colonies for LacO-p10-LacO-VP2 insertion into the FseI-cut 601 Bacmid are shown in FIGS. 14A and 14B. PCR screening showed that all of Colonies 750-758 (FIG. 14A) and Colonies 759-767 (FIG. 14B) had bands around 388 bp, indicating possible LacO-p10-LacO-VP2 insertion into the FseI-cut 601 Bacmid of those bacterial colonies.

### JS90/JS91 PCR for LacO-p10-LacO-VP2

Colonies 750-758 from Example 9 and Colonies 759-767 from Example 10 were subjected to Colony PCR screening for LacO-p10-LacO-VP2-FseI insertion using a combination of two primers: Primer JS90-gta-LP1 (SEQ ID NO: 42) and Primer JS91-gta-RP1 (SEQ ID NO: 35). Positive PCR results had a target of about 2,452 bp based on the primers used.

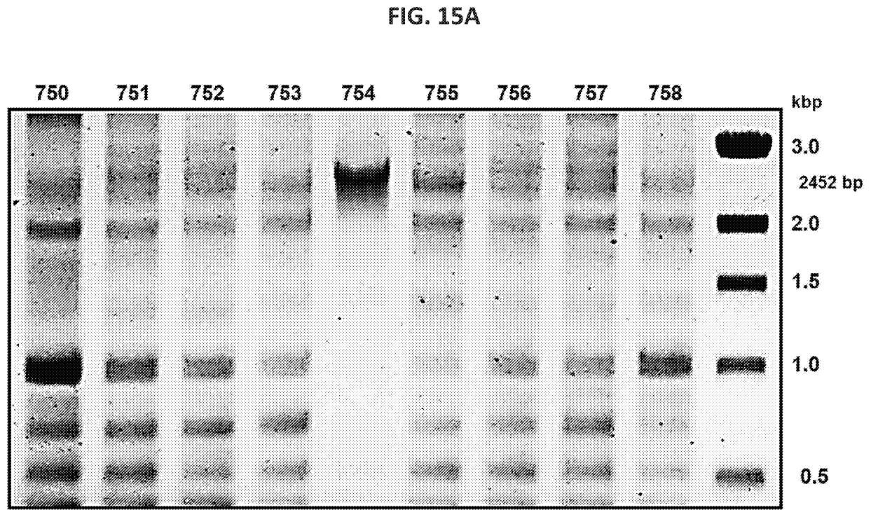

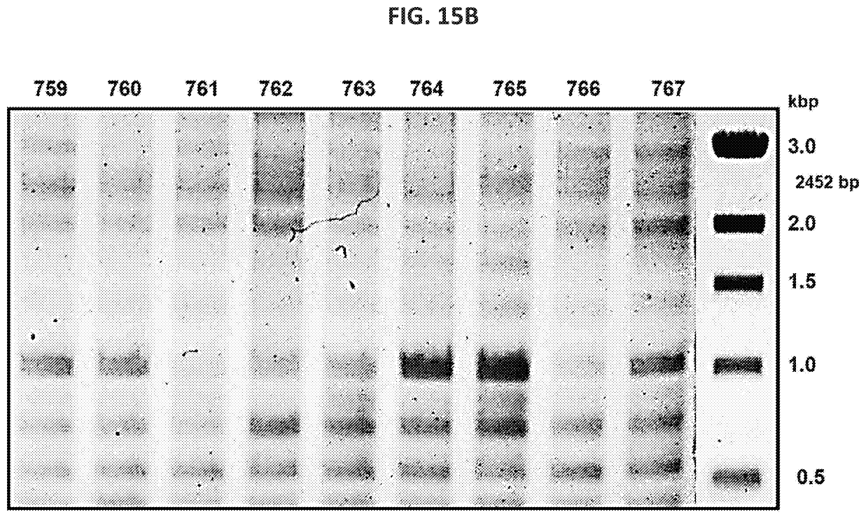

Results of PCR screening of the bacterial colonies for LacO-p10-LacO-VP2 insertion into the FseI-cut 601 Bacmid are shown in FIG. 15A and FIG. 15B. PCR screening showed that Colony 754 (FIG. 15A) had a strong concentrated band around 2452 bp, indicating likely LacO-p10-LacO-VP2 insertion into the FseI-cut 601 Bacmid of those bacterial colonies.

### JS90/JS92 PCR for LacO-p10-LacO-VP2

Colonies 754 and 758 from Example 9 and Colonies 759-767 and 795-800 from Example 10 were subjected to Colony PCR screening for LacO-p10-LacO-VP2-FseI insertion using a combination of two primers: Primer JS90-gta-LP1 (SEQ ID NO: 42) and Primer JS92-AAP-RP1 (SEQ ID NO: 32). Positive PCR results had a target of about 716 bp based on the primers used.

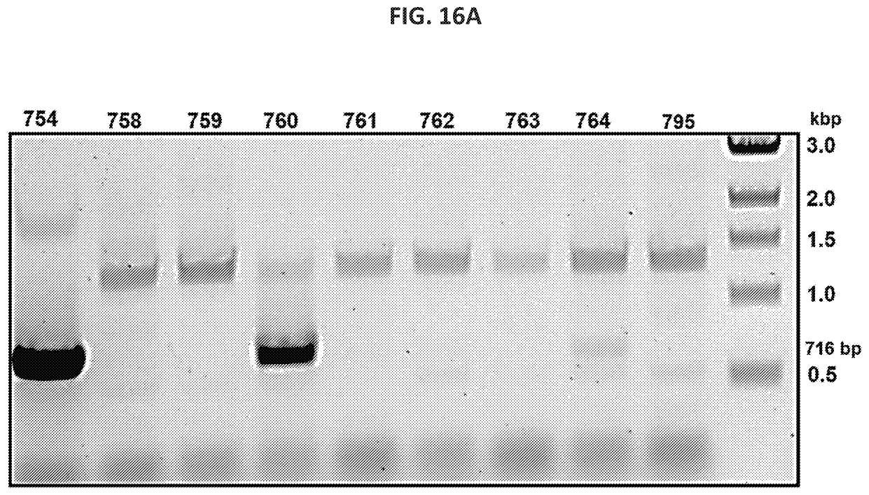

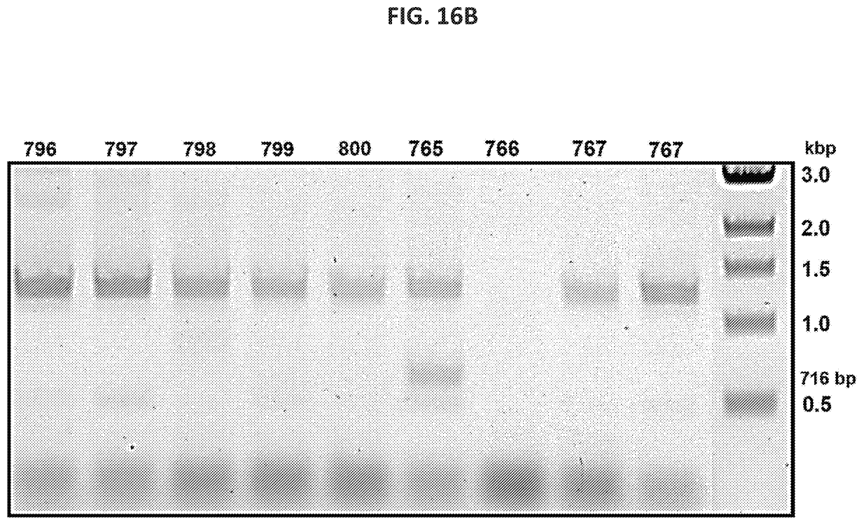

Results of PCR screening of the bacterial colonies for LacO-p10-LacO-VP2 insertion into the FseI-cut 601 Bacmid are shown in FIGS. 16A and 16B. PCR screening showed that Colonies 754 and 760 (FIG. 16A) had strong bands around 716 bp, indicating likely LacO-p10-LacO-VP2 insertion into the FseI-cut 601 Bacmid of those bacterial colonies. Colony 764 (FIG. 16A) and Colony 765 (FIG. 16B) had weak bands around 716 bp. indicating possible LacO-p10-LacO-VP2 insertion into the FseI-cut 601 Bacmid of those bacterial colonies.

### JS90/JS61 PCR for LacO-p10-LacO-VP2

Colonies 754 and 758 from Example 9 and Colonies 759-767 and 795-800 from Example 10 were subjected to Colony PCR screening for LacO-p10-LacO-VP2-FseI insertion using a combination of two primers: Primer JS90-gta-LP1 (SEQ ID NO: 42) and Primer JS61-VP3-primer2 (SEQ ID NO: 31). Positive PCR results had a target of about 516 bp based on the primers used.

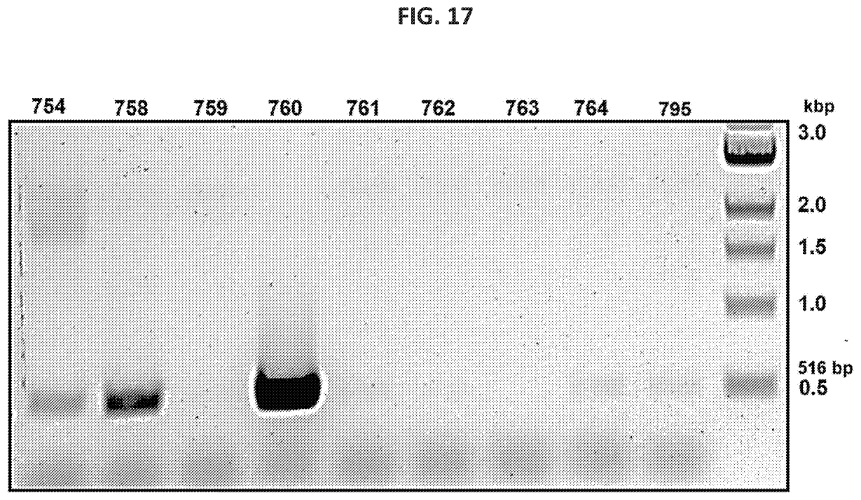

Positive results of PCR screening of the bacterial colonies for LacO-p10-LacO-VP2 insertion into the FseI-cut 601 Bacmid are shown in FIG. 17. PCR screening showed that Colonies 754, 758 and 760 (FIG. 17) had strong bands around 516 bp, indicating likely LacO-p10-LacO-VP2 insertion into the FseI-cut 601 Bacmid of those bacterial colonies. Colonies 764 and 795 (FIG. 17) had weak bands around 516 bp, indicating possible LacO-p10-LacO-VP2 insertion into the FseI-cut 601 Bacmid of those bacterial colonies.

### Example 12. Producing LacO-p10-LacO-VP1 Bacmid by Removal of LacO-p10-LacO-VP2 Insert

### Production and Digestion

A LacO-p10-LacO-VP1/LacO-p10-LacO-VP2 Bacmid is produced according to Example 10. Bacterial colonies are grown and screened by colony-pick PCR, including Colony 994. LacOVP1^{cath}-LacOVP2^{gta}-LacR^{egt} 994 Bacmids are isolated from colony cells.

The isolated 994 Bacmids are digested for FseI cutting using 10× Cut Smart Buffer (New England Biolabs, Inc.) and FseI enzyme (5 U/μL) in water (50 ng/μL final concentration) at 37° C. for 2 hours, resulting in a 994 Bacmid which is cut at the FseI locus. The LacO-p10-LacO-VP2-FseI insert is removed from the bacmid mixture, having been released from the 994 Bacmid by the FseI digestion. The FseI cut in the 994 Bacmid is then ligated closed using 10×T4 ligase buffer and T4 ligase enzyme (400U/μL), and then incubating at 37° C. for 3 hours.

The resulting aqueous phase is combined with 3M sodium acetate and of ethanol, and precipitated DNA pellets are then collected by centrifuge and resuspended in Tris-EDTA buffer. The resulting LacOVP1^{cath}-LacR^{egt} Bacmids are transformed into electroporated NEB 10-Beta E. coli (New England Biolabs, Inc.). Bacterial colonies are grown and screened by colony-pick PCR to test for LacO-p10-LacO-VP1 in the resulting Bacmid.

### Example 13. Fusion-Polynucleotide Insert which Includes FseI-LacO-p10-LacO-VP2-FseI

### First Design

A LacO-p10-LacO-VP2-FseI polynucleotide insert was engineered according to Example 8. The expression control region from Example 4 (SEQ ID NO: 25) was operably linked to a VP2-coding nucleotide sequence (VP2 only). The VP2 sequence for AAV.PHPN capsids was used as an exemplary construct. A small bridge sequence (ccc) was included between the expression control region and the AAV.PHPN VP2 sequence. The polynucleotide also included an HSV TK poly(A) signal.

The polynucleotide was engineered to be inserted/cloned into suitable baculovirus plasmids or vectors. FseI cleavage sequences (ggccggcc) were included to enable cloning of the polynucleotide into the FseI site in the gta gene ORF of a donor baculovirus plasmid. Cleavage sequences for SpeI (actagt) were also included to enable the incorporation of a gta component (and promoter) into the polynucleotide.

Components from the gta gene ORF (partial Ac-lef12 promoter and partial Ac-gta gene) were then incorporated into the polynucleotide insert at the SpeI locus of the polynucleotide insert, forming a fusion-polynucleotide which included the LacO-p10-LacO-VP2 polynucleotide insert and the partial-gta gene ORF polynucleotide. The two components had either the same orientation for translation or opposite orientation for translation. Examples of resulting fusion-polynucleotide inserts are presented as SEQ ID NO: 43 and SEQ ID NO: 44.

### Second Design

A LacO-p10-LacO-VP2-FseI polynucleotide insert was engineered by combining an FseI-LacO-p10-LacO-VP2-MluI fragment with an MluI-AClef12-ACgta-FseI fragment.

The FseI-LacO-p10-LacO-VP2-MluI fragment was produced as follows: A plasmid containing the full LacO-p10-LacO-VP2 polynucleotide from Example 8 (SEQ ID NO: 41) was produced using pUC57 production plasmid vector (Thermo Fisher Scientific Inc). An FseI-FseI-LacO-p10-LacO-VP2-FseI-FseI fragment was then produced by PCR amplification from the larger LacO-p10-LacO-VP2-pUC57 plasmid. The PCR amplification used a combination of Primer JS134-FseI (SEQ ID NO: 33) and Primer JS135-FseI (SEQ ID NO: 34). The PCR product was purified using QiaQuick PCR purification to provide a JS134-JS135 FseI-FseI-LacO-p10-LacO-VP2-FseI-FseI fragment (SEQ ID NO: 45). FIG. 17 shows clear production of the JS134-JS135 fragment (3106 bp).

The JS134-JS135 PCR product was then digested using Cut Smart buffer, FseI enzyme and MluI enzyme at 37° C. The product pool was gel purified and the FseI/MluI digestion process was repeated. The product pool was again gel purified to provide FseI-LacO-p10-LacO-VP2-MluI insert fragments.

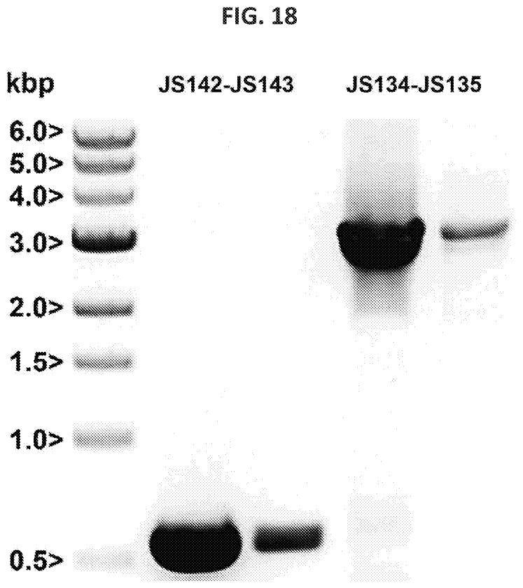

The MluI-AClef12-ACgta-FseI fragment was produced as follows: Bacmids from Colony 1140 (Example 6) were provided. An MluI-AClef12-ACgta-FseI-MluI fragment was then produced by PCR amplification from Bacmid 1140. The PCR amplification used a combination of Primer JS142-gta-UTR-LP-MluI (SEQ ID NO: 46) and Primer JS143-gta-RP-MluI (SEQ ID NO: 47). The PCR product was purified using QiaQuick PCR purification to provide a JS142-JS143 MluI-AClef12-ACgta-FseI-MluI fragment (SEQ ID NO: 48). FIG. 18 shows clear production of the JS142-JS143 fragment (575 bp).

The JS142-JS143 PCR product was then digested using Cut Smart buffer, FseI enzyme and MluI enzyme at 37° C. The product pool was gel purified and the FseI/MluI digestion process was repeated. The product pool was again gel purified to provide MluI-AClef12-ACgta-FseI insert fragments.

In one alternative, The MluI-AClef12-ACgta-FseI fragment was produced as follows: Bacmids from Colony 1140 (Example 6) were provided. An MluI-AClef12-ACgta-FseI-MluI fragment was then produced by PCR amplification from Bacmid 1140. The PCR amplification used a combination of Primer JS142-gta-UTR-LP-MluI (SEQ ID NO: 46) and Primer JS145 (SEQ ID NO: 49). The PCR product was purified using QiaQuick PCR purification to provide a similar MluI-AClef12-ACgta-FseI-MluI fragment (SEQ ID NO: 48).

### Example 14. Producing PHPN-LacOVP1^{ICeu}-LacOVP2^{FseI}-LacR^{AvrII} Bacmid

### Bacmids 1149-1178

Bacmids from Colony 1140 (Example 6) were provided, with each having an FseI region with the gta gene ORF. The 1140 Bacmids were digested using Cut Smart Buffer and FseI enzyme in water at 37° C. The product was gel purified to provide FseI-cut 1140 Bacmid. The process was repeated to collect additional FseI-cut 1140 Bacmid as needed.

The FseI-cut 1140 Bacmid was ligated with the FseI-LacO-p10-LacO-VP2-MluI insert fragment and the MluI-AClef12-ACgta-FseI insert fragment by combining 20 μL FseI-cut 1140 Bacmid (1400 ng), 30 μL of gel purified FseI-LacO-p10-LacO-VP2-MluI insert (800 ng), 12 μL of gel purified MluI-AClef12-ACgta-FseI insert fragment (408 ng), 7 μL 10×T4 ligase buffer and 3 μL T4 ligase enzyme (400U/μL), and then incubating at 37° C. The resulting bacmids were then gel purified. In one alternative, the ligation mixture included 20 μL FseI-cut 1140 Bacmid (1400 ng), 15 μL of gel purified FseI-LacO-p10-LacO-VP2-MluI insert (400 ng), 6 μL of gel purified MluI-AClef12-ACgta-FseI insert fragment (204 ng), 21 μL H₂O, 7 μL 10×T4 ligase buffer and 3 μL T4 ligase enzyme (400U/μL). In one alternative, the ligation mixture included 20 μL FseI-cut 1140 Bacmid (1400 ng), 8 μL of gel purified FseI-LacO-p10-LacO-VP2-MluI insert (200 ng), 3 μL of gel purified MluI-AClef12-ACgta-FseI insert fragment (102 ng), 30 μL H₂O, 7 μL 10×T4 ligase buffer and 3 μL T4 ligase enzyme (400U/μL). In one alternative, the ligation mixture included 20 μL FseI-cut 1140 Bacmid (1400 ng), 4 μL of gel purified FseI-LacO-p10-LacO-VP2-MluI insert (100 ng), 2 μL of gel purified MluI-AClef12-ACgta-FseI insert fragment (50 ng), 36 μL H₂O, 7 μL 10×T4 ligase buffer and 3 μL T4 ligase enzyme (400U/μL).

Bacterial colonies were grown and screened by colony-pick PCR to test for FseI-LacO-p10-LacO-VP2-MluI fragment and the MluI-AClef12-ACgta-FseI fragment insertion into the FseI-cut 1140 Bacmid. Colonies 1149-1178 were subjected to Colony PCR screening using a combination of two primers: Primer JS90-gta-LP1 (SEQ ID NO: 42) and Primer JS91-gta-RP1 (SEQ ID NO: 35). Positive PCR results had a target of about 2779 bp based on the primers used. In one alternative, Colonies 1149-1178 were subjected to Colony PCR screening using a combination of two primers: Primer JS124-gta-LP10 (SEQ ID NO: 50) and Primer JS125-gta-RP10 (SEQ ID NO: 51), with positive PCR results having a target of about 3631 bp based on the primers used.

The resulting bacmids which provided positive PCR results thus included an FseI-LacO-p10-LacO-VP2-AClef12-ACgta-FseI polynucleotide insert (SEQ ID NO: 52).

### Bacmids 1210-1224

FseI-LacO-p10-LacO-VP2-MluI insert fragment and the MluI-AClef12-ACgta-FseI insert fragment were first ligated together to form a FseI-LacO-p10-LacO-VP2-AClef12-ACgta-FseI polynucleotide insert, which was then incorporated as a single insert into the FseI-cut 1140 Bacmid. The FseI-cut 1140 Bacmid was ligated with the FseI-LacO-p10-LacO-VP2-AClef12-ACgta-FseI insert fragment by combining 25 μL FseI-cut 1140 Bacmid, 25 μL of gel purified FseI-LacO-p10-LacO-VP2-AClef12-ACgta-FseI polynucleotide insert, 5.5 μL 10×T4 ligase buffer and 2 μL T4 ligase enzyme (400U/μL), and then incubating at 19° C. for 3 hours. The resulting bacmids were then gel purified.

Bacterial colonies were grown and screened by colony-pick PCR to test for FseI-LacO-p10-LacO-VP2-AClef12-ACgta-FseI insertion into the FseI-cut 1140 Bacmid. Colonies 1210-1224 were subjected to Colony PCR screening using three combinations of two primers: (i) Primer JS61-VP3-primer2 (SEQ ID NO: 31) and Primer JS91-gta-RP1 (SEQ ID NO: 35). with positive PCR results having a target of about 715 bp based on the primers used; (ii) Primer JS124-gta-LP10 (SEQ ID NO: 50) and Primer JS92-AAP-RP1 (SEQ ID NO: 32), with positive PCR results having a target of about 1199 bp based on the primers used, and (iii) JS124-gta-LP10 (SEQ ID NO: 50) and Primer JS91-gta-RP1 (SEQ ID NO: 35), with positive PCR results having a target of about 3262 bp based on the primers used.

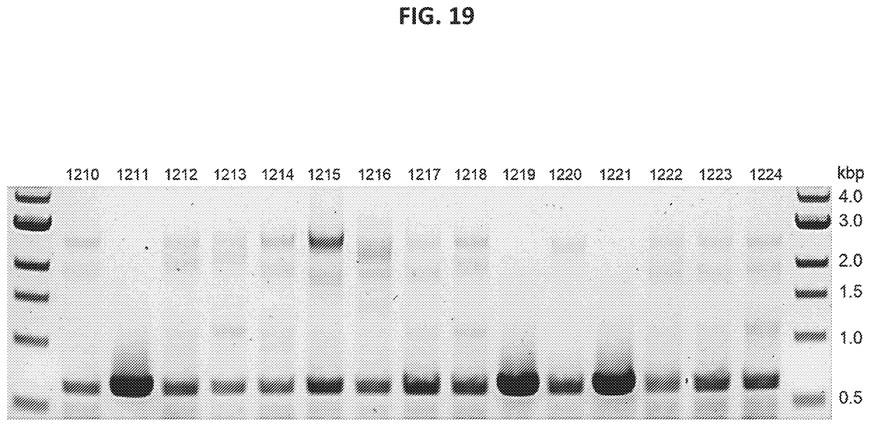

Results of JS124-JS91 PCR screening of Colonies 1210-1224 are shown in FIG. 19. PCR screening showed that none of Colonies 1210-1224 (FIG. 19) had strong bands around 3262 bp, indicating that insertion of FseI-LacO-p10-LacO-VP2-AClef12-ACgta-FseI into the I-FseI-cut 1140 Bacmid was unlikely in any of those bacterial colonies.

### Example 15. Producing LacR-Rep-VP3-LacOVP1^{ICeu}-LacOVP2^{FseI}-LacR^{AvrII} Bacmids

### Production of Bacmid 1186

LacOVP1^{ICeu}-LacOVP2^{FseI}-LacR^{AvrII} bacmids were produced according to Example 14 using Bacmid 1095 (Example 5) instead of Bacmid 1140 (Example 6). The 1095 Bacmids were digested using Cut Smart Buffer and FseI enzyme in water at 37° C. The product was gel purified to provide FseI-cut 1095 Bacmid. The process was repeated to collect additional FseI-cut 1095 Bacmid as needed.

The FseI-cut 1095 Bacmid was ligated with the FseI-LacO-p10-LacO-VP2-AClef12-ACgta-FseI polynucleotide inserts by combining 25 μL FseI-cut 1095 Bacmid, 25 μL of gel purified FseI-LacO-p10-LacO-VP2-AClef12-ACgta-FseI polynucleotide insert, 5.5 μL 10×T4 ligase buffer and 2 μL T4 ligase enzyme (400U/μL), and then incubating at 19° C. for 3 hours. The resulting bacmids were then gel purified.

Bacterial colonies were grown and screened by colony-pick PCR to test for FseI-LacO-p10-LacO-VP2-AClef12-ACgta-FseI insertion into the FseI-cut 1095 Bacmid. Colonies 1180-1194 were subjected to Colony PCR screening using three combinations of two primers: (i) Primer JS61-VP3-primer2 (SEQ ID NO: 31) and Primer JS91-gta-RP1 (SEQ ID NO: 35), with positive PCR results having a target of about 715 bp based on the primers used; (ii) Primer JS124-gta-LP10 (SEQ ID NO: 50) and Primer JS92-AAP-RP1 (SEQ ID NO: 32). with positive PCR results having a target of about 1199 bp based on the primers used; and (iii) JS124-gta-LP10 (SEQ ID NO: 50) and Primer JS91-gta-RP1 (SEQ ID NO: 35), with positive PCR results having a target of about 3262 bp based on the primers used.

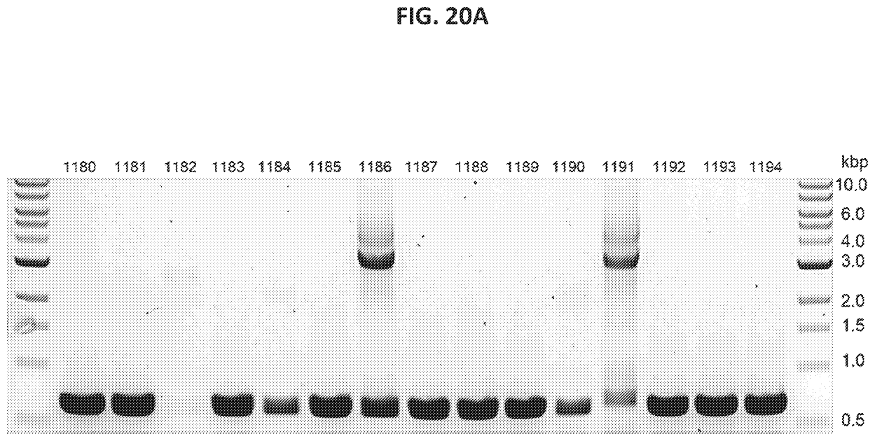

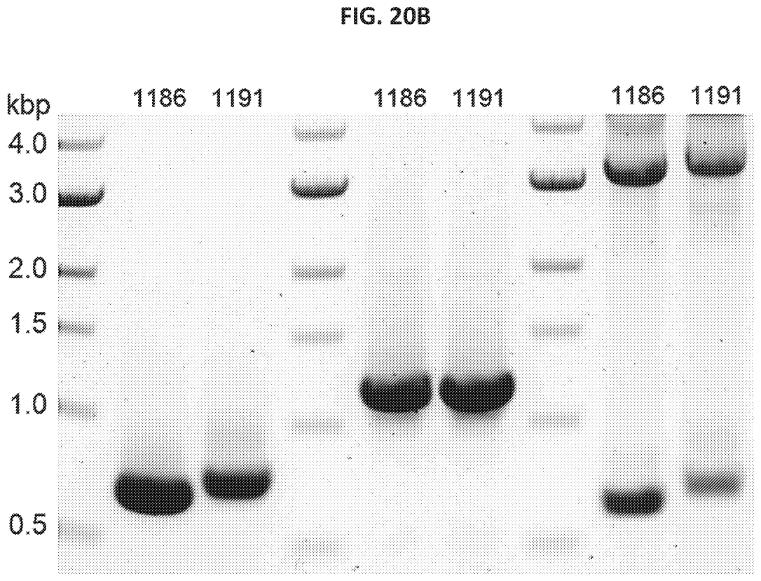

Results of JS124-JS91 PCR screening of Colonies 1180-1194 are shown in FIG. 20A. PCR screening showed that Colonies 1186 and 1191 (FIG. 20A) had strong bands around 3262 bp, indicating likely insertion of FseI-LacO-p10-LacO-VP2-AClef12-ACgta-FseI into the I-FseI-cut 1095 Bacmid of those bacterial colonies.

Results of JS61-JS91 (715 bp), JS124-JS92 (1199 bp) and JS124-JS91 (3262 bp) PCR screening of Colonies 1186 and 1191 are shown in FIG. 20B. PCR screening showed that both Colonies 1186 and 1191 (FIG. 20B) had strong bands around 715 bp, 1199 bp and 3262 bp, strongly indicating likely insertion of FseI-LacO-p10-LacO-VP2-AClef12-ACgta-FseI into Bacmids 1186 and 1191 of those bacterial colonies. PCR screening bands for Colony 1186 were slightly stronger than bands for Colony 1191.

The resulting bacmids which provided positive PCR results (including Bacmid 1186) thus included an FseI-LacO-p10-LacO-VP2-AClef12-ACgta-FseI polynucleotide insert (SEQ ID NO: 52).

### VP3-Only Constructs—Plasmid 1249

Donor plasmid containing VP3-only AAV viral expression constructs were prepared. Plasmid 1249 was prepared to include an AAV Rep sequence (encoding Rep78 and Rep52 proteins) under a polH promoter, and an AAVPHPN Cap sequence encoding VP3-only under a p10 promoter. The VP3-only sequence was produced by providing a shuttle plasmid which included an AAVPHPN VP1 sequence and then digesting it with SmaI and Bsu36I enzymes to excise VP1 and VP2 ORF 5′ ends from the VP construct in the plasmid. The SmaI is a blunt cutting restriction enzyme and the Bsu36I site was blunt filled by incubating with Q5 polymerase. The resulting cut plasmid was phosphorylated with T4 kinase, gel purified and ligated back together with T4 ligase. The resulting Plasmid 1249 included an AAVPHPN Cap sequence encoding VP3-only under a p10 promoter (SEQ ID NO: 53).

### VP3-Only Constructs Plasmid 1259

Plasmid 1259 was prepared to include an AAV Rep sequence (encoding Rep78 and Rep52 proteins) under a polH promoter, an AAVPHPN Cap sequence encoding VP3-only under a p10 promoter (SEQ ID NO: 53), and the opgp64-polH-NLS-LacR region of Example 2 (SEQ ID NO: 12). Plasmid 1259 was prepared by providing Plasmid 1249 and then digesting the plasmid with AvrII, just inside the Tn7L sequence after the Rep78 sequence in Plasmid 1249. The AvrII-cut 1249 Plasmid was ligated with the AvrII-opgp64-polH-NLS-LacR-AvrII polynucleotide insert (SEQ ID NO: 22) by combining 22.5 μL AvrII-cut 1249 Plasmid, 22.5 μL of gel purified AvrII-opgp64-polH-NLS-LacR-AvrII polynucleotide insert, 5.0 μL 10×T4 ligase buffer and 2 μL T4 ligase enzyme (400U/μL), and then incubating at 19° C. for 2 hours.

The resulting plasmid phase was combined with 1 μL 3M sodium acetate and 100 μL of ethanol. Precipitated DNA pellets were collected by centrifuge and resuspended in 100 μL Tris-EDTA buffer. The resulting ligated plasmid DNA was then transformed into electroporated NEB 10-Beta E. coli (New England Biolabs, Inc.).

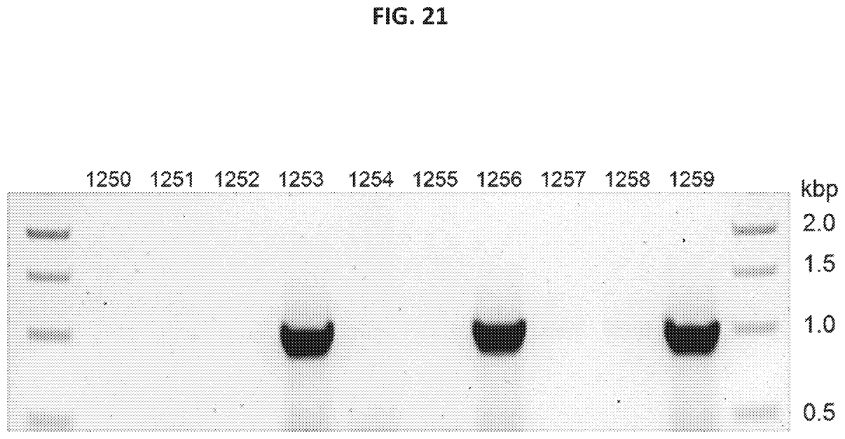

Bacterial colonies 1250-1259 were grown and screened by colony-pick PCR to test for AvrII-opgp64-polH-NLS-LacR-AvrII insertion into the AvrII-cut 1249 Plasmid. Colony PCR screening was completed using a combination of two primers: Primer JS95-LacR-RP1 (SEQ ID NO: 54) and Primer JS42-Rep78-RP_backwards (SEQ ID NO: 55), with positive PCR results having a target of about 1037 bp based on the primers used. Results of JS95-JS42 PCR screening of Colonies 1250-1259 are shown in FIG. 21. PCR screening showed that Colonies 1253, 1256 and 1259 (FIG. 21) had strong bands around 1037 bp, indicating likely insertion of opgp64-polH-NLS-LacR into the AvrII-cut 1249 Plasmid of those bacterial colonies.

Plasmid 1259 thus included the opgp64-polH-NLS-LacR-Rep-VP3 sequence of SEQ ID NO: 56.

### Production of Bacmid 1260

LacR-Rep-VP3-LacOVP1^{ICeu}-LacOVP2^{FseI}-LacR^{AvrII} Bacmids were produced by incorporating the opgp64-polH-NLS-LacR-Rep-VP3 construct (SEQ ID NO: 56) from Plasmid 1259 into the Tn7L region of Bacmid 1186 using standard Tn7 helper plasmids and cloning procedures known in the art. The resulting bacmids were then gel purified and bacterial colonies were grown and tested for opgp64-polH-NLS-LacR-Rep-VP3 insertion into the 1186 Bacmid. Bacterial colony 1260 was selected for further validation and testing.

Colony 1260 was subjected to Colony PCR screening using three combinations of two primers: (i) Primer JS95-LacR-RP1 (SEQ ID NO: 54) and Primer JS42-Rep78-RP_backwards (SEQ ID NO: 55), with positive PCR results having a target of about 1037 bp based on the primers used; (ii) Primer JS124-gta-LP10 (SEQ ID NO: 50) and Primer JS92-AAP-RP1 (SEQ ID NO: 32), with positive PCR results having a target of about 1199 bp based on the primers used; and (iii) Primer JS17-gp64UTR-RP (SEQ ID NO: 30) and Primer JS92-AAP-RP1 (SEQ ID NO: 32), with positive PCR results having a target of about 1092 bp based on the primers used.

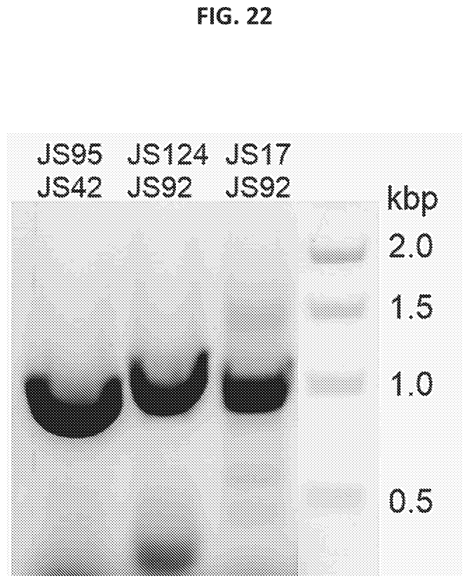

Results of JS95-JS42, JS124-JS92 and JS17-JS92 PCR screening of Colony 1260 are shown in FIG. 22. PCR screening showed that Colony 1260 (FIG. 22) had strong bands around 1037 bp (first column), 1199 bp (second column), and 1092 bp (third column) indicating likely insertion of opgp64-polH-NLS-LacR-Rep-VP3 into the 1186 Bacmid of those bacterial colonies.

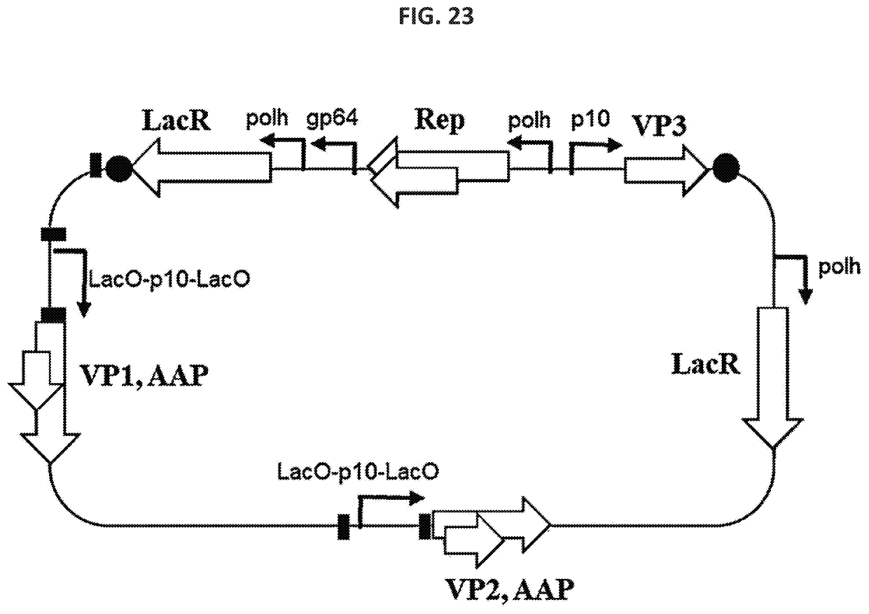

A graphical representation of certain components and coding regions in Bacmid 1260 is presented in FIG. 23, including (i) opgp64-polH-NLS-LacR region, (ii) Rep coding region under a polh promoter; (iii) VP3-only coding region under a p10 promoter, (iv) LacOVP1^{ICeu} region (v) LacOVP2^{FseI} region, and (vi) LacR^{AvrII} region under a polh promoter.

In one alternative, Rep-VP3-LacOVP1^{ICeu}-LacOVP2^{FseI}-LacR^{AvrII} Bacmids can be produced by incorporating the Rep-VP3 construct from Plasmid 1249 into the Tn7L region of Bacmid 1186.

### Testing of Bacmid 1260—IPTG Concentration

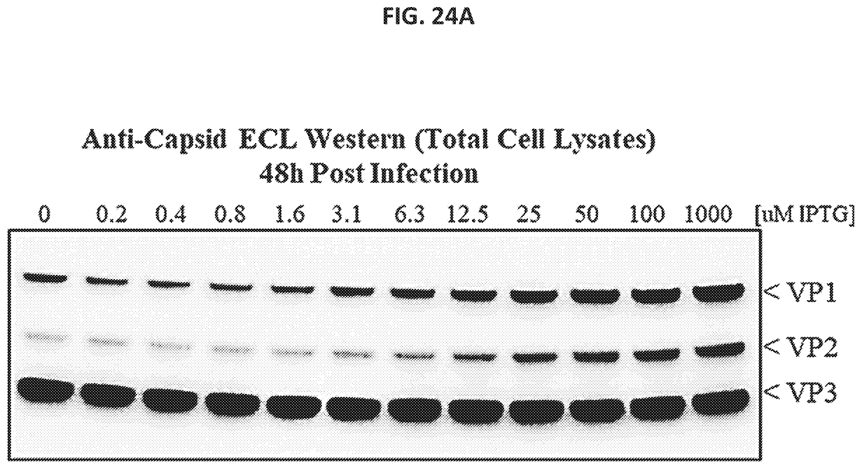

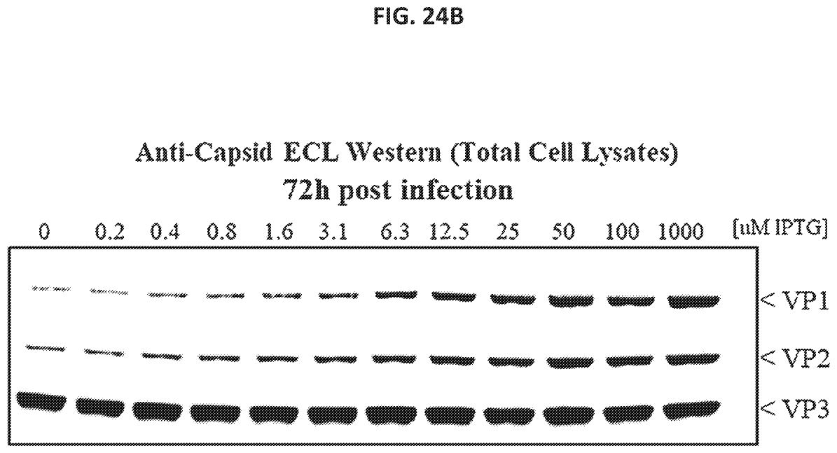

Colony 1260 was tested using an Anti-AAV Capsid ECL Western Blot, with isopropyl-β-D-thiogalactose (IPTG) being used as the inducer element. Bacmids from Colony 1260 were infected into Sf9 cells under different IPTG concentrations, and the total cell lysates were sampled and analyzed using Western Blot at 48 hours and 72 hours post infection. Results are shown in FIG. 24A (48 hours) and FIG. 24B (72 hours).

Results in FIG. 24A show that, at 48 hours post infection, VP1 and VP2 production were both being regulated by LacR. A lower concentration of IPTG resulted in lower VP1/VP2 production and a higher concentration of IPTG resulted in higher VP1/VP2 production. VP2 production was slightly lower than VP1 production, and concentrations of VP3 were consistent over the range of tested IPTG concentrations.

Results in FIG. 24B show that, at 72 hours post infection, VP1 and VP2 production were still both being regulated by LacR. A lower concentration of IPTG continued to result in lower VP1/VP2 production and a higher concentration of IPTG resulted in higher VP/VP2 production. VP2 production was slightly lower than VP1 production, and concentrations of VP3 were consistent over the range of tested IPTG concentrations.

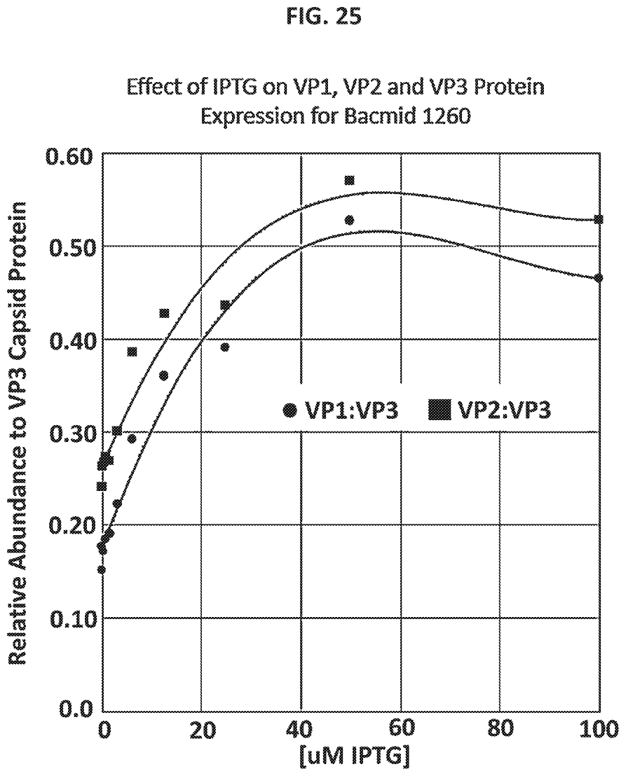

Results from the Anti-AAV Capsid ECL Western Blot at 72 hours post infection (FIG. 24B) were quantified by Raw Pixel Count Imaging Software (adjusted for local background subtraction). Results are shown in Table 5 and FIG. 25.

TABLE 5
Western Blot Quantification Results
(Local Background Subtracted)

| IPTG (μM) | VP1 | VP1:VP3 | VP2 | VP2:VP3 | VP3 |
|---|---|---|---|---|---|
| 0 | 18 | 0.18 | 26 | 0.25 | 102 |
| 0.2 | 15 | 0.16 | 24 | 0.26 | 94 |
| 0.4 | 17 | 0.17 | 27 | 0.27 | 100 |
| 0.8 | 18 | 0.19 | 27 | 0.29 | 93 |
| 1.6 | 19 | 0.21 | 27 | 0.30 | 89 |
| 3.1 | 22 | 0.24 | 30 | 0.32 | 93 |
| 6.3 | 29 | 0.28 | 38 | 0.37 | 102 |
| 12.5 | 36 | 0.36 | 43 | 0.43 | 101 |
| 25 | 39 | 0.40 | 44 | 0.45 | 97 |
| 50 | 53 | 0.53 | 57 | 0.57 | 100 |
| 100 | 46 | 0.49 | 53 | 0.57 | 93 |

Results showed a steady increase in VP1:VP3 and VP2:VP3 ratios at up to about 50 μM IPTG, with ratios leveling around 0.50-0.60 at IPTG concentrations between 50-100 μM.

### Testing of Bacmid 1260—Bacterial Cell Count

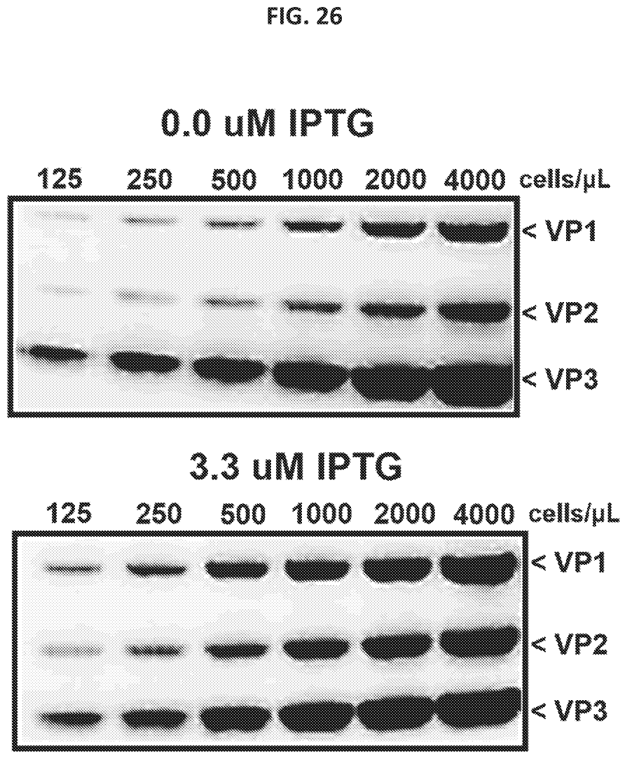

Bacmid 1260 was tested at multiple bacterial cell counts using an Anti-AAV Capsid ECL Western Blot, with isopropyl-R-D-thiogalactose (IPTG) being used as the inducer element (0.0 μM IPTG and 3.3 μM IPTG). The bacterial cell counts being tested were: 125 cells/μL, 250 cells/μL, 500 cells/μL, 1000 cells/μL, 2000 cells/μL and 4000 cells/μL. Results are shown in FIG. 26.

Results in FIG. 26 show that VP1 and VP2 production were both regulated by LacR in all bacterial cell concentrations being tested, with 3.3 μM IPTG samples showing increased VP1 and VP2 production compared to 0.0 μM IPTG samples. VP3 production was consistent between 3.3 μM IPTG samples and 0.0 μM IPTG samples.

### Example 16. Co-Infection Testing of Bacmid 1260 with ITR-GFP

Studies were conducted to evaluate the effects of IPTG regulation on AAV production using Bacmid 1260 as an expressionBac, co-infected with an ITR-GFP bacmid (Bacmid 449) as a payloadBac. Bacmid 449 includes an ITR-to-ITR payload construct which includes a GFP-encoding nucleotide sequence in the payload region. Bacmid 420 (comprising AAVPHPN Cap sequence encoding VP1, VP2 and VP3 under a p10 promoter) was used as a reference.

### Co-Infection Experiment A

40 ml of stock sf9 insect cell mixture was combined with 110 ml of ESF media to provide cell concentration of about 2.0×10⁶ cells/ml. 5 ml volumes of Sf9 cells were then dispensed into 50 ml vented tubes and placed at 28° C. at 235 rpm. A concentration of IPTG inducer element was added to each tube according to Table 6. payloadBIICs incorporating Bacmid 499 were then co-infected into each tube with expressionBIICs incorporating Bacmid 1260 (10 μl volumes of each BIIC type added to 5 ml volumes of Sf9 cells). Similar samples were also prepared for the co-infection of payloadBIICs incorporating Bacmid 499 with expressionBIICs incorporating Bacmid 420 (reference).

TABLE 6
Co-infection Experiment 1 Dilutions

| | IPTG | IPTG | 25 mM IPTG | Media |
|---|---|---|---|---|
| Sample | 1X (μM) | 100X (μM) | Volume (μl) | Volume (μl) |
| 1 | 1.7 | 167 | 7 | 993 |
| 2 | 2.2 | 223 | 9 | 991 |
| 3 | 3.0 | 297 | 12 | 988 |
| 4 | 4.0 | 396 | 16 | 984 |
| 5 | 5.3 | 528 | 21 | 979 |
| 6 | 7.0 | 704 | 28 | 972 |
| 7 | 9.4 | 939 | 38 | 962 |
| 8 | 12.5 | 1251 | 50 | 950 |
| 9 | 16.7 | 1669 | 67 | 933 |
| 10 | 22.2 | 2225 | 89 | 911 |
| 11 | 29.7 | 2966 | 119 | 881 |
| 12 | 39.6 | 3955 | 158 | 842 |
| 13 | 52.7 | 5273 | 211 | 789 |
| 14 | 70.3 | 7031 | 281 | 719 |
| 15 | 93.8 | 9375 | 375 | 625 |
| 16 | 125 | 12500 | 500 | 500 |
| 17 | 250 | 25000 | 1000 | 0 |

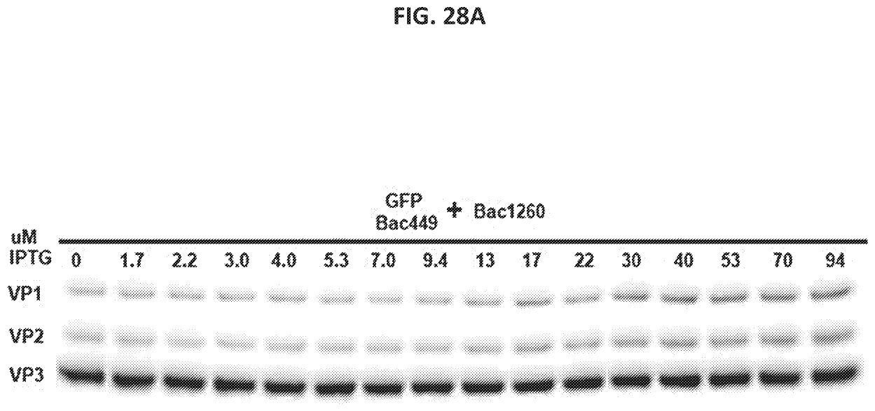

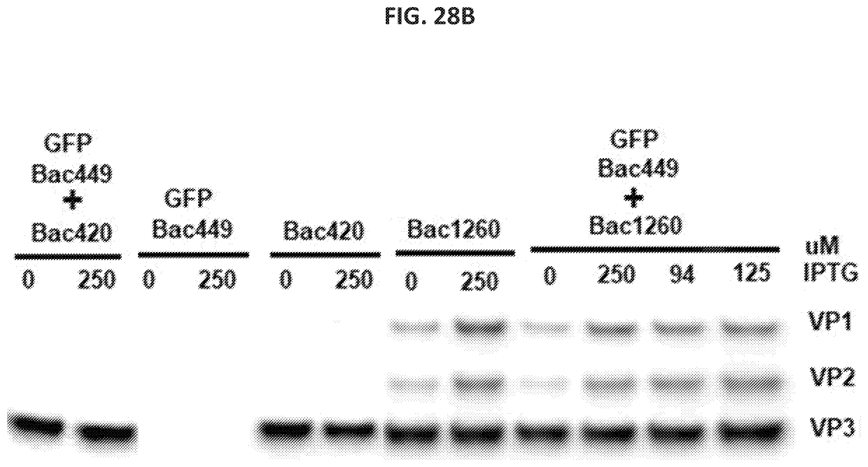

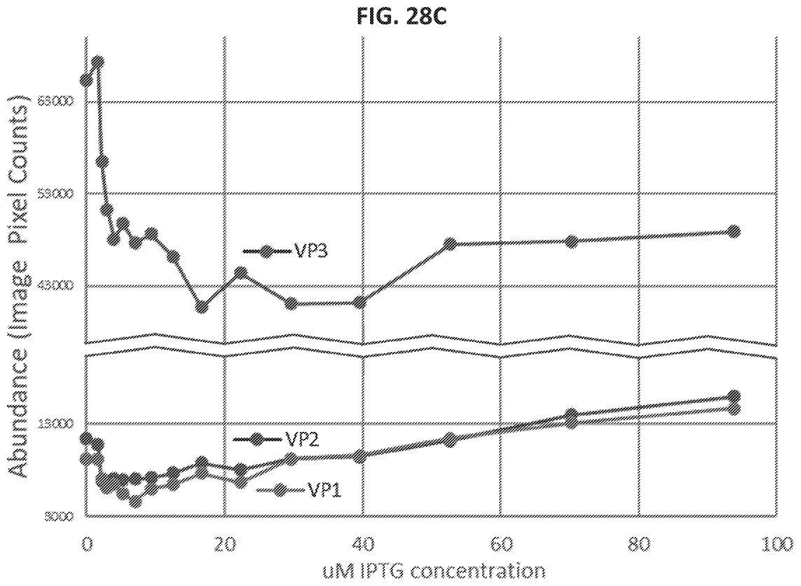

Cells were sampled at 48 hours post-infection and 72 hours post-infection. Resulting cell lysates were analyzed by Western Blot, with results shown in FIG. 28A and FIG. 28B (image lanes normalized to VP3). Results from the Western Blot (FIG. 28A) were quantified by Raw Pixel Count Imaging Software (adjusted for local background subtraction). with results shown in FIG. 28C.

Results showed that addition of IPTG was able to induce additional expression of VP1 and VP2, but overall VP expression was limited due to rapid replication of Bacmid 449 (resulting in a shift in Bac1260:Bac449 ratios).

### Co-Infection Experiment B

The general protocols for Co-infection Experiment A were expanded to include a range of expressionBIIC-to-payloadBIIC ratios. BIIC ratio conditions are shown in Table 7.

TABLE 7
expressionBIIC-to-payloadBIIC ratios

| BIIC | Target | BIIC | BIIC | Media |
|---|---|---|---|---|
| Ratio # | Ratio | 1260 (μl) | 449 (μl) | Volume (μl) |
| 1 | 16:1 | 100 | 6 | 894 |
| 2 | 8:1 | 100 | 13 | 888 |
| 3 | 4:1 | 100 | 25 | 875 |
| 4 | 2:1 | 100 | 50 | 850 |
| 5 | 1:1 | 100 | 100 | 800 |
| 6 | 1:2 | 100 | 200 | 700 |
| 7 | 1:4 | 100 | 400 | 500 |
| 8 | 1:8 | 100 | 800 | 100 |

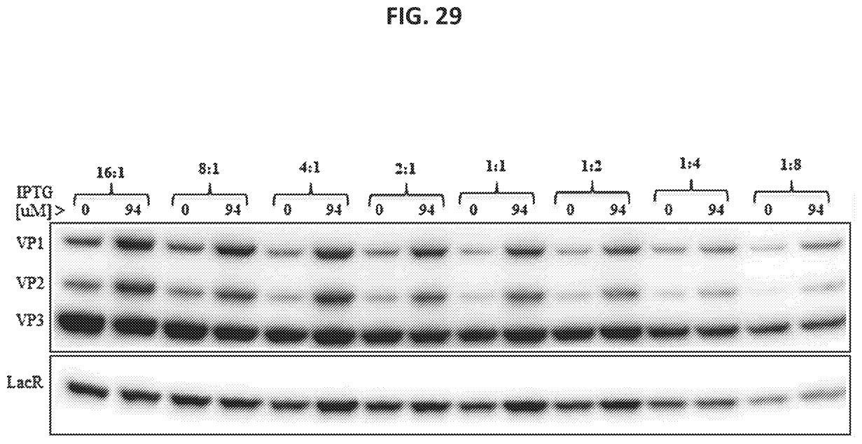

Each BIIC ratio condition was tested with the following IPTG concentrations: 0.00 μM, 1.67 μM, 2.97 μM, 5.28 μM, 9.39 μM, 17.0 μM 40.0 μM, and 94.0 μM. Cells were sampled at 90 hours post-infection. Resulting cell lysates were analyzed by Western Blot, with results for 0.00 μM and 94.0 μM samples shown in FIG. 29. Results showed that addition of IPTG was able to induce additional expression of VP1 and VP2 in all BIIC Ratio samples, and that overall VP expression and LacR production was limited at higher concentrations of Bacmid 449.

### Co-Infection Experiment C

The general protocols for Co-infection Experiment A were reused with adjustments, including the use of 35 ml of stocks/9 insect cell mixture being combined with 100 ml of ESF media to provide cell concentration of about 2.3×10⁶ cells/ml. 577 μl (0.3 MOI) of payloadBIICs incorporating Bacmid 499 were then co-infected into each tube with 138 μl (0.1 MOI) expressionBIICs incorporating Bacmid 1260, or 118 μl (0.1 MOI) expressionBIICs incorporating Bacmid 420.

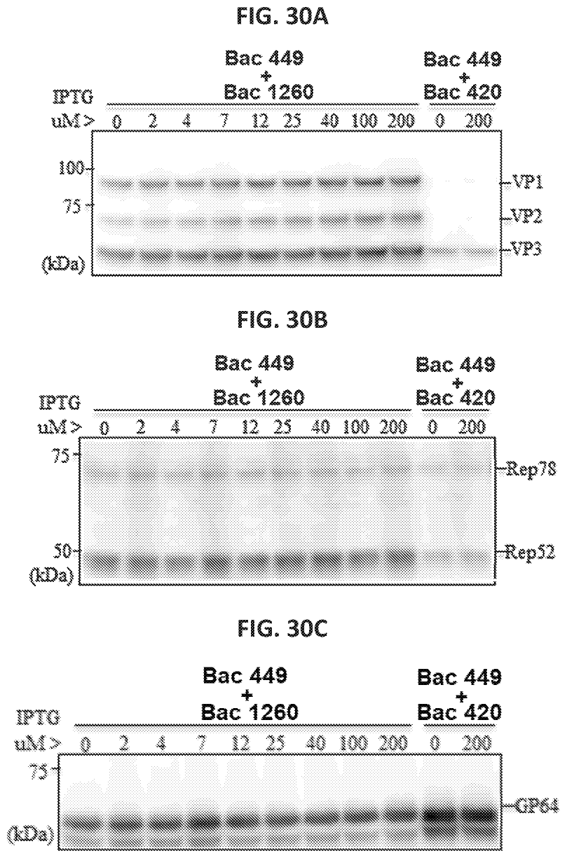

Each BIIC condition was tested with the following IPTG concentrations: 0.0 μM, 1.0 μM, 2.0 μM, 3.0 μM, 4.0 μM, 5.0 μM, 7.0 μM, 10.0 μM, 12.0 μM, 15.0 μM, 25.0 μM, 30.0 μM, 40.0 μM, 50.0 μM, 100.0 μM and 200.0 μM. Cells were sampled at 90 hours post-infection. Resulting cell lysates were analyzed by Western Blot, with results for Cap expression shown in FIG. 30A, results for Rep expression shown in FIG. 30B, and results for GP64 expression shown in FIG. 30C. Results showed that addition of IPTG was able to induce additional expression of VP1 and VP2 from Bacmid 1260 in all BIIC Ratio samples and IPTG concentrations, with consistent Rep production and reduced GP64 production. Resulting cell lysates were also analyzed for AAV titer (qPCR), with results shown in Table 8.

TABLE 8
AAV Titer for cell lysates from Co-infection Experiment C

| | |
|---|---|
| IPTG | AAV Titer |
| (μM) | (×10¹⁰ vg/ml) |

| | | |
|---|---|---|
| 0 | uM | 6.63 |
| 1 | uM | 6.63 |
| 2 | uM | 6.66 |
| 3 | uM | 6.41 |
| 4 | uM | 6.17 |
| 5 | uM | 6.11 |
| 7 | uM | 5.77 |
| 10 | uM | 5.48 |
| 12 | uM | 5.15 |
| 15 | uM | 4.49 |
| 25 | uM | 4.10 |
| 30 | uM | 3.92 |
| 40 | uM | 3.63 |
| 50 | uM | 3.53 |
| 100 | uM | 3.10 |
| 200 | uM | 2.86 |

| | |
|---|---|
| Reference | 3.1 |

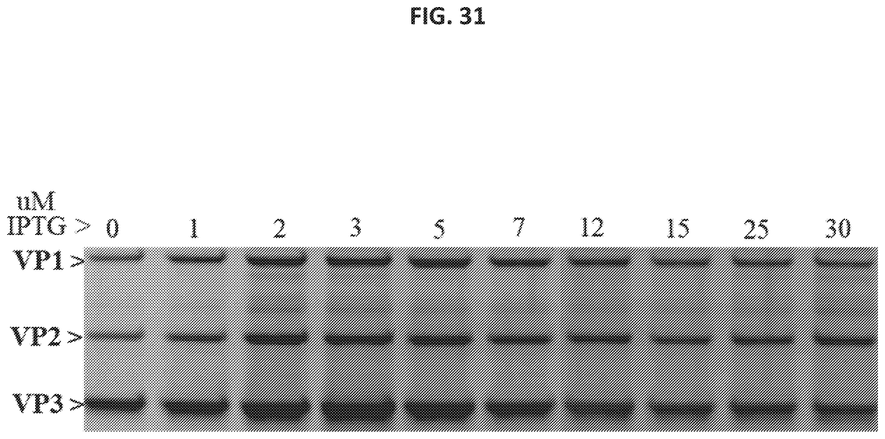

AAV capsids were then isolated from resulting cell lysates using Iodixanol Purification (See Buclez et al. Molecular Therapy-Methods & Clinical Development. 3:16035 (January 2016), the content of which is incorporated herein by reference in its entirety as related to Iodixanol Purification of AAV particles, insofar as it does not conflict with the present disclosure). Resulting iodixanol gradient isolate was buffer-exchanged into PBS F-68 buffer (PBS, 0.001% Pluronic-F68, pH 7.4) and concentrated on Vivaspin 20 columns (20 min, 4000×g). Resulting samples were analyzed with Silver Stain/SDS-PAGE, with results shown in FIG. 31. Resulting iodixanol-clarified samples were also analyzed for AAV titer (qPCR), with results shown in Table 9.

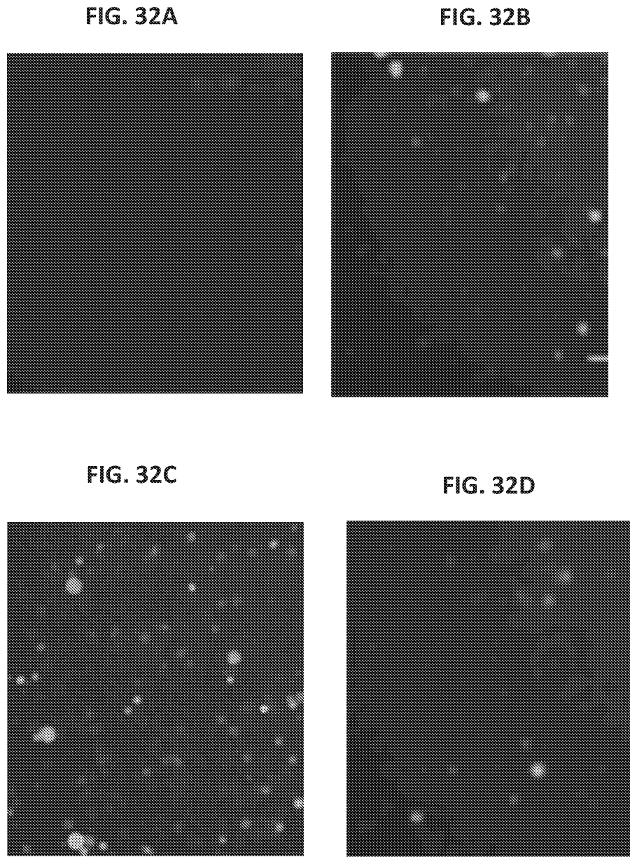

50 μl aliquots of samples were also added to 200 ul DMEM overlaid on 50% confluent HEK 293 cells in 96-well format. Relative fluorescence of cells was recorded 3 days later, with results shown in FIG. 32A for 0.0 μM IPTG, FIG. 32B for 5.0 μM, FIG. 32C for 15.0 μM and FIG. 32D for 3.0 μM. GFP cells per well were counted, with results also shown in Table 9. Relative potency to 0 uM IPTG was calculated (GFP cells per well divided by AAV titer, normalized to 0 uM IPTG).

TABLE 9
AAV Titer and GFP cell count for iodixanol clarified AAV

| | | | |
|---|---|---|---|
| IPTG | AAV Titer | GFP | Relative |
| (μM) | (×10¹¹ vg/ml) | (cells per well) | potency |

| | | | | |
|---|---|---|---|---|
| 0 | uM | 3.54 | 79 | 100.0% |
| 1 | uM | 4.21 | 104 | 110.6% |
| 2 | uM | 4.69 | 148 | 141.1% |
| 3 | uM | 4.51 | 85 | 84.5% |
| 4 | uM | 2.98 | — | — |
| 5 | uM | 5.00 | 312 | 279.6% |
| 7 | uM | 3.88 | 97 | 112.0% |
| 10 | uM | 1.04 | — | — |
| 12 | uM | 4.18 | 446 | 478.1% |
| 15 | uM | 3.35 | 587 | 785.2% |
| 25 | uM | 3.10 | 224 | 323.8% |
| 30 | uM | 3.31 | 7 | 9.5% |
| 40 | uM | 0.745 | — | — |
| 50 | uM | 0.585 | — | — |
| 100 | uM | 1.73 | — | — |
| 200 | uM | 0.682 | — | — |

| | | | |
|---|---|---|---|
| Reference | 1.50 | — | — |

Results showed that increasing addition of IPTG produced decreasing concentrations of AAV particles (qPCR titer). However, results also showed that potency (i.e. GFP expression) was strongest between 12-25 μM IPTG, and particularly strong between 12.5-22.5 μM IPTG with a peak at 15 μM IPTG.

### Co-Infection Experiment D

The general protocols for Co-infection Experiment C were reused with adjustments, including the use of with sf9 insect cell stock prepared in 100 ml of ESF media to a cell concentration of about 2.5×10⁷ cells/ml. 727 μl (0.3 MOI) of payloadBIICs incorporating Bacmid 499 were then co-infected into each tube with 175 μl (0.1 MOI) expressionBIICs incorporating Bacmid 1260, or 149 μl (0.1 MOI) expressionBIICs incorporating Bacmid 420.

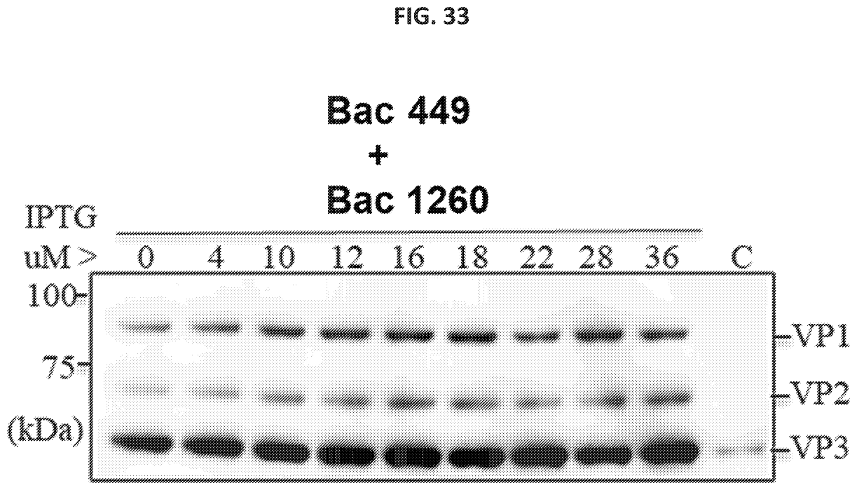

Each BIIC condition was tested with the following IPTG concentrations: 0.0 μM, 1.0 μM, 2.0 μM, 4.0 μM, 6.0 μM, 8.0 μM, 10.0 μM, 12.0 μM, 14.0 μM, 16.0 μM, 18.0 μM, 22.0 μM, 28.0 μM, 36.0 μM, 48.0 μM and 80.0 μM. Cells were sampled at 10⁸ hours post-infection. Resulting cell lysates were analyzed by Western Blot for Cap expression (FIG. 33). Results showed that addition of IPTG was able to induce additional expression of VP1 and VP2 from Bacmid 1260 in all BIIC Ratio samples. Resulting cell lysates were also analyzed for AAV titer (qPCR). with results shown in Table 10.

TABLE 10
AAV Titer for cell lysates from Co-infection Experiment D

| | |
|---|---|
| IPTG | AAV Titer |
| (μM) | (×10¹¹ vg/ml) |

| | | |
|---|---|---|
| 0 | uM | 1.39 |
| 1 | uM | 1.12 |
| 2 | uM | 1.19 |
| 4 | uM | 1.18 |
| 6 | uM | 1.05 |
| 8 | uM | 1.08 |
| 10 | uM | 1.02 |
| 12 | uM | 1.02 |
| 14 | uM | 0.900 |
| 16 | uM | 0.988 |
| 18 | uM | 0.935 |
| 22 | uM | 0.914 |
| 28 | uM | 0.813 |
| 36 | uM | 0.830 |
| 48 | uM | 0.684 |
| 80 | uM | 0.603 |

| | |
|---|---|
| Reference | 0.818 |

Results showed that the increasing addition of IPTG produced decreasing concentrations of AAV particles (qPCR titer), though AAV titer results for Co-infection Experiment D were higher than corresponding AAV titer results for Co-infection Experiment C.

### Co-Infection Experiment E

The general protocols for Co-infection Experiment D were reused with adjustments, including the use of sf9 insect cell stock prepared in 700 ml of ESF media to a cell concentration of about 8.15×10⁶ cells/ml. 920 μl (0.3 MOI) of payloadBIICs incorporating Bacmid 499 were then co-infected into each tube with 221 μl (0.1 MOI) expressionBIICs incorporating Bacmid 1260, or 188 μl (0.1 MOI) expressionBIICs incorporating Bacmid 420.

Each BIIC condition was tested with the following IPTG concentrations: 0.0 μM, 0.1 μM, 0.3 μM, 1.0 μM, 2.0 μM, 3.0 μM, 4.0 μM, 5.0 μM, 6.0 μM, 8.0 μM, 10.0 μM, 20.0 μM, 30.0 μM, 40.0 μM, 50.0 μM and 100.0 μM. Resulting cell lysates were analyzed for AAV titer (qPCR and ddPCR), with results shown in Table 11.

TABLE 11
AAV Titer for cell lysates from Co-infection Experiment E

| | | |
|---|---|---|
| IPTG | AAV Titer - qPCR | AAV Titer - ddPCR |
| (μM) | (×10¹¹ vg/ml) | (×10¹¹ vg/ml) |

| | | | |
|---|---|---|---|
| 0 | uM | 1.46 | 2.25 |
| 0.1 | uM | 1.44 | 2.22 |
| 0.3 | uM | 1.49 | 2.25 |
| 1 | uM | 1.56 | 2.32 |
| 2 | uM | 1.75 | 2.62 |
| 3 | uM | 1.8 | 2.58 |
| 4 | uM | 1.64 | 2.37 |
| 5 | uM | 1.60 | 2.45 |
| 6 | uM | 1.52 | 2.29 |
| 8 | uM | 1.49 | 2.23 |
| 10 | uM | 1.48 | 2.25 |
| 20 | uM | 1.33 | 1.98 |
| 30 | uM | 1.23 | 1.89 |
| 40 | uM | 1.17 | 1.74 |
| 50 | uM | 1.06 | 1.57 |
| 100 | uM | 0.917 | 1.31 |

| | | |
|---|---|---|
| Reference | .0711 | 0.803 |

Results showed that increasing addition of IPTG produced relatively stable AAV titer up to 10.0 μM for Co-infection Experiment E, with concentrations of AAV particles steadily decreasing at IPTG concentrations above 10.0 μM.

### Example 17. Time Course Study of Bacmid 1260 with ITR-GFP

Studies were conducted to evaluate the effect of IPTG regulation and post-infection harvest timing on AAV production using Bacmid 1260 as an expressionBac, co-infected with an ITR-GFP bacmid (Bacmid 449) as a payloadBac. Bacmid 449 includes an ITR-to-ITR payload construct which includes a GFP-encoding nucleotide sequence in the payload region. Bacmid 420 (comprising AAVPHPN Cap sequence encoding VP1, VP2 and VP3 under a p10 promoter) was used as a reference.

A 48-well plate was seeded with sf9 insect cells in ESF media and TCID50, with about 1.4×10⁶ cells/well. Two columns (16 wells) were co-infected with payloadBIICs incorporating Bacmid 499 (10 pfu/cell MOI) and expressionBIICs incorporating Bacmid 1260 (10 pfu/cell MOI); two columns (16 wells) were co-infected with payloadBIICs incorporating Bacmid 499 (10 pfu/cell MOI) and expressionBIICs incorporating Bacmid 420 (10 pfu/cell MOI); and two columns (16 wells) were infected with only payloadBIICs incorporating Bacmid 499 (10 pfu/cell MOI). IPTG was then added to alternating columns (100 uM IPTG in cells), with no IPTG added to the remaining alternating columns.

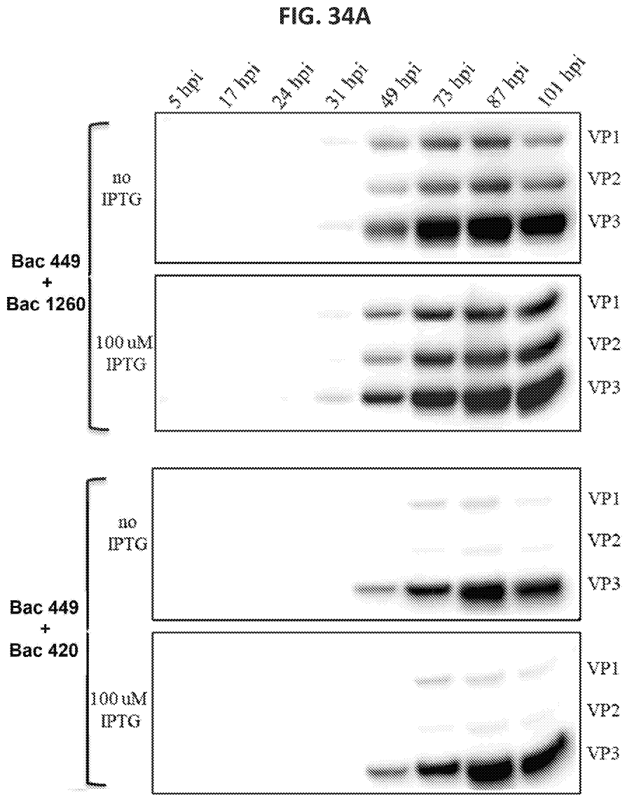

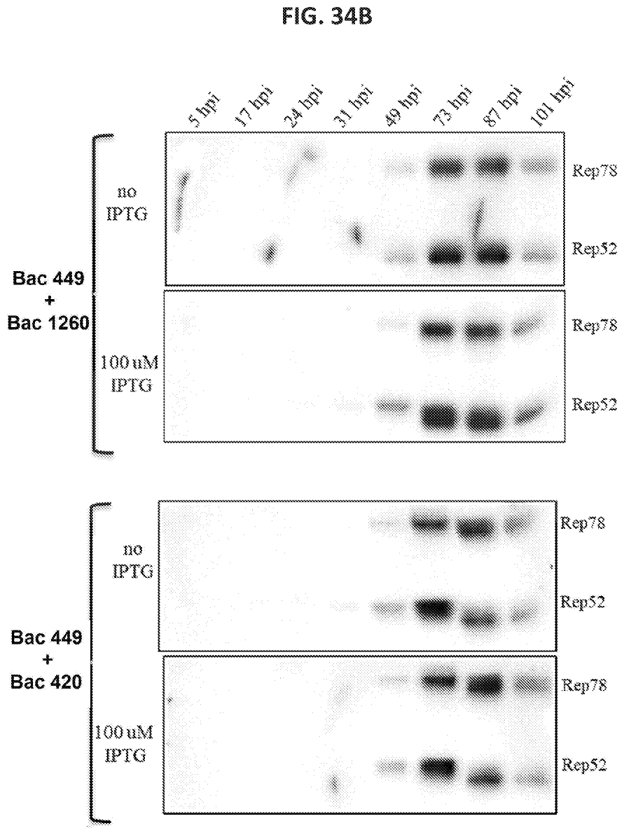

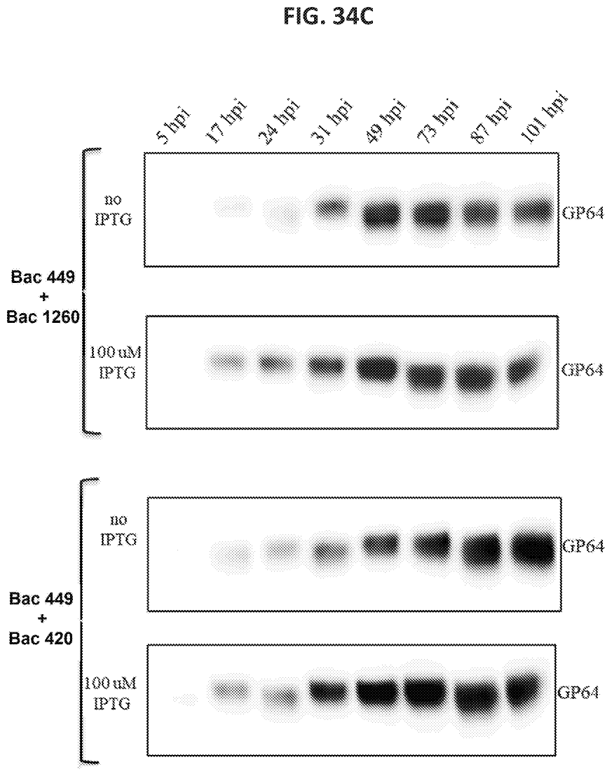

Each row of the plate (one cell for each column) was than harvested at the following post-infection times (hours post infection, hpi): 5 hpi, 17 hpi, 24 hpi, 31 hpi, 49 hpi, 73 hpi, 87 hpi, and 101 hpi. Resulting samples were analyzed by Western Blot, with results for Cap expression shown in FIG. 34A, results for Rep expression shown in FIG. 34B, and results for GP64 expression shown in FIG. 34C. Results showed that Cap, Rep and GP64 production was strongest between 49-87 hpi, and particularly strong between 73-87 hpi.

We claim:

1. A transcriptional regulatory system comprising one or more regulator elements, one or more regulator binding sequences, and one or more inducer elements.

2. The transcriptional regulatory system of claim 1, wherein the one or more regulator elements have a high affinity for binding to the one or more regulator binding sequences.

3. The transcriptional regulatory system of claim 1, wherein the transcriptional regulatory system comprises two regulator binding sequences, and wherein the one or more regulator elements have a high affinity for binding to both regulator binding sequences simultaneously.

4. The transcriptional regulatory system of claim 3, wherein the simultaneous binding of the regulator element to the two regulator binding sequences results in the formation of a loop structure in a nucleotide sequence between the two regulator binding sequences.

5. The transcriptional regulatory system of any one of claims 1-4, wherein the one or more inducer elements can bind to the one or more regulator elements, thereby reducing the affinity of the regulator element for binding to the one or more regulator binding sequences.

6. The transcriptional regulatory system of claim 5, wherein the binding of the inducer element to the regulator element causes a conformational change in the regulator element, thereby reducing the affinity of the regulator element for binding to the one or more regulator binding sequences.

7. The transcriptional regulatory system of any one of claims 1-6, wherein at least one regulator element is a Lac repressor (LacR) protein selected from a wildtype Lac repressor protein (wLacR) or an engineered Lac repressor protein (eLacr).

8. The transcriptional regulatory system of any one of claims 1-6, wherein at least one regulator element is an engineered Lac repressor protein (eLacr).

9. The transcriptional regulatory system of claim 8, wherein the engineered LacR protein is encoded by a polynucleotide comprising a nucleotide sequence selected from SEQ ID NO: 2 or a nucleotide sequence which is at least 75%, at least 80%, at least 85%, at least 90%, at least 95%, or at least 99% identical to SEQ ID NO: 2.

10. The transcriptional regulatory system of any one of claims 1-9, wherein at least one regulator binding sequence is a Lac Operator (LacO) sequence.

11. The transcriptional regulatory system of any one of claims 1-9, wherein at least one regulator binding sequence is a Lac Operator (LacO) sequence which comprises a nucleotide sequence selected from SEQ ID NO: 4 or a nucleotide sequence which is at least 75%, at least 80%, at least 85%, at least 90%, at least 95%, or at least 99% identical to SEQ ID NO: 4.

12. The transcriptional regulatory system of any one of claims 1-11, wherein at least one inducer element is selected from any one of lactose, allolactose and isopropyl-β-D-thiogalactose (IPTG); preferably wherein at least one inducer element is IPTG.

13. A viral expression construct, wherein the viral expression construct comprises a first protein-coding region and a first regulator region; wherein the first protein-coding region of the viral expression construct comprises a first protein-coding nucleotide sequence and a first expression control sequence operably linked to the first protein-coding nucleotide sequence; wherein the first expression control sequence comprises a promoter which regulates the transcription of the first protein-coding nucleotide sequence, and also comprises one or more of the regulator binding sequences of the transcriptional regulatory system of any one of claims 1-12; and wherein the first regulator region of the viral expression construct comprises a first regulator nucleotide sequence which encodes one or more of the regulator elements of the transcriptional regulatory system of any one of claims 1-12.

14. The viral expression construct of claim 13, wherein the first regulator region comprises a nucleotide sequence selected from SEQ ID NO: 5 or a nucleotide sequence which is at least 75%, at least 80%, at least 85%, at least 90%, at least 95%, or at least 99% identical to SEQ ID NO: 5.

15. The viral expression construct of claim 13, wherein the first regulator region comprises a nucleotide sequence selected from SEQ ID NO: 10 or a nucleotide sequence which is at least 75%, at least 80%, at least 85%, at least 90%, at least 95%, or at least 99% identical to SEQ ID NO: 10.

16. The viral expression construct of claim 13, wherein the first regulator region comprises a nucleotide sequence selected from SEQ ID NO: 11 or a nucleotide sequence which is at least 75%, at least 80%, at least 85%, at least 90%, at least 95%, or at least 99% identical to SEQ ID NO: 11.

17. The viral expression construct of any one of claims 13-16, wherein the viral expression construct comprises a second regulator region which comprises a second regulator nucleotide sequence, wherein the second regulator nucleotide sequence encodes one or more of the regulator elements of the transcriptional regulatory system.

18. The viral expression construct of claim 17, wherein the second regulator region comprises a nucleotide sequence selected from SEQ ID NO: 12 or a nucleotide sequence which is at least 75%, at least 80%, at least 85%, at least 90%, at least 95%, or at least 99% identical to SEQ ID NO: 12.

19. The viral expression construct of claim 17, wherein the second regulator region comprises a nucleotide sequence selected from SEQ ID NO: 22 or a nucleotide sequence which is at least 75%, at least 80%, at least 85%, at least 90%, at least 95%, or at least 99% identical to SEQ ID NO: 22.

20. The viral expression construct of any one of claims 13-19, wherein the first protein-coding region comprises a nucleotide sequence selected from SEQ ID NO: 25 or a nucleotide sequence which is at least 75%, at least 80%, at least 85%, at least 90%, at least 95%, or at least 99% identical to SEQ ID NO: 25.

21. The viral expression construct of any one of claims 13-19, wherein the first protein-coding region comprises a nucleotide sequence selected from SEQ ID NO: 28 or a nucleotide sequence which is at least 75%, at least 80%, at least 85%, at least 90%, at least 95%, or at least 99% identical to SEQ ID NO: 28.

22. The viral expression construct of any one of claims 13-21, wherein the regulator binding sequence of the first expression control sequence is within 5-100 nucleotides from one end of the promoter of first expression control sequence.

23. The viral expression construct of any one of claims 13-21, wherein the first expression control sequence comprises a first regulator binding sequence which is 5-100 nucleotides upstream of the 5′ end of the promoter of the first expression control sequence, and a second regulator binding sequence which is 5-100 nucleotides downstream from the 3′ end of the promoter of first expression control sequence.

24. The viral expression construct of claim 23, wherein the first regulator binding sequence and the second regulator binding sequence have a space interval of between 150 to 300, between 150 to 250, between 150 to 225, or between 150 to 210 nucleotides between them, as measured from the center nucleotide of each regulator binding sequence.

25. The viral expression construct of any one of claims 13-24, wherein the regulator element encoded by the regulator nucleotide sequence binds to the one or more regulator binding sequences in the first expression control sequence, and inhibits or reduces transcription of the first protein-coding nucleotide sequence from the promoter in the first expression control sequence.

26. The viral expression construct of any one of claims 23-24, wherein the regulator element encoded by the regulator nucleotide sequence binds to the first regulator binding sequence and the second regulator binding sequence in the first expression control sequence, resulting in the formation of a loop structure around the promoter in the first expression control sequence, and thereby inhibiting or reducing transcription of the first protein-coding nucleotide sequence from the promoter in the first expression control sequence.

27. The viral expression construct of any one of claims 13-26, wherein the first protein-coding nucleotide sequence encodes a structural AAV capsid protein selected from VP1, VP1 only, VP2 only, VP3 only, or a combination thereof.

28. The viral expression construct of claim 27, wherein the first protein-coding nucleotide sequence encodes VP1 only.

29. The viral expression construct of any one of claims 13-28, wherein the viral expression construct comprises a second protein-coding region which comprises a second protein-coding nucleotide sequence and a second expression control sequence operably linked to the second protein-coding nucleotide sequence; wherein the second expression control sequence comprises a promoter which regulates the transcription of the second protein-coding nucleotide sequence, and also comprises one or more of the regulator binding sequences of the transcriptional regulatory system.

30. The viral expression construct of claim 29, wherein the second protein-coding region comprises a nucleotide sequence selected from SEQ ID NO: 41 or a nucleotide sequence which is at least 75%, at least 80%, at least 85%, at least 90%, at least 95%, or at least 99% identical to SEQ ID NO: 41.

31. The viral expression construct of claim 29, wherein the second protein-coding region comprises a nucleotide sequence selected from SEQ ID NO: 43 or a nucleotide sequence which is at least 75%, at least 80%, at least 85%, at least 90%, at least 95%, or at least 99% identical to SEQ ID NO: 43.

32. The viral expression construct of claim 29, wherein the second protein-coding region comprises a nucleotide sequence selected from SEQ ID NO: 44 or a nucleotide sequence which is at least 75%, at least 80%, at least 85%, at least 90%, at least 95%, or at least 99% identical to SEQ ID NO: 44.

33. The viral expression construct of claim 29, wherein the second protein-coding region comprises a nucleotide sequence selected from SEQ ID NO: 52 or a nucleotide sequence which is at least 75%, at least 80%, at least 85%, at least 90%, at least 95%, or at least 99% identical to SEQ ID NO: 52.

34. The viral expression construct of any one of claims 29-33, wherein the second protein-coding nucleotide sequence encodes a structural AAV capsid protein selected from VP1, VP1 only, VP2 only, VP3 only, or a combination thereof.

35. The viral expression construct of claim 34, wherein the second protein-coding nucleotide sequence encodes VP2 only.

36. The viral expression construct of claim 34, wherein the second protein-coding nucleotide sequence encodes VP3 only.

37. The viral expression construct of any one of claims 29-36, wherein the viral expression construct comprises a third protein-coding region which comprises a third protein-coding nucleotide sequence and a third expression control sequence operably linked to the third protein-coding nucleotide sequence; wherein the third expression control sequence comprises a promoter which regulates the transcription of the third protein-coding nucleotide sequence.

38. The viral expression construct of claim 37, wherein the third protein-coding region comprises a nucleotide sequence selected from SEQ ID NO: 53 or a nucleotide sequence which is at least 75%, at least 80%, at least 85%, at least 90%, at least 95%, or at least 99% identical to SEQ ID NO: 53.

39. The viral expression construct of any one of claims 37-38, wherein the third protein-coding nucleotide sequence encodes a structural AAV capsid protein selected from VP1, VP1 only, VP2 only, VP3 only, or a combination thereof.

40. The viral expression construct of claim 39, wherein the third protein-coding nucleotide sequence encodes VP2 only.

41. The viral expression construct of claim 39, wherein the third protein-coding nucleotide sequence encodes VP3 only.

42. The viral expression construct of any one of claims 37-41, wherein the viral expression construct comprises a nucleotide sequence selected from SEQ ID NO: 56 or a nucleotide sequence which is at least 75%, at least 80%, at least 85%, at least 90%, at least 95%, or at least 99% identical to SEQ ID NO: 56.

43. A viral production cell which comprises a viral expression construct of any one of claims 13-42; preferably wherein the viral production cell comprises an insect cell; more preferably wherein the viral production cell comprises an sf9 insect cell.

44. A viral production system which comprises the viral expression construct of any one of claims 13-42 and the transcriptional regulatory system of any one of claims 1-12.

45. The viral production system of claim 44, wherein the one or more regulator binding sequences of the transcriptional regulatory system are comprised in the viral expression construct.

46. The viral production system of claim 44 or claim 45, wherein the one or more regulator elements of the transcriptional regulatory system are encoded by the regulator nucleotide sequences comprised in the viral expression construct.

47. The viral production system of any one of claims 44-46, wherein the one or more inducer elements bind to the one or more regulator elements and reduces the affinity of the regulator elements for binding to the one or more regulator binding sequences in the viral expression construct

48. The viral production system of claim 47, wherein the level of transcription of one or more protein-coding nucleotide sequences in the viral expression construct is increased or decreased proportionally to the concentration of the inducer elements within the viral production system.

49. The viral production system of any one of claims 44-48, wherein the one or more protein-coding nucleotide sequences in the viral expression construct encode one or more structural AAV capsid proteins; wherein the one or more inducer elements is present in the viral production system at a target concentration; and wherein the target concentration of inducer elements within the viral production system results in the production of AAV capsids which have a VP1:VP2:VP3 protein ratio of 0.5-2:0.5-2:10.

50. The viral production system of claim 49, wherein the target concentration of inducer elements within the viral production system results in the production of AAV capsids which have a VP1:VP2:VP3 protein ratio of 1-2:1-2:10.

51. The viral production system of any one of claims 44-50, wherein the inducer element is present at a concentration between about 1.0 μM to about 100 μM; preferably wherein the inducer element is present at a concentration between about 1.0 μM to about 35 μM.


SEQUENCE LISTING (56 sequences)

SEQ ID NO: 1 - DNA, 26 bp, Artificial Sequence
  source: /note=“Description of Artificial Sequence Synthetic oligonucleotide”
taactataacggtcctaaggtagcga

SEQ ID NO: 2 - DNA, 1128 bp, Artificial Sequence
  source: /note=“Description of Artificial Sequence Synthetic polynucleotide”
atgacgcaacctaagaagaagaggaaggttcccgggcaagtgactatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 3 - DNA, 1128 bp, Artificial Sequence
  source: /note=“Description of Artificial Sequence Synthetic polynucleotide”
atgacccagcccaaaaaaaagagaaaagtccctggtcaggtaactatgaagccggtcaccttgtacgacgtcgctgaatacgcaggtgtgtcctatcaaacagtgagtcgtgtggtcaaccaggccagccacgttagtgcaaaaacccgtgagaaagtggaggcggcaatggccgagcttaattacataccaaaccgtgttgcacaacagcttgcaggcaagcaatccctgctcattggcgttgcaactagtagcctggctcttcatgcgccttcccagattgtggccgctattaagagtagagcagatcagttgggagcatccgttgtcgtcagcatggttgaaaggagtggcgttgaggcttgcaaagctgcggtccataacctgctggcacaaagagtgagcggactcatcatcaactacccactggatgatcaagacgcgattgccgtcgaggctgcatgtacgaacgttccagctctttttcttgacgtgtcagaccaaacacccataaattctattatttttagtcacgaggacggtacccgcttgggtgttgaacacttggtagctttgggacatcagcagatcgcattgttggctggtccacttagtagcgtgagtgcaagacttagactggcaggttggcacaagtacttgacccgtaatcaaatacaacctatagctgagcgcgaaggtgactggagcgctatgagcggtttccagcaaacgatgcagatgctgaacgagggaattgtacccaccgctatgttggtcgctaacgaccaaatggccttgggcgccatgagagctattactgaaagcggcttgcgcgtgggcgcagatatctctgtcgtaggttacgatgatacggaagactctagctgctatatccccccaagcacgacgatcaagcaggatttccgtctccttggacagaccagtgtagacaggctccttcaactgagccagggccaagctgtgaagggcaaccaactcttgccagtgtcgttggtgaagcgcaagaccaccctggctcccaacacgcaaactgcttctcccagggctttggctgattccctgatgcagctcgcccgccaagtcagtagacttgaatcaggccagtga

SEQ ID NO: 4 - DNA, 20 bp, Artificial Sequence
  source: /note=“Description of Artificial Sequence Synthetic oligonucleotide”
gattgtgagcgctcacaatt

SEQ ID NO: 5 - DNA, 1220 bp, Artificial Sequence
  source: /note=“Description of Artificial Sequence Synthetic polynucleotide”
atcttggagataattaaattgataaccatctcgcaaataaataagtattttactgttttcgtaacagttttgtaataaaaaaacctataaatatgacgcaacctaagaagaagaggaaggttcccgggcaagtgactatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 6 - DNA, 92 bp, Artificial Sequence
  source: /note=“Description of Artificial Sequence Synthetic oligonucleotide”
atcttggagataattaaattgataaccatctcgcaaataaataagtattttactgttttcgtaacagttttgtaataaaaaaacctataaat

SEQ ID NO: 7 - DNA, 30 bp, Artificial Sequence
  source: /note=“Description of Artificial Sequence Synthetic oligonucleotide”
atgacgcaacctaagaagaagaggaaggtt

SEQ ID NO: 8 - DNA, 15 bp, Artificial Sequence
  source: /note=“Description of Artificial Sequence Synthetic oligonucleotide”
cccgggcaagtgact

SEQ ID NO: 9 - DNA, 1083 bp, Artificial Sequence
  source: /note=“Description of Artificial Sequence Synthetic polynucleotide”
atgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 10 - DNA, 1385 bp, Artificial Sequence
  source: /note=“Description of Artificial Sequence Synthetic polynucleotide”
cctagggctagcgtatacatcttggagataattaaattgataaccatctcgcaaataaataagtattttactgttttcgtaacagttttgtaataaaaaaacctataaatatgacgcaacctaagaagaagaggaaggttcccgggcaagtgactatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgaaacttgtttattgcagcttataatggttacaaataaagcaatagcatcacaaatttcacaaataaagcatttttttcactgcattctagttgtggtttgtccaaactcatcaatgtatcttatcatgtctggatcactagtcctagg

SEQ ID NO: 11 - DNA, 1379 bp, Artificial Sequence
  source: /note=“Description of Artificial Sequence Synthetic polynucleotide”
cctagggctagcatcttggagataattaaaatgataaccatctcgcaaataaataagtattttactgttttcgtaacagttttgtaataaaaaaacctataaatatgacgcaacctaagaagaagaggaaggttcccgggcaagtgactatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgaaacttgtttattgcagcttataatggttacaaataaagcaatagcatcacaaatttcacaaataaagcatttttttcactgcattctagttgtggtttgtccaaactcatcaatgtatcttatcatgtctggatcactagtcctagg

SEQ ID NO: 12 - DNA, 1398 bp, Artificial Sequence
  source: /note=“Description of Artificial Sequence Synthetic polynucleotide”
aaattatcgcaagataaggcgcacgttgattgggtcacccgagtgtacgttgataaagtcacgtgggcacccaacgcgttgataagcatcggtatataagggcctacagtgttctggtaaatcagttgcactgtgctcttcacaggaacactacaagacctacaaggctagcgtatacatcttggagataattaaattgataaccatctcgcaaataaataagtattttactgttttcgtaacagttttgtaataaaaaaacctataaatatgacgcaacctaagaagaagaggaaggttcccgggcaagtgactatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 13 - DNA, 166 bp, Artificial Sequence
  source: /note=“Description of Artificial Sequence Synthetic polynucleotide”
aaattatcgcaagataaggcgcacgttgattgggtcacccgagtgtacgttgataaagtcacgtgggcacccaacgcgttgataagcatcggtatataagggcctacagtgttctggtaaatcagttgcactgtgctcttcacaggaacactacaagacctacaag

SEQ ID NO: 14 - DNA, 30 bp, Artificial Sequence
  source: /note=“Description of Artificial Sequence Synthetic oligonucleotide”
acatcctaggtacaatcaaattatcgcaag

SEQ ID NO: 15 - DNA, 29 bp, Artificial Sequence
  source: /note=“Description of Artificial Sequence Synthetic oligonucleotide”
gtgagctagccttgtaggtcttgtagtgt

SEQ ID NO: 16 - DNA, 193 bp, Artificial Sequence
  source: /note=“Description of Artificial Sequence Synthetic polynucleotide”
acatcctaggtacaatcaaattatcgcaagataaggcgcacgttgattgggtcacccgagtgtacgttgataaagtcacgtgggcacccaacgcgttgataagcatcggtatataagggcctacagtgttctggtaaatcagttgcactgtgctcttcacaggaacactacaagacctacaaggctagctcac

SEQ ID NO: 17 - DNA, 30 bp, Artificial Sequence
  source: /note=“Description of Artificial Sequence Synthetic oligonucleotide”
acatgctagctacaatcaaattatcgcaag

SEQ ID NO: 18 - DNA, 193 bp, Artificial Sequence
  source: /note=“Description of Artificial Sequence Synthetic polynucleotide”
acatgctagctacaatcaaattatcgcaagataaggcgcacgttgattgggtcacccgagtgtacgttgataaagtcacgtgggcacccaacgcgttgataagcatcggtatataagggcctacagtgttctggtaaatcagttgcactgtgctcttcacaggaacactacaagacctacaaggctagctcac

SEQ ID NO: 19 - DNA, 41 bp, Artificial Sequence
  source: /note=“Description of Artificial Sequence Synthetic oligonucleotide”
agggctagcgtatacatcttggagataattaaattgataac

SEQ ID NO: 20 - DNA, 26 bp, Artificial Sequence
  source: /note=“Description of Artificial Sequence Synthetic oligonucleotide”
acacaggaaacagctatgaccatgat

SEQ ID NO: 21 - DNA, 1459 bp, Artificial Sequence
  source: /note=“Description of Artificial Sequence Synthetic polynucleotide”
agggctagcgtatacatcatggagataattaaaatgataaccatctcgcaaataaataagtattttactgttttcgtaacagttttgtaataaaaaaacctataaatatgacgcaacctaagaagaagaggaaggttcccgggcaagtgactatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgaaacttgtttattgcagcttataatggttacaaataaagcaatagcatcacaaatttcacaaataaagcatttttttcactgcattctagttgtggtttgtccaaactcatcaatgtatcttatcatgtctggatcactagtcctaggatcggatcccgggcccgtcgactgcagaggcctgcatgcaagcttggcgtaatcatggtcatagctgtttcctgtgt

SEQ ID NO: 22 - DNA, 1639 bp, Artificial Sequence
  source: /note=“Description of Artificial Sequence Synthetic polynucleotide”
acatcctaggtacaatcaaattatcgcaagataaggcgcacgttgattgggtcacccgagtgtacgttgataaagtcacgtgggcacccaacgcgttgataagcatcggtatataagggcctacagtgttctggtaaatcagttgcactgtgctcttcacaggaacactacaagacctacaaggctagcgtatacatcttggagataattaaattgataaccatctcgcaaataaataagtattttactgttttcgtaacagttttgtaataaaaaaacctataaatatgacgcaacctaagaagaagaggaaggttcccgggcaagtgactatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgaaacttgtttattgcagcttataatggttacaaataaagcaatagcatcacaaatttcacaaataaagcatttttttcactgcattctagttgtggtttgtccaaactcatcaatgtatcttatcatgtctggatcactagtcctaggatcggatcccgggcccgtcgactgcagaggcctgcatgcaagcttggcgtaatcatggtcatagctgtttcctgtgt

SEQ ID NO: 23 - DNA, 26 bp, Artificial Sequence
  source: /note=“Description of Artificial Sequence Synthetic oligonucleotide”
gcgtataattgcacactaacatgttg

SEQ ID NO: 24 - DNA, 19 bp, Artificial Sequence
  source: /note=“Description of Artificial Sequence Synthetic oligonucleotide”
gctggaagtgcgcagtcgc

SEQ ID NO: 25 - DNA, 228 bp, Artificial Sequence
  source: /note=“Description of Artificial Sequence Synthetic polynucleotide”
gattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaatt

SEQ ID NO: 26 - DNA, 75 bp, Artificial Sequence
  source: /note=“Description of Artificial Sequence Synthetic oligonucleotide”
tattttacaaagattcagaaatacgcatcacttacaacaagggggactttgaaattttgcatttgaggttgccgg

SEQ ID NO: 27 - DNA, 110 bp, Artificial Sequence
  source: /note=“Description of Artificial Sequence Synthetic polynucleotide”
gacctttaattcaacccaacacaatatattatagttaaataagaattattatcaaatcatttgtatattaattaaaatactatactgtaaattacattttatttacaatc

SEQ ID NO: 28 - DNA, 2705 bp, Artificial Sequence
  source: /note=“Description of Artificial Sequence Synthetic polynucleotide”
tcgctaccttaggaccgttatagttatgactaactaaactagtgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcacgcgtagctgatgcatagcatgcggtaccgggagatgggggaggctaactgaaacacggaaggagacaataccggaaggaacccgcgctatgacggcaataaaaagacagaataaaacgcacgggtgttgggtcgtttgttctctagattagttagtcatcgctaccttaggaccgttatagtta

SEQ ID NO: 29 - DNA, 28 bp, Artificial Sequence
  source: /note=“Description of Artificial Sequence Synthetic oligonucleotide”
agtctgcattttacacgcgttgttaatc

SEQ ID NO: 30 - DNA, 24 bp, Artificial Sequence
  source: /note=“Description of Artificial Sequence Synthetic oligonucleotide”
tatacgatccagagcgctacgctc

SEQ ID NO: 31 - DNA, 23 bp, Artificial Sequence
  source: /note=“Description of Artificial Sequence Synthetic oligonucleotide”
ccaagtcagcgtggagatcgagt

SEQ ID NO: 32 - DNA, 22 bp, Artificial Sequence
  source: /note=“Description of Artificial Sequence Synthetic oligonucleotide”
gccattgggaatcgcaatgcca

SEQ ID NO: 33 - DNA, 33 bp, Artificial Sequence
  source: /note=“Description of Artificial Sequence Synthetic oligonucleotide”
tataaggccggccgtatcacgaggccctttcgt

SEQ ID NO: 34 - DNA, 33 bp, Artificial Sequence
  source: /note=“Description of Artificial Sequence Synthetic oligonucleotide”
gctttggccggcctaccgcctttgagtgagctg

SEQ ID NO: 35 - DNA, 20 bp, Artificial Sequence
  source: /note=“Description of Artificial Sequence Synthetic oligonucleotide”
agtcccatgtcgtcggcaag

SEQ ID NO: 36 - DNA, 20 bp, Artificial Sequence
  source: /note=“Description of Artificial Sequence Synthetic oligonucleotide”
tcgaagagaatagccgtcgc

SEQ ID NO: 37 - DNA, 20 bp, Artificial Sequence
  source: /note=“Description of Artificial Sequence Synthetic oligonucleotide”
tggttgaggaagcggctaag

SEQ ID NO: 38 - DNA, 20 bp, Artificial Sequence
  source: /note=“Description of Artificial Sequence Synthetic oligonucleotide”
ttgttgtcggtagctgggtc

SEQ ID NO: 39 - DNA, 20 bp, Artificial Sequence
  source: /note=“Description of Artificial Sequence Synthetic oligonucleotide”
gacaacgcctacttcggcta

SEQ ID NO: 40 - DNA, 20 bp, Artificial Sequence
  source: /note=“Description of Artificial Sequence Synthetic oligonucleotide”
gtttcttccctggacagcca

SEQ ID NO: 41 - DNA, 2258 bp, Artificial Sequence
  source: /note=“Description of Artificial Sequence Synthetic polynucleotide”
ggccggcctgactaactaaactagtgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccatggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcacgcgtagctgatgcatagcatgcggtaccgggagatgggggaggctaactgaaacacggaaggagacaataccggaaggaacccgcgctatgacggcaataaaaagacagaataaaacgcacgggtgttgggtcgtttgttctctagattagttagtcaggccggcc

SEQ ID NO: 42 - DNA, 23 bp, Artificial Sequence
  source: /note=“Description of Artificial Sequence Synthetic oligonucleotide”
agagaatagccgtcgccacaatg

SEQ ID NO: 43 - DNA, 2820 bp, Artificial Sequence
  source: /note=“Description of Artificial Sequence Synthetic polynucleotide”
ggccggcctgactaactaaactagtgcgattaacattagcacagagagcgttttgccgagtcccatgtcgtcggcaagcacgccgccgttgggccggccgtttttttctctgttaatcatccactgtatgccttttttctgatgcgccaatagattgggcgtttgatgttcaaagttgggatcgtcgttgtcgggcgcttggtcaaaaaattcttgcaattgcaatttgtaattatccattgtggcgacggctattctcttcgattaaactatggttattgcgcgctcgtaatctaagatatcacatttaacacggacattatctaatcaacgtgattgacgacgagttcatttattgtatcttcaataatttgttcgtaaactggtaatgttgttacttgtatcgcaccaagttttctaacatacacacgaacatgtttgtagcttgtttctgaaaaattttgcacaaaatacggcaacccgtccgcgtacatgttagcttgtagcgtgcgcgtgtacacgaccaatttgagggccacgacgtttttaaaaagcgtgtaattcatgtacgtgtaacgcgactagttgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccatggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcacgcgtagctgatgcatagcatgcggtaccgggagatgggggaggctaactgaaacacggaaggagacaataccggaaggaacccgcgctatgacggcaataaaaagacagaataaaacgcacgggtgttgggtcgtttgttctctagattagttagtcaggccggcc

SEQ ID NO: 44 - DNA, 2405 bp, Artificial Sequence
  source: /note=“Description of Artificial Sequence Synthetic polynucleotide”
ggccggcctgactaactaaactagtgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccatggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcacgcgtagctgatgcatagcatgcggtaccgggagatgggggaggctaactgaaacacggaaggagacaataccggaaggaacccgcgctatgacggcaataaaaagacagaataaaacgcacgggtgttgggtcgtttgttctctagattagttagtcaggccggatggataattacaaattgcaattgcaagaattttttgaccaagcgcccgacaacgacgatcccaactttgaacatcaaacgcccaatctattggcgcatcagaaaaaaggcatacagtggatgattaacagagaaaaaaacggccggcc

SEQ ID NO: 45 - DNA, 3106 bp, Artificial Sequence
  source: /note=“Description of Artificial Sequence Synthetic polynucleotide”
tataaggccggccgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatcctaggggccggcctgactaactaaactagtgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccatggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcacgcgtagctgatgcatagcatgcggtaccgggagatgggggaggctaactgaaacacggaaggagacaataccggaaggaacccgcgctatgacggcaataaaaagacagaataaaacgcacgggtgttgggtcgtttgttctctagattagttagtcaggccggcccctaggatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaggccggccaaagc

SEQ ID NO: 46 - DNA, 33 bp, Artificial Sequence
  source: /note=“Description of Artificial Sequence Synthetic oligonucleotide”
gatcacgcgtcgcgttacacgtacatgaattac

SEQ ID NO: 47 - DNA, 32 bp, Artificial Sequence
  source: /note=“Description of Artificial Sequence Synthetic oligonucleotide”
gatcacgcgtgcgattaacattagcacagaga

SEQ ID NO: 48 - DNA, 575 bp, Artificial Sequence
  source: /note=“Description of Artificial Sequence Synthetic polynucleotide”
gatcacgcgtcgcgttacacgtacatgaattacacgctttttaaaaacgtcgtggccctcaaattggtcgtgtacacgcgcacgctacaagctaacatgtacgcggacgggttgccgtattttgtgcaaaatttttcagaaacaagctacaaacatgttcgtgtgtatgttagaaaacttggtgcgatacaagtaacaacattaccagtttacgaacaaattattgaagatacaataaatgaactcgtcgtcaatcacgttgattagataatgtccgtgttaaatgtgatatcttagattacgagcgcgcaataaccatagtttaatcgaagagaatagccgtcgccacaatggataattacaaattgcaattgcaagaattttttgaccaagcgcccgacaacgacgatcccaactttgaacatcaaacgcccaatctattggcgcatcagaaaaaaggcatacagtggatgattaacagagaaaaaaacggccggcccaacggcggcgtgcttgccgacgacatgggactcggcaaaacgctctctgtgctaatgttaatcgcacgcgtgatc

SEQ ID NO: 49 - DNA, 32 bp, Artificial Sequence
  source: /note=“Description of Artificial Sequence Synthetic oligonucleotide”
gatcactagtgcgattaacattagcacagaga

SEQ ID NO: 50 - DNA, 20 bp, Artificial Sequence
  source: /note=“Description of Artificial Sequence Synthetic oligonucleotide”
tctttgtgcgtgtcagacga

SEQ ID NO: 51 - DNA, 20 bp, Artificial Sequence
  source: /note=“Description of Artificial Sequence Synthetic oligonucleotide”
atgcaccatcggtttgttgc

SEQ ID NO: 52 - DNA, 2585 bp, Artificial Sequence
  source: /note=“Description of Artificial Sequence Synthetic polynucleotide”
ggccggcctgactaactaaactagtgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccatggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcacgcgtcgcgttacacgtacatgaattacacgctttttaaaaacgtcgtggccctcaaattggtcgtgtacacgcgcacgctacaagctaacatgtacgcggacgggttgccgtattttgtgcaaaatttttcagaaacaagctacaaacatgttcgtgtgtatgttagaaaacttggtgcgatacaagtaacaacattaccagtttacgaacaaattattgaagatacaataaatgaactcgtcgtcaatcacgttgattagataatgtccgtgttaaatgtgatatcttagattacgagcgcgcaataaccatagtttaatcgaagagaatagccgtcgccacaatggataattacaaattgcaattgcaagaattttttgaccaagcgcccgacaacgacgatcccaactttgaacatcaaacgcccaatctattggcgcatcagaaaaaaggcatacagtggatgattaacagagaaaaaaacggccggcc

SEQ ID NO: 53 - DNA, 1780 bp, Artificial Sequence
  source: /note=“Description of Artificial Sequence Synthetic polynucleotide”
gacctttaattcaacccaacacaatatattatagttaaataagaattattatcaaatcatttgtatattaattaaaatactatactgtaaattacattttatttacaatcactcgacgaagacttgatcaccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 54 - DNA, 23 bp, Artificial Sequence
  source: /note=“Description of Artificial Sequence Synthetic oligonucleotide”
gcgagatttaatcgccgcgacaa

SEQ ID NO: 55 - DNA, 29 bp, Artificial Sequence
  source: /note=“Description of Artificial Sequence Synthetic oligonucleotide”
aaaggcgtatcagaaactgtgctacattc

SEQ ID NO: 56 - DNA, 5834 bp, Artificial Sequence
  source: /note=“Description of Artificial Sequence Synthetic polynucleotide”
gatccagacatgataagatacattgatgagtttggacaaaccacaactagaatgcagtgaaaaaaatgctttatttgtgaaatttgtgatgctattgctttatttgtaaccattataagctgcaataaacaagtttcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatagtcacttgcccgggaaccttcctcttcttcttaggttgcgtcatatttataggtttttttattacaaaactgttacgaaaacagtaaaatacttatttatttgcgagatggttatcaatttaattatctccaagatgtatacgctagccttgtaggtcttgtagtgttcctgtgaagagcacagtgcaactgatttaccagaacactgtaggcccttatataccgatgcttatcaacgcgttgggtgcccacgtgactttatcaacgtacactcgggtgacccaatcaacgtgcgccttatcttgcgataatttgattgtacctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagcttgtcgagactgcaggctctagagtcgacctgcaggcatgcaagcttgattcactagttttattgttcaaagatgcagtcatccaaatccacattgaccagatcgcaggcagtgcaagcgtctggcacctttcccatgatatgatgaatgtagcacagtttctgatacgcctttttgacgacagaaacgggttgagattctgacacgggaaagcactctaaacagtctttctgtccgtgagtgaagcagatatttgaattctgattcattctctcgcattgtctgcagggaaacagcatcagattcatgcccacgtgacgagaacatttgttttggtacctgtctgcgtagttgatcgaagcttccgcgtctgacgtcgatggctgcgcaactgactcgcgcacccgtttgggctcacttatatctgcgtcactgggggcgggtcttttcttggctccaccctttttgacgtagaattcatgctccacctcaaccacgtgatcctttgcccaccggaaaaagtctttgacttcctgcttggtgaccttcccaaagtcatgatccagacggcgggtgagttcaaatttgaacatccggtcttgcaacggctgctggtgttcgaaggtcgttgagttcccgtcaatcacggcgcacatgttggtgttggaggtgacgatcacgggagtcgggtctatctgggccgaggacttgcatttctggtccacgcgcaccttgcttcctccgagaatggctttggccgactccacgaccttggcggtcatcttcccctcctcccaccagatcaccatcttgtcgacacagtcgttgaagggaaagttctcattggtccagtttacgcacccgtagaagggcacagtgtgggctatggcctccgcgatgttggtcttcccggtagttgcaggcccaaacagccagatggtgttcctcttgccgaactttttcgtggcccatcccagaaagacggaagccgcatattggggatcgtacccgtttagttccaaaattttataaatccgattgctggaaatgtcctccacgggctgctggcccaccaggtagtcgggggcggttttagtcaggctcataatctttcccgcattgtccaaggcagccttgatttgggaccgcgagttggaggccgcattgaaggagatgtatgaggcctggtcctcctggatccactgcttctccgaggtaatccccttgtccacgagccacccgaccagctccatgtacctggctgaagtttttgatctgatcaccggcgcgtcagaattgggattctgattctctttgttctgctcctgcgtctgcgacacgtgcgtcagatgctgcgccaccaaccgtttacgctccgtgagattcaaacaggcgcttaaatactgttctaaattagtccacgcccactggagctcaggctgggttttggggagcaagtaattggggatgtagcactcgtccaccaccttgttcccgcctccggcgccgtttctggtctttgtgaccgcgaaccagtttggcaaagtcggctcgatcccgcggtaaattctctgaatcagtttttcgcgaatctgactcaggaaacgtcccaaaactaaggatttcaccccggtggtttccacgagcacgtgtaagtggaagtagctctctcccttctcaaattgcacaaagaaaagggcctccggggccttactcacacggcgccactccgtcagaaagtcgcgctgcagcttctcggccacggtcaggggtgcctgctcaatcagattcagatccaagtcagaatctggcggcaactcccactccttctcggccacccagttcacaaagctgtcagaaatgccgggcagatgctcgtcaaggtcgctggggaccttaatcacaatctcgtaaaaccccgccagggcggcagatccgcgcccgatggtgggacggtatgaataatccggaatatttataggtttttttattacaaaactgttacgaaaacagtaaaatacttatttatttgcgagatggttatcattttaattatctccatgatctattaatattccggagtatacggacctttaattcaacccaacacaatatattatagttaaataagaattattatcaaatcatttgtatattaattaaaatactatactgtaaattacattttatttacaatcactcgacgaagacttgatcaccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcagctgatgcatagcatgcggtaccgggagatgggggaggctaactgaaacacggaaggagacaataccggaaggaacccgcgctatgacggcaataaaaagacagaataaaacgcacgggtgttgggtcgtttgttc